(19) United States
(12) Patent Application Publication
(10) Pub. No.: US 2026/0297192 A1
(43) Pub. Date: Oct. 1, 2026

(54) MOLECULE SPECIFICALLY ACTING IN A TISSUE WHERE A CELL DEATH BEING OBSERVED

(71) Applicant: CHUGAI SEIYAKU KABUSHIKI KAISHA, Tokyo (JP)

(72) Inventors: Jun Zhi ER, Singapore (SG); Tomoyuki IGAWA, Tokyo (JP)

(21) Appl. No.: 18/996,236

(22) PCT Filed: Jul. 27, 2023
(86) PCT No.: PCT/JP2023/027536

§ 371 Date: Jan. 17, 2025

(30) Foreign Application Priority Data
Jul. 28, 2022 (EP) 22187636.0

(51) Int. Cl. C07K 16/28; A61K 38/00; A61P 37/06; C07K 16/30

(52) U.S. Cl. CPC C07K 16/2809; A61K 38/00; A61P 37/06; C07K 16/2851; C07K 16/2878; C07K 16/30; C07K 2317/31; C07K 2317/52

(57) ABSTRACT

The present invention provides a molecule which binds to a component or a portion thereof of a cell which is exposed to a extracellular environment due to a cell death (e.g. a filament or a histone which forms a cytoskeleton or a nuclear skeleton), which is observed specifically in an affected tissue, and concurrently binds to a target molecule on or in a cell, which acts specifically in a tissue where a cell death is observed such as affected tissue, abnormal tissue or the like. The molecule of the present invention is useful for a medicament with reduced adverse effect while conferring significant therapeutic or preventive effect.

### TECHNICAL FIELD

The present invention relates to a molecule which acts specifically in a tissue where a cell death is observed, and confers a signal transmission or blockage into a cell in the tissue such as an immune cell or an affected cell, via binding to a component or a portion thereof constituting a cell, which is exposed to a extracellular environment due to a cell death; the molecule for use as a medicament; a use of the molecule in the preparation of a medicament; a pharmaceutical composition comprising the molecule; a method for conferring a signal transmission or blockage into a cell in the tissue; a polynucleotide encoding the molecule; a vector comprising the polynucleotide; a cell retaining the vector; and so on.

In one aspect herein, the present invention relates to an antigen-binding molecule and medical uses thereof which comprises a first moiety binding to a first antigen which is a damage-associated molecular pattern (DAMP) and a second moiety binding to an antigen different from the first antigen. In one aspect herein, the first antigen is selected from the group consisting of a) Splicing Factor 3B Subunit 3 (SF3B3); and b) Proliferating Cell Nuclear Antigen (PCNA). In one aspect herein, an antigen-binding molecule is provided and medical uses thereof which comprises a first moiety binding to a first antigen which is a damage-associated molecular pattern (DAMP) and a second moiety binding to an antigen different from the first antigen and wherein the molecule comprises at least one third moiety that binds to a third antigen; wherein said third antigen is different from the first antigen; and wherein said second and/or third antigen is a membrane protein of an immune cell, or a tumour cell. In an aspect, the first antigen is selected from the group consisting of F-actin; Phosphatidylserine (PS); Splicing Factor 3B Subunit 3 (SF3B3); and Proliferating Cell Nuclear Antigen (PCNA).

### BACKGROUND ART

Cancer therapeutic which has a topical (site specific) activity mainly in cancer tissue has been being developed by exploiting features of cancer cell itself and tumor microenvironment (TME). Some antibody therapeutics such as Herceptin, Cetuximab, or the like target cancer antigen, which is predominantly expressed in cancer cell, and exert cytotoxic activity mainly on tumor cells through ADCC, signal inhibition, etc. A method for delivering a ligand having physiological activity, such as a cytokine, to solid cancer by an immunocytokine comprising a ligand fused with an antibody molecule binding to a cancer antigen highly expressed on cancer cells is also known.

TME is known to be generally hypoxic and low nutrient state, leading to cell death especially in solid cancer and is then found to be dead cell-enriched environment. There are many molecules those are liberated or exposed to cell surface when cell dies and enriched in cancer but not in normal tissue. Some of the molecules on the dead cell are recognized by receptor expressed in phagocytic cells, such as dendritic cell or macrophage, and then the dead cell is eliminated (cleared) by phagocytic cells. For example, Clec9A is known to be expressed in a subset of dendritic cell and bind to dead cells and also F-actin ectopically exposed on cell surface of dead cells (PTL 1 and PTL 2).

There are many therapeutics modulating cell functions, such as T cell activation, B cell activation, cell death induction, signal inhibition, and so on. As reported, many antibodies targeting costimulatory molecules are under clinical development in cancer (NPL 1). For example, Utomirumab, anti-CD137 agonist antibody, and CDX-1140, anti-CD40 agonist antibody, are currently being developed in clinic. These molecules are expected to show anti-tumor efficacy by enhancing T cell activation directly or indirectly through dendritic cell, enhancing macrophage tumoricidal activity, and so on. In early clinical trials, these antibodies showed some clinical responses.

However, one of the concerns of the current molecules, those modulate cell functions, is systemic activity. These antibodies can bind to target molecules anywhere in the body including normal tissues and may cause toxicity. For example, anti-CD137 antibody (BMS) causes liver toxicity.

As one of the possible strategies to overcome the systemic toxicity, a bispecific antibody targeting tumor antigen and an antigen modulating immune cell function has been evaluated. Anti-CD137 and tumor antigen-binding bispecific antibody showed lower toxicity compared with anti-CD137 monospecific antibody (NPL 2). However, in some cases, tumor antigen loss in a cancer tissue has been reported and it may cause loss of anti-tumor effect of the tumor antigen-specific antibody.

### CITATION LIST

### Patent Literature

[PTL 1] WO 2013/053008 A2
[PTL 2] JP 2011-519959 A
[PTL3] WO2011/146902 A1
[PTL 4] WO2017/059231 A1
[PTL 5] U.S. Pat. No. 7,714,109 B2
[PTL 6] WO2018/187227 A1
[PTL 7] WO2000/002584 A2
[PTL 8] WO2009/071892 A2
[PTL 9] WO2018/031419 A1
[PTL 10] WO2010/028306 A2

### Non-Patent Literature

[NPL 1] Patrick et al., Nature Reviews Drug Discovery, Vol. 17, p. 509-527, 2018
[NPL 2] Miguel Gaspar et al, Cancer Immunol Res. 2020 June; 8 (6): 781-793
[NPL 3] Huang X et al; Cancer Res 15 Apr. 2010; 70 (8_Supplement): 1919
[NPL 4] Chang W et al. Theranostics. 2020 Jul. 23; 10 (20): 9214-9229.
[NPL 5] Silvestris F et al. Blood. 1996 Jun. 15;87 (12): 5185-95
[NPL 6] Ran S and Thorpe P E. Int J Radiat Oncol Biol Phys. 2002 Dec. 1;54 (5): 1479-84.
[NPL 7] Yamasaki S. et al. Nat Immunol. 2008 October; 9 (10): 1179-88
[NPL 8] Brown G D. Nat Immunol. 2008 October; 9 (10): 1099-100

### SUMMARY OF INVENTION

### Technical Problem

In one aspect, an objective of the present invention is to provide strategies to overcome the systemic toxicity caused by a therapeutic which targets one or more antigens expressed in an affected tissue such as a cancer, and conditionally modulate cell functions in an unique environment in an affected tissue such as TME.

In a further aspect, an objective of the present invention is to provide a molecule which acts specifically in a tissue which is sought to be treated or targeted such as affected tissue, abnormal tissue or the like, but does not act or less acts in a healthy or normal tissue, which enables to provide a molecule useful for a medicament with reduced adverse effect while conferring significant therapeutic or preventive effect.

In one aspect, it is an object of the present invention to provide an antigen binding molecule which is able to provoke an immune response in a diseased tissue but which does not or less provoke an immune response in a healthy or normal tissue.

### Solutions to Problem

In one aspect, the present invention provides a molecule which binds to a component or a portion thereof of a cell which is exposed to a extracellular environment due to a cell death (e.g. a filament or a histone which forms a cytoskeleton or a nuclear skeleton), which is observed specifically in an affected tissue (e.g., TME), and concurrently binds to a target molecule (e.g., a membrane protein) on or in a cell (e.g., an immune cell, an affected cell such as a tumor cell, an autoreactive cell, and a virus-infected cell), which acts specifically in a tissue where a cell death is observed such as affected tissue, abnormal tissue or the like. In a further aspect, a molecule which enables to crosslink between the component or a portion thereof of a cell which is exposed to a extracellular environment due to a cell death and the target molecule on or in a cell such as immune cell or an affected cell (e.g., a cancer cell, an autoreactive cell, and a virus-infected cell), and to confer a signal transmission or blockage into the cell in the tissue is provided. In a further aspect, by leading the increased crosslinking between the component or a portion thereof of a cell which is exposed to a extracellular environment due to a cell death and the target molecule on or in a cell such as immune cell or an affected cell by a plural number of the molecules, the increased signal transmission or the increased signal blockage via the target molecules into the cell is achieved.

As outlined in more detail in the description and below and the examples, the present invention provides antigen binding molecules which bind to antigens found extracellularly, in particular in the extracellular space of diseased tissues like cancer tissues and tissues affected by autoimmune diseases. Without being bound to any theory, these antigens are thought to be present extracellularly due to cell damage and destruction occurring in these diseased tissues. By being capable of binding to such antigens, the antigen binding molecules of the present invention provoke an immune response which is specific for the diseased tissue.

The following aspects and embodiments are part of the invention—wherein it will be appreciated by the skilled reader that the embodiments included in the following are independently intended as the embodiments for each and every general embodiments and aspects set forth below:

[AA1] An antigen-binding molecule which comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
   a) Splicing Factor 3B Subunit 3 (SF3B3); and
   b) Proliferating Cell Nuclear Antigen (PCNA).

[AA2] The antigen-binding molecule of [AA1], wherein the antigen-binding molecule comprises at least one third moiety that binds to a third antigen;
wherein (i) said third antigen is different from the first and second antigen, or
wherein (ii) said third antigen is different from the first but same as the second antigen; preferably wherein said third antigen is a membrane protein of an immune cell, or a tumour cell.

[AA3] The antigen-binding molecule of [AA1] or [AA2], wherein said first antigen is exposed to an extracellular environment due to cell damage, particularly due to cell death.

[AA4] The antigen-binding molecule of any one of [AA1] to [AA3], wherein the first moiety is selected from the group consisting of:
A) an Ig type binding moiety; or
B) a binding polypeptide, particularly wherein the said binding polypeptide is a non-Ig-type binding moiety.

[AA5] The antigen-binding molecule of [AA4], wherein
A) in case that the first antigen is Splicing Factor 3B Subunit 3 (SF3B3), the first moiety is an Ig type binding moiety, or
B) in case that the first antigen is Splicing Factor 3B Subunit 3 (SF3B3), the first moiety is a Clec4e polypeptide, or
C) in case that the first antigen is Proliferating Cell Nuclear Antigen (PCNA), the first moiety is an Ig type binding moiety.

[AA6] The antigen-binding molecule of any one of [AA1] to [AA5], wherein the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and/or the third antigen is a membrane protein involved in a signal transmission in a cell, preferably a signal transmission receptor.

[AA7] The antigen-binding molecule of any one of [AA1] to [AA6], wherein the second and/or the third antigen is a membrane protein of a tumour cell.

[AA8] The antigen-binding molecule of any one of [AA1] to [AA7], wherein the second and/or the third moiety is an Ig type binding moiety.

[AA9] The antigen-binding molecule of any one of [AA1] to [AA8], wherein the antigen-binding molecule is an antibody, in particular an IgG type antibody.

[AA10] The antigen-binding molecule of [AA9], wherein the antibody is capable of binding to a membrane protein of an immune cell or a tumour cell and further comprises, preferably linked to the Fc region, at least one binding polypeptide, particularly wherein the said binding polypeptide is selected from a Clec9A polypeptide and a Clec4e polypeptide;
especially wherein the antibody is selected from the group consisting of an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

[AA11] The antigen-binding molecule of any one of [AA1] to [AA8], wherein the antigen-binding molecule is an antibody, in particular an IgG type antibody, wherein said first moiety is linked to the Fc region, and is an Ig type binding moiety,
particularly wherein the antibody is selected from the group consisting of:
(A) an anti-CD3 antibody;
(B) an anti-CD137 antibody; and
(C) a bispecific anti-CD3 anti-CD137 antibody.

[AA12] The antigen-binding molecule of [AA9], wherein the antibody is a bispecific antibody,
particularly wherein the antigen binding molecule is a bispecific antibody directed against a membrane protein of an immune cell, or a tumour cell and against a first antigen selected from the group consisting of: a) Splicing Factor 3B Subunit 3 (SF3B3); and b) Proliferating Cell Nuclear Antigen (PCNA),
especially wherein the antibody is selected from the group consisting of
a bispecific anti-SF3B3 anti-CD3 antibody,
a bispecific anti-SF3B3 anti-CD137 antibody,
a bispecific anti-PCNA anti-CD3 antibody, and
a bispecific anti-PCNA anti-CD137 antibody.

[AA13] The antigen-binding molecule of [AA9], wherein the antibody is a trispecific antibody,
particularly wherein the antigen binding molecule is a trispecific antibody directed
   i) against a membrane protein of an immune cell, and against a membrane protein of a tumour cell; and against a first antigen selected from the group consisting of: a) Splicing Factor 3B Subunit 3 (SF3B3); and b) Proliferating Cell Nuclear Antigen (PCNA), or
   ii) against a first membrane protein of an immune cell, and a second membrane protein of an immune cell different from the first membrane protein; and against a first antigen selected from the group consisting of: a) Splicing Factor 3B Subunit 3 (SF3B3); and b) Proliferating Cell Nuclear Antigen (PCNA), or
   iii) against a first membrane protein of a tumour cell, and a second membrane protein of a tumour cell different from the first membrane protein; and against a first antigen selected from the group consisting of: a) Splicing Factor 3B Subunit 3 (SF3B3); and b) Proliferating Cell Nuclear Antigen (PCNA), especially wherein the antibody is selected from the group consisting of a trispecific anti-SF3B3 anti-CD3 anti-CD137 antibody, and a trispecific anti-PCNA anti-CD3 anti-CD137 antibody.

[AA14] A pharmaceutical composition comprising an antigen-binding molecule of any one of [AA1] to [AA13].

[AA15] The antigen-binding molecule of any one of [AA1] to [AA13] or the pharmaceutical composition according to [AA14] for use in medicine.

[AA16] The antigen-binding molecule of any one of [AA1] to [AA13] or the pharmaceutical composition of [AA14] for use in a method of treating or preventing a medical condition, preferably wherein the medical condition is selected from the group consisting of a cancer and an immune, preferably an autoimmune, disease.

[AA17] A method of screening an antigen-binding molecule, which comprises the steps of:
(a) selecting one or more nucleic acid(s) encoding at least one first moiety wherein the first moiety binds to a first antigen, and wherein the first antigen is a damage-associated molecular pattern (DAMP), wherein the first antigen is selected from the group consisting of: a) Splicing Factor 3B Subunit 3 (SF3B3); and b) Proliferating Cell Nuclear Antigen (PCNA);
(b) selecting one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) optionally providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c), thereby obtaining the antigen-binding molecule.

[AA18] A method of producing an antigen-binding molecule, which comprises the steps of:
(a) providing one or more nucleic acid(s) encoding at least one first moiety wherein the first moiety binds to a first antigen, and wherein the first antigen is a damage-associated molecular pattern (DAMP), wherein the first antigen is selected from the group consisting of: a) Splicing Factor 3B Subunit 3 (SF3B3); and b) Proliferating Cell Nuclear Antigen (PCNA);
(b) providing one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) optionally providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c) thereby obtaining the antigen-binding molecule.

[AA19] A method of conferring a signal transmission or blockage in a cell comprising contacting an antigen-binding molecule with a cell or administering to a subject an antigen-binding molecule, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
a) Splicing Factor 3B Subunit 3 (SF3B3); and
b) Proliferating Cell Nuclear Antigen (PCNA).

[AA20] A method of activating or preventing an immune response comprising contacting an antigen-binding molecule with a cell or administering to a subject an antigen-binding molecule, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
   a) Splicing Factor 3B Subunit 3 (SF3B3); and
   b) Proliferating Cell Nuclear Antigen (PCNA).

[AA21] A bispecific anti-Phosphatidylserine (PS) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

[AA22] The bispecific antibody or pharmaceutical composition of [AA21] for use in a method of treating or preventing a cancer disease, preferably wherein the cancer disease is a solid tumour.

[AA23] A bispecific anti-Phosphatidylserine (PS) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

[AA24] The bispecific antibody or pharmaceutical composition of [AA23] for use in a method of treating or preventing a cancer disease, preferably wherein the cancer disease is a solid tumour.

[AA25] A bispecific anti-Phosphatidylserine (PS) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

[AA26] The bispecific antibody or pharmaceutical composition of [AA25] for use in a method of treating or preventing a cancer disease, preferably wherein the cancer disease is a solid tumour.

[AA27] A bispecific anti-Phosphatidylserine (PS) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

[AA28] The bispecific antibody or pharmaceutical composition of [AA27] for use in a method of treating or preventing a cancer disease, preferably wherein the cancer disease is a solid tumour.

[AA29] A bispecific anti-Phosphatidylserine (PS) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

[AA30] The bispecific antibody or pharmaceutical composition of [AA29] for use in a method of treating or preventing a cancer disease, preferably wherein the cancer disease is a solid tumour.

[AA31] A bispecific anti-Phosphatidylserine (PS) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

[AA32] The bispecific antibody or pharmaceutical composition of [AA31] for use in a method of treating or preventing a cancer disease, preferably wherein the cancer disease is a solid tumour.

[AA33] A bispecific anti-Phosphatidylserine (PS) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

[AA34] The bispecific antibody or pharmaceutical composition of [AA33] for use in a method of treating or preventing a cancer disease, preferably wherein the cancer disease is a solid tumour. [AA35] A bispecific anti-Phosphatidylserine (PS) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

[AA36] The bispecific antibody or pharmaceutical composition of [AA35] for use in a method of treating or preventing a cancer disease, preferably wherein the cancer disease is a solid tumour.

[AA37] A bispecific anti-F-Actin anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

[AA38] The bispecific antibody or pharmaceutical composition of [AA37] for use in a method of treating or preventing a cancer disease, preferably wherein the cancer disease is a solid tumour.

[AA39] A bispecific anti-F-Actin anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

[AA40] The bispecific antibody or pharmaceutical composition of [AA39] for use in a method of treating or preventing a cancer disease, preferably wherein the cancer disease is a solid tumour.

[AA41] A bispecific anti-F-Actin anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

[AA42] The bispecific antibody or pharmaceutical composition of [AA41] for use in a method of treating or preventing a cancer disease, preferably wherein the cancer disease is a solid tumour.

[AA43] A bispecific anti-F-Actin anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

[AA44] The bispecific antibody or pharmaceutical composition of [AA43] for use in a method of treating or preventing a cancer disease, preferably wherein the cancer disease is a solid tumour.

[AA45] A bispecific anti-F-Actin anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

[AA46] The bispecific antibody or pharmaceutical composition of [AA45] for use in a method of treating or preventing a cancer disease, preferably wherein the cancer disease is a solid tumour.

[AA47] A bispecific anti-F-Actin anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

[AA48] The bispecific antibody or pharmaceutical composition of [AA47] for use in a method of treating or preventing a cancer disease, preferably wherein the cancer disease is a solid tumour.

[AA49] A bispecific anti-F-Actin anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

[AA50] The bispecific antibody or pharmaceutical composition of [AA49] for use in a method of treating or preventing a cancer disease, preferably wherein the cancer disease is a solid tumour.

[AA51] A bispecific anti-F-Actin anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

[AA52] The bispecific antibody or pharmaceutical composition of [AA51] for use in a method of treating or preventing a cancer disease, preferably wherein the cancer disease is a solid tumour.

[AA53] A bispecific antigen-binding molecule which is an anti-CD3 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

[AA54] The bispecific molecule or pharmaceutical composition of [AA53] for use in a method of treating or preventing a cancer disease, preferably wherein the cancer disease is a solid tumour.

[AA55] A bispecific antigen-binding molecule which is an anti-CD3 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

[AA56] The bispecific molecule or pharmaceutical composition of [AA55] for use in a method of treating or preventing a cancer disease, preferably wherein the cancer disease is a solid tumour.

[AA57] A bispecific antigen-binding molecule which is an anti-CD3 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

[AA58] The bispecific molecule or pharmaceutical composition of [AA57] for use in a method of treating or preventing a cancer disease, preferably wherein the cancer disease is a solid tumour.

[AA59] A bispecific antigen-binding molecule which is an anti-CD3 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

[AA60] The bispecific molecule or pharmaceutical composition of [AA59] for use in a method of treating or preventing a cancer disease, preferably wherein the cancer disease is a solid tumour.

[AA61] A bispecific antigen-binding molecule which is an anti-CD137 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

[AA62] The bispecific molecule or pharmaceutical composition of [AA61] for use in a method of treating or preventing a cancer disease, preferably wherein the cancer disease is a solid tumour.

[AA63] A bispecific antigen-binding molecule which is an anti-CD137 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

[AA64] The bispecific molecule or pharmaceutical composition of [AA63] for use in a method of treating or preventing a cancer disease, preferably wherein the cancer disease is a solid tumour.

[AA65] A bispecific antigen-binding molecule which is an anti-CD137 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

[AA66] The bispecific molecule or pharmaceutical composition of [AA65] for use in a method of treating or preventing a cancer disease, preferably wherein the cancer disease is a solid tumour.

[AA67] A bispecific antigen-binding molecule which is an anti-CD137 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

[AA68] The bispecific molecule or pharmaceutical composition of [AA67] for use in a method of treating or preventing a cancer disease, preferably wherein the cancer disease is a solid tumour.

[AA69] A bispecific anti-Splicing Factor 3B Subunit 3 (SF3B3) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

[AA70] The bispecific antibody or pharmaceutical composition of [AA69] for use in a method of treating or preventing a cancer disease, preferably wherein the cancer disease is a solid tumour.

[AA71] A bispecific anti-Splicing Factor 3B Subunit 3 (SF3B3) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

[AA72] The bispecific antibody or pharmaceutical composition of [AA71] for use in a method of treating or preventing a cancer disease, preferably wherein the cancer disease is a solid tumour.

[AA73] A bispecific anti-Splicing Factor 3B Subunit 3 (SF3B3) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

[AA74] The bispecific antibody or pharmaceutical composition of [AA73] for use in a method of treating or preventing a cancer disease, preferably wherein the cancer disease is a solid tumour.

[AA75] A bispecific anti-Splicing Factor 3B Subunit 3 (SF3B3) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

[AA76] The bispecific antibody or pharmaceutical composition of [AA75] for use in a method of treating or preventing a cancer disease, preferably wherein the cancer disease is a solid tumour.

[AA77] A bispecific anti-Splicing Factor 3B Subunit 3 (SF3B3) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

[AA78] The bispecific antibody or pharmaceutical composition of [AA77] for use in a method of treating or preventing a cancer disease, preferably wherein the cancer disease is a solid tumour.

[AA79] A bispecific anti-Splicing Factor 3B Subunit 3 (SF3B3) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

[AA80] The bispecific antibody or pharmaceutical composition of [AA79] for use in a method of treating or preventing a cancer disease, preferably wherein the cancer disease is a solid tumour.

[AA81] A bispecific anti-Splicing Factor 3B Subunit 3 (SF3B3) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

[AA82] The bispecific antibody or pharmaceutical composition of [AA81] for use in a method of treating or preventing a cancer disease, preferably wherein the cancer disease is a solid tumour.

[AA83] A bispecific anti-Splicing Factor 3B Subunit 3 (SF3B3) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

[AA84] The bispecific antibody or pharmaceutical composition of [AA83] for use in a method of treating or preventing a cancer disease, preferably wherein the cancer disease is a solid tumour.

[AA85] A bispecific antigen-binding molecule which is an anti-CD3 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

[AA86] The bispecific molecule or pharmaceutical composition of [AA85] for use in a method of treating or preventing a cancer disease, preferably wherein the cancer disease is a solid tumour.

[AA87] A bispecific antigen-binding molecule which is an anti-CD3 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

[AA88] The bispecific molecule or pharmaceutical composition of [AA87] for use in a method of treating or preventing a cancer disease, preferably wherein the cancer disease is a solid tumour.

[AA89] A bispecific antigen-binding molecule which is an anti-CD3 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

[AA90] The bispecific molecule or pharmaceutical composition of [AA89] for use in a method of treating or preventing a cancer disease, preferably wherein the cancer disease is a solid tumour.

[AA91] A bispecific antigen-binding molecule which is an anti-CD3 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

[AA92] The bispecific molecule or pharmaceutical composition of [AA91] for use in a method of treating or preventing a cancer disease, preferably wherein the cancer disease is a solid tumour.

[AA93] A bispecific antigen-binding molecule which is an anti-CD137 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

[AA94] The bispecific molecule or pharmaceutical composition of [AA93] for use in a method of treating or preventing a cancer disease, preferably wherein the cancer disease is a solid tumour.

[AA95] A bispecific antigen-binding molecule which is an anti-CD137 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

[AA96] The bispecific molecule or pharmaceutical composition of [AA95] for use in a method of treating or preventing a cancer disease, preferably wherein the cancer disease is a solid tumour.

[AA97] A bispecific antigen-binding molecule which is an anti-CD137 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

[AA98] The bispecific molecule or pharmaceutical composition of [AA97] for use in a method of treating or preventing a cancer disease, preferably wherein the cancer disease is a solid tumour.

[AA99] A bispecific antigen-binding molecule which is an anti-CD137 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

[AA100] The bispecific molecule or pharmaceutical composition of [AA99] for use in a method of treating or preventing a cancer disease, preferably wherein the cancer disease is a solid tumour.

[AA101] A bispecific anti-Proliferating Cell Nuclear Antigen (PCNA) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

[AA102] The bispecific antibody or pharmaceutical composition of [AA101] for use in a method of treating or preventing a cancer disease, preferably wherein the cancer disease is a solid tumour.

[AA103] A bispecific anti-Proliferating Cell Nuclear Antigen (PCNA) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

[AA104] The bispecific antibody or pharmaceutical composition of [AA103] for use in a method of treating or preventing a cancer disease, preferably wherein the cancer disease is a solid tumour.

[AA105] A bispecific anti-Proliferating Cell Nuclear Antigen (PCNA) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

[AA106] The bispecific antibody or pharmaceutical composition of [AA105] for use in a method of treating or preventing a cancer disease, preferably wherein the cancer disease is a solid tumour.

[AA107] A bispecific anti-Proliferating Cell Nuclear Antigen (PCNA) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

[AA108] The bispecific antibody or pharmaceutical composition of [AA107] for use in a method of treating or preventing a cancer disease, preferably wherein the cancer disease is a solid tumour.

[AA109] A bispecific anti-Proliferating Cell Nuclear Antigen (PCNA) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

[AA110] The bispecific antibody or pharmaceutical composition of [AA109] for use in a method of treating or preventing a cancer disease, preferably wherein the cancer disease is a solid tumour.

[AA111] A bispecific anti-Proliferating Cell Nuclear Antigen (PCNA) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

[AA112] The bispecific antibody or pharmaceutical composition of [AA111] for use in a method of treating or preventing a cancer disease, preferably wherein the cancer disease is a solid tumour.

[AA113] A bispecific anti-Proliferating Cell Nuclear Antigen (PCNA) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

[AA114] The bispecific antibody or pharmaceutical composition of [AA113] for use in a method of treating or preventing a cancer disease, preferably wherein the cancer disease is a solid tumour.

[AA115] A bispecific anti-Proliferating Cell Nuclear Antigen (PCNA) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

[AA116] The bispecific antibody or pharmaceutical composition of [AA115] for use in a method of treating or preventing a cancer disease, preferably wherein the cancer disease is a solid tumour.

[AA117] An antigen-binding molecule which comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the molecule comprises at least one third moiety that binds to a third antigen; wherein said third antigen is different from the first antigen; preferably wherein said third antigen is a membrane protein of an immune cell, or a tumour cell, and
wherein the first antigen is selected from the group consisting of:
   a) F-actin;
   b) Phosphatidylserine (PS);
   c) Splicing Factor 3B Subunit 3 (SF3B3); and
   d) Proliferating Cell Nuclear Antigen (PCNA).

[AA118] The antigen-binding molecule of [AA117], wherein (i) said third antigen is different from the first and second antigen, or wherein (ii) said third antigen is different from the first but same as the second antigen.

[AA119] The antigen-binding molecule of any one of [AA117] to [AA118], wherein the first moiety is selected from the group consisting of:
A) an Ig type binding moiety; or
B) a binding polypeptide, particularly wherein the said binding polypeptide is a non-Ig-type binding moiety.

[AA120] The antigen-binding molecule of [AA119], wherein
A) in case that the first antigen is Splicing Factor 3B Subunit 3 (SF3B3), the first moiety is an Ig type binding moiety, or
B) in case that the first antigen is Splicing Factor 3B Subunit 3 (SF3B3), the first moiety is a Clec4e polypeptide, or
C) in case that the first antigen is Proliferating Cell Nuclear Antigen (PCNA), the first moiety is an Ig type binding moiety, or
D) in case that the first antigen is F-actin, the first moiety is an Ig type binding moiety, or
E) in case that the first antigen is F-actin, the first moiety is a Clec9A polypeptide, or
F) in case that the first antigen is PS, the first moiety is an Ig type binding moiety.

[AA121] The antigen-binding molecule of any one of [AA117] to [AA120], wherein the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and/or the third antigen is a membrane protein involved in a signal transmission in a cell, preferably a signal transmission receptor.

[AA122] The antigen-binding molecule of any one of [AA117] to [AA121], wherein the second and/or the third antigen is a membrane protein of a tumour cell.

[AA123] The antigen-binding molecule of any one of [AA117] to [AA122], wherein the second and/or third moiety is an Ig type binding moiety.

[AA124] The antigen-binding molecule of any one of [AA117] to [AA123], wherein the antigen-binding molecule is an antibody, in particular an IgG type antibody.

[AA125] The antigen-binding molecule of [AA124], wherein the antibody is capable of binding to a membrane protein of an immune cell or a tumour cell and further comprises, preferably linked to the Fc region, at least one binding polypeptide,
particularly wherein the said binding polypeptide is selected from a Clec9A polypeptide and a Clec4e polypeptide;
especially wherein the antibody is selected from the group consisting of
an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

[AA126] The antigen-binding molecule of [AA125], wherein the antibody is a trispecific antibody, particularly wherein the antigen binding molecule is a trispecific antibody directed
i) against a membrane protein of an immune cell, and against a membrane protein of a tumour cell; and
against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
ii) against a first membrane protein of an immune cell, and a second membrane protein of an immune cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
iii) against a first membrane protein of a tumour cell, and a second membrane protein of a tumour cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA),
especially wherein the antibody is selected from the group consisting of a trispecific anti-SF3B3 anti-CD3 anti-CD137 antibody,
a trispecific anti-PCNA anti-CD3 anti-CD137 antibody,
a trispecific anti-PS anti-CD3 anti-CD137 antibody
a trispecific anti-F-Actin anti-CD3 anti-CD137 antibody
a bispecific anti-CD3 anti-CD137 antibody comprising a Clec9A polypeptide, and
a bispecific anti-CD3 anti-CD137 antibody comprising a Clec4e polypeptide.

[AA127] A method of screening an antigen-binding molecule, which comprises the steps of:
(a) selecting one or more nucleic acid(s) encoding at least one first moiety wherein the first moiety binds to a first antigen, and wherein the first antigen is a damage-associated molecular pattern (DAMP), wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) selecting one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and the at least one third moiety provided in (b1) is/are linked; and
(d) producing the antigen-binding molecule using the one or more nucleic acid(s) prepared in (c), thereby obtaining the antigen-binding molecule.

[AA128] A method of producing an antigen-binding molecule, which comprises the steps of:
(a) providing one or more nucleic acid(s) encoding at least one first moiety wherein the first moiety binds to a first antigen, and wherein the first antigen is a damage-associated molecular pattern (DAMP); wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) providing one or more nucleic acid(s) encoding at least one second moiety wherein the second moiety binds to a second antigen, and wherein the second antigen is different from the first antigen; preferably wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and the at least one third moiety provided in (b1) is/are linked; and
(d) producing the antigen-binding molecule using the one or more nucleic acid(s) prepared in (c) thereby obtaining the antigen-binding molecule.

[AA129] A method of conferring a signal transmission or blockage in a cell comprising contacting an antigen-binding molecule with a cell or administering to a subject an antigen-binding molecule, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the molecule comprises at least one third moiety that binds to a third antigen; wherein said third antigen is different from the first antigen;
preferably wherein said third antigen is a membrane protein of an immune cell, or a tumour cell, and
wherein the first antigen is selected from the group consisting of:
   a) F-actin;
   b) Phosphatidylserine (PS);
   c) Splicing Factor 3B Subunit 3 (SF3B3); and
   d) Proliferating Cell Nuclear Antigen (PCNA).

[AA130] A method of activating or preventing an immune response comprising contacting an antigen-binding molecule with a cell or administering to a subject an antigen-binding molecule, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen, preferably wherein said second antigen is a membrane protein of an immune cell, or a tumour cell, and
wherein the molecule comprises at least one third moiety that binds to a third antigen; wherein said third antigen is different from the first antigen; preferably wherein said third antigen is a membrane protein of an immune cell, or a tumour cell, and
wherein the first antigen is selected from the group consisting of:
   a) F-actin;
   b) Phosphatidylserine (PS);
   c) Splicing Factor 3B Subunit 3 (SF3B3); and
   d) Proliferating Cell Nuclear Antigen (PCNA).

Furthermore, the present disclosure provides the following aspects and embodiments.

[A1] A molecule comprising a first moiety that binds to a first antigen and a second moiety that binds to a second antigen,
wherein said first antigen is a component or a portion thereof which constitutes a cytoskeleton, a cell membrane, or an organelle, or is a cytoplasmic protein, or a metabolite, which is exposed to an extracellular environment due to a cell death, and
wherein said second antigen is an antigen different from the first antigen.

[A2] The molecule of [A1], wherein the cell death is a cell death in an affected tissue.

[A3] The molecule of [A1] or [A2], wherein the first antigen is a filament, a histone or a nucleosome comprising a histone, which forms a cytoskeleton or a nuclear skeleton.

[A4] The molecule of [A3], wherein the first antigen is an intermediate filament. [A5] The molecule of [A4], wherein the first antigen is a F-actin.

[A6] The molecule of any one of [A1] to [A5], wherein the first antigen is an antigen which is formed by multimerization of a plural number of molecules.

[A7] The molecule of [A1] or [A2], wherein the first antigen is a phosphatidylserine which constitutes a cell membrane.

[A8] The molecule of any one of [A1] to [A7], wherein the cell death is a necrosis, an apoptosis, an autophagy cell death, or an accidental cell death.

[A9] The molecule of any one of [A1] to [A8], wherein the second antigen is a membrane protein involved in a signal transmission in a cell.

[A10] The molecule of [A9], wherein the cell is an immune cell.

[A11] The molecule of [A10], wherein the immune cell is a T cell, a killer cell, a helper T cell, a regulatory T cell, a B cell, a memory B cell, a NK cell, a NKT cell, a dendritic cell, a macrophage, an eosinophil, a neutrophil cell, or a basophil.

[A12] The molecule of [A9], wherein the cell is a tumor cell or an autoreactive cell.

[A13] The molecule of any one of [A9] to [A12], which is characterized in that a complex formed by binding of one or more of the molecules with the first antigen binds to one or more of the membrane proteins via the second moieties to confer a signal transmission or blockage via the membrane protein in a cell.

[A14] The molecule of [A13], which is characterized in that the molecule confers a signal transmission via the membrane protein in a cell.

[A15] The molecule of [A13], which is characterized in that the molecule confers a signal blockage via the membrane protein in a cell.

[A16] The molecule of any one of [A9] to [A15], wherein the membrane protein is a membrane protein in a cell membrane or a membrane protein of an endosome.

[A17] The molecule of any one of [A11] to [A16], wherein the membrane protein is:
a membrane protein in a cell membrane of a T cell selected from a group consisting of T cell receptor, CD3, a costimulatory molecule, and a coinhibitory molecule; or
a membrane protein in a cell membrane of a cell other than a T cell, which binds to a costimulatory molecule or a coinhibitory molecule in a cell membrane of a T cell.

[A18] The molecule of [A17], wherein the costimulatory molecule is CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), or CD244 (2B4).

[A19] The molecule of [A17], wherein the coinhibitory molecule is CD279 (PD-1), CD152 (CTLA-4), TIGIT, BTLA, CD366 (Tim-3), CD96, CD112R, or CD200R.

[A20] The molecule of [A17], wherein the membrane protein that binds to a costimulatory molecule or a coinhibitory molecule is CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), or Gal-9.

[A21] The molecule of any one of [A1] to [A20], wherein the first moiety is an antibody variable region or a single domain antibody (VHH).

[A22] The molecule of any one of [A1] to [A20], wherein the first antigen is a F-actin, and the first moiety is a molecule that binds to a F-actin or a portion thereof.

[A23] The molecule of [A22], wherein the molecule that binds to a F-actin or a portion thereof is an extracellular region of Clec9A or a portion thereof.

[A24] The molecule of any one of [A9] to [A23], wherein the second moiety is an antibody variable region or a single domain antibody (VHH), or a ligand.

[A25] The molecule of any one of [A1] to [A9], wherein:
the first antigen is a F-actin;
the second antigen is a membrane protein involved in a signal transmission in a cell;
the second moiety is an either one or both of Fab regions of an antibody variable region comprising two Fab regions, which binds to the second antigen; and
the first moiety is a molecule that binds to the F-actin or a portion thereof, which is linked via a linker or directly to a C-terminus of a Fc region connected to the antibody variable region.

[A26] The molecule of [A25], wherein the cell is an immune cell.

[A27] The molecule of [A26], wherein the immune cell is a T cell, a killer cell, a helper T cell, a regulatory T cell, a B cell, a memory B cell, a NK cell, a NKT cell, a dendritic cell, a macrophage, an eosinophil, a neutrophil cell, or a basophil.

[A28] The molecule of [A25], wherein the cell is a tumor cell or an autoreactive cell.

[A29] The molecule of any one of [A25] to [A28], which is characterized in that a complex formed by binding of one or more of the molecules with the F-actin binds to one or more of the membrane proteins via the second moieties to confer a signal transmission or blockage via the membrane protein in a cell.

[A30] The molecule of [A29], which is characterized in that the molecule confers a signal transmission via the membrane protein in a cell.

[A31] The molecule of [A29], which is characterized in that the molecule confers a signal blockage via the membrane protein in a cell.

[A32] The molecule of any one of [A25] to [A31], wherein the membrane protein is a membrane protein in a cell membrane or a membrane protein of an endosome.

[A33] The molecule of any one of [A27] to [A32], wherein the membrane protein is:
a membrane protein in a cell membrane of a T cell selected from a group consisting of T cell receptor, CD3, a costimulatory molecule, and a coinhibitory molecule; or
a membrane protein in a cell membrane of a cell other than a T cell, which binds to a costimulatory molecule or a coinhibitory molecule in a cell membrane of a T cell.

[A34] The molecule of [A33], wherein the costimulatory molecule is CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), or CD244 (2B4).

[A35] The molecule of [A33], wherein the coinhibitory molecule is CD279 (PD-1), CD152 (CTLA-4), TIGIT, BTLA, CD366 (Tim-3), CD96, CD112R, or CD200R.

[A36] The molecule of [A33], wherein the membrane protein that binds to a costimulatory molecule or a coinhibitory molecule is CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), or Gal-9.

[A37] The molecule of any one of [A25] to [A36], wherein the first moiety is an antibody variable region or a single domain antibody (VHH).

[A38] The molecule of any one of [A25] to [A37], wherein the molecule that binds to a F-actin or a portion thereof is an extracellular region of Clec9A or a portion thereof.

[A39] The molecule of any one of [A25] to [A38], wherein the second moiety is an antibody variable region or a single domain antibody (VHH), or a ligand.

[A40] The molecule of any one of [A1] to [A20], wherein the molecule comprises or is a compound comprising the first moiety and the second moiety.

[A41] The molecule of [A40], wherein the compound is a compound that, upon receipt of a light irradiation, binds to the first antigen and the second antigen.

[B1] A pharmaceutical composition comprising the molecule of any one of [A1] to [A41].

[B2] The pharmaceutical composition of [B1], for conferring a signal transmission or blockage in a cell.

[B3] The pharmaceutical composition of [B1] or [B2], for conferring a signal transmission or blockage in an immune cell.

[B4] The pharmaceutical composition of any one of [B1] to [B3], for activating or preventing an immune.

[B5] The pharmaceutical composition of any one of [B1] to [B4], for treating or preventing cancer or autoimmune disease.

[B6] The molecule of any one of [A1] to [A41], for use as a medicament.

[B7] The molecule of [B6], for conferring a signal transmission or blockage in a cell.

[B8] The molecule of [B6] or [B7], for conferring a signal transmission or blockage in an immune cell.

[B9] The molecule of any one of [B6] to [B8], for activating or preventing an immune.

[B10] The molecule of any one of [B5] to [B9], for treating or preventing cancer or autoimmune disease.

[B11] Use of the molecule of any one of [A1] to [A41] in a preparation of a medicament.

[B12] The use of [B11], wherein the medicament is a medicament for conferring a signal transmission or blockage in a cell.

[B13] The use of [B11] or [B12], wherein the medicament is a medicament for conferring a signal transmission or blockage in an immune cell.

[B14] The use of any one of [B11] to [B13], wherein the medicament is a medicament for activating or preventing an immune.

[B15] The use of any one of [B11] to [B14], wherein the medicament is a medicament for treating or preventing cancer or autoimmune disease.

[B16] A method for conferring a signal transmission or blockage in a cell, comprising administering to a subject the molecule of any one of [A1] to [A41].

[B17] A method for activating or preventing an immune, comprising administering to a subject the molecule of any one of [A1] to [A41].

[B18] The method of any one of [B15] to [B17], wherein the cell is an immune cell, a cancer cell, or an autoreactive cell.

[B19] A method for treating or preventing cancer or autoimmune disease, comprising administering to a subject the molecule of any one of [A1] to [A41].

[B20] A method for producing the molecule of any one of [A1] to [A39].

[B21] A polynucleotide encoding the molecule of any one of [A1] to [A39].

[B22] A polynucleotide in particular a DNA encoding the molecule of any one of [A1] to [A39].

[B23] A vector comprising the polynucleotide of [B22].

[B24] A vector comprising a DNA sequence encoding for the antigen-binding molecule as defined in any one of [A1] to [A39], wherein the vector is in particular for recombinant protein expression.

[B25] A host cell retaining the vector of [B23] or [B24].

In addition, the present disclosure particularly also provides the following embodiments [C1] to [C15]:

[C1] A molecule comprising a first moiety that binds to a first antigen and a second moiety that binds to a second antigen, wherein said first antigen is a component or a portion thereof which constitutes a cytoskeleton, a cell membrane, or an organelle, or is a cytoplasmic protein, or a metabolite, which is exposed to an extracellular environment due to a cell death, and wherein said second antigen is an antigen different from the first antigen.

[C2] The molecule of [C1], wherein the cell death is a cell death in an affected tissue.

[C3] The molecule of [C1] or [C2], wherein the first antigen is a filament, a histone or a nucleosome comprising a histone, which forms a cytoskeleton or a nuclear skeleton.

[C4] The molecule of [C3], wherein the first antigen is an intermediate filament.

[C5] The molecule of [C4], wherein the first antigen is a F-actin.

[C6] The molecule of any one of [C1] to [C5], wherein the first antigen is an antigen which is formed by multimerization of a plural number of molecules.

[C7] The molecule of any one of [C1] to [C6], wherein the cell death is a necrosis, an apoptosis, an autophagy cell death, or an accidental cell death.

[C8] The molecule of any one of [C1] to [C7], wherein the second antigen is a membrane protein involved in a signal transmission in a cell. [C9] The molecule of [C8], wherein the cell is an immune cell. [C10] The molecule of any one of [C1] to [C9], wherein the first moiety is an antibody variable region or a single domain antibody (VHH).

[C11] The molecule of any one of [C1] to [C9], wherein the first antigen is a F-actin, and the first moiety is a molecule that binds to the F-actin or a portion thereof.

[C12] The molecule of any one of [C1] to [C9], wherein:
the first antigen is a F-actin;
the second antigen is a membrane protein involved in a signal transmission in a cell;
the second moiety is an either one or both of Fab regions of an antibody variable region comprising two Fab regions, which binds to the second antigen; and
the first moiety is a molecule that binds to the F-actin or a portion thereof, which is linked via a linker or directly to a C-terminus of a Fc region connected to the antibody variable region.

[C13] A pharmaceutical composition comprising the molecule of any one of [C1] to [C12].

[C14] Use of the molecule of any one of [C1] to [C12] in a preparation of a medicament.

[C15] A method for producing the molecule of any one of [C1] to [C12].

In particular, the present disclosure further also provides the following embodiments [D1] to [D44].

[D1] An antigen-binding molecule which comprises:
(1) at least one first moiety that binds to a first antigen, and
(2) at least one second moiety that binds to a second antigen;
wherein said first antigen is a damage-associated molecular pattern (DAMP), and
wherein said second antigen is an antigen different from the first antigen.

[D1.1] An antigen-binding molecule which comprises:
(1) at least one first moiety that binds to a first antigen, and
(2) at least one second moiety that binds to a second antigen;
wherein said first antigen is a tissue-derived antigen characteristic for a tissue state, wherein in particular the antigen is exposed to extracellular environment, such as a damage-associated molecular pattern (DAMP), and
wherein said second antigen is different from the first antigen.

[D2] The antigen-binding molecule of [D1] or [D1.1], wherein the said second antigen is a membrane protein of an immune cell.

[D3] The antigen-binding molecule of any one of [D1] and [D2], wherein the damage-associated molecular pattern (DAMP) is exposed to an extracellular environment due to cell damage, particularly due to cell death.

[D4] The antigen-binding molecule of any one of [D1] to [D3], wherein the molecule
A) is capable of providing a cellular contact and/or signal due to the said cell damage wherein the said contact and/or signal involves the said second antigen; and/or
B) comprises the said first moiety at an Fc terminus of the antigen-binding molecule; and/or
C) comprises the said second moiety in an antigen binding region, preferably an F(ab′)₂ region, of the antigen-binding molecule; and/or
D) comprises at least one first moiety and at least one second moiety, particularly wherein the antigen-binding molecule comprises one first moiety and one second moiety, more particularly wherein the antigen-binding molecule comprises at least two, preferably two, first moieties and at least two, preferably two, second moieties; and/or
E) comprises a compound, wherein the compound is a compound that is capable of binding to the first antigen and the second antigen upon light irradiation.

[D5] The antigen-binding molecule of any one of [D1] to [D4], wherein the said cell damage
i) involves cell death, particularly is cell death, and/or
ii) occurs in an affected tissue, particularly selected from a tissue affected by a condition or disease, particularly by a cancer or an autoimmune disease, especially in a tumor microenvironment (TME); and/or
iii) results from any one selected from the group consisting of necrosis, apoptosis, an autophagy cell death and an accidental cell death, and/or
iv) is cell death selected from the group consisting of necrosis, apoptosis, an autophagy cell death and an accidental cell death.

[D6] The antigen-binding molecule of any one of [D1] to [D5], wherein the first antigen is
i) selected from the group consisting of a cytoskeleton component or a portion thereof, a cell membrane component or a portion thereof, an organelle component or a portion thereof, a cytoplasmic protein or a portion thereof, and a metabolite; and/or
ii) located on any one selected from the group consisting of a filament (particularly an intermediate filament), a nucleosome, a cytoskeleton, and/or a nuclear skeleton; and/or
iii) capable of multimerization of a plurality of molecules; and/or
iv) present in a multimeric molecule, particularly wherein the first antigen is
i*) selected from the group consisting of a cytoskeleton component or a portion thereof, a cell membrane component or a portion thereof, an organelle component or a portion thereof, a cytoplasmic protein or a portion thereof, and a metabolite; and/or
ii*) located on any one selected from the group consisting of a filament (particularly an intermediate filament), a nucleosome, a cytoskeleton, and/or a nuclear skeleton.

[D7] The antigen-binding molecule of any one of [D1] to [D6], wherein the first antigen is selected from the group consisting of
A) an actin, particularly F-actin; or
B) a heat shock protein, particularly selected from HSP90 and GRP78, especially HSP90;
C) a phosphatidylserine (PS), particularly a phosphatidylserine that is part of a cell membrane;
D) a histone or component thereof,
E) a histone deacetylase complex subunit, particularly SAP130,
F) a HMGN protein, particularly selected from HMGB1 and HMGN1,
G) hepatoma-derived growth factor,
H) BCL-2,
I) calreticulin, and
J) cyclophilin A;
particularly wherein the first antigen is selected from the group consisting of F-actin, GRP78, phosphatidylserine, HSP90, and SAP130,
especially wherein the first antigen is selected from the group consisting of F-actin, phosphatidylserine, and HSP90,
in particular wherein the first antigen is selected from the group consisting of F-actin and HSP90,
preferably wherein the first antigen is F-actin.

[D8] The antigen-binding molecule of any one of [D1] to [D7], wherein the first moiety is selected from the group consisting of
A) an Ig type binding moiety; or
B) a binding polypeptide, particularly wherein the said binding polypeptide is a non-Ig-type binding moiety.

[D9] The antigen-binding molecule of any one of [D1] to [D8], wherein the first moiety is selected from the group consisting of
A) a moiety capable of binding to an actin, particularly F-actin;
B) a moiety capable of binding to a heat shock protein, particularly selected from HSP90 and GRP78;
C) a moiety capable of binding to a phosphatidylserine (PS), particularly a phosphatidylserine that is part of a cell membrane;
D) a moiety capable of binding to a histone or component thereof,
E) a moiety capable of binding to a histone deacetylase complex subunit, particularly SAP130,
F) a moiety capable of binding to a HMGN protein, particularly selected from HMGB1 and HMGN1,
G) a moiety capable of binding to hepatoma-derived growth factor,
H) a moiety capable of binding to BCL-2,
I) a moiety capable of binding to calreticulin, and
J) a moiety capable of binding to cyclophilin A;
particularly wherein the first moiety is selected from the group consisting of a moiety capable of binding to F-actin, a moiety capable of binding to GRP78, a moiety capable of binding to phosphatidylserine, a moiety capable of binding to HSP90, and a moiety capable of binding to SAP130,
especially wherein the first moiety is selected from the group consisting of a moiety capable of binding to F-actin, a moiety capable of binding to phosphatidylserine, and a moiety capable of binding to HSP90,
in particular wherein the first moiety is selected from the group consisting of a moiety capable of binding F-actin and a moiety capable of binding to HSP90,
preferably wherein the first moiety is a moiety capable of binding F-actin.

[D10] The antigen-binding molecule of [D9], wherein
A) the moiety capable of binding to F-actin is a binding polypeptide, preferably a Clec9A polypeptide, and/or
B) the moiety capable of binding to GRP78 is Ig-type binding moiety, preferably the Ig-type binding moiety from antibody GA20 or Mab159, and/or
C) the moiety capable of binding to phosphatidylserine is Ig-type binding moiety, preferably the Ig-type binding moiety from antibody 3G4, and/or
D) the moiety capable of binding to HSP90 is Ig-type binding moiety, preferably the Ig-type binding moiety from antibody 1.5.1 or 6H8, and/or
E) the moiety capable of binding to SAP130 is a binding polypeptide, preferably a Clec4e polypeptide;
particularly wherein the said Ig-type binding moiety is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂,
especially wherein the said Ig-type binding moiety is selected from the group consisting of an VHH, an scFv, an scFv2, a Fab, and a F(ab′)₂.

[D11] The antigen-binding molecule of any one of [D1] to [D9], wherein
A) in case that the first antigen is an actin, preferably an F-actin, the first moiety is a Clec9A polypeptide, or
B) in case that the first antigen is GRP78, phosphatidylserine, or HSP90, the first moiety is an Ig type binding moiety, or
C) in case the first antigen is SAP130, the first moiety is a Clec4e polypeptide.

[D12] The antigen-binding molecule of any one of [D1] to [D11], wherein in case that the first antigen is actin or SAP130, the first binding moiety is an Ig type binding moiety.

[D13] The antigen-binding molecule of any one of [D1] to [D12], wherein the second antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second antigen is a membrane protein involved in a signal transmission in a cell, preferably a signal transmission receptor.

[D14] The antigen-binding molecule of any one of [D1] to [D13], wherein the second moiety is an Ig type binding moiety.

[D15] The antigen-binding molecule of any one of [D1] to [D14], wherein the second antigen is
A) involved in a signal transmission in a cell;
particularly
A-1) wherein the said second antigen is a membrane protein, and/or
A-2) wherein the said second antigen is a signal transmission receptor, and/or
A-3) wherein the cell is i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, ii) a tumor cell, or iii) an autoreactive cell; and/or
B) an immune cell receptor antigen.

[D16] The antigen-binding molecule of any one of [D1] to [D15], wherein the second antigen is selected from the group consisting of
A) a membrane protein of an endosome,
B) a membrane protein in a cell membrane of a T cell, in particular a membrane protein in a cell membrane of a T cell selected from the group consisting of T cell receptor, CD3, CD137, CD40, CTLA4, a costimulatory molecule or a coinhibitory molecule, especially wherein the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9;
C) a membrane protein in a cell membrane of a cell other than a T cell, particularly which is capable of binding to a costimulatory molecule or a coinhibitory molecule in a cell membrane of a T cell,
in particular wherein the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4), and/or wherein the coinhibitory molecule is selected from the group consisting of CD279 (PD-1), CD152 (CTLA-4), TIGIT, BTLA, CD366 (Tim-3), CD96, CD112R, or CD200R, and/or
especially wherein the second antigen is selected from the group consisting of CD3, CD137, CD40 and CTLA4, in particular wherein the second antigen is selected from the group consisting of CD3 and CD137, preferably wherein the second antigen is CD3.

[D17] The antigen-binding molecule of any one of [D1] to [D16], wherein the second moiety is selected from the group consisting of
A) a moiety capable of binding to a membrane protein of an endosome,
B) a moiety capable of binding to a membrane protein in a cell membrane of a T cell,
in particular a membrane protein in a cell membrane of a T cell selected from the group consisting of T cell receptor, CD3, CD137, CD40, CTLA4, a costimulatory molecule or a coinhibitory molecule,
especially wherein the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9; and
C) a moiety capable of binding to a membrane protein in a cell membrane of a cell other than a T cell, particularly which is capable of binding to a costimulatory molecule or a coinhibitory molecule in a cell membrane of a T cell,
in particular wherein the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4), and/or wherein the coinhibitory molecule is selected from the group consisting of CD279 (PD-1), CD152 (CTLA-4), TIGIT, BTLA, CD366 (Tim-3), CD96, CD112R, or CD200R,
especially wherein the second moiety is selected from the group consisting of a moiety capable of binding to CD3, a moiety capable of binding to CD137, a moiety capable of binding to CD40 and a moiety capable of binding to CTLA4.
in particular wherein the second moiety is selected from the group consisting of a moiety capable of binding to CD3 and a moiety capable of binding to CD137.
preferably wherein the second moiety is a moiety capable of binding to CD3.

[D18] The antigen-binding molecule of [D17], wherein
A) the moiety capable of binding to CD3 is Ig-type binding moiety, and/or
B) the moiety capable of binding to CD137 is Ig-type binding moiety, and/or
C) the moiety capable of binding to CD40 is Ig-type binding moiety, and/or
D) the moiety capable of binding to CTLA4 is Ig-type binding moiety;
particularly wherein the said Ig-type binding moiety is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂,
especially wherein the said Ig-type binding moiety is selected from the group consisting of an VHH, an scFv, an scFv2, a Fab, and a F(ab′)₂.

[D19] The antigen-binding molecule of any one of [D1] to [D18], wherein the molecule is characterized in that a complex, which is formed by binding of one or more of the molecules to the first antigen, is capable of binding to more than one membrane proteins via the second moieties, particularly wherein
i) the complex, which is formed by binding of one or more of the molecules to the first antigen, is capable of binding to one or more of the membrane proteins via the second moieties to confer a signal transmission via the said one or more membrane protein in a cell, or
ii) the complex, which is formed by binding of one or more of the molecules to the first antigen, is capable of binding to one or more of the membrane proteins via the second moieties to confer a signal blockage via the said one or more membrane proteins in a cell.

[D20] The antigen-binding molecule of any one of [D1] to [D19], wherein the molecule is selected from the group consisting of
A) a polypeptide complex, which optionally is a fusion protein,
B) a polynucleotide (preferably composed of two entities, preferably connected with a linker), and
C) a medium-sized chemical compound (preferably composed of two entities, preferably connected with a linker),
D) a small-sized chemical compound (preferably composed of two entities, preferably connected with a linker), particularly wherein
A-1) the said molecule is a polypeptide complex,
especially wherein the said molecule is an antibody or an antibody fragment thereof,
in particular wherein the said molecule is an antibody,
preferably wherein said antibody is an IgG type antibody and/or a bispecific antibody; or
A-2) the said molecule is a polypeptide complex, which is a fusion protein, especially wherein the said molecule is an antibody, which is a fusion protein, or an antibody fragment thereof,
in particular wherein the said molecule is an antibody, which is a fusion protein,
preferably wherein said antibody is a) an IgG type antibody having at least one binding polypeptide, preferably two binding polypeptides, fused to its Fc domain and/or b) a bispecific antibody having at least one binding polypeptide, preferably two binding polypeptides, fused to its Fc domain.

[D21] The antigen-binding molecule of any one of [D1] to [D20], wherein the molecule is an antibody.

[D22] The antigen-binding molecule of [D20] or [D21], wherein the molecule is an IgG type antibody.

[D23] The antigen-binding molecule of any one of [D20] to [D22], wherein the antibody is capable of binding to a membrane protein of an immune cell and further comprises, preferably linked to the Fc region, at least one binding polypeptide,
particularly wherein the said binding polypeptide is selected from a Clec9A polypeptide and a Clec4e polypeptide;
especially wherein the antibody is selected from the group consisting of
an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

[D24] The antigen-binding molecule of any one of [D20] to [D23], wherein the antibody is a bispecific antibody,
particularly wherein the antigen binding molecule is a bispecific antibody directed against a membrane protein of an immune cell and against a DAMP,
especially wherein the antibody is selected from the group consisting of
a bispecific anti-HSP90 anti-CD3 antibody,
a bispecific anti-HSP90 anti-CD3 antibody;
a bispecific anti-GRP78 anti-CD3 antibody,
a bispecific anti-GRP78 anti-CD137 antibody,
a bispecific anti-phosphatidylserine anti-CD3 antibody, and
a bispecific anti-phosphatidylserine anti-CD137 antibody.

[D25] The antigen-binding molecule of any one of [D1] to [D24], wherein
i-1) the first moiety is Ig-type binding moiety, or
i-2) wherein the first moiety is a binding polypeptide, and/or
ii) the second moiety is an Ig-type binding moiety;
particularly wherein
a) the first moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂; and/or
b) the second moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂; and/or
c) the said binding polypeptide is a non-Ig-type binding moiety;
preferably wherein
   a*) the first moiety is a binding domain selected from the group consisting of an VHH, an scFv, an scFv2, a Fab, and a F(ab′)₂, and/or
   b*) the second moiety is a binding domain selected from the group consisting of an VHH, an scFv, an scFv2, a Fab, and a F(ab′)₂; and/or
   c*) wherein the binding polypeptide is a non-Ig-type binding moiety, especially a non-Ig-type binding moiety attached to an Fc domain of the said antigen-binding molecule.

[D26] The antigen-binding molecule of any one of [D20] to [D25], wherein the molecule is selected from the group consisting of
A) an antibody capable of binding to F-actin, as well as to an antigen involved in a signal transmission in a cell;
B) an antibody capable of binding to F-actin, as well as to an immune cell receptor; or
C) an antibody capable of binding to HSP90, as well as to an antigen involved in a signal transmission in a cell;
D) an antibody capable of binding to HSP90, as well as to an immune cell receptor; or
E) an antibody capable of binding to GRP78, as well as to an antigen involved in a signal transmission in a cell;
F) an antibody capable of binding to GRP78, as well as to an immune cell receptor; or
G) an antibody capable of binding to phosphatidylserine, as well as to an antigen involved in a signal transmission in a cell;
H) an antibody capable of binding to phosphatidylserine, as well as to an immune cell receptor;
I) an antibody capable of binding to SAP130, as well as to an antigen involved in a signal transmission in a cell; and
J) an antibody capable of binding to SAP130, as well as to an immune cell receptor, preferably wherein the molecule is selected from the group consisting of
A-1) an antibody capable of binding to F-actin, as well as to CD3;
A-2) an antibody capable of binding to F-actin, as well as to CD137;
C-1) an antibody capable of binding to HSP90, as well as to CD3;
C-2) an antibody capable of binding to HSP90, as well as to CD137;
E-1) an antibody capable of binding to GRP78, as well as to CD3;
E-2) an antibody capable of binding to GRP78, as well as to CD137;
G-1) an antibody capable of binding to phosphatidylserine, as well as to CD3;
G-2) an antibody capable of binding to phosphatidylserine, as well as to CD137;
I-1) an antibody capable of binding to SAP130, as well as to CD3; and
I-2) an antibody capable of binding to SAP130, as well as to CD137,

[D27] The antigen-binding molecule of any one of [D20] to [D26], wherein the molecule is selected from the group consisting of
A) an antibody comprising two first moieties capable of binding to F-actin, especially wherein the first moieties are binding polypeptides, preferably Clec9A polypeptides, preferably attached to the Fc domain of the antibody, as well as two second moieties involved in a signal transmission in a cell, especially wherein the second moieties are Ig-type binding moieties;
B) an antibody comprising two first moieties capable of binding to F-actin, especially wherein the first moieties are binding polypeptides, preferably Clec9A polypeptides, preferably attached to the Fc domain of the antibody, as well as two second moieties which are an immune cell receptor antigen, especially wherein the second moieties are Ig-type binding moieties;
C) an antibody comprising two first moieties capable of binding to HSP90, especially wherein the first moieties are Ig-type binding moieties, as well as two second moieties involved in a signal transmission in a cell, especially wherein the second moieties are Ig-type binding moieties;
D) an antibody comprising two first moieties capable of binding to HSP90, especially wherein the first moieties are Ig-type binding moieties, as well as two second moieties, which are an immune cell receptor antigen, especially wherein the second moieties are Ig-type binding moieties;
E) an antibody comprising two first moieties capable of binding to GRP78, especially wherein the first moieties are Ig-type binding moieties, as well as two second moieties involved in a signal transmission in a cell, especially wherein the second moieties are Ig-type binding moieties;
F) an antibody comprising two first moieties capable of binding to GRP78, especially wherein the first moieties are Ig-type binding moieties, as well as two second moieties, which are an immune cell receptor antigen, especially wherein the second moieties are Ig-type binding moieties;
G) an antibody comprising two first moieties capable of binding to phosphatidylserine, especially wherein the first moieties are Ig-type binding moieties, as well as two second moieties involved in a signal transmission in a cell, especially wherein the second moieties are Ig-type binding moieties;
H) an antibody comprising two first moieties capable of binding to phosphatidylserine, especially wherein the first moieties are Ig-type binding moieties, as well as two second moieties, which are an immune cell receptor antigen, especially wherein the second moieties are Ig-type binding moieties;
I) an antibody comprising two first moieties capable of binding to SAP130, especially wherein the first moieties are binding polypeptides, preferably Clec4e polypeptides, preferably attached to the Fc domain of the antibody, as well as two second moieties involved in a signal transmission in a cell, especially wherein the second moieties are Ig-type binding moieties;
J) an antibody comprising two first moieties capable of binding to SAP130, especially wherein the first moieties are binding polypeptides, preferably Clec4e polypeptides, preferably attached to the Fc domain of the antibody, as well as two second moieties, which are an immune cell receptor antigen, especially wherein the second moieties are Ig-type binding moieties, preferably wherein the molecule is selected from the group consisting of
A1) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD3, especially wherein the second moieties are Ig-type binding moieties;
A2) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD137, especially wherein the second moieties are Ig-type binding moieties;
C1) an antibody comprising two first moieties capable of binding to HSP90, especially wherein the first moieties are Ig-type binding moieties, as well as two second moieties capable of binding to CD3, especially wherein the second moieties are Ig-type binding moieties;
C2) an antibody comprising two first moieties capable of binding to HSP90, especially wherein the first moieties are Ig-type binding moieties, as well as two second moieties capable of binding to CD137, especially wherein the second moieties are Ig-type binding moieties;
E1) an antibody comprising two first moieties capable of binding to GRP78, especially wherein the first moieties are Ig-type binding moieties, as well as two second moieties capable of binding to CD3, especially wherein the second moieties are Ig-type binding moieties;
E2) an antibody comprising two first moieties capable of binding to GRP78, especially wherein the first moieties are Ig-type binding moieties, as well as two second moieties capable of binding to CD137, especially wherein the second moieties are Ig-type binding moieties;
G1) an antibody comprising two first moieties capable of binding to phosphatidylserine, especially wherein the first moieties are Ig-type binding moieties, as well as two second moieties capable of binding to CD3, especially wherein the second moieties are Ig-type binding moieties;
G2) an antibody comprising two first moieties capable of binding to phosphatidylserine, specially wherein the first moieties are Ig-type binding moieties, as well as two second moieties capable of binding to CD137, especially wherein the second moieties are Ig-type binding moieties;
I1) an antibody comprising two first moieties capable of binding to SAP130, especially wherein the first moieties are binding polypeptides, preferably Clec4e polypeptides, preferably attached to the Fc domain of the antibody, as well as two second moieties capable of binding to CD3, especially wherein the second moieties are Ig-type binding moieties;
I2) an antibody comprising two first moieties capable of binding to SAP130, especially wherein the first moieties are binding polypeptides, preferably Clec4e polypeptides, preferably attached to the Fc domain of the antibody, as well as two second moieties capable of binding to CD137, especially wherein the second moieties are Ig-type binding moieties.

[D28] A pharmaceutical composition comprising an antigen-binding molecule of any one of [D1] to [D27].

[D29] The antigen-binding molecule or pharmaceutical composition according to any one of [D1] to [D28], for use in
i) medicine, and/or
ii) a method of treating a medical condition, particularly a medical condition involving cell death, and/or
iii) a method of treating a medical condition in a subject, the method comprising contacting the said antigen-binding molecule with a damaged cell,
iv) a method of treating a medical condition in a subject, the method comprising contacting the said antigen-binding molecule with a damage-associated molecular pattern (DAMP) in a patient, particularly further comprising contacting the said antigen-binding molecule with the said second antigen,
v) a method of treating a medical condition in a subject, the method comprising administering the said antigen-binding molecule to a patient suffering from a condition involving cell damage, particularly cell death;
especially wherein the method comprises contacting a plurality of antigen-binding molecules in close proximity with the first and second antigens.

[D30] The antigen-binding molecule or pharmaceutical composition according to any one of [D1] to [D29] for use in medicine.

[D31] The antigen-binding molecule or the pharmaceutical composition of any of [D1] to [D27] for use in a method of treating or preventing a medical condition.

[D32] The antigen-binding molecule or the pharmaceutical composition for use of [D31], wherein the medical condition is selected from the group consisting of a cancer and an immune, preferably an autoimmune, disease.

[D33] The antigen-binding molecule or pharmaceutical composition for use according to any one of [D29]-[D32], wherein
i) the method comprises administering an antigen-binding molecule according to any one of [D1] to [D27] to a patient, and/or
ii) the method is a method for activating or preventing an immune response, and/or
iii) the method is for conferring a signal transmission or blockage in a cell; particularly wherein
a) the method comprises contacting the antigen-binding molecule with a damage-associated molecular pattern (DAMP), which is exposed to an extracellular environment due to cell damage, especially wherein the DAMP is selected from the group consisting of F-actin, GRP78, phosphatidylserine, HSP90, and SAP130, and/or
b) the method comprises contacting the antigen-binding molecule with a cell especially selected from a-i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, a-ii) a tumor cell, and a-iii) an autoreactive cell, and/or
c) the antigen-binding molecule comprises at least one, preferably two, Clec9A polypeptide(s), especially wherein the antigen-binding molecule further comprises c-1) at least two, preferably two, moieties, which are involved in a signal transmission in a cell or c-2) at least two, preferably two, moieties, which are an immune cell receptor antigen.

[D34] The antigen-binding molecule or pharmaceutical composition for use according to any one of [D18] and [D19], wherein the method is a method for treating or preventing a cancer or an immune (preferably an autoimmune) disease,
especially wherein the method is a method for treating or preventing a cancer, in particular wherein the method is a method for treating a cancer.

[D35] A polynucleotide encoding antigen-binding molecule according to any one of [D1] to [D27].

[D36] A polynucleotide in particular a DNA encoding the molecule of any one of [D1] to [D27].

[D37] A vector comprising the polynucleotide of [D35] or [D36].

[D38] A vector comprising a DNA sequence encoding for the antigen-binding molecule as defined in any one of [D1] to [D27], wherein the vector is in particular for recombinant protein expression.

[D39] A host cell comprising the said vector of [D37] or [D38].

[D40] A host cell comprising the vector of [D37] or [D38], wherein the host cell is in particular for recombinant protein expression of the antigen-binding molecule encoded in the vector.

[D41] Use of at least a first moiety and at least a second moiety for the production of an antigen-binding molecule comprising the at least first moiety and the at least a second moiety, wherein
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell.

[D42] Use of an antigen-binding molecule for targeting a cell in a tissue where cell death is observed or to be detected, wherein
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell.

[D43] Use of an antigen-binding molecule for induction or blockage of signal transduction in a cell in a tissue where cell death is observed, wherein
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell.

[D44] A kit for producing a molecule specifically acting in a tissue where a cell death is being observed, the molecule comprising
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell, wherein
the kit comprises
at least a first container comprising the least first moiety and
at least a second container comprising the at least second moiety.

In addition, the present disclosure particularly also provides the following embodiments [E1] to [E32]:

[E1] An antigen-binding molecule which comprises:
(1) a first moiety that binds to a first antigen, wherein said first antigen is selected from the group consisting of an actin, a heat shock protein, a phosphatidylserine, a histone, a histone deacetylase complex subunit, a HMGN protein, a hepatoma-derived growth factor, BCL-2, calreticulin, and cyclophilin A and
(2) a second moiety that binds to a second antigen, wherein the second antigen is different from the first antigen and is selected from a membrane protein on Tcell or antigen presenting cells (APCs).

[E2] The antigen-binding molecule of [E1], wherein the actin is F-actin. [E3] The antigen-binding molecule of [E1], wherein the heat shock protein is HSP90, GRP78, HSP70, HSP60, HSP72, or GP96.

[E4] The antigen-binding molecule of [E3], wherein the heat shock protein is HSP90 or GRP78.

[E5] The antigen-binding molecule of [E1], wherein the phosphatidylserine is part of a cell membrane.

[E6] The antigen-binding molecule of [E1], wherein the histone deacetylase complex subunit is SAP130.

[E7] The antigen-binding molecule of [E1], wherein the HMGN protein is HMGB1 or HMGN1.

[E8] The antigen-binding molecule of [E1], wherein the first antigen is selected from the group consisting of F actin, GRP78, phosphatidylserine, HSP90, and SAP130.

[E9] The antigen-binding molecule of any one of s [E1] to [E8], wherein the first moiety is an Ig type binding moiety selected from the group consisting of IgG type antibody, VHH, VH, VL, sdAb, scFv, single-chain antibody, Fab, and F(ab′)₂.

[E10] The antigen-binding molecule of any one of [E1] to [E8], wherein the first moiety is a non-Ig-type binding moiety.

[E11] The antigen-binding molecule of [E1], wherein the first antigen is F-actin and the first moiety is a Clec9A polypeptide.

[E12] The antigen-binding molecule of [E1], wherein the first antigen is selected from the group consisting of GRP78, phosphatidylserine, and HSP90 and the first moiety is an Ig type binding moiety.

[E13] The antigen-binding molecule of [E1], wherein the first antigen is SAP130 and the first moiety is a Clec4e polypeptide.

[E14] The antigen-binding molecule of any one of [E1] to [E13], wherein the second antigen is selected from the group consisting of a T cell receptor, CD3, CD137, CD40, and CTLA4.

[E15] The antigen-binding molecule of any one of [E1] to [E14], wherein the second moiety is an Ig type binding moiety.

[E16] The antigen-binding molecule of any one of [E1] to [E15], wherein the molecule is an IgG type antibody that contains both the first moiety and the second moiety.

[E17] The antigen-binding molecule of [E16], wherein the IgG type antibody is capable of binding to a membrane protein of an immune cell and comprises at least one binding polypeptide.

[E18] The antigen-binding molecule of [E17], wherein the binding polypeptide is linked to the Fc region of the IgG type antibody,

[E19] The antigen-binding molecule of [E17] or [E18], wherein the binding polypeptide comprises Clec9A polypeptide or a Clec4e polypeptide.

[E20] The antigen-binding molecule of [E17], wherein the IgG type antibody is selected from:
an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

[E21] The antigen-binding molecule of [E16], wherein the IgG type antibody is a bispecific antibody,

[E22] The antigen-binding molecule of [E21], wherein the bispecific antibody is directed against a membrane protein of an immune cell and against a DAMP.

[E23] The antigen-binding molecule of [E21], wherein the bispecific antibody is selected from
a bispecific anti-HSP90 anti-CD3 antibody,
a bispecific anti-HSP90 anti-CD3 antibody;
a bispecific anti-GRP78 anti-CD3 antibody,
a bispecific anti-GRP78 anti-CD137 antibody,
a bispecific anti-phosphatidylserine anti-CD3 antibody, and
a bispecific anti-phosphatidylserine anti-CD137 antibody.

[E24] A pharmaceutical composition comprising an antigen-binding molecule of any one of [E1] to [E23].

[E25] An isolated polynucleotide encoding the antigen binding molecule of any one of [E1] to [E23].

[E26] A vector comprising the polynucleotide of [E25]. [E27] A host cell comprising the polynucleotide of [E25].

[E28] A method of treating a medical condition involving cell damage or cell death in a subject suffering therefrom, comprising administering the antigen-binding molecule of any one of [E1] to [E23] to the subject.

[E29] A method of treating a cancer in a subject in need thereof, comprising administering the antigen-binding molecule of any one of [E1] to [E23] to the subject.

[E30] A method of treating an immune disease in a subject in need thereof, comprising administering the antigen-binding molecule of any one of [E1] to [E23] to the subject.

[E31] The method of [E30], wherein the immune disease is an autoimmune disease.

[E32] A method comprising culturing the host cell of [E27] under conditions suitable for expression of the antigen binding molecule and optionally recovering the antigen binding molecule.

### BRIEF DESCRIPTION OF DRAWINGS

FIG. 1. A drawing schematically depicting a technical concept encompassed by and achieved by the present invention in the case that a molecule of the present invention is an antibody-like molecule comprising a moiety which binds to a F-actin which is exposed to a extracellular environment due to a cell death, and also schematically depicting that the molecules form a complex with the F-actin, and the complex binds one or more of membrane protein in a cell via said molecules to confer a signal transmission or blockage into the cell.

FIG. 2. A drawing schematically depicting a technical feature of one embodiment of a molecule of the present invention, which is an antibody-like molecule comprising a moiety which binds to a component or a portion thereof of a cell which is exposed to a extracellular environment due to a cell death.

FIG. 3. A drawing schematically illustrating technical feature of various embodiments of a molecule of the present invention, which is an antibody-like molecule or Ig-like molecule comprising a moiety which binds to a component or a portion thereof of a cell which is exposed to a extracellular environment due to a cell death. In more detail, the drawing schematically illustrates various embodiments of an antibody-like molecule or Ig-like molecule of the present invention comprising at least one “first moiety” and at least one “second moiety” and a Fc region, and embodiments comprising at least one “first moiety”, at least one “second moiety”, at least one “third moiety” and a Fc region.

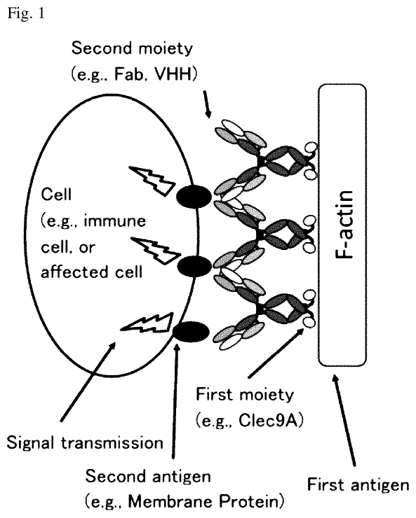

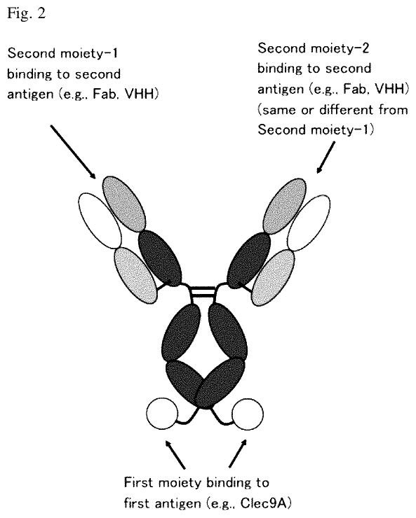

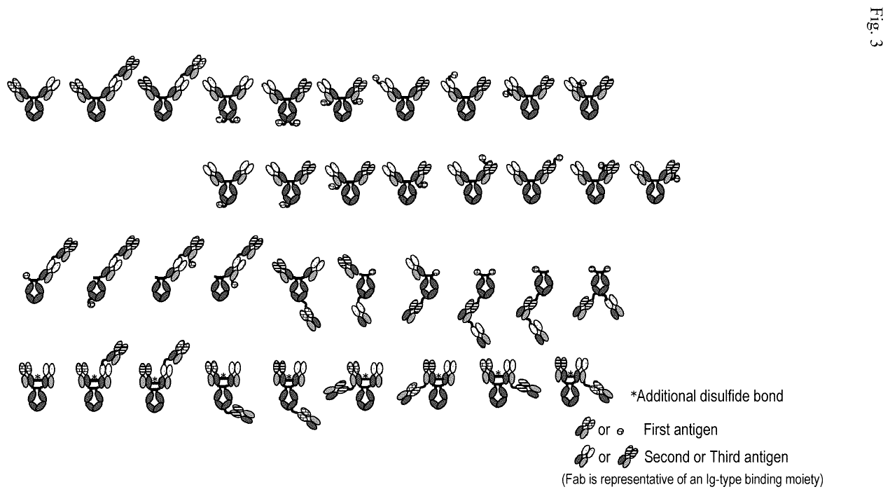

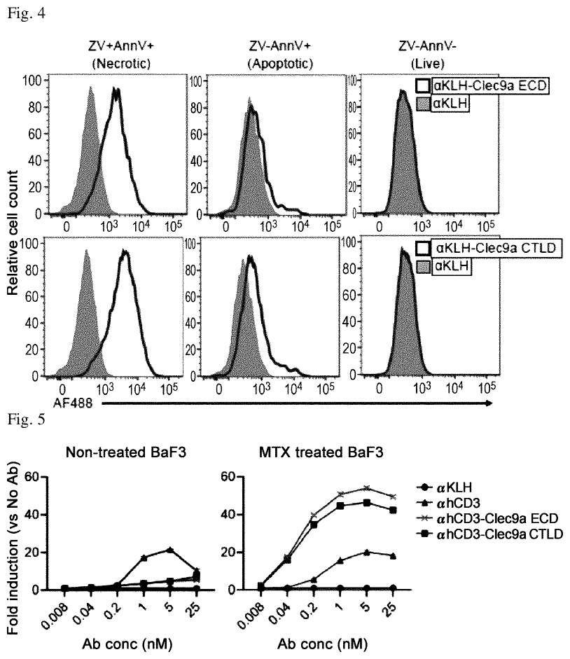

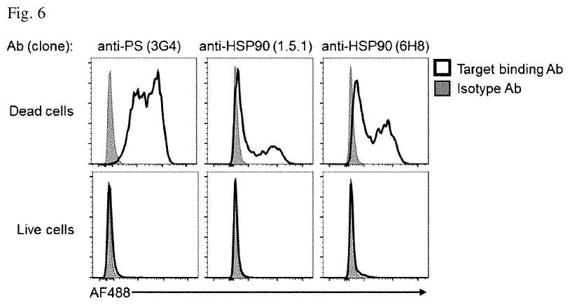

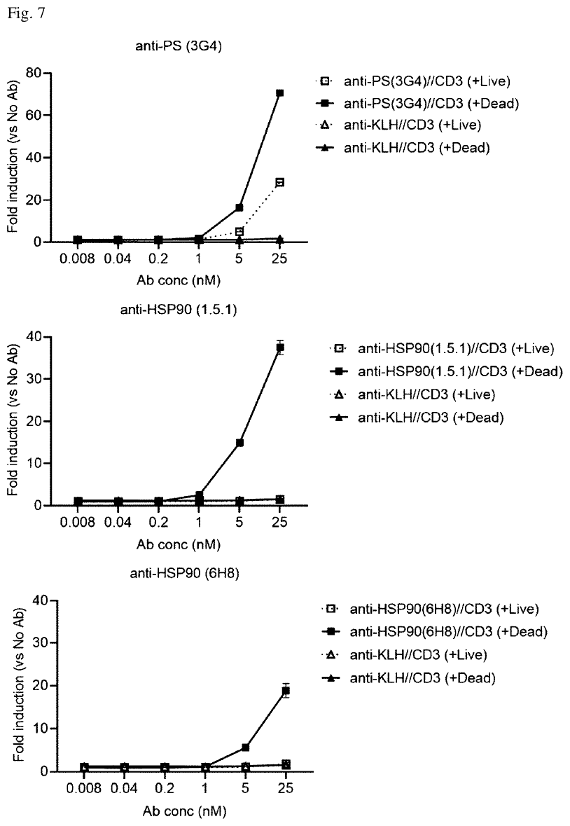

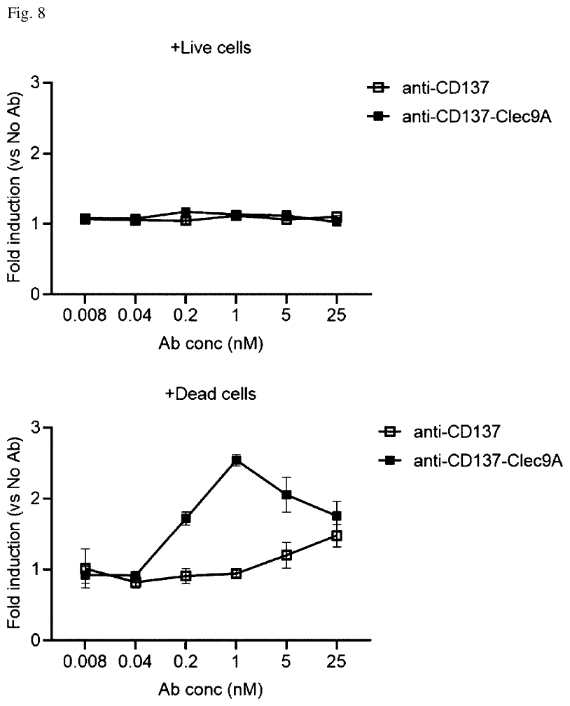

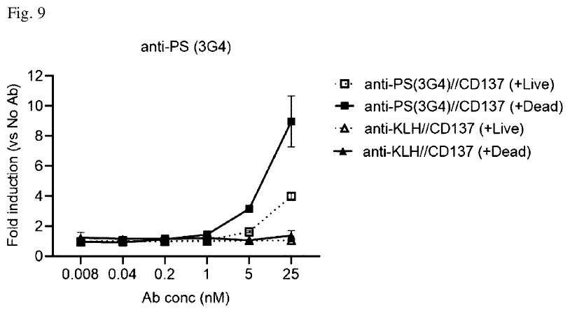

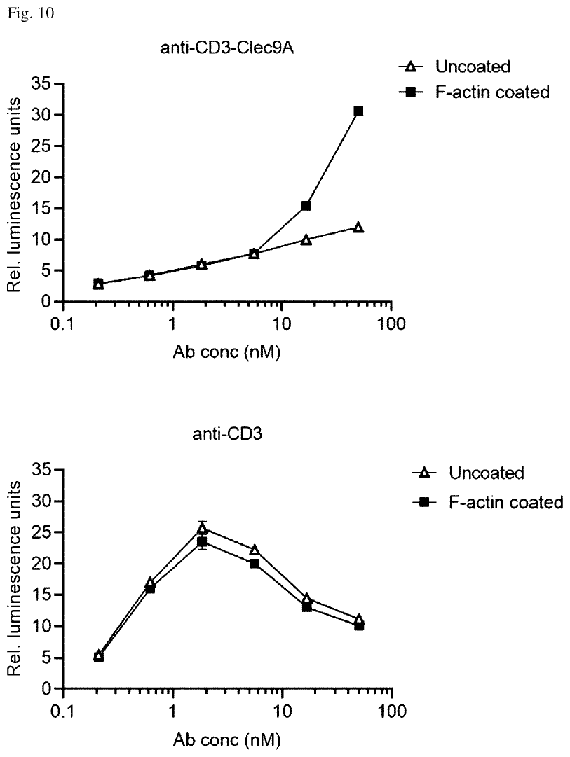

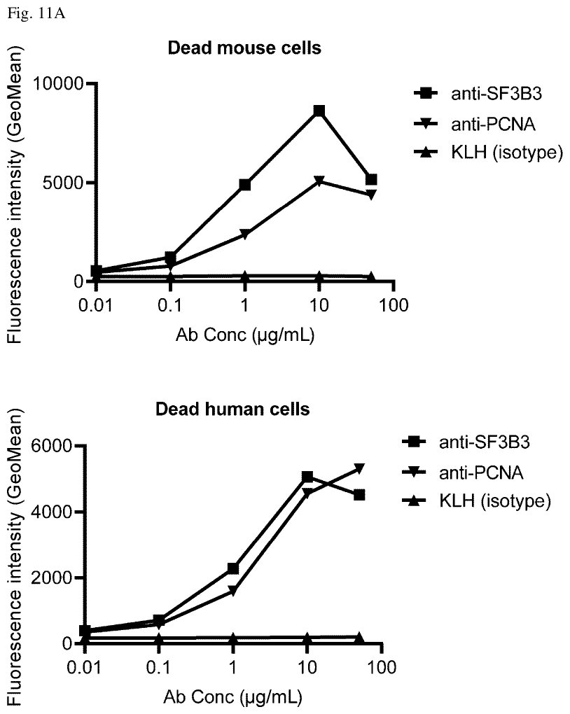

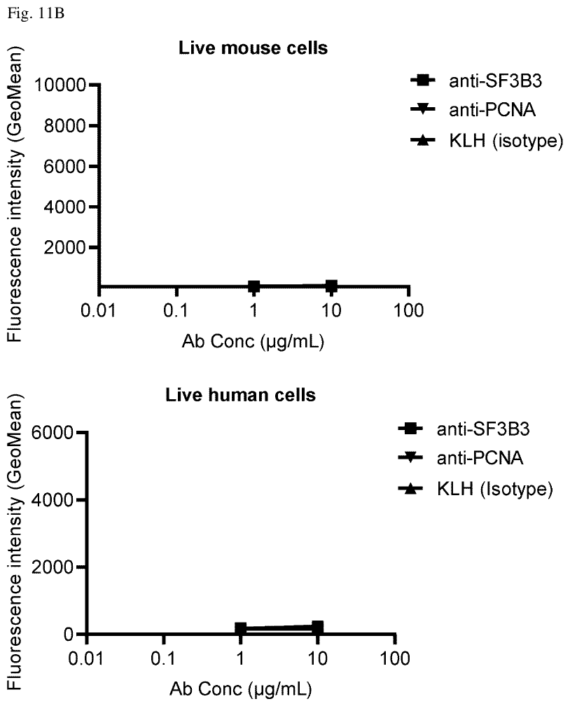

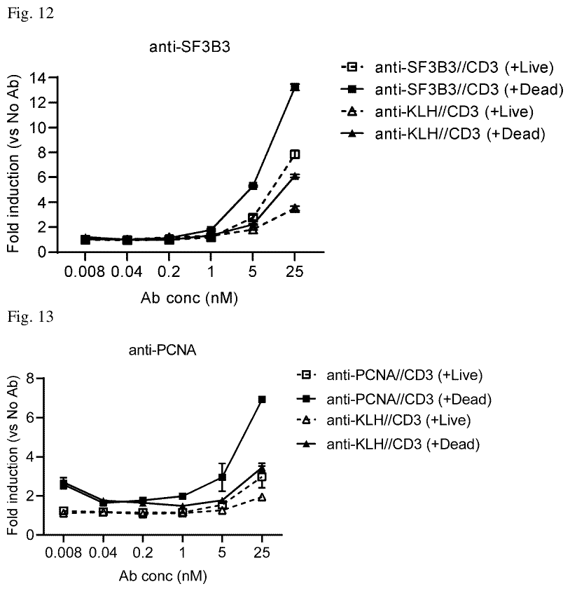

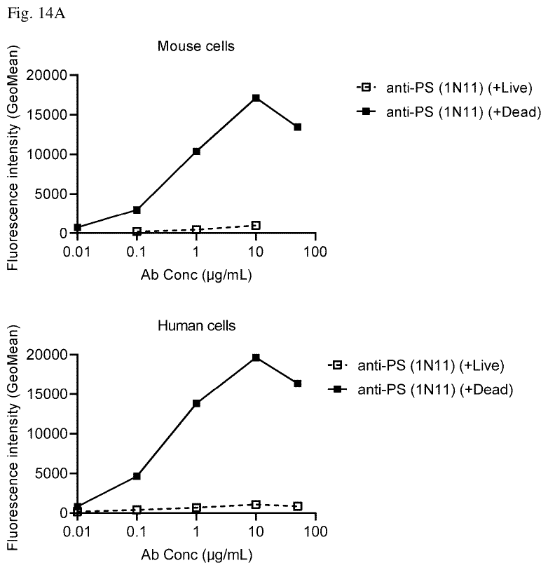

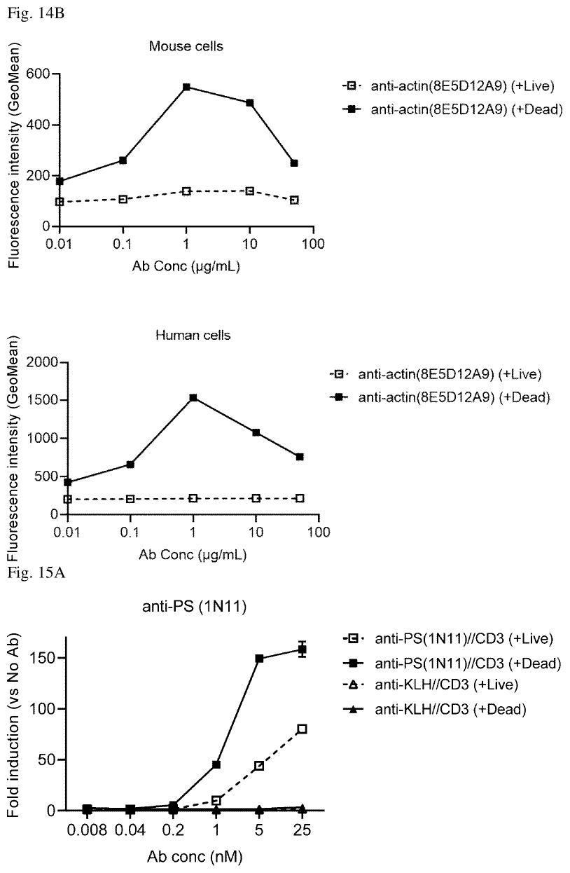

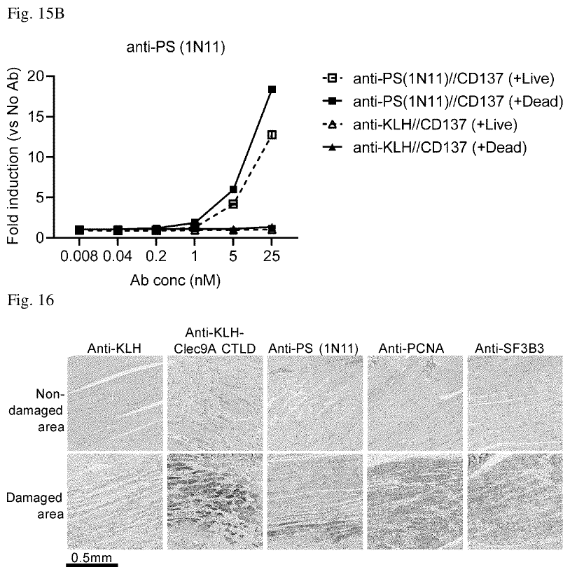

For example, a circle (white horizontal brick) means “a first moiety” that binds to “a first antigen”. In further example, in case that two first moiety (white horizontal brick circle) are conjugated, at least one moiety binds to “a first antigen” and the other can bind to any different antigen from “a first antigen” and “a second antigen”. In another example, at least one Fab arm binds to “a second antigen” and the other Fab arm can bind to any different antigen from “a first antigen” and “a second antigen”, in particular the third Fab arm can bind to a “third antigen”. In further example, at least one Fab arm binds to “a second antigen” and the other Fab arm can bind to “a first antigen”, and first moiety (white horizontal brick circle) conjugated can bind any different antigen from “a first antigen” and “a second antigen”, in particular a third Fab arm can bind to a “third antigen”. In an example, at least one Fab arm binds to “a first antigen” and a second Fab arm can bind to “a second antigen”, and a third Fab arm can bind to any different antigen from “a first antigen” and “a second antigen”, in particular the third Fab arm can bind to a “third antigen”. In another example, at least one first moiety binds to “a first antigen” and a second Fab arm can bind to “a second antigen”, and a third Fab arm can bind to any different antigen from “a first antigen” and “a second antigen”, in particular the third Fab arm can bind to a “third antigen”. Further embodiments schematically depicted in FIG. 3 are described below in more detail.

FIG. 4. A drawing showing results of the measurement of the binding of Clec9A conjugated antibody to live cells, and necrotic cells or its components respectively.

FIG. 5. A drawing showing results of the measurement of T cell activation though clustering of Clec9A-conjugated anti-CD3 antibodies onto the T-cell in the presence of dead cells.

FIG. 6. A drawing showing results of the measurement of anti-phosphatidyl serine or anti-HSP90 binding to live or dead cells.

FIG. 7. A drawing showing results of the measurement of CD3+ T cell activation through clustering of anti-phosphatidylserine//CD3 or anti-HSP90//CD3 bispecific antibodies onto the T-cell in the presence of live or dead cells.

FIG. 8. A drawing showing results of the measurement of CD137+ T cell activation though clustering of Clec9A-conjugated anti-CD137 antibodies onto T cells in the presence of live or dead cells.

FIG. 9. A drawing showing results of the measurement of CD137+ T cell activation through clustering of anti-phosphatidylserine//CD137 antibodies onto T cells in the presence of live or dead cells.

FIG. 10. A drawing showing results of CD3 activation by Clec9A conjugated antibodies in the presence or absence of F-actin.

FIG. 11A. A drawing showing results of the measurement of anti-SF3B3 and anti-PCNA antibodies binding to dead cells.

FIG. 11B. A drawing showing results of the measurement of anti-SF3B3 and anti-PCNA antibodies binding to live cells.

FIG. 12. A drawing showing results of the measurement of CD3+ T cell activation through clustering of anti-SF3B3//CD3 bispecific antibody onto the T-cell in the presence of live or dead cells.

FIG. 13. A drawing showing results of the measurement of CD3+ T cell activation through clustering of anti-PCNA//CD3 bispecific antibody onto the T-cell in the presence of live or dead cells.

FIG. 14A. A drawing showing results of the measurement of anti-PS (1N11) antibody binding to live or dead cells.

FIG. 14B. A drawing showing results of the measurement of anti-actin (8E5D12A9) antibody binding to live or dead cells.

FIG. 15A. A drawing showing results of the measurement of CD3+ T cell activation through clustering of anti-PS (1N11)//CD3+bispecific antibody onto the T-cell in the presence of live or dead cells.

FIG. 15B. A drawing showing results of the measurement of CD137+ T cell activation through clustering of anti-PS (1N11)//CD137+bispecific antibody onto the T-cell in the presence of live or dead cells.

FIG. 16. A drawing showing results of in vivo distribution of dead cell binding molecules in a muscle injury model.

### DESCRIPTION OF EMBODIMENTS

The definitions and detailed description below are provided to facilitate understanding of the present invention illustrated herein. Especially, these definitions and detailed description serve to interpret the aspects and embodiments of group [AA] and embodiments of groups [A], [B], [C], [D] and [E] above.

### Cell Death

There are several types of cell death reported and categorized into two types, programmed cell death (PCD) and non-PCD (Cell Research volume 29, pages347-364, 2019; Cell Biol. Int., 2019 June; 43 (6): 582-592; Nature Reviews Cancer, Vol. 12, p. 860-875, 2012). PCD is strictly regulated by signaling cascade and molecularly defined mechanisms. Apoptosis is the most well-known PCD and recently many non-apoptotic type of PCD, pyroptosis, NETosis, Necroptosis, etc, are reported. On the other hand, non-PCD is a biologically uncontrollable process and necrosis is categorized as non-PCD.

In the present invention, “a cell death” refers to, including but not limited to, an apoptosis which is one of embodiments of a programmed cell death (PCD); a necrosis; an accidental cell death; or a regulated cell death (e.g., an apoptosis, a regulated necrosis, an autophagy cell death, necroptosis, ferroptosis, or pyroptosis), of any sort of or any type of cells, including but not limited to, a somatic cell (e.g., epithelial cell, fibroblastic cell, interstitial cell, bone cell, muscle cell, nerve cell, blood cell or the like), a reproductive cell, or an affected or abnormal cell (e.g., a tumor cell, an autoreactive cell). Such cells may be located in any tissue, including but not limited to, epithelial tissue, muscle tissue, nerve tissue, connective tissue. Here, necrosis, a form of non-programmed cell death resulting from injury or inflammation, also results in cell death increase. Specifically, in the present invention, “a cell death” can be a cell death observed in an affected tissue due to, e.g., a cancer or autoimmune disease.

### Cell Damage

Likewise, in the present invention a “cell damage” is not particularly limited and involves any cell damage resulting in the exposure of DAMP. It particularly includes all the above forms of cell death.

### A First Antigen/a Damage-Associated Molecular Pattern (DAMP)

In the present invention, the term “a first antigen” which is also referred to herein according to one general aspect of the invention as a “damage-associated molecular pattern” or “DAMP”, respectively, and which is used interchangeably herein with a “substance which is exposed to a extracellular environment due to a cell damage”, means a biomolecule that is associated with a cell damage, and, in the present invention, it may also be termed as “a damaged cell-derived molecule”, or “a damaged cell-derived substance”, “a damaged cell associated molecule”, “a damaged cell associated substance”, and “a danger associated molecular patterns”, which are used interchangeably. These biomolecule and substance associated with a cell damage can be upregulated in expression, expressed on a cell surface, modified, passively diffused or actively secreted extracellularly upon damage of cells. These changes are correlated positively with the presence or increase of the damaged cells, or with decrease of intact cells. In other words, in a cell getting toward a cell damage, a component or a portion thereof which constitutes a cytoskeleton, a cell membrane, a organelle, a cytoplasmic protein, or a metabolite in the cell is exposed to a extracellular environment, and any of these is encompassed by the substance or biomolecule associated with a cell damage.

At any rates, “damage-associated molecular patterns” (“DAMP”) are well known to the person skilled in the art as such “a substance which is exposed to a extracellular environment due to a cell damage”.

In the present invention, the terms “damage-associated molecular pattern”, “DAMP” and “a substance which is exposed to a extracellular environment due to a cell damage” can be referred as “a component or a portion, respectively, of a cytoskeleton, of a cell membrane, of an organelle, of a cytoplasmic protein, or of a metabolite, which is exposed to a extracellular environment due to a cell damage”, and also as “a first antigen” interchangeably. That is, in the present invention, unless otherwise defined to mean differently, the term “a first antigen” means including but not limited to as “a component or a portion, respectively, of a cytoskeleton, of a cell membrane, of an organelle, of a cytoplasmic protein, or of a metabolite, which is exposed to a extracellular environment due to a cell damage”.

In one aspect herein, the DAMP is selected from the group consisting of A) an actin, particularly F-actin; B) a heat shock protein, particularly selected from HSP90 and GRP78, especially HSP90; C) a phosphatidylserine (PS), particularly a phosphatidylserine that is part of a cell membrane; D) a histone or component thereof, E) a histone deacetylase complex subunit, particularly SAP130, F) a HMGN protein, particularly selected from HMGB1 and HMGN1, G) hepatoma-derived growth factor, H) BCL-2, I) calreticulin, and J) cyclophilin A.

In one aspect herein, the first antigen is selected from the group consisting of a) Splicing Factor 3B Subunit 3 (SF3B3); and b) Proliferating Cell Nuclear Antigen (PCNA).

The terms “SF3B3 splicing factor 3b subunit 3” and “SF3B3” are used interchangeably herein and refer to the subunit 3 of the splicing factor 3b protein complex. The sequence of human SF3B3 is disclosed as translated protein sequence in NCBI Reference Sequence: NP_036558.3, version of 22 May 2022.

The terms “Proliferating cell nuclear antigen” and “PCNA” are used interchangeably herein and refer to the protein known in the art as Proliferating cell nuclear antigen.

Proliferating cell nuclear antigen or PCNA has been described to function as a DNA clamp that acts as a processivity factor for DNA polymerase delta in eukaryotic cells and is essential for replication. In vivo, PCNA is described to be a homotrimer. PCNA has been described to act as a scaffold to recruit proteins involved in DNA replication, DNA repair, chromatin remodelling and epigenetics.

A sequence of human PCNA is disclosed as translated protein sequence in NCBI Reference Sequence: NP_872590.1, version of Jun. 19, 2022.

In the present invention, cell damage preferably involves cell death. In preferred embodiments invention, cell damage is cell death. Accordingly, in a further general aspect of the invention, the following applies:

A First Antigen, and a Substance which is Exposed to a Extracellular Environment Due to a Cell Death

In the present invention, the term “a first antigen” which is also referred as “a substance which is exposed to a extracellular environment due to a cell death” means a biomolecule that is associated with a cell death, and, in the present invention, it may also be termed as “a dead cell-derived molecule”, or “a dead cell-derived substance”, “a dead cell associated molecule”, “a dead cell associated substance”, and “a danger associated molecular patterns”, which are used interchangeably. These biomolecule and substance associated with a cell death can be upregulated in expression, expressed on a cell surface, modified, passively diffused or actively secreted extracellularly upon death of cells. These changes are correlated positively with the presence or increase of the dead cells, or with decrease of live cells. In other words, in a cell getting toward a cell death, a component or a portion thereof which constitutes a cytoskeleton, a cell membrane, a organelle, a cytoplasmic protein, or a metabolite in the cell is exposed to a extracellular environment, and any of these is encompassed by the substance or biomolecule associated with a cell death.

That is, the term “a substance which is exposed to a extracellular environment due to a cell death” (or, a substance which is exposed to a extracellular environment due to a cell damage” a “damage-associated molecular pattern” or “DAMP”) can be referred as “a component or a portion thereof which constitutes a cytoskeleton, a cell membrane, a organelle, a cytoplasmic protein, or a metabolite, which is exposed to a extracellular environment due to a cell death”, and also as “a first antigen” interchangeably (or as “a component or a portion, respectively, of a cytoskeleton, of a cell membrane, of an organelle, of a cytoplasmic protein, or of a metabolite, which is exposed to a extracellular environment due to a cell damage”, respectively). That is, in the present invention, unless otherwise defined to mean differently, the term “a first antigen” means including but not limited to “a component or a portion thereof which constitutes a cytoskeleton, a cell membrane, a organelle, a cytoplasmic protein, or a metabolite, which is exposed to a extracellular environment due to a cell death” or “a component or a portion, respectively, of a cytoskeleton, of a cell membrane, of an organelle, of a cytoplasmic protein, or of a metabolite, which is exposed to a extracellular environment due to a cell damage”, respectively.

In any case, in the present invention, all examples and embodiments, respectively for “dead cell-derived molecule” and suchlike are particularly included within the DAMP in context with the invention.

In the present invention, the term “a cytoskeleton” means a filament structure in a cytoplasm which generates physical power needed for intracellular and extracellular motion of a cell, and also for maintaining a morphology of a cell. In the present invention, “a filament” includes but not limited to an intermediate filament, an actin filament, a myosin filament, and a keratin filament. A eukaryotic cell has, as cytoskeleton, an actin filament, an intermediate filament, a myosin filament, a keratin filament, and a microtubule. The actin filament is composed of an actin; the intermediate filament is composed of vimentin, glial fibrillary proteins, neuro-filament proteins, nuclear lamin(s), and so on; and the microtubule is composed of alpha-tubulin and beta-tubulin. In the present invention, the “cytoskeleton” which is exposed to a extracellular environment due to a cell death is not limited to the protein components which form the above cytoskeleton, and those can be conjugated with one or more or any other proteins.

Biomembranes such as cell membranes are composed of the lipid bilayer in which proteins have been buried. In the present invention, the components of the biomembranes such as cell membranes includes but not limited to lipids and proteins. The lipid includes but not limited to a phospholipid, a glycolipid, and a sterol. The protein includes but not limited to a transmembrane protein such as an ion-channel, a G-protein coupled receptor (GPCR), a proton pump and so on, a lipid-anchor protein such as G-protein and so on, and a superficial membrane protein such as an enzyme, a hormone, and so on.

Generally, in the present invention, a “membrane protein” refers to a membrane protein as it is generally well known to a person skilled in the art, as common proteins that are part of, or interact with, biological membranes, and that include several broad categories depending on their location, which particularly include integral membrane proteins are a permanent part of a cell membrane and can either penetrate the membrane (transmembrane) or associate with one or the other side of a membrane (integral monotopic) and peripheral membrane proteins, which transiently associated with the cell membrane (e.g. via a so-called anchor or by a certain type or combination of non-covalent interaction). The term especially includes any of the membrane proteins described herein.

In the present invention, “a cell membrane or an organelle which is exposed to a extracellular environment due to a cell death” includes but is not limited to the above-phospholipids or proteins, any biomembrane which composes an organelle such as a nuclear body, a nucleus, a ribosome, an endoplasmic reticulum, a Golgi-body, a mitochondria, and so on, and also a protein buried in the biomembrane.

In the present invention, “a substance/molecule which is exposed to a extracellular environment due to a cell death” (“a dead cell-derived molecule”) as well as a DAMP includes but not limited to the above-described component or a portion thereof, and any substance or molecule which is exposed to a extracellular environment due to a cell death may be utilized in the present invention. Further, the dead cell-derived molecule may be a complex of one or more of the above-components or the portions thereof. The dead cell-derived molecule may be a whole of or a truncated form of a protein. The dead cell-derived molecule includes but is not limited to a cytoskeleton, a organelle, a nuclear protein, and a cytoplasmic protein in a cell as a preferable embodiment. A cell membrane and any fragment of the cell membrane are also included in the dead cell-derived molecule as a preferable embodiment. In the present invention, more preferable embodiments of the dead cell-derived molecule include but are not limited to a filament which composes a cytoskeleton or nucleus skeleton, and a nucleosome or a molecule which forms a nucleosome. In the present invention, further preferable embodiments of the dead cell-derived molecule include but are not limited to a protein which is a constituent of the spliceosome.

In the present invention, the dead cell-derived molecules include, as non-limited embodiments, a molecule which is released from a cell into extracellular environment due to an immunogenic cell death, i.e. a damage-associated molecular patterns (DAMPs) released due to an immunogenic cell death. The DAMPs include a molecule reported by Dmitri et al, Nature Reviews Cancer, Vol. 12, p. 860-875, 2012. In other words, all DAMPs described in Dmitri et al are particularly included as embodiments for the DAMP in accordance with the present invention.

Saying further and somewhat repeatedly, in the present invention, specific embodiments of “a substance which is exposed to a extracellular environment due to a cell death” or “a component or a portion thereof which constitutes a cytoskeleton, a cell membrane, a organelle, a cytoplasmic protein, or a metabolite, which is exposed to a extracellular environment due to a cell death” as well as a “DAMP” include but are not limited to, a filament or histone which forms a cytoskeleton or a nuclear skeleton (e.g., an intermediate filament such as F-actin), a phosphatidylserine, a heat shock protein, and further a protein, an RNA, a DNA, or a metabolite or a combinations thereof which results in the modified molecule or a complex. Here, the protein can be a whole length protein or a fragment thereof.

Yet further, in some preferred embodiments, the organelle may be mitochondria, nucleus, lysosome, golgi apparatus, or endoplasmic reticulum.

As further examples, and not limiting thereto, a dead cell-derived molecule as described above in the present invention includes molecules that normally reside in the cytosol or nucleus, including Amyloid beta, Adenosine triphosphate (ATP), Uric acid crystal(s), Myosin light chain, Alpha1-microglobulin, Alpha2-microglobulin, Cyclophilin A, F-actin, HSP70, HSP90, gp96, GRP94, HSP110, HSP170, Calgranulin A, Calgranulin B, Calreticulin, Gc-globulin, SF3B3, HMBG1, HMGN1, IL1a, IL1b, IL33, Serum amyloid protein etc.

As, further examples, a “DAMP” may be Mitochondria DNA (mtDNA), Mitochondria ROS (mROS) or Mitochondria TF A (TFAM), or a formyl peptide.

In addition, molecules such as glypicans and syndecans can be expressed on the surface of live cells, but modified by a dead cell rich microenvironment, and these molecules are also included in the dead cell-derived molecule. Further, biglycans, decorins and lumicans, fibrinogens, fibronectins, EDA domain of fibronectin, heparan sulfates, perlecans, low molecular weight hyaluronans, versicans, tenascin C, can also be sequestered in extracellular matrices under normal physiological conditions, but proteolytically released along with increase in dead cells. Further, granules originally existing intracellularly in living cells can be released and localised in the dead cell rich microenvironment such as cathelicidin (LL37), defensins, eosinophil-derived neurotoxin (EDN), and granulysin are also included in the dead cell-derived molecule. These molecules are also included in the present invention as a dead cell associated molecule in the present invention.

Dead cell-derived molecule can also be a molecule released, or exposed or abundantly expressed at cell surface when cell death is induced. All cells, such as somatic cell and reproductive cells, are possible to produce a dead cell-derived molecule. A common feature of solid tumor is the presence of hypoxia and low nutrient state derived from reduced blood flow. These hypoxic condition and low nutrient state stress tumor cells and result in abundant cell death in solid tumor. In addition to tumor, cell death is usually seen in many types of autoimmune diseases, such as vitiligo, type I diabetes, and so on because cell death is involved in the pathogenesis of the autoimmune diseases. For example, vitiligo is the autoimmune disease induced by the destruction of melanocytes. In vitiligo, it is reported that there are many types of autoantibodies recognizing lamin A, tyrosinase-related protein 1, HSP90, HSP70, and so on (Hamid et al., Pigmentary Disorders 2015, 2:6). These molecules are generally localized in cytosol or nucleus in steady state condition. Because antibody is generated against the molecules localized outside of the cell, these molecules must be released or exposed to plasma membrane upon cell death.

In the present invention, a source of a dead cell-derived molecule is not to be limited to the source as described above. Any molecule or substance of or in a cell which is exposed to extracellular environment where a cell death is observed can be a source of a dead cell-derived molecule. Whether or not, a certain molecule or a substance is a dead cell-derived molecule can be confirmed by examining that the molecule or substance is a molecule or a substance that is exposed to extracellular environment where cell death has been induced.

A method of the following, including but not limited to, to examine and confirm whether a cell death is induced can be adopted.

Methods for detecting dead cells or dead cell-derived molecule or substance may involve detecting an increase in proportion of one or more of dead cell-associated properties, processes, or reduced proportion of viable cells. Such methods include, but are not particularly limited to, the detection of cells that stain positive for live cell impermeable DNA binding dyes, live cell impermeable amine reactive dyes, measurement of enzyme activity such as lactate dehydrogenase in dead cell supernatants, or reduced levels intracellular ATP associated with metabolically active live cells. Further, a method for detecting dead cells as reported by Riss may also used (Riss, Cytotoxicity Assays: In Vitro Methods to Measure Dead Cells; May 1, 2019).

In the present invention, whether certain molecule is a dead cell-derived molecule may be determined or confirmed by comparing a supernatant of cell culture in which a drug having cytotoxicity such as Mitoxantrone or Mitomycin C is added, with a supernatant of cell culture without the drug treatment. By conducting a proteome analysis and/or metabolome analysis of proteins and/or metabolites contained in each of the supernatant of the cell culture which is cultured in the presence of the cytotoxic drug, and that of the cell culture with no addition of the drug, molecules detected relatively in high amount in the supernatant of the cell culture which is cultured with addition of a cytotoxic drug may be determined as a dead cell-derived molecule (i.e., a substance which is exposed to a extracellular environment due to a cell death). Here, the drug to be added to the cell culture is not limited to Mitoxantrone or Mitomycin C, and any drug as long as it has cytotoxicity may be used.

In the present invention, a method to induce a cell death is not limited to the above-described method using a cytotoxic drug. For example, the cell death can be induced physically by a freeze-thaw treatment of cells.

Further, in the present invention, whether certain molecule or substance is a dead cell-derived molecule or a dead cell-derived substance may be determined or confirmed, for example, by preparing the cells treated or untreated with the above-drug, which the treatment with the drug induces a cell death, and measuring and comparing a degree of the expression of one or more membrane proteins in each group of the cells using a flow cytometry. The membrane protein which an increased expression is found in the drug-treated cells relatively to the expression in the non-treated cells is determined as a molecule that may be used as the dead cell-derived molecule.

In the present invention, the dead cell-derived molecule such as, including but not limited to, RNA, DNA, filamentous action, histones, phosphatidylserine or heat shock proteins, as described above, may be quantified using any means and/or methods for quantification well known by persons of skill in the art, e.g., a column chromatograph, mass spectrography (which term is used interchangeably herein with mass spectrometry), ELISA or the like. As long as the dead cell-derived molecule in the present invention is able to be quantified, any other means and/or methods for quantification may be used.

In one aspect of the present invention, the first antigen is selected from the group consisting of a) Splicing Factor 3B Subunit 3 (SF3B3); and b) Proliferating Cell Nuclear Antigen (PCNA).

The terms “SF3B3 splicing factor 3b subunit 3” and “SF3B3” are used interchangeably herein and refer to the subunit 3 of the splicing factor 3b protein complex. The sequence of human SF3B3 is disclosed as translated protein sequence in NCBI Reference Sequence: NP_036558.3, version of 22 May 2022.

In one embodiment, SF3B3 is human SF3B3.

In one embodiment, SF3B3 is human SF3B3 disclosed as translated protein sequence in NCBI Reference Sequence: NP_036558.3, version of May 22, 2022.

Splicing factor 3b, together with splicing factor 3a and a 12S RNA unit, forms the U2 small nuclear ribonucleoproteins complex (U2 snRNP). The splicing factor 3b/3a complex binds pre-mRNA upstream of the intron's branch site in a sequence independent manner and may anchor the U2 snRNP to the pre-mRNA. Splicing factor 3b is also a component of the minor U12-type spliceosome. Subunit 3 has also been identified as a component of the STAGA (SPT3-TAF (II) 31-GCN5L acetylase) transcription coactivator-HAT (histone acetyltransferase) complex, and the TFTC (TATA-binding-protein-free TAF (II)-containing complex). These complexes may function in chromatin modification, transcription, splicing, and DNA repair.

The terms “Proliferating cell nuclear antigen” and “PCNA” are used interchangeably herein and refer to the protein known in the art as Proliferating cell nuclear antigen.

Proliferating cell nuclear antigen or PCNA has been described to function as a DNA clamp that acts as a processivity factor for DNA polymerase delta in eukaryotic cells and is essential for replication. In vivo, PCNA is described to be a homotrimer. PCNA has been described to act as a scaffold to recruit proteins involved in DNA replication, DNA repair, chromatin remodeling and epigenetics.

A sequence of human PCNA is disclosed as translated protein sequence in NCBI Reference Sequence: NP_872590.1, version of Jun. 19, 2022. Further, a sequence of human PCNA is disclosed as translated protein sequence in NCBI Reference Sequence: NM_182649.2, version of Jun. 19, 2022.

In one embodiment, PCNA is human PCNA.

In one embodiment, PCNA is human PCNA disclosed as translated protein sequence in NCBI Reference Sequence: NP_872590.1, version of Jun. 19, 2022.

### Capable of Binding

In the context of the present invention, the term “capable of binding to” and “binding to” are used interchangeably and denote the capacity of a moiety to bind to an antigen in particular under physiological conditions, i.e. under conditions found in the animal, in particular human body. Particular exemplary methods for determining said capability are described herein below. As used herein, “capability of binding to an antigen” and suchlike may also be designated as “binding affinity for an antigen” and suchlike terms. Furthermore, in case of antibodies, said capability may also be described by using the term “an antibody directed against” or by using the term “an anti-antigen X antibody”.

First Moiety that Binds to a First Antigen

In the present invention, “a first moiety” of “a molecule comprising a first moiety that binds to a first antigen and a second moiety that binds to a second antigen” binds to “a first antigen”, which, as described above, is also referred as “a substance which is exposed to a extracellular environment due to a cell death”, and “a component or a portion thereof which constitutes a cytoskeleton, a cell membrane, a organelle, a cytoplasmic protein, or a metabolite, which is exposed to a extracellular environment due to a cell death” interchangeably.

“A first moiety” of the molecule in the present invention may have any structure as long as it binds to “a first antigen” as described above. The structure of “a first moiety” may include but is not limited to, a polypeptide or a portion thereof, or a small or medium chemical compound or a portion thereof, or a polynucleotide or a portion thereof. The polypeptide or a portion thereof includes but is not limited to a cell membrane protein expressed on a cell (e.g., an immune cell such as a dendritic cell) or a portion thereof (e.g., an extracellular domain, any unique domain thereof; an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region,), a single-domain antibody (sdAb), a single-chain Fv (scFv), a single-chain antibody, a Fv, a single-chain Fv2 (scFv2), a Fab, and F(ab′)₂.

The polypeptide or a portion thereof may also be an antigen binding polypeptides such as a module called A domain of Avimer, which has approximately 35 amino acids contained in an in vivo cell membrane protein (WO2004/044011 and WO2005/040229), adnectin having a 10Fn3 domain serving as a protein binding domain, which is derived from a glycoprotein fibronectin expressed on cell membranes (WO2002/032925), Affibody having an IgG binding domain scaffold constituting a three-helix bundle composed of 58 amino acids of protein A (WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (WO2002/020565), anticalin having four loop regions connecting eight antiparallel strands bent toward the central axis in one end of a barrel structure highly conserved in lipocalin molecules such as neutrophil gelatinase-associated lipocalin (NGAL) (WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (WO2008/016854).

One embodiment of a polypeptide or a portion thereof which belongs to “a first moiety that binds to a first antigen” as described above in the present invention includes but is not limited to a cell membrane protein expressed on a cell (e.g., a receptor) or a portion thereof (e.g., an extracellular domain, any unique domain thereof). One representative of the receptor or a portion thereof that belongs to “a first moiety that binds to a first antigen” in the present invention includes but is not limited to a scavenger receptor, a toll-like receptor (TLR), a C-type lectin (CLEC) such as Clec9A expressed on a dendritic cell, NOD-like receptor (NLR) (J Biol Chem. 2014 Dec. 19;289 (51): 35237-45), and so on, or a portion thereof. As examples, and not limiting thereto, a polypeptide or a portion thereof which belongs to “a first moiety that binds to a first antigen” as described above in the present invention includes CD36, RAGE, NLRP1, NLRP3, CD147, CD91, TLR2, TLR3, TLR4, TLR7, TLR8, TLR9, AIM2, TIM3, SREC1, FEEL1, LOX-1, LRP1, CD14, IL-1R, ST2, RIG-I, MDA5, NKP44 etc.

One of preferable embodiments of “a first moiety that binds to a first antigen” in the present invention is Clec9A, which is a C-type lectin expressed on a dendritic cell (DC), and binds to a complex of cytoskeleton-forming filament, F-actin, when, due to a cell death, the F-actin complex is exposed to a extracellular environment. In the present invention, “a first moiety that binds to a first antigen” in the present invention includes but is not limited to a whole polypeptide, an extracellular domain (ECD) having any amino acid length, a C-type lectin domain (CTLD) or any portion of Clec9A (GenBank: NP_001192292.1). One of preferable embodiments of “a first moiety that binds to a first antigen” in the present invention is Clec4e, which is also known as “C-type lectin domain family 4 member E”, or “Macrophage inducible Ca2+dependent lectin receptor”, or “Mincle”. The Splicing Factor 3B Subunit 3 (SF3B3) is reportedly released during cell death and signals through Mincle. In the present invention, “a first moiety that binds to a first antigen” in the present invention includes but is not limited to a whole polypeptide, or an antigen-binding portion of Clec4e as described in UniProt Accession number Q9ULY5 (version as in force on Jul. 28, 2022).

In a preferred embodiment herein, Clec4e is human Clec4e. In one preferred embodiment, “a first moiety that binds to a first antigen” in the present invention is the whole polypeptide, or an antigen-binding portion of human Clec4e, e.g. as described in UniProt Accession number Q9ULY5 (version as in force on Jul. 28, 2022).

Second Moiety that Binds to a Second Antigen

In the present invention, “a second moiety” of “a molecule comprising a first moiety that binds to a first antigen and a second moiety that binds to a second antigen” binds to “a second antigen” which is different from the “first antigen” as described above. As long as it is different from the first antigen, “a second antigen” may be any antigen. In the present invention, “a second antigen” is also referred as “a target molecule” which is targeted by “a second moiety” of “a molecule comprising a first moiety that binds to a first antigen and a second moiety that binds to a second antigen”. Upon the targeting of “a second antigen” by “a second moiety” of the molecule in the present invention, the target molecule, i.e., “a second antigen”, lead to a desirable outcome for the disease indicated, for instance, immune activation for combating of infection or cancer, immune inhibition for autoimmune diseases and/or cytokine release syndrome (CRS), direct inhibition of cell growth of cancer, or promotion of cell regeneration following tissue damage.

In the present invention, one common embodiment of the immune activation target includes but is not limited to CD3 (“CD epsilon”), CD4, CD8, CD28, OX40, 4-1BB and T-cell receptors (TCRs) expressed on T cells, which result in activating cytotoxic or T-helper functions for elimination of pathogens, infected or transformed cells. The target molecule (i.e., “a second antigen”) may also be expressed on antigen presenting cells (APCs), including XCR1, Clec9A, DEC-205, DCIR2, TLR3, TLR5, Flt3 on dendritic cells (DCs). The activation of DCs may indirectly lead to elimination of pathogens and cancer cells through priming and activation of adaptive immune cell types, including cytotoxic and helper T cells. A case in point would be targeting of CD40 (ESMO Open. 2019; 4 (Suppl 3): e000510) by CD40 agonists for anti-tumor effects through enhancement of antigen cross presentation and co-stimulatory capacities for more effective activation of cytotoxic T cells. Furthermore, synergistic effects leading to stronger antigen processing immune cell priming abilities could be achieved with parallel activation of the polyinosinic: polycytidylic acid (poly IC) sensing receptor, TLR3, also expressed by DCs (J Clin Invest. 2018; 128 (10): 4387-4396). In other cases, the inhibition of target molecule may be desired, such as antagonizing CTLA-4, PD-1, LAG-3, TIM-3, VISTA on T cells to prevent suppression of immune responses against tumor cells. The molecules to be targeted (i.e., “a second antigen”) may also be any molecule(s) expressed on or in other immune cell types, such as natural killer cells, macrophages neutrophils and their corresponding activating or inhibitory receptors. Oncogenic receptors on cancer cells or immune evasion molecules on transformed or infected cells may also be targeted (i.e., “a second antigen”).

In the present invention, “a second antigen” as briefly described above includes but is not limited to a membrane protein expressed on a cell membrane and/or an endosome membrane in a cell. One embodiment of the membrane protein (i.e., a second antigen) to which “a second moiety” binds includes but is not limited to a membrane protein involved in a signal transmission in a cell which is expressed on a cell membrane and/or an endosome of an immune cell such as a T cell, a killer cell, a helper T cell, a regulatory T cell, a B cell, a memory B cell, a NK cell, a NKT cell, a dendritic cell, a macrophage, an eosinophil, a neutrophil cell, and a basophil. One further embodiment of the membrane protein (i.e., a second antigen) to which “a second moiety” binds includes but is not limited to a membrane protein expressed on a tumor cell or an autoreactive cell.

In the present invention, the membrane protein that belongs to “a second antigen” as described above includes but is not limited to a costimulatory molecule such as CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), or CD244 (2B4); or a coinhibitory molecule such as CD279 (PD-1), CD152 (CTLA-4), TIGIT, BTLA, CD366 (Tim-3), CD96, CD112R, or CD200R; or a costimulatory molecule or a coinhibitory molecule such as CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), or Gal-9.

As further embodiments of the membrane protein that belongs to “a second antigen” as described above includes but is not limited to CCR, CCR1, CCR10, CCR10, CCR2, CCR3, CCR4, CCR5, CCR6, CCR7, CCR8, CCR9, CD1, CD2, CD3 (e.g., CD3 epsilon), CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD29, CD30L, CD32, CD33 (p67 protein), CD34, CD36, CD38, CD40L, CD44, CD45, CD46, CD49a, CD52, CD54, CD55, CD56, CD61, CD64, CD66e, CD74, CD89, CD91, CD95, CD123, CD137, CD138, CD140a, CD146, CD147, CD148, CD152, CD164, CEACAM5, CFTR, CXCR, CXCR1, CXCR2, CXCR3, CXCR4, CXCR5, CXCR6; fibroblast-activating protein (FAP), Fas, cytokine receptors such as IL-1R, IL-2R, IL-4R, IL-5R, IL-6R, IL-1OR, IL-12R, IL-15R, IL-17R, IL-18R, IL-20R, or IL-31R; IL1RL1 (ST2), integrin family such as alpha 1 beta 1 (VLA-1), alpha 2 beta 1 (VLA-2), alpha 3 beta 1 (VLA-3), alpha 4 beta 1 (VLA-4), alpha 5 beta 1 (VLA-5), alpha 6 beta 1 (VLA-6), alpha 7 beta 1, alpha 8 beta 1, alpha 9 beta 1, alpha 11b beta 3 (GPIIb/IIIa), alpha V beta 1, alpha V beta 3, alpha V beta 5, alpha V beta 6, alpha V beta 8, alpha L beta 2 (LFA-1), alpha M beta 2 (Mac-1), alpha X beta 2, alpha D beta 2, alpha 4 beta 7; LOX-1, MDA5 (melanoma differentiation-associated protein 5), TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TLR (Toll-like receptor) 1, TLR2, TLR3, TLR4, TLR5, TLR6, TLR7, TLR8, TLR9, and TLR10, RIG-I; RAGE, P2Y2, P2X7, secreted frizzled related protein 1 (FRP1), and Interferon-inducible protein (AIM2).

In the present invention, “a second antigen” also includes but is not limited to a soluble protein which may bind to the membrane protein as described above. In the present invention, the soluble protein that belongs to “a second antigen” includes but is not limited to cytokines such as IL-1, IL-2, IL-4, Il-5, IL-6, IL-8, IL-9, IL-10, IL-12, IL-13, IL-15, IFN beta, IFN gamma, IL-18, IL21, IL-23, and IL-27.

In the present invention, the soluble protein that belongs to “a second antigen” further includes but is not limited to the following molecules or any soluble form of the molecule (e.g., a truncated form of a membrane protein): 17-IA, 4-1BB, 4Dc, 6-keto-PGF1a, 8-iso-PGF2a, 8-oxo-dG, A1 adenosine receptor, A33, ACE, ACE-2, activin, activin A, activin AB, activin B, activin C, activin RIA, activin RIA ALK-2, activin RIBALK-4, activin RIIA, activin RIIB, ADAM, ADAM10, ADAM12, ADAM15, ADAM17/TACE, ADAM8, ADAM9, ADAMTS, ADAMTS4, ADAMTS5, addressin, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, artemin, anti-Id, ASPARTIC, atrial natriuretic factor, av/b3 integrin, Axl, b2M, B7-1, B7-2, B7-H, B lymphocyte stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 osteogenin, BMP-4, BMP-2b, BMP-5, BMP-6 Vgr-1,BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMP, b-NGF, BOK, bombesin, bone-derived neurotrophic factor, BPDE, BPDE-DNA, BTC, complement factor 3 (C3), C3a, C4, C5, C5a,C10, CA125, CAD-8, calcitonin, cAMP, carcinoembryonic antigen (CEA), cancer-associated antigens, cathepsin A, cathepsin B, cathepsin C/DPPI, cathepsin D, cathepsin E, cathepsin H, cathepsin L, cathepsin O, cathepsin S, cathepsin V, cathepsin X/Z/P, CBL, CCI, CCK2, CCL, CCL1, CCL11, CCL12, CCL13, CCL14, CCL15, CCL16, CCL17, CCL18, CCL19,CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67protein), CD34, CD36, CD38, CD40, CD40L, CD44, CD45, CD46, CD49a, CD52, CD54, CD55, CD56, CD61, CD64, CD66e, CD74, CD80 (B7-1), CD89, CD91, CD95, CD123, CD137, CD138, CD140a, CD146, CD147, CD148, CD152, CD164, CEACAM5, CFTR, cGMP, CINC, botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, PD-1, PD-L1, LAG3,TIM3, galectin-9, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigens, DAN, DCC, DcR3, DC-SIGN, decay accelerating factor, des (1-3)-IGF-I (brain IGF-1), Dhh, digoxin, DNAM-1, Dnase, Dpp, DPPIV/CD26,Dtk, ECAD, EDA, EDA-A1, EDA-A2, EDAR, EGF, EGFR (ErbB-1), EMA, EMMPRIN,ENA, endothelin receptor, enkephalinase, eNOS, Eot, eotaxin 1, EpCAM, ephrin B2/EphB4,ePO, ERCC, E-selectin, ET-1, factor IIa, factor VII, factor VIIIc, factor IX, fibroblast, activating protein (FAP), Fas, FcR1, FEN-1, ferritin, FGF, FGF-19, FGF-2, FGF3, FGF-8,FGFR, FGFR-3, fibrin, FL, FLIP, Flt-3, Flt-4, follicle-stimulating hormone, fractalkine, FZD1, FZD2, FZD3, FZD4, FZD5, FZD6, FZD7, FZD8, FZD9, FZD10, G250, Gas6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (myostatin), GDF-9, GDF-15 (MIC-1), GDNF, GDNF, GFAP, GFRa-1, GFR-alpha 1, GFR-alpha 2, GFR-alpha 3, gITR, glucagon, Glut4, glycoprotein IIb/IIIa (GPIIb/IIIa), GM-CSF, gp130, gp72, GRO, growth hormone-releasing factor, hapten (NP-cap or NIP-cap), HB-EGF, HCC, HCMV gB envelope glycoprotein, HCMV gH envelope glycoprotein, HCMV UL, hematopoietic growth factor (HGF), Hep B gp120, heparanase, Her2, Her2/neu (ErbB-2), Her3 (ErbB-3), Her4 (ErbB-4),herpes simplex virus (HSV) gB glycoprotein, HSV gD glycoprotein, HGFA, high-molecular weight melanoma-associated antigen (HMW-MAA), HIV gp120, HIV IIIB gp 120 V3 loop, HLA, HLA-DR, HM1.24, HMFG PEM, HRG, Hrk, human heart myosin, human cytomegalovirus (HCMV), human growth hormone (HGH), HVEM, I-309, IAP, ICAM,ICAM-1, ICAM-3, ICE, ICOS, IFNg, Ig, IgA receptor, IgE, IGF, IGF binding protein, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-21, IL-23, IL-27, interferon (INF)-alpha, INF-beta, INF-gamma, inhibin, iNOS, insulin chain A, insulin chain B, insulin like growth factor 1, integrin alpha 2, integrin alpha 3, integrin alpha 4, integrin alpha 4/betal, integrin alpha 4/beta 7, integrin alpha 5 (alpha V), integrin alpha 5/beta 1, integrin alpha5/beta 3, integrin alpha 6, integrin beta 1, integrin beta 2, interferon gamma, IP-10, I-TAC,JE, kallikrein 2, kallikrein 5, kallikrein 6, kallikrein 11, kallikrein 12, kallikrein 14, kallikrein15, kallikrein L1, kallikrein L2, kallikrein L3, kallikrein L4, KC, KDR, keratinocyte growth factor (KGF), laminin 5, LAMP, LAP, LAP (TGF-1), latent TGF-1, latent TGF-1 bp1, LBP, LDGF, LECT2, lefty, Lewis-Y antigen, Lewis-Y-related antigen, LFA-1, LFA-3, Lfo, LIF,LIGHT, lipoprotein, LIX, LKN, Lptn, L-selectin, LT-a, LT-b, LTB4, LTBP-1, lung surface, luteinizing hormone, lymphotoxin beta receptor, Mac-1, MAdCAM, MAG, MAP2, MARC,MCAM, MCAM, MCK-2, MCP, M-CSF, MDC, Mer, metalloproteinases, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Mullerian inhibiting substance, Mug, MuSK, NAIP, NAP, NCAD, N—C adherin, NCA 90, NCAM, neprilysin, neurotrophin-3,-4, or-6, neurturin, nerve growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, parathyroid hormone, PARC, PARP, PBR, PBSF,PCAD, P-cadherin, PDGF, PDGF, PDK-1, PECAM, PEM, PF4, PGE, PGF, PGI2, PGJ2, PIN, PLA2, placental alkaline phosphatase (PLAP), PIGF, PLP, PP14, proinsulin, prorelaxin, protein C, PS, PSA, PSCA, prostate-specific membrane antigen (PSMA), PTEN, PTHrp, Ptk, PTN, R51, RANK, RANKL, RANTES, RANTES, relaxin A chain, relaxin Bchain, renin, respiratory syncytial virus (RSV) F, RSV Fgp, Ret, rheumatoid factor, RLIP76,RPA2, RSK, S100, SCF/KL, SDF-1, SERINE, serum albumin, sFRP-3, Shh, SIGIRR, SK-1,SLAM, SLPI, SMAC, SMDF, SMOH, SOD, SPARC, Stat, STEAP, STEAP-II, TACE,TACI, TAG-72 (tumor-associated glycoprotein-72), TARC, TCA-3, T cell receptor (e.g., Tcell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta 1, TGF-beta2, TGF-beta 3, TGF-beta 4, TGF-beta 5, thrombin, thymus Ck-1, thyroid stimulating hormone, Tie, TIMP, TIQ, tissue factor, TMEFF2, Tmpo, TMPRSS2, TNF, TNF-alpha, TNF-alpha/beta, TNF-beta 2, TNFc, TNF-RI, TNF-RII, TNFRSF10A (TRAIL R1 Apo-2,DR4), TNFRSF10B (TRAIL R2 DR5, KILLER, TRICK-2A, TRICK-B), TNFRSF10C (TRAIL R3 DcR1, LIT, TRID), TNFRSF1OD (TRAIL R4 DcR2, TRUNDD), TNFRSF11A (RANK ODF R, TRANCE R), TNFRSF11B (OPG OCIF, TR1), TNFRSF12 (TWEAK RFN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITRAITR), TNFRSF19 (TROY TAJ, TRADE), TNFRSF19L (RELT), TNFRSF1A (TNF RICD120a, p55-60), TNFRSF1B (TNF RII CD120b, p75-80), TNFRSF26 (TNFRH3),TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6),TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 ligand, TL2), TNFSF11 (TRANCE/RANK ligand ODF, OPG ligand), TNFSF12 (TWEAK Apo-3 ligand,DR3 ligand), TNFSF13 (APRIL TALL2), TNFSF13B (BAFF BLYS, TALL1, THANK,TNFSF20), TNFSF14 (LIGHT HVEM ligand, LTg), TNFSF15 (TL1A/VEGI), TNFSF18 (GITR ligand AITR ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 ligand gp34, TXGP1), TNFSF5 (CD40 ligand CD154, gp39, HIGM1, IMD3, TRAP), TNFSF6 (Fas ligand Apo-1ligand, APT1 ligand), TNFSF7 (CD27 ligand CD70), TNFSF8 (CD30 ligand CD153),TNFSF9 (4-1BB ligand CD137 ligand), TP-1, t-PA, Tpo, TRAIL, TRAIL R, TRAIL-R1,TRAIL-R2, TRANCE, transferrin receptor, TRF, Trk, TROP-2, TLR (toll-like receptor) 1,TLR2, TLR3, TLR4, TLR5, TLR6, TLR7, TLR8, TLR9, TLR10, TSG, TSLP, tumor associated antigen CA125, tumor-associated antigen-expressing Lewis-Y-related carbohydrate, TWEAK, TXB2, Ung, uPAR, uPAR-1, urokinase, VCAM, VCAM-1, VECAD, VE-cadherin, VE-cadherin-2, VEFGR-1 (flt-1), VEGF, VEGFR, VEGFR-3 (flt-4), VEGI, VIM, viral antigens, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, HMGB1, IgA,CD81, CD97, CD98, DDR1, DKK1, EREG, Hsp90, IL-17/IL-17R, IL-20/IL-20R, oxidized LDL, PCSK9, prekallikrein, RON, TMEM16F, SOD1, chromogranin A, chromogranin B, tau, VAP1, high-molecular-weight kininogen, IL-31, IL-31R, ILIRL1 (ST2), Nav1.1, Nav1.2, Nav1.3,Nav1.4, Nav1.5, Nav1.6, Nav1.7, Nav1.8, Nav1.9, EPCR, C1, C1q, Clr, Cls, C2, C2a, C2b, C3, C3a, C3b, C4, C4a, C4b, C5, C5a, C5b, C6, C7, C8, C9, factor B, factor D, factor H, properdin, sclerostin, fibrinogen, fibrin, prothrombin, thrombin, tissue factor, factor V, factor Va, factor VII, factor VIIa, factor VIII, factor VIIIa, factor IX, factor IXa, factor X, factorXa, factor XI, factor XIa, factor XII, factor XIIa, factor XIII, factor XIIIa, TFPI, antithrombin III, EPCR, thrombomodulin, TAPI, tPA, plasminogen, plasmin, PAI-1, PAI-2, GPC3, syndecan-1, syndecan-2, syndecan-3, syndecan-4, LPA, S1P, and receptors for hormones or growth factors.

Although the examples of the molecule listed above also include receptors (membrane proteins), these receptors (membrane proteins) even existing in a soluble form in a body fluid (e.g., as a truncated form) can be used as “a second antigen” (a target molecule) to which “a second moiety” in the present invention binds. One non-limiting example the soluble form of such a receptor (membrane protein) can include the protein represented by soluble IL-6R as described by Mullberg et al. (J. Immunol. (1994) 152 (10), 4958-4968).

In the present invention, in case that “a second antigen” is a soluble protein, for example, IL-6, a molecule comprising a first moiety and a second moiety of the present invention binds to the dead cell-derived molecule (i.e., a first antigen), e.g., F-actin complex, via first moiety of the molecule, and binds to IL-6 (i.e., a second antigen) via second moiety of the molecule to neutralize IL-6 in an affected tissue more specifically where the dead cell-derived molecules are occurred due to a cell death than in non-affected tissue. Upon the neutralization of IL-6, an immune response caused by immune cells is indirectly regulated.

In the present invention, “a second moiety” that binds to “a second antigen” as described above may have any structure as long as it binds to “a second antigen” as described above. The structure of “a second moiety” may include but is not limited to, a polypeptide or a portion thereof, or a small or medium chemical compound or a portion thereof, or a polynucleotide or a portion thereof. The polypeptide or a portion thereof includes but is not limited to an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region,), a single-domain antibody (sdAb), a single-chain Fv (scFv), a single-chain antibody, a Fv, a single-chain Fv2 (scFv2), a Fab, and F(ab′)₂.

Molecule Comprising a First Moiety that Binds to a First Antigen and a Second Moiety that Binds to a Second Antigen

In the present invention, one embodiment of “a molecule comprising a first moiety that binds to a first antigen and a second moiety that binds to a second antigen” includes but is not limited to a molecule having the following feature.

i. An antibody which “a first moiety that binds to a first antigen” has been conjugated to a Fc region, and at least one of the two Fabs binds “a second antigen”. One schematic example of this embodiment is as shown in FIG. 2. Alternatively, “a first moiety that binds to a first antigen” may be conjugated to one of Fab arms instead of, or in addition to the conjugation to Fc region. Further, more than two “a first moiety that binds to a first antigen” may be conjugated to an Fc region. Further various examples of the embodiments of a molecule of the present invention are schematically illustrated in FIG. 3. The two Fab arms can conjugated to Fc region in various formats as illustrated in FIG. 3. In this embodiment, one example of “a first moiety that binds to a first antigen” is a whole or a portion of Clec9A or a VHH.

ii. An antibody which one Fab arm binds “a first antigen” and another Fab arm binds “a second antigen”. One example of this embodiment may be a bispecific antibody or a bispecific antigen-binding domain, which one Fab arm binds to the dead-cell derived molecule (e.g., F-actin complex) as described above, and another Fab arm binds to a membrane protein expressed on a cell membrane of a cell (e.g., an immune cell, an cancer cell, an autoreactive cell). The antibody or antibody-like molecule in this example can have various formats as illustrated in FIG. 3.

iii. A polypeptide comprising “a first moiety that binds to a first antigen” and “a second moiety that binds to a second antigen”, which the first moiety and the second moiety are fused with or without a linker. One example of this embodiment is a fusion protein comprising a extracellular domain of Clec9A and a ligand for any membrane protein expressed on a cell membrane of a cell (e.g., an immune cell, an cancer cell, an autoreactive cell).

iv. A chemical compound comprising “a first moiety that binds to a first antigen” and “a second moiety that binds to a second antigen”. One example of this embodiment may be a chemical compound comprising at least two moieties, which one moiety binds to the dead-cell derived molecule (e.g., F-actin complex, a nucleosome which is a complex of a histone and a nucleic acid) as described above, and other moiety arm binds to a membrane protein expressed on a cell membrane of a cell (e.g., an immune cell, an cancer cell, an autoreactive cell). Such chemical compounds may be carried out by using a bioconjugate technique (Bioconjugate Techniques, 3rd Edition, 2013, by Greg T. Hermanson). For example, in case that “a first antigen” is a nucleosome which is a complex of a histone and a nucleic acid, and “a second antigen” is a mTOR (mechanistic Target Of Rapamycin), the chemical compound may be a compound comprising Distamycine as “a first moiety” that binds to a nucleic acid and Rapamycin as “a second moiety” that binds to mTOR, and further comprising a photoreactive group (e.g., aryl azide, diazirine, psolalen). Upon receipt of a photo (light) energy by the compound, the first antigen and the second antigen is crosslinked by the compound, and, the binding of the second moiety to the second antigen on a cell membrane confers a signal transmission or a signal blockage into the cell.

### Conferring a Signal Transmission or a Signal Blockade

In the present invention, “conferring a signal transmission or a signal blockade” by “a molecule comprising a first moiety that binds to a first antigen and a second moiety that binds to a second antigen” into a cell may be achieved by providing a modulation into the cell, e.g., agonistic activity, an antagonistic activity, an allosteric modulation, allosteric modulation, conformational change, an internalization, a stabilization, or whatever the target molecule (i.e., a second antigen) is affected by the molecule of the present invention.

### Affected Tissue

In the present invention, the term “an affected tissue” means a tissue where any different condition or feature from that of a normal tissue and is unique for a disease is found. One example of the affected tissue includes but is not limited to a tumor tissue, an inflammatory tissue, a tissue associated with an autoimmune disease, and so on. In such affected tissues, a cell death as described above occurs more frequently than normal tissue, to generate the dead cell derived molecules as described above.

### Tumor Tissue

In the present invention, the term “tumor tissue” means a tissue that comprises at least one tumor cell. Generally, a tumor tissue is made of a population of tumor cells constituting the tumor main body (parenchyma) and connective tissues and blood vessels existing in between tumor cells and supporting the tumor (stroma). In some cases, these are clearly distinguishable, but there are cases where these are mixed up. In some cases, there are cells such as immune cells that have infiltrated into the tumor tissue. In contrast, “non-tumor tissue” means a tissue in the living body other than tumor tissue(s). Non-diseased healthy tissues/normal tissues are representatives of such non-tumor tissues.

### Inflammatory Tissue

In the present invention, “an inflammatory tissue” includes but is not limited to the following.
i. A joint tissue associated with rheumatoid arthritis or osteoarthritis.
ii. A lung tissue associated with bronchial asthma.
iii. A digestive organ tissue associated with inflammatory bowel disease, Crohn disease, or ulcerative colitis.
iv. A fibrotic tissue associated with a fibrosis in liver, kidney or lung.
v. A tissue associated with an immune rejection due to an organ plant.
vi. A blood vessel tissue or a heart tissue associated with arteriosclerosis or heart failure.
vii. A visceral fat tissue associated with metabolic syndrome.
viii. A skin tissue associated with atopic dermatitis or any other dermatitis.
ix. A spinal nerve tissue associated with disc hernia or chronic back pain.
x. A bone tissue associated with a bone fracture.
xi. A tissue associated with a burn.
xii. A tissue of injured organ tissue by mechanical, physical or chemical external force.
   Antigen which is Formed by Multimerization of a Plural Number of Molecules

In the present invention, one embodiment of “a first antigen” as described includes but is not limited to “an antigen which is formed by multimerization of a plural number of molecules”. In the present invention, “an antigen which is formed by multimerization of a plural number of molecules” includes but is not limited to an antigen formed by multimerization of a plural number of proteins or portions thereof and/or lipids, which constitute a cytoskeleton, a biomembrane such as a cell membrane, a nucleosome, a chaperone molecule or the like.

The proteins or portions thereof include but are not limited to proteins or portions thereof which constitute a cytoskeleton which is composed of an actin filament, an intermediate filament, a myosin filament, a keratin filament, a microtubule and so on. The biomembrane such as a cell membrane is composed of various complexes formed by different proteins, different lipids, and/or one or more proteins and one or more lipids. Such proteins include but are not limited to transmembrane proteins such as ion channels, G-protein coupled receptors (GPCRs), proton pumps, lipid-anchor proteins such as G-protein and so on, and superficial membrane proteins such as enzymes, hormones, and so on. The lipids include but are not limited to phospholipids, glycolipids, sterols and so on. As described above, in an affected tissue such as a tissue associated with a cancer, an inflammation, an autoimmune disease or the like, due to a cell death, such complexes of proteins and/or lipids in the biomembrane such as a cell membrane, or a portion thereof are exposed to a extracellular environment. Such complex or a portion thereof is also included as “a first antigen” (also referred as a dead cell-derived molecule) as described above.

The nucleosome is a complex of a histone and a nucleic acid, and the chaperone molecule is composed of mainly heat shock proteins (HSPs). Those are also included as “a first antigen” (also referred as a dead cell-derived molecule).

Cell which a Membrane Protein as “a Second Antigen” is Expressed

In the present invention, as described above, “a second antigen” includes but is not limited to a membrane protein expressed on a cell membrane and/or an endosome membrane involved in a signal transmission in a cell. In the present invention, such cell which a membrane protein as “a second antigen” is expressed may be any sort of or any type of cells, including but not limited to an immune cell (such as a T cell, a killer cell, a helper T cell, a regulatory T cell, a B cell, a memory B cell, a NK cell, a NKT cell, a dendritic cell, a macrophage, an eosinophil, a neutrophil cell, and a basophil), a somatic cell (e.g., epithelial cell, fibroblastic cell, interstitial cell, bone cell, muscle cell, nerve cell, blood cell or the like), a reproductive cell, or an affected or abnormal cell (e.g., a tumor cell, an autoreactive cell). Such cells may be located in any tissue, including but not limited to, epithelial tissue, muscle tissue, nerve tissue, connective tissue, or an affected tissue associated with a cancer, an inflammation, and autoimmune disease or the like. Preferable embodiments of the cell which “a second antigen” (also referred as a target molecule) is expressed may be an immune cell such as a T cell, a killer cell, a helper T cell, a regulatory T cell, a B cell, a memory B cell, a NK cell, a NKT cell, a dendritic cell, a macrophage, an eosinophil, a neutrophil cell, and a basophil. Other preferable embodiments of the cell which “a second antigen” (also referred as a target molecule) is expressed may be a cell in an affected tissue associated with a cancer, an inflammation, and autoimmune disease or the like.

In the present invention, the binding of “a molecule comprising a first moiety that binds to a first antigen and a second moiety that binds to a second antigen” of the present invention as described above to “a substance (a molecule) which is exposed to a extracellular environment due to a cell death” (“a dead cell-derived molecule”) as also described above can be examined, for example, by preparing the cells treated or untreated with a drug having cytotoxicity such as Mitoxantrone or Mitomycin, which the treatment with the drug induces a cell death, and measuring and comparing a degree of the binding of the molecule to each of the drug-treated cells or non-treated cells using a flow cytometry. The increased binding degree of the molecule of the present invention as described above to the drug-treated cells shows that the molecule of the present invention specifically binds to the dead cell-derived molecule as described above.

The present invention provides a molecule which binds to a component or a portion thereof of a cell which is exposed to a extracellular environment due to a cell death (“a first antigen”, also referred as a dead cell-derived molecule) as described above, and concurrently binds to a target molecule (“a second antigen”; e.g., a membrane protein, as described above) on or in a cell (e.g., an immune cell, an affected cell such as a tumor cell, an autoreactive cell, and an virus-infected cell). The molecule enables to crosslink between the component or a portion thereof of a cell which is exposed to a extracellular environment due to a cell death and the target molecule on or in a cell such as immune cell or an affected cell (e.g., a cancer cell, an autoreactive cell, and an virus-infected cell), and confers a signal transmission or blockage into the cell in the tissue.

In the present invention, the above-described signal transmission or blockage into a cell by the molecule of the present invention can be examined, for example, as follows.
i. Providing cells treated or untreated with a drug having cytotoxicity, which the treatment with the drug induces a cell death.
ii. Providing a molecule (e.g., antibody) that binds to, e.g., CD3 or a costimulatory or coinhibitory molecule expressed in an immune cell (“a second antigen”) [Control molecule], and a molecule having a moiety that binds to the same (“a second antigen), and a moiety that binds to, e.g., F-actin (“a first antigen “; one of the embodiment of a dead cell derived molecule) [Molecule of the present invention].
iii. Providing reporter cells which have been modified to give a detectable signal when a signal transmission occurred by binding the molecule of (ii).
iv. Culturing the reporter cells in the presence of the drug-treated cells or a supernatant of the drug-treated cell culture, or non-treated cells or a supernatant of the non-treated cells of (i), with each of the molecule of (ii).
v. Measuring a reporter signal in each cell culture of (iv), and comparing the reporter signal so measured of each cell culture.
vi. Higher reporter signal be detected in the cell culture of the Molecule of the present invention of (ii) in the presence of the drug-treated cells or a supernatant of the drug-treated cell culture than that in the cell culture of the Molecule of the present invention of (ii) in the non-treated cells or a supernatant of the non-treated cell culture shows that the Molecule of the present invention of (ii) conferred a signal transmission into cells by binding to an antigen, e.g., CD3 or a costimulatory or coinhibitory molecule expressed in an immune cell (“a second antigen”).
vii. Higher reporter signal be detected in the cell culture of the Molecule of the present invention of (ii) in the presence of the drug-treated cells or a supernatant of the drug-treated cell culture than that in the cell culture of the Control of (ii) in the drug-treated cells or a supernatant of the drug-treated cell culture shows that the Molecule of the present invention of (ii) conferred stronger signal transmission into cells than the Control molecule which cannot bind to a dead cell derived molecule, e.g., F-actin (“a first antigen”).

In the present invention, as long as a signal transmission into a cell which is induced by binding of the Molecule of the present invention (above (ii)) to “a second antigen”, e.g., CD3 or a costimulatory or coinhibitory molecule expressed in an immune cell can be detected, any sort and/or any type of reporter, and any sort and/or any type of reporter cells may be used depending on “a second antigen”.

In the present invention, the “stronger” signal in the above (vii) means a difference in the maximum activity of at least 1.5 times, 2 times, 3 times, 4 times, 5 times, 10 times, 15 times, 20 times, 50 times, 100 times, 200 times, or 1,000 times. The “stronger” signal in the above (vii) can also be a difference in EC50 value or IC50 of at least 1.5 times, 2 times, 3 times, 4 times, 5 times, 10 times, 15 times, 20 times, 50 times, 100 times, 200 times, or 1,000 times.

When the terms described or referenced below are used in the present invention, those have, including but not limited to, such meaning as described or referred below.

Further, the techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F.M. Ausubel, et al. eds., (2003)); the series Methods in Enzymology (Academic Press, Inc.): PCR 2: A Practical Approach (M.J. MacPherson, B. D. Hames and G. R. Taylor eds. (1995)), Harlow and Lane, eds. (1988) Antibodies, A Laboratory Manual, and Animal Cell Culture (R.I. Freshney, ed. (1987)); Oligonucleotide Synthesis (M.J. Gait, ed., 1984); Methods in Molecular Biology, Humana Press; Cell Biology: A Laboratory Notebook (J. E. Cellis, ed., 1998) Academic Press; Animal Cell Culture (R.I. Freshney), ed., 1987); Introduction to Cell and Tissue Culture (J. P. Mather and P.E. Roberts, 1998) Plenum Press; Cell and Tissue Culture: Laboratory Procedures (A. Doyle, J. B. Griffiths, and D.G. Newell, eds., 1993-8) J. Wiley and Sons; Handbook of Experimental Immunology (D.M. Weir and C. C. Blackwell, eds.); Gene Transfer Vectors for Mammalian Cells (J.M. Miller and M.P. Calos, eds., 1987); PCR: The Polymerase Chain Reaction, (Mullis et al., eds., 1994); Current Protocols in Immunology (J.E. Coligan et al., eds., 1991); Short Protocols in Molecular Biology (Wiley and Sons, 1999); Immunobiology (C.A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V.T. DeVita et al., eds., J.B. Lippincott Company, 1993).

Third Moiety that Binds to a Third Antigen

In the present invention, “a third moiety” of an “antigen-binding molecule comprising at least one third moiety that binds to a third antigen” binds to “a third antigen” which is different from the “first antigen” as described above. Said third antigen is a membrane protein of an immune cell, or a tumour cell, which are described above. As long as it is different from the first antigen, “a third antigen” may be any membrane protein of an immune cell, or a tumour cell. In the present invention, “a third antigen” is also referred as “a target molecule” which is targeted by “a third moiety” of an “antigen-binding molecule comprising at least one third moiety that binds to a third antigen. Upon the targeting of “a third antigen” by “a third moiety” of the molecule in the present invention which comprises a third moiety, the target molecule, i.e., “a third antigen”, lead to a desirable outcome for the disease indicated, for instance, immune activation for combating of infection or cancer, immune inhibition for autoimmune diseases and/or cytokine release syndrome (CRS), direct inhibition of cell growth of cancer, or promotion of cell regeneration following tissue damage.

In embodiments of some aspects of the present invention, said third antigen is different from the first and second antigen. In embodiments of some aspects of the present invention, said third antigen is different from the first but same as the second antigen.

In the present invention, one common embodiment of the immune activation target includes but is not limited to CD3 (“CD epsilon”), CD4, CD8, CD28, OX40, 4-1BB and T-cell receptors (TCRs) expressed on T cells, which result in activating cytotoxic or T-helper functions for elimination of pathogens, infected or transformed cells. The target molecule (i.e., “a second antigen”) may also be expressed on antigen presenting cells (APCs), including XCR1, Clec9A, DEC-205, DCIR2, TLR3, TLR5, Flt3 on dendritic cells (DCs). The activation of DCs may indirectly lead to elimination of pathogens and cancer cells through priming and activation of adaptive immune cell types, including cytotoxic and helper T cells. A case in point would be targeting of CD40 (ESMO Open. 2019; 4 (Suppl 3): e000510) by CD40 agonists for anti-tumor effects through enhancement of antigen cross presentation and co-stimulatory capacities for more effective activation of cytotoxic T cells. Furthermore, synergistic effects leading to stronger antigen processing immune cell priming abilities could be achieved with parallel activation of the polyinosinic: polycytidylic acid (poly IC) sensing receptor, TLR3, also expressed by DCs (J Clin Invest. 2018; 128 (10): 4387-4396). In other cases, the inhibition of target molecule may be desired, such as antagonizing CTLA-4, PD-1, LAG-3, TIM-3, VISTA on T cells to prevent suppression of immune responses against tumor cells. The molecules to be targeted (i.e., “a third antigen”) may also be any molecule(s) expressed on or in other immune cell types, such as natural killer cells, macrophages neutrophils and their corresponding activating or inhibitory receptors. Oncogenic receptors on cancer cells or immune evasion molecules on transformed or infected cells may also be targeted (i.e., “a third antigen”).

In the present invention, “a third antigen” as briefly described above includes but is not limited to a membrane protein expressed on a cell membrane and/or an endosome membrane in a cell. One embodiment of the membrane protein (i.e., a third antigen) to which “a third moiety” binds includes but is not limited to a membrane protein involved in a signal transmission in a cell which is expressed on a cell membrane and/or an endosome of an immune cell such as a T cell, a killer cell, a helper T cell, a regulatory T cell, a B cell, a memory B cell, a NK cell, a NKT cell, a dendritic cell, a macrophage, an eosinophil, a neutrophil cell, and a basophil. One further embodiment of the membrane protein (i.e., a third antigen) to which “a third moiety” binds includes but is not limited to a membrane protein expressed on a tumor cell or an autoreactive cell.

In the present invention, the membrane protein that belongs to “a third antigen” as described above includes but is not limited to a costimulatory molecule such as CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), or CD244 (2B4); or a coinhibitory molecule such as CD279 (PD-1), CD152 (CTLA-4), TIGIT, BTLA, CD366 (Tim-3), CD96, CD112R, or CD200R; or a costimulatory molecule or a coinhibitory molecule such as CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), or Gal-9.

As further embodiments of the membrane protein that belongs to “a third antigen” as described above includes but is not limited to CCR, CCR1, CCR10, CCR10, CCR2, CCR3, CCR4, CCR5, CCR6, CCR7, CCR8, CCR9, CD1, CD2, CD3 (e.g., CD3 epsilon), CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD29, CD30L, CD32, CD33 (p67 protein), CD34, CD36, CD38, CD40L, CD44, CD45, CD46, CD49a, CD52, CD54, CD55, CD56, CD61, CD64, CD66e, CD74, CD89, CD91, CD95, CD123, CD137, CD138, CD140a, CD146, CD147, CD148, CD152, CD164, CEACAM5, CFTR, CXCR, CXCR1, CXCR2, CXCR3, CXCR4, CXCR5, CXCR6; fibroblast-activating protein (FAP), Fas, cytokine receptors such as IL-1R, IL-2R, IL-4R, IL-5R, IL-6R, IL-10R, IL-12R, IL-15R, IL-17R, IL-18R, IL-20R, or IL-31R; IL1RL1 (ST2), integrin family such as alpha 1 beta 1 (VLA-1), alpha 2 beta 1 (VLA-2), alpha 3 beta 1 (VLA-3), alpha 4 beta 1 (VLA-4), alpha 5 beta 1 (VLA-5), alpha 6 beta 1 (VLA-6), alpha 7 beta 1, alpha 8 beta 1, alpha 9 beta 1, alpha 11b beta 3 (GPIIb/IIIa), alpha V beta 1, alpha V beta 3, alpha V beta 5, alpha V beta 6, alpha V beta 8, alpha L beta 2 (LFA-1), alpha M beta 2 (Mac-1), alpha X beta 2, alpha D beta 2, alpha 4 beta 7; LOX-1, MDA5 (melanoma differentiation-associated protein 5), TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF1OD (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TLR (Toll-like receptor) 1, TLR2, TLR3, TLR4, TLR5, TLR6, TLR7, TLR8, TLR9, and TLR10, RIG-I; RAGE, P2Y2, P2X7, secreted frizzled related protein 1 (FRP1), and Interferon-inducible protein (AIM2) . . .

In the present invention, “a third antigen” also includes but is not limited to a soluble protein which may binds to the membrane protein as described above. In the present invention, the soluble protein that belongs to “a third antigen” includes but is not limited to cytokines such as IL-1, IL-2, IL-4, IL-5, IL-6, IL-8, IL-9, IL-10, IL-12, IL-13, IL-15, IFN beta, IFN gamma, IL-18, IL21, IL-23, and IL-27.

In the present invention, the soluble protein that belongs to “a third antigen” further includes but is not limited to the following molecules or any soluble form of the molecule (e.g., a truncated form of a membrane protein): 17-IA, 4-1BB, 4Dc, 6-keto-PGF1a, 8-iso-PGF2a, 8-oxo-dG, A1 adenosine receptor, A33, ACE, ACE-2, activin, activin A, activin AB, activin B, activin C, activin RIA, activin RIA ALK-2, activin RIBALK-4, activin RIIA, activin RIIB, ADAM, ADAM10, ADAM12, ADAM15,ADAM17/TACE, ADAM8, ADAM9, ADAMTS, ADAMTS4, ADAMTS5, addressin, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, artemin, anti-Id, ASPARTIC, atrial natriuretic factor, av/b3 integrin, Axl, b2M, B7-1, B7-2, B7-H, B lymphocyte stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 osteogenin, BMP-4, BMP-2b, BMP-5, BMP-6 Vgr-1,BMP-7 (OP-1), BMP-8 (BMP-8a, OP-2), BMPR, BMPR-IA (ALK-3), BMPR-IB (ALK-6),BRK-2, RPK-1, BMPR-II (BRK-3), BMP, b-NGF, BOK, bombesin, bone-derived neurotrophic factor, BPDE, BPDE-DNA, BTC, complement factor 3 (C3), C3a, C4, C5, C5a,C10, CA125, CAD-8, calcitonin, cAMP, carcinoembryonic antigen (CEA), cancer-associated antigens, cathepsin A, cathepsin B, cathepsin C/DPPI, cathepsin D, cathepsin E, cathepsin H, cathepsin L, cathepsin O, cathepsin S, cathepsin V, cathepsin X/Z/P, CBL, CCI, CCK2,CCL, CCL1, CCL11, CCL12, CCL13, CCL14, CCL15, CCL16, CCL17, CCL18, CCL19,CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19,CD20, CD21, CD22, CD23, CD25, CD27L, CD28, CD29, CD30, CD30L, CD32, CD33 (p67protein), CD34, CD36, CD38, CD40, CD40L, CD44, CD45, CD46, CD49a, CD52, CD54, CD55, CD56, CD61, CD64, CD66e, CD74, CD80 (B7-1), CD89, CD91, CD95, CD123, CD137, CD138, CD140a, CD146, CD147, CD148, CD152, CD164, CEACAM5, CFTR, cGMP, CINC, botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, PD-1, PD-L1, LAG3,TIM3, galectin-9, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigens, DAN, DCC, DcR3, DC-SIGN, decay accelerating factor, des (1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, enkephalinase, eNOS, Eot, eotaxin 1, EpCAM, ephrin B2/EphB4,ePO, ERCC, E-selectin, ET-1, factor IIa, factor VII, factor VIIIc, factor IX, fibroblast, activating protein (FAP), Fas, FcR1, FEN-1, ferritin, FGF, FGF-19, FGF-2, FGF3, FGF-8,FGFR, FGFR-3, fibrin, FL, FLIP, Flt-3, Flt-4, follicle-stimulating hormone, fractalkine, FZD1, FZD2, FZD3, FZD4, FZD5, FZD6, FZD7, FZD8, FZD9, FZD10, G250, Gas6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (myostatin), GDF-9, GDF-15 (MIC-1), GDNF, GDNF, GFAP, GFRa-1, GFR-alpha 1, GFR-alpha 2, GFR-alpha 3, gITR, glucagon, Glut4, glycoprotein IIb/IIIa (GPIIb/IIIa), GM-CSF, gp130, gp72, GRO, growth hormone-releasing factor, hapten (NP-cap or NIP-cap), HB-EGF, HCC, HCMV gB envelope glycoprotein, HCMV gH envelope glycoprotein, HCMV UL, hematopoietic growth factor (HGF), Hep B gp120, heparanase, Her2, Her2/neu (ErbB-2), Her3 (ErbB-3), Her4 (ErbB-4),herpes simplex virus (HSV) gB glycoprotein, HSV gD glycoprotein, HGFA, high-molecular weight melanoma-associated antigen (HMW-MAA), HIV gp120, HIV IIIB gp 120 V3 loop, HLA, HLA-DR, HM1.24, HMFG PEM, HRG, Hrk, human heart myosin, human cytomegalovirus (HCMV), human growth hormone (HGH), HVEM, I-309, IAP, ICAM,ICAM-1, ICAM-3, ICE, ICOS, IFNg, Ig, IgA receptor, IgE, IGF, IGF binding protein, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-21, IL-23, IL-27, interferon (INF)-alpha, INF-beta, INF-gamma, inhibin, iNOS, insulin chain A, insulin chain B, insulin likegrowth factor 1, integrin alpha 2, integrin alpha 3, integrin alpha 4, integrin alpha 4/beta1, integrin alpha 4/beta 7, integrin alpha 5 (alpha V), integrin alpha 5/beta 1, integrin alpha5/beta 3, integrin alpha 6, integrin beta 1, integrin beta 2, interferon gamma, IP-10, I-TAC,JE, kallikrein 2, kallikrein 5, kallikrein 6, kallikrein 11, kallikrein 12, kallikrein 14, kallikrein15, kallikrein L1, kallikrein L2, kallikrein L3, kallikrein L4, KC, KDR, keratinocyte growth factor (KGF), laminin 5, LAMP, LAP, LAP (TGF-1), latent TGF-1, latent TGF-1 bp1, LBP, LDGF, LECT2, lefty, Lewis-Y antigen, Lewis-Y-related antigen, LFA-1, LFA-3, Lfo, LIF, LIGHT, lipoprotein, LIX, LKN, Lptn, L-selectin, LT-a, LT-b, LTB4, LTBP-1, lung surface, luteinizing hormone, lymphotoxin beta receptor, Mac-1, MAdCAM, MAG, MAP2, MARC,MCAM, MCAM, MCK-2, MCP, M-CSF, MDC, Mer, metalloproteinases, MGDF receptor, MGMT, MHC (HLA-DR), MIF, MIG, MIP, MIP-1-alpha, MK, MMAC1, MMP, MMP-1,MMP-10, MMP-11, MMP-12, MMP-13, MMP-14, MMP-15, MMP-2, MMP-24, MMP-3,MMP-7, MMP-8, MMP-9, MPIF, Mpo, MSK, MSP, mucin (Muc1), MUC18, Mullerian inhibiting substance, Mug, MuSK, NAIP, NAP, NCAD, N—C adherin, NCA 90, NCAM, neprilysin, neurotrophin-3,-4, or-6, neurturin, nerve growth factor (NGF), NGFR, NGF-beta, nNOS, NO, NOS, Npn, NRG-3, NT, NTN, OB, OGG1, OPG, OPN, OSM,OX40L, OX40R, p150, p95, PADPr, parathyroid hormone, PARC, PARP, PBR, PBSF,PCAD, P-cadherin, PDGF, PDGF, PDK-1, PECAM, PEM, PF4, PGE, PGF, PGI2, PGJ2, PIN, PLA2, placental alkaline phosphatase (PLAP), PIGF, PLP, PP14, proinsulin, prorelaxin, protein C, PS, PSA, PSCA, prostate-specific membrane antigen (PSMA), PTEN, PTHrp, Ptk, PTN, R51, RANK, RANKL, RANTES, RANTES, relaxin A chain, relaxin Bchain, renin, respiratory syncytial virus (RSV) F, RSV Fgp, Ret, rheumatoid factor, RLIP76,RPA2, RSK, S100, SCF/KL, SDF-1, SERINE, serum albumin, sFRP-3, Shh, SIGIRR, SK-1,SLAM, SLPI, SMAC, SMDF, SMOH, SOD, SPARC, Stat, STEAP, STEAP-II, TACE,TACI, TAG-72 (tumor-associated glycoprotein-72), TARC, TCA-3, T cell receptor (e.g., Tcell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta 1, TGF-beta2, TGF-beta 3, TGF-beta 4, TGF-beta 5, thrombin, thymus Ck-1, thyroid stimulating hormone, Tie, TIMP, TIQ, tissue factor, TMEFF2, Tmpo, TMPRSS2, LOX-1, MDA5 (melanoma differentiation-associated protein 5), TNF, TNF-alpha, TNF-alpha/beta, TNF-beta 2, TNFc, TNF-RI, TNF-RII, TNFRSF10A (TRAIL R1 Apo-2,DR4), TNFRSF10B (TRAIL R2 DR5, KILLER, TRICK-2A, TRICK-B), TNFRSF10C (TRAIL R3 DcR1, LIT, TRID), TNFRSF1OD (TRAIL R4 DcR2, TRUNDD), TNFRSF11A (RANK ODF R, TRANCE R), TNFRSF11B (OPG OCIF, TR1), TNFRSF12 (TWEAK RFN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITRAITR), TNFRSF19 (TROY TAJ, TRADE), TNFRSF19L (RELT), TNFRSF1A (TNF RICD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6),TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 ligand, TL2), TNFSF11 (TRANCE/RANK ligand ODF, OPG ligand), TNFSF12 (TWEAK Apo-3 ligand,DR3 ligand), TNFSF13 (APRIL TALL2), TNFSF13B (BAFF BLYS, TALL1, THANK,TNFSF20), TNFSF14 (LIGHT HVEM ligand, LTg), TNFSF15 (TL1A/VEGI), TNFSF18 (GITR ligand AITR ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 ligand gp34, TXGP1), TNFSF5 (CD40 ligand CD154, gp39, HIGM1, IMD3, TRAP), TNFSF6 (Fas ligand Apo-1ligand, APT1 ligand), TNFSF7 (CD27 ligand CD70), TNFSF8 (CD30 ligand CD153),TNFSF9 (4-1BB ligand CD137 ligand), TP-1, t-PA, Tpo, TRAIL, TRAIL R, TRAIL-R1,TRAIL-R2, TRANCE, transferrin receptor, TRF, Trk, TROP-2, TLR (toll-like receptor) 1,TLR2, TLR3, TLR4, TLR5, TLR6, TLR7, TLR8, TLR9, TLR10, RIG-I; RAGE, P2Y2, P2X7, secreted frizzled related protein 1 (FRP1), and Interferon-inducible protein (AIM2). TSG, TSLP, tumor associated antigen CA125, tumor-associated antigen-expressing Lewis-Y-related carbohydrate, TWEAK, TXB2, Ung, uPAR, uPAR-1, urokinase, VCAM, VCAM-1, VECAD, VE-cadherin, VE-cadherin-2, VEFGR-1 (flt-1), VEGF, VEGFR, VEGFR-3 (flt-4), VEGI, VIM, viral antigens, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, HMGB1, IgA,CD81, CD97, CD98, DDR1, DKK1, EREG, Hsp90, IL-17/IL-17R, IL-20/IL-20R, oxidized LDL, PCSK9, prekallikrein, RON, TMEM16F, SOD1, chromogranin A, chromogranin B, tau, VAP1, high-molecular-weight kininogen, IL-31, IL-31R, ILIRL1 (ST2), Nav1.1, Nav1.2, Nav1.3,Nav1.4, Nav1.5, Nav1.6, Nav1.7, Nav1.8, Nav1.9, EPCR, C1, C1q, Clr, Cls, C2, C2a, C2b,C3, C3a, C3b, C4, C4a, C4b, C5, C5a, C5b, C6, C7, C8, C9, factor B, factor D, factor H, properdin, sclerostin, fibrinogen, fibrin, prothrombin, thrombin, tissue factor, factor V, factorVa, factor VII, factor VIIa, factor VIII, factor VIIIa, factor IX, factor IXa, factor X, factor Xa, factor XI, factor XIa, factor XII, factor XIIa, factor XIII, factor XIIIa, TFPI, antithrombin III, EPCR, thrombomodulin, TAPI, tPA, plasminogen, plasmin, PAI-1, PAI-2,GPC3, syndecan-1, syndecan-2, syndecan-3, syndecan-4, LPA, S1P, and receptors for hormones or growth factors.

Although the examples of the molecule listed above also include receptors (membrane proteins), these receptors (membrane proteins) even existing in a soluble form in a body fluid (e.g., as a truncated form) can be used as “a third antigen” (a target molecule) to which “a third moiety” in the present invention binds. One non-limiting example the soluble form of such a receptor (membrane protein) can include the protein represented by soluble IL-6R as described by Mullberg et al. (J. Immunol. (1994) 152 (10), 4958-4968).

In the present invention, in case that “a third antigen” is a soluble protein, for example, IL-6, a molecule comprising a first moiety and a third moiety of the present invention binds to the dead cell-derived molecule (i.e., a first antigen), e.g., F-actin complex, via first moiety of the molecule, and binds to IL-6 (i.e., a third antigen) via third moiety of the molecule to neutralize IL-6 in an affected tissue more specifically where the dead cell-derived molecules are occurred due to a cell death than in non-affected tissue. Upon the neutralization of IL-6, an immune response caused by immune cells is indirectly regulated. The second moiety may bind the second target, wherein the second target may be the same target as the third target (in this case IL-6) or a different target, such as CD3 or CD137.

In the present invention, “a third moiety” that binds to “a third antigen” as described above may have any structure as long as it binds to “a third antigen” as described above. The structure of “a third moiety” may include but is not limited to, a polypeptide or a portion thereof, or a small or medium chemical compound or a portion thereof, or a polynucleotide or a portion thereof. The polypeptide or a portion thereof includes but is not limited to an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region,), a single-domain antibody (sdAb), a single-chain Fv (scFv), a single-chain antibody, a Fv, a single-chain Fv2 (scFv2), a Fab, and F(ab′)₂.

Cell which a Membrane Protein as “a Third Antigen” is Expressed

In the present invention, as described above, “a third antigen” is a membrane protein of an immune cell, or a tumour cell and includes but is not limited to a membrane protein expressed on a cell membrane and/or an endosome membrane involved in a signal transmission in a cell. In the present invention, such cell which a membrane protein as “a third antigen” is expressed may be any sort of or any type of immune cell or tumour cell, including but not limited to an immune cell such as a T cell, a killer cell, a helper T cell, a regulatory T cell, a B cell, a memory B cell, a NK cell, a NKT cell, a dendritic cell, a macrophage, an eosinophil, a neutrophil cell, and a basophil, a somatic cell (e.g., epithelial cell, fibroblastic cell, interstitial cell, bone cell, muscle cell, nerve cell, blood cell or the like), a reproductive cell, or an affected or abnormal cell (e.g., a tumor cell, an autoreactive cell) . . . . Such cells may be located in any tissue, including but not limited to, epithelial tissue, muscle tissue, nerve tissue, connective tissue, or an affected tissue associated with a cancer, an inflammation, and autoimmune disease or the like. Preferable embodiments of the cell which “a third antigen” (also referred as a target molecule) is expressed may be an immune cell selected from a T cell, a killer cell, a helper T cell, a regulatory T cell, a B cell, a memory B cell, a NK cell, a NKT cell, a dendritic cell, a macrophage, an eosinophil, a neutrophil cell, and a basophil. Other preferable embodiments of the cell which “a third antigen” (also referred as a target molecule) is expressed may be a cell in an affected tissue associated with a cancer, an inflammation, and autoimmune disease or the like.

The embodiments disclosed above for the “second antigen” also apply to the “third antigen”.

A Molecule Comprising a First Moiety that Binds to a First Antigen and a Second Moiety that Binds to a Second Antigen Wherein the Molecule Comprises at Least One Third Moiety that Binds to a Third Antigen

In the present invention, one embodiment of “molecule comprising a first moiety that binds to a first antigen and a second moiety that binds to a second antigen wherein the molecule comprises at least one third moiety that binds to a third antigen” includes but is not limited to a molecule having the following feature.

i. An antibody in which one of the two Fabs binds “a second antigen” and one of the two Fabs binds “a third antigen”. The two Fab arms may form a F(ab′)₂ conjugated to the Fc. The “a first moiety that binds to a first antigen” may in embodiments be conjugated to both chains or a single chain at the C-terminus of an Fc region. A further example is an antibody in which “a first moiety that binds to a first antigen” has been conjugated to the C-terminus of one or both light chain regions. Yet alternatively, “a first moiety that binds to a first antigen” may be conjugated to the one or both of the two Fab arms. The “a first moiety that binds to a first antigen” may be conjugated to the one or both of the two Fab arms in different ways. In exemplary embodiments, “a first moiety that binds to a first antigen” may be conjugated to the one or both of the two Fab arms by conjugation to the N-terminus of the light chain of one Fab arm. In exemplary embodiments, “a first moiety that binds to a first antigen” may be conjugated to the one or both of the two Fab arms by conjugation to the N-terminus of the heavy chain of one Fab arm or both Fab arms. In certain embodiments, “a first moiety that binds to a first antigen” is a further Ig-like moiety, such as scFv or VHH, or is a whole or an antigen-binding portion of Clec9A or Clec4e. In exemplary embodiments, “a first moiety that binds to a first antigen” may be conjugated to the one or both of the two Fab arms by conjugation to the VL and CL domain of one or both Fab arms. In exemplary embodiments, “a first moiety that binds to a first antigen” may be conjugated to the one or both of the two Fab arms by conjugation to the VH and CH1 domain of one or both Fab arms. In exemplary embodiments, “a first moiety that binds to a first antigen” may be conjugated to the one or both of the two Fab arms by conjugation to the VL domain of one or both Fab arms. In exemplary embodiments, “a first moiety that binds to a first antigen” may be conjugated to the one or both of the two Fab arms by conjugation to the VH domain of one or both Fab arms. In exemplary embodiments, “a first moiety that binds to a first antigen” may be conjugated to the one or both of the two Fab arms by conjugation to the VH and CH1 domain of one or both Fab arms. In exemplary embodiments, “a first moiety that binds to a first antigen” may be conjugated to the one or both of the two Fab arms by conjugation to the VH and CH1 domain of one or both Fab arms. Still alternatively, “a first moiety that binds to a first antigen” may be conjugated to one of Fab arms instead of, or in addition to the conjugation to Fc region, and one of the two Fabs binds “a third antigen” and the “third moiety” is conjugated to a Fc region. One example of this embodiment may be a trispecific antibody or a trispecific antigen-binding domain, which one Fab arm binds to the dead-cell derived molecule (e.g., F-actin complex) as described above, and another Fab arm binds to a membrane protein expressed on a cell membrane of a cell (e.g., an immune cell, an cancer cell, an autoreactive cell) and a further Ig-like moiety, such as scFv or VHH binds to a membrane protein expressed on a cell membrane of a cell (e.g., an immune cell, an cancer cell, an autoreactive cell), which may be the same or a different membrane protein. Further, more than two “a first moiety that binds to a first antigen” may be conjugated to an Fc region. The two Fab arms may conjugate to Fc region in various formats. One example of “a first moiety that binds to a first antigen” in certain embodiments is a whole or an antigen-binding portion of Clec9A or Clec4e or a Ig-like moiety such as scFv or VHH. Another example of “a first moiety that binds to a first antigen” in certain embodiments is a Ig-like moiety, such as scFv or VHH, that binds to PCNA. Another example of “a first moiety that binds to a first antigen” in certain embodiments is a Ig-like moiety, such as scFv or VHH, that binds to SF3B3. Another example of “a first moiety that binds to a first antigen” in certain embodiments is a Ig-like moiety, such as scFv or VHH, that binds to PS. Another example of “a first moiety that binds to a first antigen” in certain embodiments is a Ig-like moiety, such as scFv or VHH, that binds to F-Actin. In any of the embodiments above, it is possible that two Fab arms are linked with each other via one or more bonds, such as disulfide bonds. In embodiments, the amino acid residues from which the bonds between the antigen-binding domains originate are present within the variable regions or constant regions. Exemplary embodiments are schematically shown in FIG. 3. In one preferred embodiment, the antibodies may have their first H-chain CH3 region and second H-chain CH3 region crosslinked by disulfide bonds.

ii. An antibody in which one of the two Fabs binds “a first antigen” and one of the two Fabs binds “a second antigen” (or “a third antigen”). The two Fab arms may form a F(ab′)₂ conjugated to the Fc. The “a third moiety that binds to a third antigen” (or “a second moiety that binds to a second antigen”) may in embodiments be conjugated to both chains or a single chain at the C-terminus of an Fc region. A further example is an antibody in which “a third moiety that binds to a third antigen” (or “a second moiety that binds to a second antigen”) has been conjugated to the C-terminus of one or both light chain regions. Yet alternatively, “a third moiety that binds to a third antigen” (or “a second moiety that binds to a second antigen”) may be conjugated to the one or both of the two Fab arms. The “a third moiety that binds to a third antigen” (or “a second moiety that binds to a second antigen”) may be conjugated to the one or both of the two Fab arms in different ways. In exemplary embodiments, “a third moiety that binds to a third antigen” (or “a second moiety that binds to a second antigen”) may be conjugated to the one or both of the two Fab arms by conjugation to the N-terminus of the light chain of one Fab arm. In exemplary embodiments, “a third moiety that binds to a third antigen” (or “a second moiety that binds to a second antigen”) may be conjugated to the one or both of the two Fab arms by conjugation to the N-terminus of the heavy chain of one Fab arm or both Fab arms. In certain embodiments, “a third moiety that binds to a third antigen” (or “a second moiety that binds to a second antigen”) is a further Ig-like moiety, such as scFv or VHH, or is a whole or an antigen-binding portion of e.g. a receptor ligand or a cytokine. In exemplary embodiments, “a third moiety that binds to a third antigen” (or “a second moiety that binds to a second antigen”) may be conjugated to the one or both of the two Fab arms by conjugation to the VL and CL domain of one or both Fab arms. In exemplary embodiments, “a third moiety that binds to a third antigen” (or “a second moiety that binds to a second antigen”) may be conjugated to the one or both of the two Fab arms by conjugation to the VH and CH1 domain of one or both Fab arms. In exemplary embodiments, “a third moiety that binds to a third antigen” (or “a second moiety that binds to a second antigen”) may be conjugated to the one or both of the two Fab arms by conjugation to the VL domain of one or both Fab arms. In exemplary embodiments, “a third moiety that binds to a third antigen” (or “a second moiety that binds to a second antigen”) may be conjugated to the one or both of the two Fab arms by conjugation to the VH domain of one or both Fab arms. In exemplary embodiments, “a third moiety that binds to a third antigen” (or “a second moiety that binds to a second antigen”) may be conjugated to the one or both of the two Fab arms by conjugation to the VH and CH1 domain of one or both Fab arms. In exemplary embodiments, “a third moiety that binds to a third antigen” (or “a second moiety that binds to a second antigen”) may be conjugated to the one or both of the two Fab arms by conjugation to the VH and CH1 domain of one or both Fab arms. Still alternatively, “a third moiety that binds to a third antigen” (or “a second moiety that binds to a second antigen”) may be conjugated to one of Fab arms instead of, or in addition to the conjugation to Fc region, and one of the two Fabs binds “a first antigen” and the “first moiety” is conjugated to a Fc region. One example of this embodiment may be a trispecific antibody or a trispecific antigen-binding domain, which one Fab arm binds to the dead-cell derived molecule (e.g., F-actin complex) as described above, and another Fab arm binds to a membrane protein expressed on a cell membrane of a cell (e.g., an immune cell, an cancer cell, an autoreactive cell) and a further Ig-like moiety, such as scFv or VHH binds to a membrane protein expressed on a cell membrane of a cell (e.g., an immune cell, an cancer cell, an autoreactive cell), which may be the same or a different membrane protein. Further, more than two “a third moiety that binds to a third antigen” (or “a second moiety that binds to a second antigen”) may be conjugated to an Fc region. The two Fab arms may be conjugated to the Fc region in various formats.

One example of “a first moiety that binds to a first antigen” in certain embodiments is a whole or an antigen-binding portion of Clec9A or Clec4e or a Ig-like moiety such as scFv or VHH. Another example of “a first moiety that binds to a first antigen” in certain embodiments is a Ig-like moiety, such as scFv or VHH, that binds to PCNA. Another example of “a first moiety that binds to a first antigen” in certain embodiments is a Ig-like moiety, such as scFv or VHH, that binds to SF3B3. Another example of “a first moiety that binds to a first antigen” in certain embodiments is a Ig-like moiety, such as scFv or VHH, that binds to PS. Another example of “a first moiety that binds to a first antigen” in certain embodiments is a Ig-like moiety, such as scFv or VHH, that binds to F-Actin.

In any of the embodiments above, it is possible that two Fab arms are linked with each other via one or more bonds, such as disulfide bonds. In embodiments, the amino acid residues from which the bonds between the antigen-binding domains originate are present within the variable regions or the constant regions. Exemplary embodiments are schematically shown in FIG. 3. In one preferred embodiment, the antibodies may have their first H-chain CH3 region and second H-chain CH3 region crosslinked by disulfide bonds.

iii. A polypeptide comprising “a first moiety that binds to a first antigen” and “a second moiety that binds to a second antigen” and “a third moiety that binds to a third antigen”, wherein the first moiety and the second moiety and the third moiety are fused with or without a linker. One example of this embodiment is a fusion protein comprising a extracellular domain of Clec9A or Clec4e and a first ligand for any membrane protein expressed on a cell membrane of a cell (e.g., an immune cell, an cancer cell, an autoreactive cell) and a second ligand for any membrane protein expressed on a cell membrane of a cell (e.g., an immune cell, an cancer cell, an autoreactive cell), wherein the first ligand and second ligand may bind to the same or to a different membrane protein. For example, the second moiety may be an Ig-like moiety, such as scFv or VHH, which binds to CD3 and the third moiety may be an Ig-like moiety, such as scFv or VHH, which binds to CD137. Another example of this embodiment is a fusion protein comprising an Ig-like moiety, such as scFv or VHH, which binds to PCNA, and a first ligand for any membrane protein expressed on a cell membrane of a cell (e.g., an immune cell, an cancer cell, an autoreactive cell) and a second ligand for any membrane protein expressed on a cell membrane of a cell (e.g., an immune cell, an cancer cell, an autoreactive cell), wherein the first ligand and second ligand may bind to the same or to a different membrane protein. For example, the second moiety may be an Ig-like moiety, such as scFv or VHH, which binds to CD3 and the third moiety may be an Ig-like moiety, such as scFv or VHH, which binds to CD137. Another example of this embodiment is a fusion protein comprising an Ig-like moiety, such as scFv or VHH, which binds to SF3B3, and a first ligand for any membrane protein expressed on a cell membrane of a cell (e.g., an immune cell, an cancer cell, an autoreactive cell) and a second ligand for any membrane protein expressed on a cell membrane of a cell (e.g., an immune cell, an cancer cell, an autoreactive cell), wherein the first ligand and second ligand may bind to the same or to a different membrane protein. Another example of this embodiment is a fusion protein comprising an Ig-like moiety, such as scFv or VHH, which binds to PS, and a first ligand for any membrane protein expressed on a cell membrane of a cell (e.g., an immune cell, an cancer cell, an autoreactive cell) and a second ligand for any membrane protein expressed on a cell membrane of a cell (e.g., an immune cell, an cancer cell, an autoreactive cell), wherein the first ligand and second ligand may bind to the same or to a different membrane protein. For example, the second moiety may be an Ig-like moiety, such as scFv or VHH, which binds to CD3 and the third moiety may be an Ig-like moiety, such as scFv or VHH, which binds to CD137. Exemplary embodiments comprising a Fc region are schematically depicted in FIG. 3. For example, a third moiety which may be an Ig-like moiety, such as scFv or VHH, and a second moiety which may be an Ig-like moiety, such as scFv or VHH, may be fused (or conjugated) and may be in turn conjugated to a Fc region via one of two chains. A first moiety may then be conjugated thereto in different ways. For example, “a first moiety that binds to a first antigen” which is not an Ig-like moiety, such as a whole or an antigen-binding portion of Clec9A or Clec4e, is conjugated to the second chain of the Fc region, such as the N-terminus or C-terminus of the Fc region, or to an internal side chain of the Fc region. Such embodiments are shown in FIG. 3. In another exemplary embodiment shown in FIG. 3, “a first moiety that binds to a first antigen” is conjugated to a VL and/or CL region of the second or third moiety. In further alternative embodiments, as shown schematically in FIG. 3, a “second moiety” (or “third moiety”) representing a Fab is conjugated to a Fc region via one of two chains of the Fc region, and the “third moiety” (or “second moiety”) may be conjugated to a CH2 or CH3 region of the one chain of the Fc region. The “a first moiety that binds to a first antigen” which is not an Ig-like moiety, such as a whole or an antigen-binding portion of Clec9A or Clec4e, is conjugated to the second chain of the Fc region, such as the N-terminus of the Fc region (or C-terminus of the Fc region, or to an internal side chain of the Fc region). In yet further alternative embodiments, which are also shown schematically in FIG. 3, “a first moiety that binds to a first antigen” which is not an Ig-like moiety, such as a whole or an antigen-binding portion of Clec9A or Clec4e, is conjugated to one chain of a Fc region, such as the N-terminus of the Fc region or C-terminus of the Fc region, or to an internal side chain of the Fc region. In some embodiments, second “a first moiety that binds to a first antigen” which is not an Ig-like moiety, such as a whole or an antigen-binding portion of Clec9A or Clec4e, is conjugated to the second chain of a Fc region, such as the N-terminus of the Fc region or C-terminus of the Fc region, or to an internal side chain of the Fc region. For example, a third moiety which may be an Ig-like moiety, such as scFv or VHH, and a second moiety which may be an Ig-like moiety, such as scFv or VHH, may be fused (or conjugated) and may be in turn conjugated to the CH2 or CH3 domain of an Fc region via one of two chains. Alternatively, a third moiety which may be an Ig-like moiety, such as scFv or VHH may be in turn conjugated to the CH2 or CH3 domain of an Fc region via one of two chains, and a second moiety which may be an Ig-like moiety, such as scFv or VHH may be in turn conjugated to the CH2 or CH3 domain of an Fc region via the second of two chains.

In any of the embodiments above, it is possible that two Fab arms are linked with each other via one or more bonds, such as disulfide bonds. In embodiments, the amino acid residues from which the bonds between the antigen-binding domains originate are present within the variable regions or the constant regions. Exemplary embodiments are schematically shown in FIG. 3. In one preferred embodiment, the antibodies may have their first H-chain CH3 region and second H-chain CH3 region crosslinked by disulfide bonds.

iv. A chemical compound comprising “a first moiety that binds to a first antigen” and “a second moiety that binds to a second antigen”. One example of this embodiment may be a chemical compound comprising at least two moieties, which one moiety binds to the dead-cell derived molecule (e.g., F-actin complex, a nucleosome which is a complex of a histone and a nucleic acid) as described above, and other moiety arm binds to a membrane protein expressed on a cell membrane of a cell (e.g., an immune cell, an cancer cell, an autoreactive cell). Such chemical compounds may be carried out by using a bioconjugate technique (Bioconjugate Techniques, 3rd Edition, 2013, by Greg T. Hermanson). For example, in case that “a first antigen” is a nucleosome which is a complex of a histone and a nucleic acid, and “a second antigen” is a mTOR (mechanistic Target Of Rapamycin), the chemical compound may be a compound comprising Distamycine as “a first moiety” that binds to a nucleic acid and Rapamycin as “a second moiety” that binds to mTOR, and further comprising a photoreactive group (e.g., aryl azide, diazirine, psolalen). Upon receipt of a photo (light) energy by the compound, the first antigen and the second antigen is crosslinked by the compound, and, the binding of the second moiety to the second antigen on a cell membrane confers a signal transmission or a signal blockage into the cell.

In one aspect, an antigen-binding molecule is provided which comprises:
(1) at least one first moiety that binds to a first antigen, and
(2) at least one second moiety that binds to a second antigen;
wherein said first antigen is a damage-associated molecular pattern (DAMP), and wherein said second antigen is different from the first antigen, preferably wherein said second antigen is a membrane protein of an immune cell, or a tumour cell, and wherein the first antigen is selected from the group consisting of:
a) Splicing Factor 3B Subunit 3 (SF3B3); and
b) Proliferating Cell Nuclear Antigen (PCNA).

In one embodiment thereof, the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In one embodiment, said third antigen is different from the first and second antigen. For example, the first antigen is PCNA or SF3B3, the second antigen is CD3 and the third antigen is CD137. For example, the second antigen is CD137 and the third antigen is CD3. In one embodiment, said third antigen is different from the first but same as the second antigen. For example, the first antigen is PCNA or SF3B3, the second antigen is CD3 and the third antigen is CD3. For example, the first antigen is PCNA or SF3B3, the second antigen is CD137 and the third antigen is CD137.

In embodiments, the first moiety is selected from the group consisting of:
A) an Ig type binding moiety; or
B) a binding polypeptide, particularly wherein the said binding polypeptide is a non-Ig-type binding moiety.

In embodiments, in case that the first antigen is Splicing Factor 3B Subunit 3 (SF3B3), the first moiety is an Ig type binding moiety. In embodiments, in case that the first antigen is Splicing Factor 3B Subunit 3 (SF3B3), the first moiety is a Clec4e polypeptide. In embodiments, in case that the first antigen is Proliferating Cell Nuclear Antigen (PCNA), the first moiety is an Ig type binding moiety. In embodiments, the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule, especially wherein the second and/or the third antigen is a membrane protein involved in a signal transmission in a cell, preferably a signal transmission receptor. In an embodiment, the second and the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and the third antigen is a membrane protein involved in a signal transmission in a cell, preferably a signal transmission receptor. In an embodiment, the second or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second or the third antigen is a membrane protein involved in a signal transmission in a cell, preferably a signal transmission receptor.

In embodiments, the second and/or the third antigen is a membrane protein of a tumour cell. In an embodiment, the second and the third antigen is a membrane protein of a tumour cell. In an embodiment, the second or the third antigen is a membrane protein of a tumour cell. In an embodiment, the second antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second antigen is a membrane protein involved in a signal transmission in a cell, preferably a signal transmission receptor; and the third antigen is a membrane protein of a tumour cell. In an embodiment, the second antigen is a membrane protein of a tumour cell and the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second antigen is a membrane protein involved in a signal transmission in a cell, preferably a signal transmission receptor.

In an embodiment, the second and/or the third moiety is an Ig type binding moiety.

In an embodiment, the antigen-binding molecule is an antibody, in particular an IgG type antibody. In an embodiment, the antibody is capable of binding to a membrane protein of an immune cell or a tumour cell and further comprises, preferably linked to the Fc region, at least one binding polypeptide, particularly wherein the said binding polypeptide is selected from a Clec9A polypeptide and a Clec4e polypeptide; especially wherein the antibody is selected from the group consisting of an anti-CD3 antibody comprising a Clec9A polypeptide, an anti-CD137 antibody comprising a Clec9A polypeptide, an anti-CD3 antibody comprising a Clec4e polypeptide, and an anti-CD137 antibody comprising a Clec4e polypeptide.

In an embodiment, the antigen-binding molecule is an antibody, in particular an IgG type antibody, wherein said first moiety is linked to the Fc region, and is an Ig type binding moiety, particularly wherein the antibody is selected from the group consisting of
(A) an anti-CD3 antibody;
(B) an anti-CD137 antibody; and
(C) a bispecific anti-CD3 anti-CD137 antibody.

In an embodiment, the antigen-binding molecule is an antibody, in particular an IgG type antibody, wherein the antigen-binding molecule comprises:
(1) one first moiety wherein said first moiety is linked to the Fc region, in particular wherein said first moiety is an Ig type binding moiety,
(2) one second moiety wherein said second moiety is linked to the Fc region, and is an Ig type binding moiety, and
(3) one third moiety wherein said third moiety is linked to the Fc region, and is an Ig type binding moiety.

In an embodiment, the one second moiety and the one third moiety together form a F(ab′)₂.

In an embodiment, the antigen-binding molecule is an antibody, in particular an IgG type antibody, wherein said first moiety is linked to the Fc region, and is an Ig type binding moiety, the antibody is a bispecific antibody, particularly wherein the antigen binding molecule is a bispecific antibody directed against a membrane protein of an immune cell, or a tumour cell and against a first antigen selected from the group consisting of: a) Splicing Factor 3B Subunit 3 (SF3B3); and b)

Proliferating Cell Nuclear Antigen (PCNA),
especially wherein the antibody is selected from the group consisting of
a bispecific anti-SF3B3 anti-CD3 antibody,
a bispecific anti-SF3B3 anti-CD137 antibody,
a bispecific anti-PCNA anti-CD3 antibody, and
a bispecific anti-PCNA anti-CD137 antibody.

In an embodiment, the antigen-binding molecule is an antibody, in particular an IgG type antibody, wherein said first moiety is linked to the Fc region, and is an Ig type binding moiety, the antibody is a trispecific antibody, particularly wherein the antigen binding molecule is a trispecific antibody directed i) against a membrane protein of an immune cell, and against a membrane protein of tumour cell; and against a first antigen selected from the group consisting of: a) Splicing Factor 3B Subunit 3 (SF3B3); and b) Proliferating Cell Nuclear Antigen (PCNA), or ii) against a first membrane protein of an immune cell, and a second membrane protein of an immune cell different from the first membrane protein; and against a first antigen selected from the group consisting of: a) Splicing Factor 3B Subunit 3 (SF3B3); and b) Proliferating Cell Nuclear Antigen (PCNA), or iii) against a first membrane protein of a tumour cell, and a second membrane protein of a tumour cell different from the first membrane protein; and against a first antigen selected from the group consisting of: a) Splicing Factor 3B Subunit 3 (SF3B3); and b) Proliferating Cell Nuclear Antigen (PCNA), especially wherein the antibody is selected from the group consisting of a trispecific anti-SF3B3 anti-CD3 anti-CD137 antibody, and a trispecific anti-PCNA anti-CD3 anti-CD137 antibody.

In another aspect, an antigen-binding molecule is provided which comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
wherein said second antigen is a membrane protein of an immune cell, or a tumour cell, and wherein the molecule comprises at least one third moiety that binds to a third antigen; wherein said third antigen is different from the first antigen; and wherein said third antigen is a membrane protein of an immune cell, or a tumour cell,
wherein the first antigen is selected from the group consisting of:
a) F-actin; b) Phosphatidylserine (PS); c) Splicing Factor 3B Subunit 3 (SF3B3); and d) Proliferating Cell Nuclear Antigen (PCNA).

In an embodiment, said third antigen is different from the first and second antigen. In an embodiment, said third antigen is different from the first but same as the second antigen. In an embodiment, said third antigen is different from the first and second antigen. For example, the first antigen is F-Actin, the second antigen is CD3 and the third antigen is CD137. For example, the second antigen is CD137 and the third antigen is CD3. In one embodiment, said third antigen is different from the first but same as the second antigen. For example, the first antigen is F-Actin, the second antigen is CD3 and the third antigen is CD3. For example, the first antigen is F-Actin, the second antigen is CD137 and the third antigen is CD137. For example, the first antigen is Phosphatidylserine (PS), the second antigen is CD3 and the third antigen is CD3. For example, the first antigen is Phosphatidylserine (PS), the second antigen is CD137 and the third antigen is CD137. For example, the first antigen is Phosphatidylserine (PS), the second antigen is CD3 and the third antigen is CD137. For example, the second antigen is CD137 and the third antigen is CD3. For example, the first antigen is Proliferating Cell Nuclear Antigen (PCNA), the second antigen is CD3 and the third antigen is CD3. For example, the first antigen is Proliferating Cell Nuclear Antigen (PCNA), the second antigen is CD137 and the third antigen is CD137. For example, the first antigen is Proliferating Cell Nuclear Antigen (PCNA), the second antigen is CD3 and the third antigen is CD137. For example, the second antigen is CD137 and the third antigen is CD3. For example, the first antigen is Splicing Factor 3B Subunit 3 (SF3B3), the second antigen is CD3 and the third antigen is CD3. For example, the first antigen is Splicing Factor 3B Subunit 3 (SF3B3), the second antigen is CD137 and the third antigen is CD137. For example, the first antigen is Splicing Factor 3B Subunit 3 (SF3B3), the second antigen is CD3 and the third antigen is CD137. For example, the second antigen is CD137 and the third antigen is CD3.

In embodiment, the first moiety is selected from the group consisting of: A) an Ig type binding moiety; or B) a binding polypeptide, particularly wherein the said binding polypeptide is a non-Ig-type binding moiety. In embodiments,
A) in case that the first antigen is Splicing Factor 3B Subunit 3 (SF3B3), the first moiety is an Ig type binding moiety, or
B) in case that the first antigen is Splicing Factor 3B Subunit 3 (SF3B3), the first moiety is a Clec4e polypeptide, or
C) in case that the first antigen is Proliferating Cell Nuclear Antigen (PCNA), the first moiety is an Ig type binding moiety, or
D) in case that the first antigen is F-actin, the first moiety is an Ig type binding moiety, or
E) in case that the first antigen is F-actin, the first moiety is a Clec9A polypeptide, or
F) in case that the first antigen is PS, the first moiety is an Ig type binding moiety.

In embodiments, the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second and/or the third antigen is a membrane protein involved in a signal transmission in a cell, preferably a signal transmission receptor. In an embodiment, the second and the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and the third antigen is a membrane protein involved in a signal transmission in a cell, preferably a signal transmission receptor. In an embodiment, the second or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second or the third antigen is a membrane protein involved in a signal transmission in a cell, preferably a signal transmission receptor.

In embodiments, the second and/or the third antigen is a membrane protein of a tumour cell. In an embodiment, the second and the third antigen is a membrane protein of a tumour cell. In an embodiment, the second or the third antigen is a membrane protein of a tumour cell. In an embodiment, the second antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second antigen is a membrane protein involved in a signal transmission in a cell, preferably a signal transmission receptor; and the third antigen is a membrane protein of a tumour cell. In an embodiment, the second antigen is a membrane protein of a tumour cell and the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule; especially wherein the second antigen is a membrane protein involved in a signal transmission in a cell, preferably a signal transmission receptor.

In an embodiment, the second and/or third moiety is an Ig type binding moiety.

In an embodiment, the antigen-binding molecule is an antibody, in particular an IgG type antibody. For example, an antigen-binding molecule can be provided wherein an anti-PS and anti-CD3 moiety together form a F(ab′)₂, linked to a Fc. A further Ig-like moiety, like a scFv, VHH or variable domain, binding to a membrane protein of an immune cell, or a tumour cell, can be linked to the Fc. In an embodiment, the antibody is capable of binding to a membrane protein of an immune cell or a tumour cell and further comprises, preferably linked to the Fc region, at least one binding polypeptide,
particularly wherein the said binding polypeptide is selected from a Clec9A polypeptide and a Clec4e polypeptide;
especially wherein the antibody is selected from the group consisting of an anti-CD3 antibody comprising a Clec9A polypeptide, an anti-CD137 antibody comprising a Clec9A polypeptide,
an anti-CD3 antibody comprising a Clec4e polypeptide, and
an anti-CD137 antibody comprising a Clec4e polypeptide.

In embodiments, the antibody is a trispecific antibody, particularly wherein the antigen binding molecule is a trispecific antibody directed
i) against a membrane protein of an immune cell, and against a membrane protein of a tumour cell; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
ii) against a first membrane protein of an immune cell, and a second membrane protein of an immune cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
iii) against a first membrane protein of a tumour cell, and a second membrane protein of a tumour cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA),
especially wherein the antibody is selected from the group consisting of
a trispecific anti-SF3B3 anti-CD3 anti-CD137 antibody,
a trispecific anti-PCNA anti-CD3 anti-CD137 antibody,
a trispecific anti-PS anti-CD3 anti-CD137 antibody
a trispecific anti-F-Actin anti-CD3 anti-CD137 antibody
a bispecific anti-CD3 anti-CD137 antibody comprising a Clec9A polypeptide, and
a bispecific anti-CD3 anti-CD137 antibody comprising a Clec4e polypeptide.

In an embodiment, the antigen-binding molecule is an antibody, in particular an IgG type antibody, wherein the antigen-binding molecule comprises:
(1) one first moiety wherein said first moiety is linked to the Fc region, in particular wherein said first moiety is an Ig type binding moiety,
(2) one second moiety wherein said second moiety is linked to the Fc region, and is an Ig type binding moiety, and
(3) one third moiety wherein said third moiety is linked to the Fc region, and is an Ig type binding moiety.

In an embodiment herein, the one second moiety and the one third moiety together form a F(ab′)₂. For example, an antigen-binding molecule can be provided wherein a F(ab′)₂ binding to CD3 and CD137 is linked to a Fc, which is further linked to a Ig-like moiety, like a scFv, VHH or variable domain, that binds to SF3B3, PCNA, PS or F-actin.

In an aspect, a pharmaceutical composition is provided comprising an antigen-binding molecule of any one of the embodiments herein. In one embodiment, the antigen-binding molecule is as specified in any one of the embodiments of invention group [AA].

Further provided herein is an antigen-binding molecule of an embodiment or aspect herein, or a pharmaceutical composition comprising an antigen-binding molecule of an embodiment or aspect herein for use in medicine. In one embodiment, the antigen-binding molecule is as specified in any one of the embodiments of invention group [AA].

In an embodiment, an antigen-binding molecule of an embodiment or aspect herein, or a pharmaceutical composition comprising an antigen-binding molecule of an embodiment or aspect herein is provided for use in a method of treating or preventing a medical condition, preferably wherein the medical condition is selected from the group consisting of a cancer and an immune, preferably an autoimmune, disease. The cancer disease is in one embodiment as provided herein. In one embodiment, the antigen-binding molecule is as specified in any one of the embodiments of invention group [AA].

In embodiments, in any of the antigen-binding molecules of any of the aspects and embodiment herein, the antigen-binding molecule:
A) is capable of providing a cellular contact and/or signal due to the said cell
B) comprises the said first moiety at an Fc terminus of the antigen-binding molecule; and/or
C) comprises the said second moiety in an Ig-type binding moiety, preferably an F(ab′)₂ region, of the antigen-binding molecule; and/or
D) comprises at least one first moiety and at least one second moiety, wherein (i) the antigen-binding molecule comprises one first moiety and one second moiety, or wherein (ii) the antigen-binding molecule comprises at least two, preferably two, first moieties and at least two, preferably two, second moieties; and/or
E) comprises the said third moiety in Ig-type binding moiety, preferably an F(ab′)2 region, of the antigen-binding molecule; and/or
F) comprises at least one first moiety and at least one second moiety and at least one third moiety, particularly wherein (i) the antigen-binding molecule comprises one first moiety and one second moiety and one third moiety, or wherein (ii) the antigen-binding molecule comprises at least two, preferably two, first moieties and at least two, preferably two, second moieties and at least two, preferably two, third moieties; and/or
G) wherein the antigen-binding molecule is capable of binding to the first antigen and the second antigen upon light irradiation.

In embodiments of any of the aspects herein, said cell damage
i) involves cell death, particularly is cell death, and/or
ii) occurs in an affected tissue, particularly selected from a tissue affected by a condition or disease, particularly by a cancer or an autoimmune disease, especially in a tumor microenvironment (TME); and/or
iii) results from any one selected from the group consisting of necrosis, apoptosis, an autophagy cell death and an accidental cell death, and/or
iv) is cell death selected from the group consisting of necrosis, apoptosis, an autophagy cell death and an accidental cell death.

In embodiments of any of the aspects or embodiments herein, the antigen-binding molecule is characterized in that a complex, which is formed by binding of one or more of the antigen-binding molecules to the first antigen, is capable of binding to more than one membrane proteins via the at least one second moiety and/or the at least one third moiety, particularly wherein
i) the complex, which is formed by binding of one or more of the antigen-binding molecules to the first antigen, is capable of binding to one or more of the membrane proteins via the at least one second moiety to confer a signal transmission via the said one or more membrane protein in a cell, or
ii) the complex, which is formed by binding of one or more of the antigen-binding molecules to the first antigen, is capable of binding to one or more of the membrane proteins via the at least one second moiety to confer a signal blockage via the said one or more membrane proteins in a cell, or
iii) the complex, which is formed by binding of one or more of the antigen-binding molecules to the first antigen, is capable of binding to one or more of the membrane proteins via the at least one third moiety to confer a signal transmission via the said one or more membrane protein in a cell, or
iv) the complex, which is formed by binding of one or more of the antigen-binding molecules to the first antigen, is capable of binding to one or more of the membrane proteins via the at least one third moiety to confer a signal blockage via the said one or more membrane proteins in a cell.

In embodiments of any of the aspects or embodiments herein, the antigen-binding molecule is selected from the group consisting of:
A) a polypeptide complex, which optionally is a fusion protein,
B) a polynucleotide (preferably composed of two or more entities, preferably connected with a linker),
C) a medium-sized chemical compound (preferably composed of two or more entities, preferably connected with a linker),
D) a small-sized chemical compound (preferably composed of two or more entities, preferably connected with a linker), particularly wherein
A-1) the said antigen-binding molecule is a polypeptide complex, especially wherein the said antigen-binding molecule is an antibody or an antibody fragment thereof, in particular wherein the said antigen-binding molecule is an antibody, preferably wherein said antibody is an IgG type antibody and/or a bispecific antibody; or
A-2) the said antigen-binding molecule is a polypeptide complex, which is a fusion protein,
especially wherein the said antigen-binding molecule is an antibody, which is a fusion protein, or an antibody fragment thereof, in particular wherein the said antigen-binding molecule is an antibody, which is a fusion protein,
preferably wherein said antibody is a) an IgG type antibody having at least one binding polypeptide, preferably two binding polypeptides, fused to its Fc domain, and/or b) a bispecific antibody having at least one binding polypeptide, preferably two binding polypeptides, fused to its Fc domain, and/or c) a trispecific antibody having at least one binding polypeptide, preferably two binding polypeptides or three binding polypeptides, fused to its Fc domain; or
A-3) the said antigen-binding molecule is a polypeptide complex, which is a fusion protein,
especially wherein the said antigen-binding molecule is an antibody, which is a fusion protein, or an antibody fragment thereof,
in particular wherein the said antigen-binding molecule is an antibody, which is a fusion protein, preferably said antibody is a) an IgG type antibody having at least one Ig type binding moiety, preferably two Ig type binding moieties, fused to its Fc domain or three Ig type binding moieties, fused to its Fc domain and/or b) a bispecific antibody having at least one Ig type binding moiety, preferably two Ig type binding moieties, fused to its Fc domain and/or c) a trispecific antibody having at least one Ig type binding moiety, preferably two Ig type binding moieties, fused to its Fc domain or three Ig type binding moieties, fused to its Fc domain.

In embodiments, the Ig type binding moiety is selected from the group consisting of IgG type antibody, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)2, especially wherein the said Ig-type binding moiety is selected from the group consisting of an VHH, an scFv, an scFv2, a Fab, and a F(ab′)2.

In embodiments of any of the aspects or embodiments herein, the antigen-binding molecule is characterized in that
i-1) the first moiety is Ig-type binding moiety, or
i-2) the first moiety is a binding polypeptide; and/or
ii) the second moiety is an Ig-type binding moiety; and/or
iii) the third moiety is an Ig-type binding moiety;
particularly wherein
a) the first moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)2; and/or
b) the second moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)2; and/or
c) the said binding polypeptide is a non-Ig-type binding moiety; and/or
d) the third moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)2; preferably wherein
   a*) the first moiety is a binding domain selected from the group consisting of an VHH, an scFv, an scFv2, a Fab, and a F(ab′)2, and/or
   b*) the second moiety is a binding domain selected from the group consisting of an VHH, an scFv, an scFv2, a Fab, and a F(ab′)2; and/or
   c*) wherein the binding polypeptide is a non-Ig-type binding moiety, especially a non-Ig-type binding moiety attached to an Fc domain of the said antigen-binding molecule; and/or
   d*) wherein the third moiety is a binding domain selected from the group consisting of an VHH, an scFv, an scFv2, a Fab, and a F(ab′)2.

In embodiments of any of the aspects herein, the antigen-binding molecule is characterized in that the antigen-binding molecule is a bispecific antibody wherein two antigen-binding domains of the bispecific antibodies of any of the embodiments herein are linked with each other via one or more bonds. Methods for producing such molecules are for example disclosed in EP3831854. In embodiments, at least one of amino acid residues from which the bonds between the antigen-binding domains originate is present within a variable region. In embodiments, at least one of amino acid residues from which the bonds between the antigen-binding domains originate is present within a hinge region.

In embodiments of any of the aspects herein, the antigen-binding molecule is characterized in that the first moiety is an Ig-type binding moiety or binding polypeptide; and the second moiety is an Ig-type binding moiety, and/or the third moiety is an Ig-type binding moiety, and the second and/or third moiety is capable of binding to CD3 and CD137, but does not bind to CD3 and CD137 at the same time. Such molecules and methods for producing the same are for example disclosed in EP3720963.

In embodiments of any of the aspects herein, the antigen-binding molecule is characterized in that the second and/or the third antigen is
A) involved in a signal transmission in a cell;
particularly
   A-1) wherein the said second and/or third antigen is a membrane protein, and/or
   A-2) wherein the said second and/or third antigen is a signal transmission receptor, and/or
   A-3) wherein the cell is i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, ii) a tumor cell, or iii) an autoreactive cell; and/or
B) an immune cell receptor antigen.

In embodiments of any of the aspects herein, the antigen-binding molecule is characterized in that the second and/or the third antigen is independently selected from the group consisting of
A) a membrane protein of an endosome,
B) a membrane protein in a cell membrane of a T cell, in particular a membrane protein in a cell membrane of a T cell selected from the group consisting of T cell receptor, CD3, CD137, CD40, CTLA4, a costimulatory molecule or a coinhibitory molecule, especially wherein the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9;
C) a membrane protein in a cell membrane of a cell other than a T cell, particularly which is capable of binding to a costimulatory molecule or a coinhibitory molecule in a cell membrane of a T cell
in particular wherein the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4), and/or wherein the coinhibitory molecule is selected from the group consisting of CD279 (PD-1), CD152 (CTLA-4), TIGIT, BTLA, CD366 (Tim-3), CD96, CD112R, or CD200R, and/or
especially wherein the second and/or third antigen is selected from the group consisting of CD3, CD137, CD40 and CTLA4,
in particular wherein the second and/or the third antigen is independently selected from the group consisting of CD3 and CD137, preferably wherein the second and/or the third antigen is CD3.

In embodiments, the second and/or the third moiety is independently selected from the group consisting of
A) a moiety capable of binding to a membrane protein of an endosome,
B) a moiety capable of binding to a membrane protein in a cell membrane of a T cell,
in particular a membrane protein in a cell membrane of a T cell selected from the group consisting of T cell receptor, CD3, CD137, CD40, CTLA4, a costimulatory molecule or a coinhibitory molecule,
especially wherein the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9; and
C) a moiety capable of binding to a membrane protein in a cell membrane of a cell other than a T cell, particularly which is capable of binding to a costimulatory molecule or a coinhibitory molecule in a cell membrane of a T cell,
in particular wherein the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4), and/or wherein the coinhibitory molecule is selected from the group consisting of CD279 (PD-1), CD152 (CTLA-4), TIGIT, BTLA, CD366 (Tim-3), CD96, CD112R, or CD200R, especially wherein the second and/or third moiety is selected from the group consisting of a moiety capable of binding to CD3, a moiety capable of binding to CD137, a moiety capable of binding to CD40 and a moiety capable of binding to CTLA4.
in particular wherein the second and/or the third moiety is independently selected from the group consisting of a moiety capable of binding to CD3 and/or a moiety capable of binding to CD137, preferably wherein the second and/or third moiety is a moiety capable of binding to CD3.

In embodiments of any of the aspects herein, the antigen-binding molecule is characterized in that
A) the moiety capable of binding to CD3 is Ig-type binding moiety, and/or
B) the moiety capable of binding to CD137 is Ig-type binding moiety, and/or
C) the moiety capable of binding to CD40 is Ig-type binding moiety, and/or
D) the moiety capable of binding to CTLA4 is Ig-type binding moiety;
particularly wherein the said Ig-type binding moiety is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)2, especially wherein the said Ig-type binding moiety is selected from the group consisting of an VHH, an scFv, an scFv2, a Fab, and a F(ab′)2.

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration (also described as “amino acid substitution” or “amino acid mutation” within this description) in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present invention, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Recombinant Methods and Compositions

Antibodies, antigen-binding molecules, antigen-binding domains, molecules including those which belong to the present invention may be produced using recombinant methods and compositions, e.g., as described in U.S. Pat. No. 4,816,567. In case that the molecule of the present invention is an antibody, in one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making the molecule of the present invention is provided, wherein the method comprises culturing a host cell comprising a nucleic acid encoding the molecule (e.g., antibody) as described above, under conditions suitable for expression of the molecule (e.g., antibody) as described above, and optionally recovering the molecule (e.g., antibody) as described above, from the host cell (or host cell culture medium).

For recombinant production of the molecule (e.g., antibody) as described above, nucleic acid encoding the molecule (e.g., antibody) as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody, in case that the molecule is an antibody.).

Suitable host cells for cloning or expression of the vectors encoding the molecule of the present invention include prokaryotic or eukaryotic cells described herein. For example, the molecule of the present invention may be produced in bacteria, in particular when glycosylation and Fc effector function are not needed. For expression of the molecule of the present invention in bacteria, see, e.g., U.S. Pat. Nos. 5,648,237, 5,789,199, and 5,840,523 (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the molecule of the present invention may be isolated from the bacterial cell paste in a soluble fraction and can be further purified.

In addition to prokaryotes, eukaryotic microbes such as filamentous fungi or yeast are suitable cloning or expression hosts for the vectors encoding the molecule of the present invention, including fungi and yeast strains whose glycosylation pathways have been “humanized,” resulting in the production of the molecule of the present invention with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated molecule of the present invention are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

Recombinant production of the molecule of the present invention could be done with methods similar to those described above, by using a host cell comprises (e.g., has been transformed with) one or plural vectors comprising nucleic acid that encodes an amino acid sequence comprising a whole molecule or a portion thereof.

In the present invention, as described above, one embodiment of the molecule of the present invention, that is, “a molecule comprising a first moiety that binds to a first antigen and a second moiety that binds to a second antigen “, as descried above, is an antibody or an antigen-binding domain/molecule thereof, which may comprise an additional moiety conjugated to, e.g., Fc region. In the present invention, such molecule may also be referred as an antigen-binding molecule. In case that the molecule is, for example, an antigen-binding molecule having multispecificities for two or more different antigens, it may be termed as multispecific antigen-binding molecule.

In one aspect, a polynucleotide or two or more polynucleotides encoding an antigen-binding molecule according to any one of the embodiments or aspects herein is provided. For example, in case the antigen-binding molecule comprises or consists of more than one polypeptide chain, the antigen-binding molecule may be encoded by one polynucleotide, or the antigen-binding molecule may be encoded by two or more polynucleotides. Such two or more polynucleotides may for example be present on the same single vectors or separately, on two or more vectors. For example, two or more vectors may be expressed in a single host cell, or they may be expressed separately.

In one aspect, a polynucleotide, in particular a DNA encoding the antigen-binding molecule of any one of the embodiments or aspects herein is provided. In one aspect, a vector comprising the polynucleotide of the embodiments or aspects herein is provided.

In one aspect, a vector comprising a DNA sequence encoding for the antigen-binding molecule as defined in any one of the embodiments or aspects herein is provided, wherein the vector is in particular for recombinant protein expression. In one aspect, a host cell comprising the said vector of the embodiments or aspects herein is provided.

In one aspect, a host cell comprising the vector of the embodiments or aspects herein is provided, wherein the host cell is in particular for recombinant protein expression of the antigen-binding molecule encoded in the vector.

In one aspect, a method of producing an antigen-binding molecule is provided, which comprises the steps of:
(a) providing one or more nucleic acid(s) encoding at least one first moiety wherein the first moiety binds to a first antigen, and wherein the first antigen is a damage-associated molecular pattern (DAMP), wherein the first antigen is selected from the group consisting of: a) Splicing Factor 3B Subunit 3 (SF3B3); and b) Proliferating Cell Nuclear Antigen (PCNA);
(b) providing one or more nucleic acid(s) encoding at least one second moiety wherein the second moiety binds to a second antigen, and wherein the second antigen is different from the first antigen, preferably wherein said second antigen is a membrane protein of an immune cell, or a tumour cell,
(b1) optionally providing one or more nucleic acid(s) encoding at least one third moiety wherein the third moiety binds to a third antigen, wherein the third antigen is different from the first antigen preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c) thereby obtaining the antigen-binding molecule.

In embodiments, the first moiety in step (a) is as defined in any of one of embodiments [AA1]-[AA13]. In embodiments, the second moiety in step (b) is as defined in any of one of embodiments [AA1]-[AA13]. In embodiments, the third moiety in step (b1) is as defined in any of one of embodiments [AA2]-[AA13]. In embodiments, the one or more nucleic acid(s) is RNA or DNA. In embodiments, the one or more nucleic acid(s) obtained in step (c) are one nucleic acid or two nucleic acids, optionally wherein the two nucleic acids are on a single expression vector or on separate expression vectors.

In embodiments, step (d) comprises recombinant expression of the antigen-binding molecule in a host cell.

In another aspect, a method of producing an antigen-binding molecule is provided, which comprises the steps of:
(a) providing one or more nucleic acid(s) encoding at least one first moiety wherein the first moiety binds to a first antigen, and wherein the first antigen is a damage-associated molecular pattern (DAMP); wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) providing one or more nucleic acid(s) encoding at least one second moiety wherein the second moiety binds to a second antigen, and wherein the second antigen is different from the first antigen; preferably wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and the at least one first moiety provided in (b1) is/are linked; and
(d) producing the antigen-binding molecule using the one or more nucleic acid(s) prepared in (c) thereby obtaining the antigen-binding molecule.

In embodiments, said third antigen is different from the first and second antigen. In embodiments, said third antigen is different from the first but same as the second antigen. In embodiments, the antigen-binding molecule obtained in step (b) is as defined in any of one of embodiments [AA117]-[AA126]. In embodiments, the first moiety in step (a) is as defined in any of one of embodiments [AA117]-[AA126]. In embodiments, the second moiety in step (b) is as defined in any of one of embodiments [AA117]-[AA126]. In embodiments, the third moiety in step (b′) is as defined in any of one of embodiments [AA117]-[AA126]. In embodiments, the one or more nucleic acid(s) is RNA or DNA. In embodiments, the one or more nucleic acid(s) obtained in step (c) are one nucleic acid or two nucleic acids, optionally wherein the two nucleic acids are on a single expression vector or on separate expression vectors.

In embodiments, step (d) comprises recombinant expression of the antigen-binding molecule in a host cell.

In one aspect, provided is the use of at least one first moiety and at least one second moiety for the production of an antigen-binding molecule comprising the at least one first moiety and the at least one second moiety, wherein
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell, or a tumour cell, and wherein the first antigen is selected from the group consisting of:
a) Splicing Factor 3B Subunit 3 (SF3B3); and
b) Proliferating Cell Nuclear Antigen (PCNA).

In an embodiment, the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In an embodiment, the use is an in vitro use. In an embodiment, the use is an in vivo use. In an embodiment, the use is an ex vivo use. In embodiments, the first moiety is as defined in any of one of embodiments [AA1]-[AA13]. In embodiments, the second moiety is as defined in any of one of embodiments [AA1]-[AA13]. In embodiments, the third moiety is as defined in any of one of embodiments [AA2]-[AA13].

In one aspect, provided is the use of at least one first moiety, at least one second moiety and at least one third moiety for the production of an antigen-binding molecule which comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
wherein said second antigen is a membrane protein of an immune cell, or a tumour cell, and wherein the molecule comprises at least one third moiety that binds to a third antigen; wherein said third antigen is different from the first antigen; preferably wherein said third antigen is a membrane protein of an immune cell, or a tumour cell,
wherein the first antigen is selected from the group consisting of:
a) F-actin;
b) Phosphatidylserine (PS);
c) Splicing Factor 3B Subunit 3 (SF3B3); and
d) Proliferating Cell Nuclear Antigen (PCNA).

In embodiments, (i) said third antigen is different from the first and second antigen, or wherein (ii) said third antigen is different from the first but same as the second antigen.

In embodiments, the first moiety is as defined in any one of embodiments [AA117]-[AA126]. In embodiments, the second moiety is as defined in any of one of embodiments [AA117]-[AA126]. In embodiments, the third moiety is as defined in any of one of embodiments [AA117]-[AA126].

### Antigen-Binding Molecules and Multispecific Antigen-Binding Molecules

The term “antigen-binding molecule”, as used herein, refers to any molecule that comprises an antigen-binding site or any molecule that has binding activity to an antigen, and may further refer to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such. Scaffold molecules comprising known stable conformational structure such as alpha/beta barrel as scaffold, and in which part of the molecule is made into antigen-binding site, is also one embodiment of the antigen binding molecule described herein.

“Multispecific antigen-binding molecules” refers to antigen-binding molecules that bind specifically to more than one antigens. The term “bispecific” means that the antigen binding molecule is able to specifically bind to at least two distinct antigenic determinants. The term “trispecific” means that the antigen binding molecule is able to specifically bind to at least three distinct antigenic determinants.

### Antigen Binding Domain

As described above, in case that the molecule of the present invention, that is, “a molecule comprising a first moiety that binds to a first antigen and a second moiety that binds to a second antigen” is, for example, an antigen-binding molecule such as an antibody, which is also referred as an antigen-binding molecule as described above, in some embodiments, “a second moiety that binds to a second antigen” and, if appropriate, further “a first moiety that binds to a first antigen” may be an antigen binding domain of or derived from an antibody.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F(ab′)2”.

### Ig Type Binding Moiety

The terms “Ig type binding moiety”, “Ig-type binding moiety”, “Ig-like moiety”, “Ig-like binding moiety” and the like, as used interchangeably herein, are well understood by the person skilled in the art and generally refer to an antigen binding moiety of the Ig type (immunoglobulin type), which is well known to the skilled person. In certain embodiments herein, it includes all “antigen binding domains” described herein (provided those included at least one immunoglobulin structure)—and it particularly includes all examples of the latter term described herein. Preferably herein, the said Ig-type binding moiety is selected from the group consisting of, a single domain antibody (VHH), an antibody heavy chain variable (VH) region, an antibody light chain variable (VL) region, a single-domain antibody (sdAb), a single-chain Fv (scFv), a single-chain antibody, an Fv, a single-chain Fv2 (scFv2), a Fab, and a F(ab′)₂ . . . . More preferably the said Ig-type binding moiety is selected from the group consisting of an VHH, an scFv, an scFv2, a Fab, and a F(ab′)₂. It especially includes any of the group consisting of “single-chain Fv (scFv)”, “single-chain antibody”, “Fv”, “single-chain Fv2 (scFv2)”, “Fab”, and “F(ab′)₂”—and especially the particular embodiments of any of those described elsewhere herein.

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3).

Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra. HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

### Capable of Binding to an Antigen

Whether the antibody variable region is “capable of binding to an antigen” can be determined by a method known in the art. This can be determined by, for example, an electrochemiluminescence method (ECL method) (BMC Research Notes 2011, 4:281).

Specifically, for example, a low-molecular antibody composed of a region capable of binding to an antigen, for example, a Fab region, of a biotin-labeled antigen-binding molecule to be tested, or a monovalent antibody (antibody lacking one of the two Fab regions carried by a usual antibody) thereof is mixed with an antigen labeled with sulfo-tag (Ru complex), and the mixture is added onto a streptavidin-immobilized plate. In this operation, the biotin-labeled antigen-binding molecule to be tested binds to streptavidin on the plate. Light is developed from the sulfo-tag, and the luminescence signal can be detected using Sector Imager 600 or 2400 (MSD K.K.) or the like to thereby confirm the binding of the aforementioned region of the antigen-binding molecule to be tested to an antigen. Alternatively, this assay may be conducted by ELISA, FACS (fluorescence activated cell sorting), ALPHAScreen (amplified luminescent proximity homogeneous assay screen), the BIACORE method based on a surface plasmon resonance (SPR) phenomenon, etc. (Proc. Natl. Acad. Sci. USA (2006) 103 (11), 4005-4010).

Specifically, the assay can be conducted using, for example, an interaction analyzer Biacore (GE Healthcare Japan Corp.) based on a surface plasmon resonance (SPR) phenomenon. The Biacore analyzer includes any model such as Biacore T100, T200, X100, A100, 4000, 3000, 2000, 1000, or C. Any sensor chip for Biacore, such as a CM7, CM5, CM4, CM3, C1, SA, NTA, L1, HPA, or Au chip, can be used as a sensor chip. Proteins for capturing the antigen-binding molecule of the present invention, such as protein A, protein G, protein L, anti-human IgG antibodies, anti-human IgG-Fab, anti-human L chain antibodies, anti-human Fc antibodies, antigenic proteins, or antigenic peptides, are immobilized onto the sensor chip by a coupling method such as amine coupling, disulfide coupling, or aldehyde coupling. An antigen is injected thereon as an analyte, and the interaction is measured to obtain a sensorgram. In this operation, the concentration of an antigen can be selected within the range of a few micro M to a few pM according to the interaction strength (e.g., KD) of the assay sample.

Alternatively, an antigen may be immobilized instead of the antigen-binding molecule onto the sensor chip, with which the antibody sample to be evaluated is in turn allowed to interact. Whether the antibody variable region of the antigen-binding molecule of the present invention has binding activity against an antigen can be confirmed on the basis of a dissociation constant (KD) value calculated from the sensorgram of the interaction or on the basis of the degree of increase in the sensorgram after the action of the antigen-binding molecule sample over the level before the action.

In some embodiments, binding activity or affinity of the antibody variable region of the present invention to the antigen of interest (i.e. an antigen) are assessed at 37 degrees C. or 25 degrees C. (for an antigen) using e.g., Biacore T200 instrument (GE Healthcare) or Biacore 8K instrument (GE Healthcare). Anti-human Fc (e.g., GE Healthcare) is immobilized onto all flow cells of a CM4 sensor chip using amine coupling kit (e.g, GE Healthcare). The antigen binding molecules or antibody variable regions are captured onto the anti-Fc sensor surfaces, then the antigen is injected over the flow cell. The capture levels of the antigen binding molecules or antibody variable regions may be aimed at 200 resonance unit (RU). Recombinant human antigen may be injected at 400 to 25 nM prepared by two-fold serial dilution, followed by dissociation. All antigen binding molecules or antibody variable regions and analytes are prepared in ACES pH 7.4 containing 20 mM ACES, 150 mM NaCl, 0.05% Tween 20, 0.005% NaN₃. Sensor surface is regenerated each cycle with 3M MgCl₂. Binding affinity are determined by processing and fitting the data to 1:1 binding model using e.g., Biacore T200 Evaluation software, version 2.0 (GE Healthcare) or Biacore 8K Evaluation software (GE Healthcare). The KD values are calculated for assessing the specific binding activity or affinity of the antigen binding domains.

The ALPHAScreen is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). The amount of the analyte bound to the ligand captured on the sensor chip surface (amount of change in response on the sensorgram between before and after the interaction of the analyte) can be determined from the sensorgram. However, since the amount bound also depends on the amount of the ligand, the comparison must be performed under conditions where substantially the same amounts of the ligand are used. Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

For example, a biotin-labeled antigen-binding molecule to be tested is allowed to bind to streptavidin on the donor bead, while an antigen tagged with glutathione S transferase (GST) is allowed to bind to the acceptor bead. The antigen-binding molecule to be tested interacts with an antigen in the absence of the competing second antigen to generate signals of 520 to 620 nm. The untagged second antigen competes with an antigen for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. an antigen can be tagged with GST by an appropriately adopted method which involves, for example: fusing a polynucleotide encoding an antigen in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

The tagging is not limited to the GST tagging and may be carried out with any tag such as, but not limited to, a histidine tag, MBP, CBP, a Flag tag, an HA tag, a V5 tag, or a c-myc tag. The binding of the antigen-binding molecule to be tested to the donor bead is not limited to the binding using biotin-streptavidin reaction. Particularly, when the antigen-binding molecule to be tested comprises Fc, a possible method involves allowing the antigen-binding molecule to be tested to bind via an Fc-recognizing protein such as protein A or protein G on the donor bead.

### Fab Molecule/Fab Region/Fab Arm

In the present invention, the term “Fab molecule”, “Fab region” or “Fab arm” which may be used interchangeably means a protein consisting of the VH and CH1 domain of the heavy chain (the “Fab heavy chain”) and the VL and CL domain of the light chain (the “Fab light chain”) of an immunoglobulin.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Fused/Fusion

The term “fused” and “fusion” mean that two or more different polypeptides (e.g. a first polypeptide that binds to a first antigen and a second polypeptide that binds to a second antigen; a Fab arm and a Fc domain) are linked by peptide bonds, either directly or via one or more peptide linkers.

Consequently, as used herein, the term “fusion protein” (which may be used interchangeably herein with the term “fusion polypeptide”) preferably means that two or more different polypeptides are covalently linked by directly or via one or more peptide linkers. Particular examples of fusion proteins herein are antigen-binding molecules of the invention, in which an antibody (or at least an Ig-type binding moiety) is linked to a binding polypeptide, which is a non-Ig-type binding moiety.

### Crossover Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

### Conventional Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma1 (IgG1), gamma2 (IgG2), gamma3 (IgG3), gamma4 (IgG4), alpha1 (IgA1) and alpha2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

### Affinity

“Affinity” refers to the strength of the sum total of noncovalent interactions between a single binding site of a molecule (e.g., a first moiety or a second moiety; an antibody or an antigen binding domain) and its binding partner (e.g., a first antigen or a second antigen; or an antigen). Unless indicated otherwise, as used herein, “binding affinity” refers to intrinsic binding affinity which reflects a 1:1 interaction between members of a binding pair (e.g., a first moiety and a first antigen; a second moiety and a second antigen; an antigen-binding molecule and antigen, or antibody and antigen). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

### Methods to Determine Affinity

In certain embodiments, each of a first moiety or a second moiety of “a molecule comprising a first moiety that binds to a first antigen and a second moiety that binds to a second antigen” of the present invention, and the antigen-binding molecule or antibody as described above has a dissociation constant (KD) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³ M, 10⁻⁹ M to 10⁻¹³ M) for its antigen (i.e., a first antigen, a second antigen). In certain embodiments, the KD value of the molecule of the present invention for a first antigen and a second antigen respectively falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). For example, solution binding affinity of the molecule of the present invention for antigen (i.e., a first antigen, a second antigen) is measured by equilibrating the molecule with a minimal concentration of (1251)-labeled antigen in the presence of a titration series of unlabeled antigen, then capturing bound antigen with a plate on which an antibody that binds to the molecule is coated (see, e.g., Chen et al., J. Mol. Biol. 293:865-881 (1999)). To establish conditions for the assay, MICROTITER (registered trademark) multi-well plates (Thermo Scientific) are coated overnight with 5 micro g/ml of a capturing an antibody that binds to the molecule (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The molecule of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro L/well of scintillant (MICROSCINT-20 TM; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE (registered trademark)-3000 (BIAcore, Inc., Piscataway, NJ) is performed at 25 degrees C. with immobilized antigen CM5 chips at ~10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (~0.2 micro M) before injection at a flow rate of 5 micro L/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro L/min. Association rates (kon) and dissociation rates (koff) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM an antibody that binds to the molecule in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the molecule of the present invention for antigen(s) described above, persons skilled in art can carry out affinity measurement for any other sort or type of the molecule of the present invention, towards various kind of antigens.

### Antibody

As described above, in case that the molecule of the present invention, that is, “a molecule comprising a first moiety that binds to a first antigen and a second moiety that binds to a second antigen” is, for example, an antigen-binding molecule such as an antibody, which is also referred as an antigen-binding molecule as described above, in some embodiments, “a second moiety that binds to a second antigen” and, if appropriate, further “a first moiety that binds to a first antigen” may be an antigen binding domain of or derived from an antibody.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure or having heavy chains that contain an Fc region as defined herein.

### Antibody Fragment

An “antibody fragment” (which may also be referred a portion of an antibody) refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

In particular embodiments herein, an antibody fragment is an Ig type binding moiety.

### Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240 (4855), 1038-1041). In the Fv prepared from the periplasm fraction, VH associates with VL in a manner so as to bind to an antigen.

scFv, Single-Chain Antibody, and Sc (Fv) 2

Herein, the terms “scFv”, “single-chain antibody”, and “sc(Fv) 2” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an single chain low molecule weight antibody in which VH and VL forming Fv are linked together by a peptide linker (Proc. Natl. Acad. Sci. U.S.A. (1988) 85 (16), 5879-5883). VH and VL can be retained in close proximity by the peptide linker.

sc(Fv) 2 is a single chain antibody in which four variable regions of two VL and two VH are linked by linkers such as peptide linkers to form a single chain (J Immunol. Methods (1999) 231 (1-2), 177-189). The two VH and two VL may be derived from different monoclonal antibodies. Such sc(Fv) 2 preferably includes, for example, a bispecific sc(Fv) 2 that recognizes two epitopes present in a single antigen as disclosed in the Journal of Immunology (1994) 152 (11), 5368-5374. sc(Fv) 2 can be produced by methods known to those skilled in the art. For example, sc(Fv) 2 can be produced by linking scFv by a linker such as a peptide linker.

Herein, an sc(Fv) 2 includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.
[VL]-linker-[VH]-linker-[VH]-linker-[VL] [VH]
linker-[VL]-linker-[VL]-linker-[VH] [VH]
linker-[VH]-linker-[VL]-linker-[VL] [VL]
linker-[VL]-linker-[VH]-linker-[VH] [VL]
linker-[VH]-linker-[VL]-linker-[VH]

The molecular form of sc(Fv) 2 is also described in detail in WO 2006/132352. According to these descriptions, those skilled in the art can appropriately prepare desired sc(Fv) 2 to produce the polypeptide complexes disclosed herein.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, MD, 1991.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P (O) S (“thioate”), P(S) S (“dithioate”), (O) NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

### Subject

In the present invention, a subject is not particularly limited. According to preferred embodiments herein, the subject is an animal, preferably a mammal, more preferably a human.

In general preferred embodiments herein, the subject-matter (including but not limited to any medical uses) relate to an animal subject, preferably a mammalian subject, more preferably a human subject.

Consequently, in preferred embodiments herein, the first antigen is an antigen derived from an animal, preferably a mammal, more preferably a human. Consequently, in preferred embodiments herein, the second antigen is an antigen derived from an animal, preferably a mammal, more preferably a human. Consequently, in preferred embodiments herein, the antibody herein is an antibody derived from an animal, preferably a mammal, more preferably a human. In alternative embodiments, the antibody herein is a humanized antibody.

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

Furthermore, the molecule of the present invention (i.e., “a molecule comprising a first moiety that binds to a first antigen and a second moiety that binds to a second antigen”) may be conjugated with a carrier polymer such as PEG or an organic compound such as an anticancer agent. Alternatively, a sugar chain addition sequence is preferably inserted into the molecule such that the sugar chain produces a desired effect.

### Linkers

The linkers to be used for linking two or more different components (e.g., polypeptides, peptides) comprise arbitrary peptide linkers that can be introduced by genetic engineering, synthetic linkers, and linkers disclosed in, for example, Protein Engineering, 9 (3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. For example, when sc(Fv) 2 contains three peptide linkers, their length may be all the same or different.

For example, such peptide linkers include:

Ser,
Gly-Ser,
Gly-Gly-Ser,
Ser-Gly-Gly,
(SEQ ID NO: 11)
Gly-Gly-Gly-Ser,
(SEQ ID NO: 12)
Ser-Gly-Gly-Gly,
(SEQ ID NO: 13)
Gly-Gly-Gly-Gly-Ser,
(SEQ ID NO: 14)
Ser-Gly-Gly-Gly-Gly,
(SEQ ID NO: 15)
Gly-Gly-Gly-Gly-Gly-Ser,
(SEQ ID NO: 16)
Ser-Gly-Gly-Gly-Gly-Gly,
(SEQ ID NO: 17)
Gly-Gly-Gly-Gly-Gly-Gly-Ser,
(SEQ ID NO: 18)
Ser-Gly-Gly-Gly-Gly-Gly-Gly,
(Gly-Gly-Gly-Gly-Ser (SEQ ID NO: 13))n,
and
(Ser-Gly-Gly-Gly-Gly (SEQ ID NO: 14))n,

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl]sulfone (BSOCOES), and
bis[2-(sulfosuccinimidoxycarbonyloxy)ethyl]sulfone (sulfo-BSOCOES). These crosslinking agents are commercially available.

For example, in general, three linkers are required to link four antibody variable regions together. The linkers to be used may be of the same type or different types.

### Fc Region

The term “Fc region” or “Fc domain” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)₂ under appropriately set reaction conditions (e.g., pH) of the enzyme. Examples thereof can include pepsin and papain.

An Fc region derived from, for example, naturally occurring IgG can be used as the “Fc region” of the present invention. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present invention. Particularly, the sequence of human IgG1 may have DEL or EEM as an amino acid sequence of EU numbering positions 356 to 358.

In some embodiments, the Fc domain of the antibody which belongs to a molecule of the present invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment, the molecule of the present invention may comprises not more than one Fc domain.

In one embodiment, the Fc domain that the molecule may comprise is an IgG Fc domain. In a particular embodiment the Fc domain is an IgG1 Fc domain. In another embodiment the Fc domain is an IgG1 Fc domain. In a further particular embodiment the Fc domain is a human IgG1 Fc region.

### Production and Purification of Multispecific Antibodies

As described above, in case that the molecule of the present invention, that is, “a molecule comprising a first moiety that binds to a first antigen and a second moiety that binds to a second antigen” is, for example, an antigen-binding molecule such as an antibody, which is also referred as an antigen-binding molecule as described above, in some embodiments, “a second moiety that binds to a second antigen” and, if appropriate, further “a first moiety that binds to a first antigen” may be an antigen binding domain of or derived from an antibody. In one embodiment, the molecule, that is, an antigen-binding molecule such as an antibody may be a multispecific antigen binding molecule such as a bispecific, a trispecific, and a tetraspecific antibody or antigen binding molecule.

In one example, the multispecific antigen binding molecules described herein comprise two different antigen binding moieties (e.g. the “first antigen binding moiety” (i.e., a first moiety) and the “second antigen binding moiety” (i.e., a second moiety), fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of multispecific antigen binding molecules in recombinant production, it will thus be advantageous to introduce in the Fc domain of the multispecific antigen binding molecule a modification promoting the association of the desired polypeptides.

Accordingly, in particular embodiments the Fc domain of the multispecific antigen binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the multispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

In addition, other known techniques can also be used for formation of multispecific antibodies. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32 (2): 191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing multispecific antibodies composed of two types of light chains and one type of heavy chain as described in WO2012/023053; technologies for producing multispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

Multispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the multispecific antigen binding molecule binds. For example, for affinity chromatography purification of multispecific antigen binding molecules a matrix with protein A or protein G may be used. Sequential Protein A or G affinity chromatography and size exclusion chromatography can be used to isolate a multispecific antigen binding molecule. The purity of the multispecific antigen binding molecule can be determined by any of a variety of well-known analytical methods including gel electrophoresis, high pressure liquid chromatography, and the like.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164:4178-4184 (2000).

### Pharmaceutical Composition

In one aspect, the present disclosure provides a pharmaceutical composition comprising a molecule of the present invention as described above. e.g., “a molecule comprising a first moiety that binds to a first antigen and a second moiety that binds to a second antigen”.

In certain embodiments, the pharmaceutical composition of the present invention is for conferring a signal transmission or a signal blockade into a cell (e.g., an immune cell, a cell in an affected tissue, as described above), in other words, activating, regulating, suppressing, or inhibiting an immune cells directly or indirectly, or suppressing or inhibiting cells in an affected tissue (e.g., tumor cells). In one embodiment, the pharmaceutical composition of the present invention is a therapeutic agent for inducing cellular cytotoxicity. In certain embodiments, the pharmaceutical composition of the disclosure is a pharmaceutical composition used for treatment and/or prevention of cancer, autoimmune disease, inflammation, viral infection and so on.

In the present invention, the pharmaceutical composition for a treatment may also be referred as a therapeutic agent (including but not limited to a cell growth-suppressing agent, an anticancer agent, or the like).

In the present invention, the terms “cancer”, “carcinoma”, and “cancerous” refer to or describe the physiological condition in mammals that is typically characterized by unregulated cell growth/proliferation. In the present invention, the term “tumor” refers to all neoplastic cell growth and proliferation, whether malignant or benign, and all pre-cancerous and cancerous cells and tissues. The terms “carcinoma”, “cancer”, “cancerous”, “cell proliferative disorder”, “proliferative disorder” and “tumor” are not mutually exclusive as referred to herein.

The terms “cell proliferative disorder” and “proliferative disorder” refer to disorders that are associated with some degree of abnormal cell proliferation. In one embodiment, the cell proliferative disorder is cancer.

In the present invention, the cancer includes but is not limited to, for example, gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor), but are not limited thereto. More preferred cancer types include gastric cancer, colorectal cancer, lung cancer, mesothelioma, liver cancer, breast cancer, skin cancer, lymphoma, and myeloid leukemia, but are not limited thereto (Tumori. (2012) 98, 478-484; Tumor Biol. (2015) 36, 4671-4679; Am J Clin Pathol (2008) 130, 224-230; Adv Anat Pathol (2014) 21, 450-460; Med Oncol (2012) 29, 663-669; Clinical Cancer Research (2004) 10, 6612-6621; Appl Immunohistochem Mol Morphol (2009) 17, 40-46; Eur J Pediatr Surg (2015) 25, 138-144; J Clin Pathol (2011) 64, 587-591; Am J Surg Pathol (2006) 30, 1570-1575; Oncology (2007) 73, 389-394; Diagnostic Pathology (2010) 64, 1-6; Diagnostic Pathology (2015) 34, 1-6; Am J Clin Pathol (2008) 129, 899-906; Virchows Arch (2015) 466, 67-76). More preferable cancer types include gastric cancer, lung cancer, mesothelioma, liver cancer, breast cancer, skin cancer, lymphoma, and myeloid leukemia.

In the present invention, “autoimmune disease” includes but is not limited to vitiligo, type I diabetes, and so on.

In the present invention, “inflammation” includes but is not limited to an inflammation in a tissue of the following.
i. A joint tissue associated with rheumatoid arthritis or osteoarthritis.
ii. A lung tissue associated with bronchial asthma.
iii. A digestive organ tissue associated with inflammatory bowel disease, Crohn disease, or ulcerative colitis.
iv. A fibrotic tissue associated with a fibrosis in liver, kidney or lung.
v. A tissue associated with an immune rejection due to an organ plant.
vi. A blood vessel tissue or a heart tissue associated with arteriosclerosis or heart failure.
vii. A visceral fat tissue associated with metabolic syndrome.
viii. A skin tissue associated with atopic dermatitis or any other dermatitis.
ix. A spinal nerve tissue associated with disc hernia or chronic back pain.
x. A bone tissue associated with a bone fracture.
xi. A tissue associated with a burn.
xii. A tissue of injured organ tissue by mechanical, physical or chemical external force.

A pharmaceutical composition of the present invention may be formulated with one or more of the molecules of the present invention, if needed. For example, the activation, regulation, suppression, or inhibition of an immune response of immune cells, or a cytotoxic action against cells expressing an antigen can be enhanced by a cocktail of two or more of the molecules of the present invention.

A pharmaceutical composition comprising a molecule of the present invention is prepared by mixing the molecule of the present invention as described above having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

If necessary, the molecule of the present invention as described above may be encapsulated in microcapsules (microcapsules made from hydroxymethylcellulose, gelatin, poly [methylmethacrylate], and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for preparing agents as sustained-release agents are known, and these can be applied to the antigen-binding molecules of the present disclosure (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

The pharmaceutical compositions comprising the molecule of the present invention as described above may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions comprising the molecule of the present invention as described above can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present invention is not limited to these doses.

The present invention provides a method for conferring a signal transmission or a signal blockade into a cell (e.g., an immune cell, a cell in an affected tissue, as described above), in other words, activating, regulating, suppressing, or inhibiting an immune cells directly or indirectly, or suppressing or inhibiting cells in an affected tissue (e.g., tumor cells), comprising an administration of a molecule of the present invention as described above into a subject in need thereof. In one embodiment, the method of the present invention is for inducing cellular cytotoxicity in an affected tissue as described above. The present invention further provides a method for treatment and/or prevention of cancer, autoimmune disease, inflammation, viral infection and so on, comprising an administration of a molecule of the present invention as described above into a subject in need thereof.

In an aspect, a method of conferring a signal transmission or blockage in a cell is provided, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen, preferably wherein said second antigen is a membrane protein of an immune cell, or a tumour cell, wherein the first antigen is selected from the group consisting of:
a) Splicing Factor 3B Subunit 3 (SF3B3); and
b) Proliferating Cell Nuclear Antigen (PCNA).

In embodiments, the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In one embodiment, said third antigen is different from the first and second antigen. In one embodiment, said third antigen is different from the first but same as the second antigen.

In embodiments, the method comprises contacting the antigen-binding molecule, or one or more nucleic acid(s) encoding the antigen-binding molecule, a host cell expressing the antigen-binding molecule with the cell. In embodiments, the contacting is performed in vitro. In embodiments, the contacting is performed ex vivo. In embodiments, the contacting is performed in vivo. In embodiments, the first moiety is as defined in any of one of embodiments [AA1]-[AA13]. In embodiments, the second moiety is as defined in any of one of embodiments [AA1]-[AA13]. In embodiments, the third moiety is as defined in any of one of embodiments [AA2]-[AA13].

In another aspect, a method of activating or preventing an immune response is provided, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen, preferably wherein said second antigen is a membrane protein of an immune cell, or a tumour cell, and wherein the first antigen is selected from the group consisting of:
a) Splicing Factor 3B Subunit 3 (SF3B3); and
b) Proliferating Cell Nuclear Antigen (PCNA).

In embodiments, the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In one embodiment, said third antigen is different from the first and second antigen. In one embodiment, said third antigen is different from the first but same as the second antigen.

In embodiments, the method comprises administering the antigen-binding molecule, or one or more nucleic acid(s) encoding the antigen-binding molecule, or a host cell expressing the antigen-binding molecule or a pharmaceutical composition comprising the antigen-binding molecule, or one or more nucleic acid(s) encoding the antigen-binding molecule, or a host cell expressing the antigen-binding molecule to a subject.

In embodiments, the subject is a mammal. In embodiments, the subject is a human.

In embodiments, the subject suffers from cancer or an immune, preferably an autoimmune, disease. In embodiments, the first moiety is as defined in any of one of embodiments [AA1]-[AA13]. In embodiments, the second moiety is as defined in any of one of embodiments [AA1]-[AA13]. In embodiments, the third moiety is as defined in any of one of embodiments [AA2]-[AA13].

In a further aspect, a method of conferring a signal transmission or blockage in a cell is provided, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen, preferably wherein said second antigen is a membrane protein of an immune cell, or a tumour cell, and wherein the molecule comprises at least one third moiety that binds to a third antigen; wherein said third antigen is different from the first antigen; preferably wherein said third antigen is a membrane protein of an immune cell, or a tumour cell, wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA).

In embodiments, said third antigen is different from the first and second antigen. In embodiments, said third antigen is different from the first but same as the second antigen. In embodiments, the first moiety is as defined in any of one of embodiments [AA117]-[AA126]. In embodiments, the second moiety is as defined in any of one of embodiments [AA117]-[AA126]. In embodiments, the third moiety is as defined in any of one of embodiments [AA117]-[AA126].

In embodiments, the method comprises contacting the antigen-binding molecule, or one or more nucleic acid(s) encoding the antigen-binding molecule, or a host cell expressing the antigen-binding molecule with the cell. In embodiments, the contacting is performed in vitro. In embodiments, the contacting is performed ex vivo. In embodiments, the contacting is performed in vivo.

In another aspect, a method of activating or preventing an immune response is provided, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen, preferably wherein said second antigen is a membrane protein of an immune cell, or a tumour cell, and wherein the molecule comprises at least one third moiety that binds to a third antigen; wherein said third antigen is different from the first antigen; preferably wherein said third antigen is a membrane protein of an immune cell, or a tumour cell, and
wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA). In embodiments, said third antigen is different from the first and second antigen. In embodiments, said third antigen is different from the first but same as the second antigen. In embodiments, the first moiety is as defined in any of one of embodiments [AA117]-[AA126]. In embodiments, the second moiety is as defined in any of one of embodiments [AA117]-[AA126]. In embodiments, the third moiety is as defined in any of one of embodiments [AA117]-[AA126]. In embodiments, the method comprises administering the antigen-binding molecule, or one or more nucleic acid(s) encoding the antigen-binding molecule, or a host cell expressing the antigen-binding molecule or a pharmaceutical composition comprising the antigen-binding molecule, or one or more nucleic acid(s) encoding the antigen-binding molecule, or a host cell expressing the antigen-binding molecule to a subject. In embodiments, the subject is a mammal. In embodiments, the subject is a human. In embodiments, the subject suffers from cancer or an immune, preferably an autoimmune, disease.

In embodiments of any of the aspects herein, the antigen-binding molecule:
A) is capable of providing a cellular contact and/or signal due to the said cell damage wherein the said contact and/or signal involves the said second antigen; and/or
B) comprises the said first moiety at an Fc terminus of the antigen-binding molecule; and/or
C) comprises the said second moiety in an Ig-type binding moiety, preferably an F(ab′)₂ region, of the antigen-binding molecule; and/or
D) comprises at least one first moiety and at least one second moiety, wherein (i) the antigen-binding molecule comprises one first moiety and one second moiety, or wherein (ii) the antigen-binding molecule comprises at least two, preferably two, first moieties and at least two, preferably two, second moieties; and/or
E) comprises the said third moiety in Ig-type binding moiety, preferably an F(ab′)2 region, of the antigen-binding molecule; and/or
F) comprises at least one first moiety and at least one second moiety and at least one third moiety, particularly wherein (i) the antigen-binding molecule comprises one first moiety and one second moiety and one third moiety, or wherein (ii) the antigen-binding molecule comprises at least two, preferably two, first moieties and at least two, preferably two, second moieties and at least two, preferably two, third moieties; and/or
G) wherein the antigen-binding molecule is capable of binding to the first antigen and the second antigen upon light irradiation.

In embodiments of any of the aspects herein, said cell damage
i) involves cell death, particularly is cell death, and/or
ii) occurs in an affected tissue, particularly selected from a tissue affected by a condition or disease, particularly by a cancer or an autoimmune disease, especially in a tumor microenvironment (TME); and/or
iii) results from any one selected from the group consisting of necrosis, apoptosis, an autophagy cell death and an accidental cell death, and/or
iv) is cell death selected from the group consisting of necrosis, apoptosis, an autophagy cell death and an accidental cell death.

The present disclosure also provides kits for use in a method of the present invention, which contain a molecule of the present invention as described above. The kits may be packaged with an additional pharmaceutically acceptable carrier or medium, or instruction manual describing how to use the kits, etc.

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention.

The label or package insert indicates that the composition is used for treating the condition of choice. Moreover, the article of manufacture may comprise (a) a first container with a composition contained therein, wherein the composition comprises a molecule of the present invention as described above; and (b) a second container with a composition contained therein, wherein the composition comprises a further cytotoxic or otherwise therapeutic agent.

The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

### Pharmaceutical Formulation

The term “pharmaceutical formulation” or “pharmaceutical composition” is used interchangeably meaning a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”, which are used interchangeably herein) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules or antibodies of the present disclosure are used to delay development of a disease or to slow the progression of a disease.

### Other Agents and Treatments

The molecule of the present invention as described above may be administered in combination with one or more other agents in therapy. For instance, the molecule of the present invention as described above may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of the molecule of the present invention as described above used, the type of disorder or treatment, and other factors discussed above. The molecule of the present invention as described above is generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the molecule of the present invention as described above can occur prior to, simultaneously, and/or following, administration of the additional therapeutic agent and/or adjuvant. The molecule of the present invention as described above can also be used in combination with radiation therapy.

In the following, particular embodiments of the invention are set forth in further detail, which embodiments in part relate to—but are not limited to—the embodiments outlined above as D 1 to D 37: All definitions and further detailed description given above in particular also relate to the following embodiments.

That is, the present invention particularly relates to an antigen-binding molecule which comprises (1) at least one first moiety that binds to a first antigen, and (2) at least one second moiety that binds to a second antigen; wherein said first antigen is a damage-associated molecular pattern (DAMP), and wherein said second antigen is an antigen different from the first antigen.

Furthermore, the present invention particularly relates to an antigen-binding molecule which comprises:
(1) at least one first moiety that binds to a first antigen, and
(2) at least one second moiety that binds to a second antigen;
wherein said first antigen is a tissue-derived antigen characteristic for a tissue state,
wherein in particular the antigen is exposed to extracellular environment, such as a damage-associated molecular pattern (DAMP), and
wherein said second antigen is different from the first antigen.

According to the present invention, the term “a tissue-derived antigen characteristic for a tissue state” relates to an antigen which is exposed to a extracellular environment due to various different cellular death programs characterized in organs and tissues as consequence of microbes infection, cell stress, injury, and chemotherapeutics exposure. As various cellular death programs apoptosis, necroptosis, and pyroptosis are well known. Dying and death cells release various self-proteins and bioactive chemicals originated from cytosol, nucleus, endoplasmic reticulum, and mitochondria. These endogenous factors are named as pathogen-associated molecular pattern (PAMPs), damage-associated molecular pattern (DAMPs), cell-death associated molecular patterns (CDAMPs), and alarmins (Beatriz Sangiuliano et al, Mediators Inflammation (2014) 2014: Article ID 821043, pages 1-14).

Generally herein, the damage-associated molecular pattern (DAMP) is preferably exposed to an extracellular environment due to cell damage, particularly due to cell death. Preferably, the molecule is capable of providing a cellular contact and/or signal due to the said cell damage wherein the said contact and/or signal involves the said second antigen.

Generally herein, the said cell damage preferably i) involves cell death, particularly is cell death, and/or ii) occurs in an affected tissue, particularly selected from a tissue affected by a condition or disease, particularly by a cancer or an autoimmune disease, especially in a tumor microenvironment (TME); and/or iii) results from any one selected from the group consisting of necrosis, apoptosis, an autophagy cell death and an accidental cell death, and/or iv) is cell death selected from the group consisting of necrosis, apoptosis, an autophagy cell death and an accidental cell death.

Consequently, in certain embodiments herein, the said cell damage preferably involves cell death, particularly is cell death. Consequently, in certain embodiments herein, the said cell damage preferably occurs in an affected tissue, particularly selected from a tissue affected by a condition or disease, particularly by a cancer or an autoimmune disease, especially in a tumor microenvironment (TME).

Consequently, in certain embodiments herein, the said cell damage preferably results from any one selected from the group consisting of necrosis, apoptosis, an autophagy cell death and an accidental cell death. Consequently, in certain embodiments herein, the said cell damage preferably is cell death selected from the group consisting of necrosis, apoptosis, an autophagy cell death and an accidental cell death.

The first antigen as defined herein is not particularly limited- and certain preferred embodiments correspond to those described herein above.

In fact, the first antigen may be any DAMP. DAMP are generally well known and particularly include all DAMP described elsewhere herein, including those molecules described elsewhere herein that are exposed to an extracellular environment due to a cell death.

In certain preferred embodiments, the said first antigen is a cytoskeleton component or a portion thereof, a cell membrane component or a portion thereof, an organelle component or a portion thereof, a cytoplasmic protein or a portion thereof, and a metabolite.

In certain preferred embodiments, the said first antigen is located on any one selected from the group consisting of a filament (particularly an intermediate filament), a nucleosome, a cytoskeleton, and/or a nuclear skeleton.

In certain preferred embodiments, the said first antigen is capable of multimerization of a plurality of molecules. In certain additional/alternative preferred embodiments, the said first antigen is present in a multimeric molecule.

In particular embodiments, the said first antigen is selected from the group consisting of a cytoskeleton component or a portion thereof, a cell membrane component or a portion thereof, an organelle component or a portion thereof, a cytoplasmic protein or a portion thereof, and a metabolite and is located on any one selected from the group consisting of a filament (particularly an intermediate filament), a nucleosome, a cytoskeleton, and a nuclear skeleton.

In certain preferred embodiments, the said first antigen is selected from the group consisting of A) an actin, particularly F-actin; B) a heat shock protein, particularly selected from HSP90 and GRP78, especially HSP90; C) a phosphatidylserine (PS), particularly a phosphatidylserine that is part of a cell membrane; D) a histone or component thereof, E) a histone deacetylase complex subunit, particularly SAP130, F) a HMGN protein, particularly selected from HMGB1 and HMGN1, G) hepatoma-derived growth factor, H) BCL-2, I) calreticulin, and J) cyclophilin A.

In certain preferred embodiments, the first antigen is selected from the group consisting of F-actin, GRP78, phosphatidylserine, HSP90, and SAP130; more preferably from the group consisting of F-actin, phosphatidylserine, and HSP90; even more preferably selected from the group consisting of F-actin and HSP90, and in particular the first antigen is F-actin.

The first moiety as defined herein is not particularly limited- and certain preferred embodiments correspond to those described herein above.

The present embodiments relating to the first antigen may—additionally or

alternatively—be further defined by reference to the first moiety.

In certain preferred embodiments, the first moiety is an Ig type binding moiety.

In other preferred embodiments, the first moiety is an a binding polypeptide. Preferably, the said binding polypeptide is a non-Ig-type binding moiety.

According to certain preferred embodiments, the first moiety is selected from the group consisting of A) a moiety capable of binding to an actin, particularly F-actin; B) a moiety capable of binding to a heat shock protein, particularly selected from HSP90 and GRP78; C) a moiety capable of binding to a phosphatidylserine (PS), particularly a phosphatidylserine that is part of a cell membrane; D) a moiety capable of binding to a histone or component thereof, E) a moiety capable of binding to a histone deacetylase complex subunit, particularly SAP130, F) a moiety capable of binding to a HMGN protein, particularly selected from HMGB1 and HMGN1, G) a moiety capable of binding to hepatoma-derived growth factor, H) a moiety capable of binding to BCL-2, I) a moiety capable of binding to Calreticulin, and J) a moiety capable of binding to Cyclophilin A.

In particular preferred embodiments, the first moiety is selected from the group consisting of a moiety capable of binding to F-actin, a moiety capable of binding to GRP78, a moiety capable of binding to phosphatidylserine, a moiety capable of binding to HSP90, and a moiety capable of binding to SAP130. More preferably, the first moiety is selected from the group consisting of a moiety capable of binding to F-actin, a moiety capable of binding to phosphatidylserine, and a moiety capable of binding to HSP90. In particular, the first moiety is selected from the group consisting of a moiety capable of binding to F-actin and a moiety capable of binding to HSP90, and the first moiety is especially a moiety capable of binding to an actin, preferably an F-actin.

In certain embodiments, in which the first antigen is an actin, preferably an F-actin, the first moiety is an Ig type binding moiety. In other preferred embodiments, in which the first antigen is an actin, preferably an F-actin, the first moiety is a binding polypeptide, preferably a Clec9A polypeptide.

In certain embodiments, in which the first antigen is GRP78, the first moiety is an Ig type binding moiety, preferably the Ig-type binding moiety from any respective antibody set forth herein, more preferably an Ig-type binding moiety from antibody GA20 or Mab159.

In certain embodiments, in which the first antigen is phosphatidylserine, the first moiety is an Ig type binding moiety, preferably the Ig-type binding moiety from any respective antibody set forth herein, more preferably the Ig-type binding moiety from antibody3G4.

In certain embodiments, in which the first antigen is HSP90, the first moiety is an Ig type binding moiety, preferably the Ig-type binding moiety from any respective antibody set forth herein, more preferably from antibody 1.5.1 or 6H8.

In certain embodiments, in which the first antigen is SAP130, the first moiety is an Ig type binding moiety. In other preferred embodiments, in which the first antigen is SAP130, the first moiety is a binding polypeptide, preferably a Clec4e polypeptide.

The term “Clec4e” is also known as “C-type lectin domain family 4 member E” or “Mincle”. The sequence of human Clec4e protein is described in UniProt Accession number Q9ULY5 (version as in force on Jul. 28, 2022).

Preferably, also in the above embodiments, the said Ig-type binding moiety is selected from the group consisting of, a single domain antibody (VHH), an antibody heavy chain variable (VH) region, an antibody light chain variable (VL) region, a single-domain antibody (sdAb), a single-chain Fv (scFv), a single-chain antibody, an Fv, a single-chain Fv2 (scFv2), a Fab, and a F(ab′)₂.

More preferably the said Ig-type binding moiety is selected from the group consisting of an VHH, an scFv, an scFv2, a Fab, and a F(ab′)₂.

In preferred general embodiments, the molecule comprises the said first moiety at an Fc terminus of the antigen-binding molecule.

The second antigen as defined herein is not particularly limited- and certain preferred embodiments correspond to those described herein above.

In particularly preferred embodiments, the said second antigen is a membrane protein of an immune cell. Preferably, the said second antigen is a membrane protein in a cell membrane of a T cell selected from the group consisting of a T cell receptor, CD3, CD137, CD40, CTLA4, a costimulatory molecule and a coinhibitory molecule.

In preferred embodiments, the second antigen herein is a membrane protein involved in a signal transmission in a cell, preferably wherein the cell is i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, ii) a tumor cell, or iii) an autoreactive cell.

In certain preferred embodiments, the second antigen is a signal transmission receptor. In certain preferred embodiments, the second antigen an immune cell receptor antigen. In certain preferred embodiments, the second antigen is a signal transmission receptor and an immune cell receptor antigen.

In particular embodiments, the second antigen is a membrane protein of an endosome. In particular embodiments, the second antigen is a membrane protein in a cell membrane of a T cell, in particular a membrane protein in a cell membrane of a T cell selected from the group consisting of T cell receptor, CD3, CD137, CD40, CTLA4, a costimulatory molecule or a coinhibitory molecule. In particular embodiments, the second antigen is a membrane protein in a cell membrane of a cell other than a T cell, particularly which is capable of binding to a costimulatory molecule or a coinhibitory molecule in a cell membrane of a T cell.

In certain preferred embodiments, the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4).

In certain preferred embodiments, the said coinhibitory molecule is selected from the group consisting of CD279 (PD-1), CD152 (CTLA-4), TIGIT, BTLA, CD366 (Tim-3), CD96, CD112R, or CD200R.

In certain preferred embodiments, the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9.

In particularly preferred embodiments, the second antigen is selected from the group consisting of CD3, CD137, CD40 and CTLA4, preferably from the group consisting of CD3 and CD137, more preferably the second antigen is CD3.

The second moiety as defined herein is not particularly limited- and certain preferred embodiments correspond to those described herein above.

The present embodiments relating to the second antigen may—additionally or alternatively—be further defined by reference to the second moiety.

In preferred embodiments, the second moiety is an Ig type binding moiety. Consequently, in a particular preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said second antigen is different from the first antigen, and
wherein said second antigen is a membrane protein of an immune cell, and wherein
the second moiety is an Ig type binding moiety.

In an especially preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said first moiety is an Ig type binding moiety,
wherein said second antigen is different from the first antigen,
wherein said second antigen is a membrane protein of an immune cell, and
wherein the second moiety is an Ig type binding moiety.

In a further especially preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said first moiety is Clec9A or Clec4e,
wherein said second antigen is different from the first antigen,
wherein said second antigen is a membrane protein of an immune cell, and
wherein the second moiety is an Ig type binding moiety.

In particular embodiments, the second moiety is a moiety capable of binding to a membrane protein of an endosome.

In particular embodiments, the second moiety is a moiety capable of binding to a membrane protein in a cell membrane of a T cell, preferably a membrane protein in a cell membrane of a T cell selected from the group consisting of T cell receptor, CD3, CD137, CD40, CTLA4, a costimulatory molecule or a coinhibitory molecule.

In certain preferred embodiments, the membrane protein that is capable of binding to a costimulatory molecule or a coinhibitory molecule is selected from CD274 (PD-L1), CD273 (PD-L2), CD80, CD86, CD276 (ICOSL), CD276 (VICN1), VISTA, HHLA2, a member of butyrophilin family member, CD270 (HVEM), CD137L (TNFSF-9), CD252 (OX40L), CD70 (TNFSF-7), CD40 (TNFRSF-7), GIRL (TNFSF-18), CD155 (PVR, NECL5), CD112 (NECTIN-2), CD200 (MOX1), CD48 (BCM-1), and Gal-9.

In particular embodiments, the second moiety is a moiety capable of binding to a membrane protein in a cell membrane of a cell other than a T cell, particularly which is capable of binding to a costimulatory molecule or a coinhibitory molecule in a cell membrane of a T cell.

In certain preferred embodiments, the said costimulatory molecule is selected from the group consisting of CD28 (TP44), CD278 (ICOS), CD27 (TNFRSF-7), CD30, CD134 (OX40), CD357 (GITR). Tim-2, CD28H (TMIGD2), CD137 (4-1BB, TNFRSF-9), CD226 (DNAM-1), and CD244 (2B4).

In certain preferred embodiments, the said coinhibitory molecule is selected from the group consisting of CD279 (PD-1), CD152 (CTLA-4), TIGIT, BTLA, CD366 (Tim-3), CD96, CD112R, or CD200R.

In particularly preferred embodiments, the second moiety is selected from the group consisting of a moiety capable of binding to CD3, a moiety capable of binding to CD137, a moiety capable of binding to CD40 and a moiety capable of binding to CTLA4, more preferably from the group consisting of a moiety capable of binding to CD3 and a moiety capable of binding to CD137, even more preferably the second moiety is a moiety capable of binding to CD3.

In the above embodiments, preferably, the moiety capable of binding to CD3 is Ig-type binding moiety. In the above embodiments, preferably, the moiety capable of binding to CD137 is Ig-type binding moiety. In the above embodiments, preferably, the moiety capable of binding to CD40 is Ig-type binding moiety. In the above embodiments, preferably, the moiety capable of binding to CTLA4 is Ig-type binding moiety.

Preferably, the said Ig-type binding moiety is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂.

More preferably, the said Ig-type binding moiety is selected from the group consisting of an VHH, an scFv, an scFv2, a Fab, and a F(ab′)₂.

In preferred general embodiments, the molecule comprises the said second moiety in an antigen binding region, preferably an F(ab′)₂ region, of the antigen-binding molecule.

In preferred general embodiments, the molecule comprises at least one first moiety and at least one second moiety, particularly wherein the antigen-binding molecule comprises one first moiety and one second moiety, more particularly wherein the antigen-binding molecule comprises at least two, preferably two, first moieties and at least two, preferably two, second moieties.

In particular embodiments herein, the antigen-binding molecule is capable of binding to the first antigen and the second antigen upon light irradiation.

In preferred general embodiments, the antigen-binding molecule is characterized in that a complex, which is formed by binding of one or more of the molecules to the first antigen, is capable of binding more than one membrane proteins via the second moieties. In particular embodiments, the complex, which is formed by binding of one or more of the molecules to the first antigen, is capable of binding to one or more of the membrane proteins via the second moieties to confer a signal transmission via the said one or more membrane protein in a cell. In other particular embodiments, the complex, which is formed by binding of one or more of the molecules to the first antigen, is capable of binding to one or more of the membrane proteins via the second moieties to confer a signal blockage via the said one or more membrane proteins in a cell.

Generally, the antigen-binding molecule herein may be selected from the group consisting of A) a polypeptide complex, which optionally is a fusion protein, B) a polynucleotide (preferably composed of two entities, preferably connected with a linker), C) a medium-sized chemical compound (preferably composed of two entities, preferably connected with a linker), and D) a small-sized chemical compound (preferably composed of two entities, preferably connected with a linker).

In preferred general embodiments herein, the antigen-binding molecule is a polypeptide complex.

In particularly preferred embodiments herein, the antigen-binding molecule is an antibody or a fragment of an antibody, in particular an antibody. In particular embodiments, said antibody is further characterized as described elsewhere herein.

In certain preferred embodiments, antibody the antigen-binding molecule is a IgG type antibody. In certain preferred embodiments, antibody the antigen-binding molecule is a bispecific IgG type antibody.

In other preferred general embodiments herein, the antigen-binding molecule is a polypeptide complex, which is a fusion protein. Preferably, the said molecule is an antibody, which is a fusion protein, or a fragment thereof, in particular an antibody, which is a fusion protein.

Preferably, said antibody is an IgG type antibody having at least one binding polypeptide, preferably two binding polypeptides, fused to its Fc domain, particularly an antibody having at least one binding polypeptide, preferably two binding polypeptides, fused to its Fc domain.

The said binding polypeptide may be fused to the antigen-binding molecule using one or more linker(s), preferably any of the linkers described elsewhere herein.

Generally, in certain preferred embodiments, in which the antigen-binding molecule is an antibody, i-1) the first moiety is Ig-type binding moiety, or i-2) the first moiety is a binding polypeptide, and/or ii) the second moiety is an Ig-type binding moiety.

In certain preferred embodiments, in which the antigen-binding molecule is an antibody (in particular a fusion protein), the antibody is preferably capable of binding to a membrane protein of an immune cell and further comprises, preferably linked to the Fc region, at least one binding polypeptide.

Preferably, the said binding polypeptide is selected from a Clec9A polypeptide and a Clec4e polypeptide—and, more preferably, the said antibody is selected from the group consisting of an anti-CD3 antibody comprising a Clec9A polypeptide, an anti-CD137 antibody comprising a Clec9A polypeptide, an anti-CD3 antibody comprising a Clec4e polypeptide, and an anti-CD137 antibody comprising a Clec4e polypeptide.

In particular embodiments, the antigen-binding molecule comprises at least one, preferably two, Clec9A polypeptide(s), especially wherein the antigen-binding molecule further comprises i) at least two, preferably two, moieties, which are involved in a signal transmission in a cell or ii) at least two, preferably two, moieties, which are an immune cell receptor antigen.

Similarly, in particular embodiments, the antigen-binding molecule comprises at least one, preferably two, Clec4e polypeptide(s), especially wherein the antigen-binding molecule further comprises i) at least two, preferably two, moieties, which are involved in a signal transmission in a cell or ii) at least two, preferably two, moieties, which are an immune cell receptor antigen.

In general preferred embodiments, in which the antigen-binding molecule is an antibody, the antibody is a bispecific antibody directed against a membrane protein of an immune cell and against a DAMP—and, more preferably, the said antibody is selected from the group consisting of a bispecific anti-HSP90 anti-CD3 antibody, a bispecific anti-HSP90 anti-CD3 antibody; a bispecific anti-GRP78 anti-CD3 antibody, a bispecific anti-GRP78 anti-CD137 antibody, a bispecific anti-phosphatidylserine anti-CD3 antibody, and a bispecific anti-phosphatidylserine anti-CD137 antibody.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the constant regions. Exemplary embodiments are schematically shown in FIG. 3. In one preferred embodiment, the antibodies may have their first H-chain CH3 region and second H-chain CH3 region crosslinked by disulfide bonds.

Generally, in certain preferred embodiments, in which the antigen-binding molecule is an antibody, i-1) the first moiety is Ig-type binding moiety, or i-2) the first moiety is a binding polypeptide, and ii) the second moiety is an Ig-type binding moiety.

In particular embodiments herein a) the first moiety is Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂; and/or b) the second moiety is an Ig-type binding moiety, which is selected from the group consisting of, a single domain antibody (VHH), a combination of an antibody heavy chain variable (VH) region and an antibody light chain variable (VL) region, a single-domain antibody (sdAb), a single-chain Fv (scFv), a single-chain antibody, an Fv, a single-chain Fv2 (scFv2), a Fab, and a F(ab′)₂; and/or c) the said binding polypeptide is a non-Ig-type binding moiety.

In further preferred particular embodiments herein a*) the first moiety is a binding domain selected from the group consisting of an VHH, an scFv, an scFv2, a Fab, and a F(ab′)₂, and/or b*) the second moiety is a binding domain selected from the group consisting of an VHH, an scFv, an scFv2, a Fab, and a F(ab′)₂; and/or c*) wherein the binding polypeptide is a non-Ig-type binding moiety, especially a non-Ig-type binding moiety attached to an Fc domain of the said antigen-binding molecule.

In preferred particular embodiments herein, the antigen-binding molecule is an antibody, selected from the group consisting of A) an antibody capable of binding to F-actin, as well as to an antigen involved in a signal transmission in a cell; B) an antibody capable of binding to F-actin, as well as to an immune cell receptor; C) an antibody capable of binding to HSP90, as well as to an antigen involved in a signal transmission in a cell; D) an antibody capable of binding to HSP90, as well as to an immune cell receptor; E) an antibody capable of binding to GRP78, as well as to an antigen involved in a signal transmission in a cell; F) an antibody capable of binding to GRP78, as well as to an immune cell receptor; G) an antibody capable of binding to phosphatidylserine, as well as to an antigen involved in a signal transmission in a cell; H) an antibody capable of binding to phosphatidylserine, as well as to an immune cell receptor; I) an antibody capable of binding to SAP130, as well as to an antigen involved in a signal transmission in a cell; and J) an antibody capable of binding to SAP130, as well as to an immune cell receptor.

More preferably, the said antibody is selected from the group consisting of A-1) an antibody capable of binding to F-actin, as well as to CD3; A-2) an antibody capable of binding to F-actin, as well as to CD137; C-1) an antibody capable of binding to HSP90, as well as to CD3; C-2) an antibody capable of binding to HSP90, as well as to CD137; E-1) an antibody capable of binding to GRP78, as well as to CD3; E-2) an antibody capable of binding to GRP78, as well as to CD137; G-1) an antibody capable of binding to phosphatidylserine, as well as to CD3; G-2) an antibody capable of binding to phosphatidylserine, as well as to CD137; I-1) an antibody capable of binding to SAP130, as well as to CD3; I-2) an antibody capable of binding to SAP130, as well as to CD137.

In preferred particular embodiments herein, the antibody is selected from the group consisting of
A) an antibody comprising two first moieties capable of binding to F-actin, especially wherein the first moieties are binding polypeptides, preferably Clec9A polypeptides, preferably attached to the Fc domain of the antibody, as well as two second moieties involved in a signal transmission in a cell, especially wherein the second moieties are Ig-type binding moieties;
B) an antibody comprising two first moieties capable of binding to F-actin, especially wherein the first moieties are binding polypeptides, preferably Clec9A polypeptides, preferably attached to the Fc domain of the antibody, as well as two second moieties which are an immune cell receptor antigen, especially wherein the second moieties are Ig-type binding moieties;
C) an antibody comprising two first moieties capable of binding to HSP90, especially wherein the first moieties are Ig-type binding moieties, as well as two second moieties involved in a signal transmission in a cell, especially wherein the second moieties are Ig-type binding moieties;
D) an antibody comprising two first moieties capable of binding to HSP90, especially wherein the first moieties are Ig-type binding moieties, as well as two second moieties, which are an immune cell receptor antigen, especially wherein the second moieties are Ig-type binding moieties;
E) an antibody comprising two first moieties capable of binding to GRP78, especially wherein the first moieties are Ig-type binding moieties, as well as two second moieties involved in a signal transmission in a cell, especially wherein the second moieties are Ig-type binding moieties;
F) an antibody comprising two first moieties capable of binding to GRP78, especially wherein the first moieties are Ig-type binding moieties, as well as two second moieties, which are an immune cell receptor antigen, especially wherein the second moieties are Ig-type binding moieties;
G) an antibody comprising two first moieties capable of binding to phosphatidylserine, especially wherein the first moieties are Ig-type binding moieties, as well as two second moieties involved in a signal transmission in a cell, especially wherein the second moieties are Ig-type binding moieties;
H) an antibody comprising two first moieties capable of binding to phosphatidylserine, especially wherein the first moieties are Ig-type binding moieties, as well as two second moieties, which are an immune cell receptor antigen, especially wherein the second moieties are Ig-type binding moieties;
I) an antibody comprising two first moieties capable of binding to SAP130, especially wherein the first moieties are binding polypeptides, preferably Clec4e polypeptides, preferably attached to the Fc domain of the antibody, as well as two second moieties involved in a signal transmission in a cell, especially wherein the second moieties are Ig-type binding moieties;
J) an antibody comprising two first moieties capable of binding to SAP130, especially wherein the first moieties are binding polypeptides, preferably Clec4e polypeptides, preferably attached to the Fc domain of the antibody, as well as two second moieties, which are an immune cell receptor antigen, especially wherein the second moieties are Ig-type binding moieties.

More preferably, the said antibody is selected from the group consisting of
A1) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD3, especially wherein the second moieties are Ig-type binding moieties;
A2) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD137, especially wherein the second moieties are Ig-type binding moieties;
C1) an antibody comprising two first moieties capable of binding to HSP90, especially wherein the first moieties are Ig-type binding moieties, as well as two second moieties capable of binding to CD3, especially wherein the second moieties are Ig-type binding moieties;
C2) an antibody comprising two first moieties capable of binding to HSP90, especially wherein the first moieties are Ig-type binding moieties, as well as two second moieties capable of binding to CD137, especially wherein the second moieties are Ig-type binding moieties;
E1) an antibody comprising two first moieties capable of binding to GRP78, especially wherein the first moieties are Ig-type binding moieties, as well as two second moieties capable of binding to CD3, especially wherein the second moieties are Ig-type binding moieties;
E2) an antibody comprising two first moieties capable of binding to GRP78, especially wherein the first moieties are Ig-type binding moieties, as well as two second moieties capable of binding to CD137, especially wherein the second moieties are Ig-type binding moieties;
G1) an antibody comprising two first moieties capable of binding to phosphatidylserine, especially wherein the first moieties are Ig-type binding moieties, as well as two second moieties capable of binding to CD3, especially wherein the second moieties are Ig-type binding moieties;
G2) an antibody comprising two first moieties capable of binding to phosphatidylserine, especially wherein the first moieties are Ig-type binding moieties, as well as two second moieties capable of binding to CD137, especially wherein the second moieties are Ig-type binding moieties;
I1) an antibody comprising two first moieties capable of binding to SAP130, especially wherein the first moieties are binding polypeptides, preferably Clec4e polypeptides, preferably attached to the Fc domain of the antibody, as well as two second moieties capable of binding to CD3, especially wherein the second moieties are Ig-type binding moieties;
I2) an antibody comprising two first moieties capable of binding to SAP130, especially wherein the first moieties are binding polypeptides, preferably Clec4e polypeptides, preferably attached to the Fc domain of the antibody, as well as two second moieties capable of binding to CD137, especially wherein the second moieties are Ig-type binding moieties.

A further general aspect of the present invention relates to a pharmaceutical composition comprising an antigen-binding molecule as described herein. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

A further general aspect, the present invention relates to an antigen-binding molecule of the present invention for use i) in medicine, and/or ii) in a method of treating a medical condition, particularly a medical condition involving cell death, and/or iii) in a method of treating a medical condition in a subject, the method comprising contacting the said antigen-binding molecule with a damaged cell, and/or iv) in a method of treating a medical condition in a subject, the method comprising contacting the said antigen-binding molecule with a damage-associated molecular pattern (DAMP) in a patient, particularly further comprising contacting the said antigen-binding molecule with the said second antigen and/or further comprising contacting the said antigen-binding molecule with the said third antigen, and/or v) in a method of treating a medical condition in a subject, the method comprising administering the said antigen-binding molecule to a patient suffering from a condition involving cell damage, particularly cell death, especially wherein the method comprises contacting a plurality of antigen-binding molecules in close proximity with the first and second antigens, and/or third antigens.

In one embodiment, the antigen-binding molecule is as defined in any of the aspects or embodiments specified in invention group [AA] above.

Consequently, in certain preferred embodiments, there is provided an antigen-binding molecule of the present invention for use in medicine. Consequently, in certain preferred embodiments, there is provided an antigen-binding molecule of the present invention for use in a method of treating a medical condition, particularly a medical condition involving cell damage, particularly involving cell death. Consequently, in certain preferred embodiments, there is provided an antigen-binding molecule of the present invention for use in a method of treating a medical condition in a subject, the method comprising contacting the said antigen-binding molecule with a damaged cell. Consequently, in certain preferred embodiments, there is provided an antigen-binding molecule of the present invention for use in a method of treating a medical condition in a subject, the method comprising contacting the said antigen-binding molecule with a damage-associated molecular pattern (DAMP) in a patient, particularly further comprising contacting the said antigen-binding molecule with the said second antigen. Consequently, in certain preferred embodiments, there is provided an antigen-binding molecule of the present invention for use in a method of treating a medical condition in a subject, the method comprising administering the said antigen-binding molecule to a patient suffering from a condition involving cell damage, particularly cell death.

In one aspect, an antigen-binding molecule or pharmaceutical composition according to any one of the embodiments or aspects herein is provided for use in medicine. In one aspect, an antigen-binding molecule or the pharmaceutical composition of any of the embodiments or aspects herein is provided for use in a method of treating or preventing a medical condition. In one aspect, an antigen-binding molecule or the pharmaceutical composition for use of any of the embodiments or aspects herein is provided, wherein the medical condition is selected from the group consisting of a cancer and an immune, preferably an autoimmune, disease.

In one aspect, an antigen-binding molecule or the pharmaceutical composition for use of any of the embodiments or aspects herein is provided, wherein
i) the method comprises administering an antigen-binding molecule according to any one of the embodiments or aspects to a patient, and/or
ii) the method is a method for activating or preventing an immune response, and/or
iii) the method is for conferring a signal transmission or blockage in a cell; particularly wherein a) the method comprises contacting the antigen-binding molecule with a damage-associated molecular pattern (DAMP), which is exposed to an extracellular environment due to cell damage, especially wherein the DAMP is selected from the group consisting of SF3B3, PCNA, F-actin, and phosphatidylserine,
and/or b) the method comprises contacting the antigen-binding molecule with a cell especially selected from a-i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, a-ii) a tumor cell, and a-iii) an autoreactive cell, and/or c) the antigen-binding molecule comprises at least one, preferably two, Clec4e polypeptide(s), especially wherein the antigen-binding molecule further comprises c-1) at least two, preferably two, moieties, which are involved in a signal transmission in a cell or c-2) at least two, preferably two, moieties, which are an immune cell receptor antigen.

In one aspect, an antigen-binding molecule or pharmaceutical composition for use of any of the embodiments or aspects herein is provided, wherein the method is a method for treating or preventing a cancer or an immune, preferably an autoimmune, disease,
especially wherein the method is a method for treating or preventing a cancer, in particular wherein the method is a method for treating a cancer.

In a related further general aspect, the present invention relates to a pharmaceutical composition in any of the uses described above for a molecule of the invention.

In certain preferred embodiments, the method of treating in context with the invention comprises contacting a plurality of antigen-binding molecules in close proximity with the first and second antigens.

In certain preferred embodiments, the method of treating in context with the invention is a method for activating or preventing an immune response. Preferably, the method of treating in context with the invention is a method for activating an immune response.

In certain preferred embodiments, the method of treating in context with the invention is for conferring a signal transmission or blockage in a cell, preferably for conferring a signal transmission in a cell.

In certain preferred embodiments herein, the method of treating comprises administering an antigen-binding molecule according of the invention a patient, particularly a human patient. Preferably, the said patient is characterized as described elsewhere herein.

In certain preferred embodiments herein, the method of treating is a method for treating or preventing a cancer or an immune (preferably an autoimmune) disease, especially wherein the method is a method for treating or preventing a cancer, in particular wherein the method is a method for treating a cancer.

Consequently, in a particular preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said second antigen is different from the first antigen, and
wherein said second antigen is a membrane protein of an immune cell, and wherein
the second moiety is an Ig type binding moiety
for use in a method for treating a cancer.

In an especially preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said first moiety is an Ig type binding moiety,
wherein said second antigen is different from the first antigen,
wherein said second antigen is a membrane protein of an immune cell, and
wherein the second moiety is an Ig type binding moiety
for use in a method for treating a cancer.

In a further especially preferred embodiment, the present invention relates to an antigen-binding molecule, which comprises:
(1) at least one first moiety that binds to a first antigen, and
(2) at least one second moiety that binds to a second antigen;
wherein said first antigen is a damage-associated molecular pattern (DAMP),
wherein said first moiety is Clec9A or Clec4e,
wherein said second antigen is different from the first antigen,
wherein said second antigen is a membrane protein of an immune cell, and
wherein the second moiety is an Ig type binding moiety.
for use in a method for treating a cancer.

In particular embodiments, the method for treating in context with the invention comprises contacting the antigen-binding molecule with a damage-associated molecular pattern (DAMP), which is exposed to an extracellular environment due to cell damage, especially wherein the DAMP is selected from the group consisting of F-actin, GRP78, phosphatidylserine, HSP90, and SAP130.

In particular embodiments, the method for treating in context with the invention comprises contacting the antigen-binding molecule with a cell especially selected from a-i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, a-ii) a tumor cell, and a-iii) an autoreactive cell.

In particular preferred embodiments, there is provided an antigen-binding molecule or pharmaceutical composition of the invention for use in medicine.

In particular preferred embodiments, there is provided an antigen-binding molecule or pharmaceutical composition of the invention for use in a method of treating or preventing a medical condition.

In preferred general embodiments herein, the medical condition is selected from the group consisting of a cancer and an immune, preferably an autoimmune, disease. Consequently, in certain preferred embodiments herein, the medical condition is a cancer, particularly a cancer as described elsewhere herein. Consequently, in certain preferred embodiments herein, the medical condition is an immune disease, particularly an immune disease as described elsewhere herein. Preferably, the said is an immune disease is an autoimmune disease, particularly an autoimmune disease as described elsewhere herein.

A further general aspect of the present invention relates to a polynucleotide encoding antigen-binding molecule of the invention.

A further general aspect of the present invention relates to a vector comprising a polynucleotide of the invention.

A further general aspect of the present invention relates to a cell comprising the vector of the invention.

In addition, the present invention also relates to the use of at least a first moiety and at least a second moiety for the production of an antigen-binding molecule comprising the at least first moiety and the at least a second moiety, wherein
(1) the at least one first moiety binds to a first antigen, and
(2) the at least one second moiety binds to a second antigen;
wherein said first antigen is a damage-associated molecular pattern (DAMP), and wherein said second antigen is different from the first antigen, preferably wherein said second antigen is a membrane protein of an immune cell.

The invention further relates to the use of an antigen-binding molecule for targeting a cell in a tissue where cell death is observed or to be detected, wherein
(1) the at least one first moiety binds to a first antigen, and
(2) the at least one second moiety binds to a second antigen;
wherein said first antigen is a damage-associated molecular pattern (DAMP), and
wherein said second antigen is different from the first antigen, preferably wherein said second antigen is a membrane protein of an immune cell.

In embodiments, the use is provided of an antigen-binding molecule for targeting a cell in a tissue where cell death is observed or to be detected, wherein (1) the at least one first moiety binds to a first antigen, and (2) the at least one second moiety binds to a second antigen; wherein said first antigen is a damage-associated molecular pattern (DAMP), and wherein said second antigen is different from the first antigen, preferably wherein said second antigen is a membrane protein of an immune cell, or a tumour cell, and wherein the first antigen is selected from the group consisting of: a) Splicing Factor 3B Subunit 3 (SF3B3); and b) Proliferating Cell Nuclear Antigen (PCNA). In an embodiment, the antigen-binding molecule comprises at least one third moiety that binds to a third antigen; wherein (i) said third antigen is different from the first and second antigen, or wherein (ii) said third antigen is different from the first but same as the second antigen; and wherein said third antigen is a membrane protein of an immune cell, or a tumour cell. In an embodiment, the use is an in vitro use. In an embodiment, the use is an in vivo use. In an embodiment, the use is an ex vivo use. In embodiments, the first moiety is as defined in any of one of embodiments [AA1]-[AA13]. In embodiments, the second moiety is as defined in any of one of embodiments [AA1]-[AA13]. In embodiments, the third moiety is as defined in any of one of embodiments [AA2]-[AA13].

In embodiments, the use is provided of an antigen-binding molecule for targeting a cell in a tissue where cell death is observed or to be detected, wherein the antigen-binding molecule comprises: (1) at least one first moiety that binds to a first antigen, and (2) at least one second moiety that binds to a second antigen; wherein said first antigen is a damage-associated molecular pattern (DAMP), and wherein said second antigen is different from the first antigen, preferably wherein said second antigen is a membrane protein of an immune cell, or a tumour cell, wherein the molecule comprises at least one third moiety that binds to a third antigen; wherein said third antigen is different from the first antigen; preferably wherein said third antigen is a membrane protein of an immune cell, or a tumour cell, wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA). In embodiments, (i) said third antigen is different from the first and second antigen, or wherein (ii) said third antigen is different from the first but same as the second antigen. In embodiments, the first moiety is as defined in any of one of embodiments [AA117]-[AA126]. In embodiments, the second moiety is as defined in any of one of embodiments [AA117]-[AA126]. In embodiments, the third moiety is as defined in any of one of embodiments [AA117]-[AA126].

In addition, the invention also relates to the use of an antigen-binding molecule for induction or blockage of signal transduction in a cell in a tissue where cell death is observed, wherein
(1) the at least one first moiety binds to a first antigen, and
(2) the at least one second moiety binds to a second antigen;
wherein said first antigen is a damage-associated molecular pattern (DAMP), and
wherein said second antigen is different from the first antigen, preferably wherein said second antigen is a membrane protein of an immune cell.

In one aspect, provided is the use of an antigen-binding molecule for induction or blockage of signal transduction in a cell in a tissue where cell death is observed, wherein (1) the at least one first moiety binds to a first antigen, and (2) the at least one second moiety binds to a second antigen; wherein said first antigen is a damage-associated molecular pattern (DAMP), and wherein said second antigen is different from the first antigen, preferably wherein said second antigen is a membrane protein of an immune cell, or a tumour cell, and wherein the first antigen is selected from the group consisting of: a) Splicing Factor 3B Subunit 3 (SF3B3); and b) Proliferating Cell Nuclear Antigen (PCNA). In an embodiment, the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In an embodiment, the use is an in vitro use. In an embodiment, the use is an in vivo use. In an embodiment, the use is an ex vivo use. In embodiments, the first moiety is as defined in any of one of embodiments [AA1]-[AA13]. In embodiments, the second moiety is as defined in any of one of embodiments [AA1] [AA13]. In embodiments, the third moiety is as defined in any of one of embodiments [AA2]-[AA13].

In one aspect, provided is the use of an antigen-binding molecule for induction or blockage of signal transduction in a cell in a tissue where cell death is observed, wherein the antigen-binding molecule comprises: (1) at least one first moiety that binds to a first antigen, and (2) at least one second moiety that binds to a second antigen;
wherein said first antigen is a damage-associated molecular pattern (DAMP), and wherein said second antigen is different from the first antigen, preferably wherein said second antigen is a membrane protein of an immune cell, or a tumour cell, and wherein the molecule comprises at least one third moiety that binds to a third antigen; wherein said third antigen is different from the first antigen; preferably wherein said third antigen is a membrane protein of an immune cell, or a tumour cell, wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA).

In embodiments, (i) said third antigen is different from the first and second antigen, or wherein (ii) said third antigen is different from the first but same as the second antigen.

In embodiments, the first moiety is as defined in any of one of embodiments [AA117]-[AA126]. In embodiments, the second moiety is as defined in any of one of embodiments [AA117]-[AA126]. In embodiments, the third moiety is as defined in any of one of embodiments [AA117]-[AA126].

Furthermore, the present invention related to a kit for producing a molecule specifically acting in a tissue where a cell death is being observed, the molecule comprising
(1) the at least one first moiety binds to a first antigen, and
(2) the at least one second moiety binds to a second antigen;
wherein said first antigen is a damage-associated molecular pattern (DAMP), and
wherein said second antigen is different from the first antigen, preferably wherein said second antigen is a membrane protein of an immune cell, wherein the kit comprises
at least a first container comprising the least first moiety and
at least a second container comprising the at least second moiety.

In an aspect, a kit is provided for producing an antigen-binding molecule specifically acting in a tissue where a cell death is being observed, the molecule comprising
(1) the at least one first moiety binds to a first antigen, and
(2) the at least one second moiety binds to a second antigen;
wherein said first antigen is a damage-associated molecular pattern (DAMP), and
wherein said second antigen is different from the first antigen, preferably wherein said second antigen is a membrane protein of an immune cell or a tumour cell, and wherein the first antigen is selected from the group consisting of:
a) Splicing Factor 3B Subunit 3 (SF3B3); and
b) Proliferating Cell Nuclear Antigen (PCNA).
and, wherein the kit comprises
at least a first container comprising the at least first moiety and
at least a second container comprising the at least second moiety.

In an embodiment, the kit comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell. In embodiments, the first moiety is as defined in any of one of embodiments [AA1]-[AA13]. In embodiments, the second moiety is as defined in any of one of embodiments [AA1]-[AA13]. In embodiments, the third moiety is as defined in any of one of embodiments [AA2]-[AA13].

In an aspect, a kit is provided for producing an antigen-binding molecule specifically acting in a tissue where a cell death is being observed, the antigen-binding molecule comprising: (1) at least one first moiety that binds to a first antigen, and (2) at least one second moiety that binds to a second antigen; wherein said first antigen is a damage-associated molecular pattern (DAMP), and wherein said second antigen is different from the first antigen, preferably wherein said second antigen is a membrane protein of an immune cell, or a tumour cell, and wherein the molecule comprises at least one third moiety that binds to a third antigen; wherein said third antigen is different from the first antigen; preferably wherein said third antigen is a membrane protein of an immune cell, or a tumour cell, wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA), and, wherein
the kit comprises
at least a first container comprising the at least one first moiety, and at least a second container comprising the at least one second moiety, and at least a third container comprising the at least one third moiety.

In embodiments, (i) said third antigen is different from the first and second antigen, or wherein (ii) said third antigen is different from the first but same as the second antigen.

In embodiments, the first moiety is as defined in any of one of embodiments [AA117]-[AA126]. In embodiments, the second moiety is as defined in any of one of embodiments [AA117]-[AA126]. In embodiments, the third moiety is as defined in any of one of embodiments [AA117]-[AA126].

Also for the above uses and kits of the invention, all embodiments and definitions discussed above also apply.

### Method of Screening

Provided in one aspect is a method of screening an antigen-binding molecule, which comprises the steps of:
(a) selecting one or more nucleic acid(s) encoding at least one first moiety wherein the first moiety binds to a first antigen, and wherein the first antigen is a damage-associated molecular pattern (DAMP);
(b) selecting one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) optionally providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c), thereby obtaining the antigen-binding molecule.

Also for the above method of the invention, all embodiments and definitions discussed above also apply.

In embodiments, the first antigen is selected from the group consisting of: a) Splicing Factor 3B Subunit 3 (SF3B3); and b) Proliferating Cell Nuclear Antigen (PCNA).

In embodiments, the antigen-binding molecule obtained in step (b) is as defined in any one of embodiments [AA1]-[AA13]. In embodiments, the first moiety in step (a) is as defined in any of one of embodiments [AA1]-[AA13]. In embodiments, the second moiety in step (b) is as defined in any of one of embodiments [AA1]-[AA13]. In embodiments, the method does not include step (b1). In embodiments, the method includes step (b1). In embodiments, the third moiety in step (b1) is as defined in any of one of embodiments [AA2]-[AA13]. In embodiments, the one or more nucleic acid(s) is RNA or DNA. In embodiments, the one or more nucleic acid(s) obtained in step (c) are one nucleic acid or two nucleic acids, optionally wherein the two nucleic acids are on a single expression vector or on separate expression vectors. In embodiments, step (d) comprises recombinant expression of the antigen-binding molecule in a host cell. Suitable vectors, expression vectors and method for recombinant expression are well known in the art and are for example described above.

In another aspect, a method of screening an antigen-binding molecule is provided, which comprises the steps of:
(a) selecting one or more nucleic acid(s) encoding at least one first moiety wherein the first moiety binds to a first antigen, and wherein the first antigen is a damage-associated molecular pattern (DAMP), wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) selecting one or more nucleic acid(s) encoding at least one second moiety wherein the second moiety binds to a second antigen, wherein the second antigen is different from the first antigen, and wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and the at least one third moiety provided in (b1) is/are linked; and
(d) producing the antigen-binding molecule using the one or more nucleic acid(s) prepared in (c), thereby obtaining the antigen-binding molecule.

In embodiments, said third antigen is different from the first and second antigen. In embodiments, said third antigen is different from the first but same as the second antigen. In embodiments, the antigen-binding molecule obtained in step (b) is as defined in any of one of embodiments [AA117]-[AA126]. In embodiments, the first moiety in step (a) is as defined in any of one of embodiments [AA117]-[AA126]. In embodiments, the second moiety in step (b) is as defined in any of one of embodiments [AA117]-[AA126]. In embodiments, the third moiety in step (b1) is as defined in any of one of embodiments [AA117]-[AA126]. In embodiments, the one or more nucleic acid(s) is RNA or DNA. In embodiments, the one or more nucleic acid(s) obtained in step (c) are one nucleic acid or two nucleic acids, optionally wherein the two nucleic acids are on a single expression vector or on separate expression vectors. In embodiments, step (d) comprises recombinant expression of the antigen-binding molecule in a host cell. Suitable vectors, expression vectors and method for recombinant expression are well known in the art and are for example described above.

Further, specific bispecific antibodies and related compositions and uses are provided herein below:

In one aspect, provided is a bispecific anti-Phosphatidylserine (PS) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD3 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD3 antibody comprising a heavy chain of IgG1 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution.

In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Phosphatidylserine (PS) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD3 antibody comprises a heavy chain of IgG1 isotype and a light chain of kappa isotype. In one embodiment, the bispecific anti-Phosphatidylserine (PS) anti-CD3 antibody comprises a heavy chain of IgG1 isotype and a light chain of lambda isotype.

In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD3 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD3 antibody comprising a heavy chain of IgG2 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Phosphatidylserine (PS) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD3 antibody comprises a heavy chain of IgG2 isotype and a light chain of kappa isotype. In one embodiment, the bispecific anti-Phosphatidylserine (PS) anti-CD3 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD3 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD3 antibody comprising a heavy chain of IgG3 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Phosphatidylserine (PS) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD3 antibody comprises a heavy chain of IgG3 isotype and a light chain of kappa isotype. In one embodiment, the bispecific anti-Phosphatidylserine (PS) anti-CD3 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD3 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD3 antibody comprising a heavy chain of IgG4 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Phosphatidylserine (PS) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD3 antibody comprises a heavy chain of IgG4 isotype and a light chain of kappa isotype. In one embodiment, the bispecific anti-Phosphatidylserine (PS) anti-CD3 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD137 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD137 antibody comprising a heavy chain of IgG1 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Phosphatidylserine (PS) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD137 antibody comprises a heavy chain of IgG1 isotype and a light chain of kappa isotype. In one embodiment, the bispecific anti-Phosphatidylserine (PS) anti-CD137 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD137 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD137 antibody comprising a heavy chain of IgG2 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20. In embodiment, the present invention relates to a bispecific anti-Phosphatidylserine (PS) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD137 antibody comprises a heavy chain of IgG2 isotype and a light chain of kappa isotype. In one embodiment, the bispecific anti-Phosphatidylserine (PS) anti-CD137 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD137 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD137 antibody comprising a heavy chain of IgG3 isotype and a light chain of lambda isotype, or a pharmaceutical composition comprising the bispecific antibody and one or more pharmaceutically acceptable carriers.

In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds.

In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Phosphatidylserine (PS) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD137 antibody comprises a heavy chain of IgG3 isotype and a light chain of kappa isotype. In one embodiment, the bispecific anti-Phosphatidylserine (PS) anti-CD137 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Phosphatidylserine (PS) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Phosphatidylserine (PS) anti-CD137 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Phosphatidylserine (PS) anti-CD137 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Phosphatidylserine (PS) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Phosphatidylserine (PS) anti-CD137 antibody comprises a heavy chain of IgG4 isotype and a light chain of kappa isotype. In one embodiment, the bispecific anti-Phosphatidylserine (PS) anti-CD137 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD3 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD3 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-F-Actin anti-CD3 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-F-Actin anti-CD3 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD3 antibody comprises a heavy chain of IgG1 isotype and a light chain of kappa isotype. In one embodiment, the bispecific anti-F-Actin anti-CD3 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD3 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD3 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-F-Actin anti-CD3 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD3 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD3 antibody comprises a heavy chain of IgG2 isotype and a light chain of kappa isotype. In one embodiment, the bispecific anti-F-Actin anti-CD3 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD3 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD3 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-F-Actin anti-CD3 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD3 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD3 antibody comprises a heavy chain of IgG3 isotype and a light chain of kappa isotype. In one embodiment, the bispecific anti-F-Actin anti-CD3 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD3 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD3 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-F-Actin anti-CD3 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD3 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD3 antibody comprises a heavy chain of IgG4 isotype and a light chain of kappa isotype. In one embodiment, the bispecific anti-F-Actin anti-CD3 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD137 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD137 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-F-Actin anti-CD137 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-F-Actin anti-CD137 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD137 antibody comprises a heavy chain of IgG1 isotype and a light chain of kappa isotype. In one embodiment, the bispecific anti-F-Actin anti-CD137 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD137 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD137 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-F-Actin anti-CD137 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD137 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD137 antibody comprises a heavy chain of IgG2 isotype and a light chain of kappa isotype. In one embodiment, the bispecific anti-F-Actin anti-CD137 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD137 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD137 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-F-Actin anti-CD137 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD137 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD137 antibody comprises a heavy chain of IgG3 isotype and a light chain of kappa isotype. In one embodiment, the bispecific anti-F-Actin anti-CD137 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-F-Actin anti-CD137 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-F-Actin anti-CD137 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-F-Actin anti-CD137 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-F-Actin anti-CD137 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-F-Actin anti-CD137 antibody comprises a heavy chain of IgG4 isotype and a light chain of kappa isotype. In one embodiment, the bispecific anti-F-Actin anti-CD137 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec9A polypeptide, wherein the molecule is an IgG type antibody comprising a heavy chain of IgG1 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec9A polypeptide, wherein the molecule is an IgG type antibody comprising a heavy chain of IgG1 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD3 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec9A polypeptide, wherein the molecule is an IgG type antibody, comprises a heavy chain of IgG1 isotype and a light chain of kappa isotype. In one embodiment, the bispecific antigen-binding molecule which is an anti-CD3 antibody comprising a Clec9A polypeptide, wherein the molecule is an IgG type antibody, comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec9A polypeptide, wherein the molecule is an IgG type antibody, comprising a heavy chain of IgG2 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec9A polypeptide, wherein the molecule is an IgG type antibody, comprising a heavy chain of IgG2 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec9A polypeptide, wherein the molecule is an IgG type antibody, comprising a heavy chain of IgG2 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific antigen-binding molecule which is an anti-CD3 antibody comprising a Clec9A polypeptide, wherein the molecule is an IgG type antibody, comprising a heavy chain of IgG2 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec9A polypeptide, wherein the molecule is an IgG type antibody, comprises a heavy chain of IgG2 isotype and a light chain of kappa isotype. In one embodiment, the bispecific antigen-binding molecule which is an anti-CD3 antibody comprising a Clec9A polypeptide, wherein the molecule is an IgG type antibody, comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec9A polypeptide, wherein the molecule is an IgG type antibody, comprising a heavy chain of IgG3 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec9A polypeptide, wherein the molecule is an IgG type antibody, comprising a heavy chain of IgG3 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec9A polypeptide, wherein the molecule is an IgG type antibody, comprising a heavy chain of IgG3 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific antigen-binding molecule which is an anti-CD3 antibody comprising a Clec9A polypeptide, wherein the molecule is an IgG type antibody, comprising a heavy chain of IgG3 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec9A polypeptide, wherein the molecule is an IgG type antibody, comprises a heavy chain of IgG3 isotype and a light chain of kappa isotype. In one embodiment, the bispecific antigen-binding molecule which is an anti-CD3 antibody comprising a Clec9A polypeptide, wherein the molecule is an IgG type antibody, comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec9A polypeptide, wherein the molecule is an IgG type antibody, comprising a heavy chain of IgG4 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec9A polypeptide, wherein the molecule is an IgG type antibody, comprising a heavy chain of IgG4 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec9A polypeptide, wherein the molecule is an IgG type antibody, comprising a heavy chain of IgG4 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20. In embodiment, the present invention relates to a bispecific antigen-binding molecule which is an anti-CD3 antibody comprising a Clec9A polypeptide, wherein the molecule is an IgG type antibody, comprising a heavy chain of IgG4 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec9A polypeptide, wherein the molecule is an IgG type antibody, comprises a heavy chain of IgG4 isotype and a light chain of kappa isotype. In one embodiment, the bispecific antigen-binding molecule which is an anti-CD3 antibody comprising a Clec9A polypeptide, wherein the molecule is an IgG type antibody, comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec9A polypeptide, wherein the molecule is an IgG type antibody, comprising a heavy chain of IgG1 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec9A polypeptide, wherein the molecule is an IgG type antibody comprising a heavy chain of IgG1 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec9A polypeptide, wherein the molecule is an IgG type antibody comprising a heavy chain of IgG1 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD137 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec9A polypeptide, wherein the molecule is an IgG type antibody comprises a heavy chain of IgG1 isotype and a light chain of kappa isotype. In one embodiment, the bispecific antigen-binding molecule which is an anti-CD137 antibody comprising a Clec9A polypeptide, wherein the molecule is an IgG type antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec9A polypeptide, wherein the molecule is an IgG type antibody comprising a heavy chain of IgG2 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec9A polypeptide, wherein the molecule is an IgG type antibody comprising a heavy chain of IgG2 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific antigen-binding molecule which is an anti-CD137 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec9A polypeptide, wherein the molecule is an IgG type antibody comprises a heavy chain of IgG2 isotype and a light chain of kappa isotype. In one embodiment, the bispecific antigen-binding molecule which is an anti-CD137 antibody comprising a Clec9A polypeptide, wherein the molecule is an IgG type antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec9A polypeptide, wherein the molecule is an IgG type antibody comprising a heavy chain of IgG3 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec9A polypeptide, wherein the molecule is an IgG type antibody comprising a heavy chain of IgG3 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific antigen-binding molecule which is an anti-CD137 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec9A polypeptide, wherein the molecule is an IgG type antibody comprises a heavy chain of IgG3 isotype and a light chain of kappa isotype. In one embodiment, the bispecific antigen-binding molecule which is an anti-CD137 antibody comprising a Clec9A polypeptide, wherein the molecule is an IgG type antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec9A polypeptide, wherein the molecule is an IgG type antibody comprising a heavy chain of IgG4 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec9A polypeptide, wherein the molecule is an IgG type antibody comprising a heavy chain of IgG4 isotype and a light chain of lambda isotype, or a pharmaceutical composition comprising the bispecific antigen-binding molecule and one or more pharmaceutically acceptable carriers. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific antigen-binding molecule which is an anti-CD137 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec9A polypeptide, wherein the molecule is an IgG type antibody comprises a heavy chain of IgG4 isotype and a light chain of kappa isotype. In one embodiment, the bispecific antigen-binding molecule which is an anti-CD137 antibody comprising a Clec9A polypeptide, wherein the molecule is an IgG type antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD3 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD3 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Splicing Factor 3B Subunit 3 (SF3B3) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD3 antibody comprises a heavy chain of IgG1 isotype and a light chain of kappa isotype. In one embodiment, the bispecific anti-Splicing Factor 3B Subunit 3 (SF3B3) anti-CD3 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD3 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD3 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD3 antibody comprises a heavy chain of IgG2 isotype and a light chain of kappa isotype. In one embodiment, the bispecific anti-Splicing Factor 3B Subunit 3 (SF3B3) anti-CD3 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD3 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD3 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CHI regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD3 antibody comprises a heavy chain of IgG3 isotype and a light chain of kappa isotype. In one embodiment, the bispecific anti-Splicing Factor 3B Subunit 3 (SF3B3) anti-CD3 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD3 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD3 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD3 antibody comprises a heavy chain of IgG4 isotype and a light chain of kappa isotype. In one embodiment, the bispecific anti-Splicing Factor 3B Subunit 3 (SF3B3) anti-CD3 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD137 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD137 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Splicing Factor 3B Subunit 3 (SF3B3) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD137 antibody comprises a heavy chain of IgG1 isotype and a light chain of kappa isotype. In one embodiment, the bispecific anti-Splicing Factor 3B Subunit 3 (SF3B3) anti-CD137 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD137 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD137 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD137 antibody comprises a heavy chain of IgG2 isotype and a light chain of kappa isotype. In one embodiment, the bispecific anti-Splicing Factor 3B Subunit 3 (SF3B3) anti-CD137 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD137 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD137 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD137 antibody comprises a heavy chain of IgG3 isotype and a light chain of kappa isotype. In one embodiment, the bispecific anti-Splicing Factor 3B Subunit 3 (SF3B3) anti-CD137 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Splicing Factor 3B Subunit 3 (SF3B3) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Splicing Factor 3B Subunit 3 (SF3B3) anti-CD137 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Splicing Factor 3B Subunit 3 (SF3B3) anti-CD137 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers.

In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Splicing Factor 3B Subunit 3 (SF3B3) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Splicing Factor 3B Subunit 3 (SF3B3) anti-CD137 antibody comprises a heavy chain of IgG4 isotype and a light chain of kappa isotype. In one embodiment, the bispecific anti-Splicing Factor 3B Subunit 3 (SF3B3) anti-CD137 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec4e polypeptide, wherein the molecule is an IgG type antibody comprising a heavy chain of IgG1 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec4e polypeptide, wherein the molecule is an IgG type antibody comprising a heavy chain of IgG1 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD3 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec4e polypeptide, wherein the molecule is an IgG type antibody, comprises a heavy chain of IgG1 isotype and a light chain of kappa isotype. In one embodiment, the bispecific antigen-binding molecule which is an anti-CD3 antibody comprising a Clec4e polypeptide, wherein the molecule is an IgG type antibody, comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec4e polypeptide, wherein the molecule is an IgG type antibody comprising a heavy chain of IgG2 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec4e polypeptide, wherein the molecule is an IgG type antibody comprising a heavy chain of IgG2 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD3 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec4e polypeptide, wherein the molecule is an IgG type antibody, comprises a heavy chain of IgG2 isotype and a light chain of kappa isotype. In one embodiment, the bispecific antigen-binding molecule which is an anti-CD3 antibody comprising a Clec4e polypeptide, wherein the molecule is an IgG type antibody, comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec4e polypeptide, wherein the molecule is an IgG type antibody comprising a heavy chain of IgG3 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec4e polypeptide, wherein the molecule is an IgG type antibody comprising a heavy chain of IgG3 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD3 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec4e polypeptide, wherein the molecule is an IgG type antibody, comprises a heavy chain of IgG3 isotype and a light chain of kappa isotype. In one embodiment, the bispecific antigen-binding molecule which is an anti-CD3 antibody comprising a Clec4e polypeptide, wherein the molecule is an IgG type antibody, comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD3 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD3 antibody comprising a Clec4e polypeptide, wherein the molecule is an IgG type antibody comprising a heavy chain of IgG4 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD3 antibody comprising a Clec4e polypeptide, wherein the molecule is an IgG type antibody comprising a heavy chain of IgG4 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD3 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD3 antibody comprising a Clec4e polypeptide, wherein the molecule is an IgG type antibody, comprises a heavy chain of IgG4 isotype and a light chain of kappa isotype. In one embodiment, the bispecific antigen-binding molecule which is an anti-CD3 antibody comprising a Clec4e polypeptide, wherein the molecule is an IgG type antibody, comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec4e polypeptide, wherein the molecule is an IgG type antibody comprising a heavy chain of IgG1 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec4e polypeptide, wherein the molecule is an IgG type antibody comprising a heavy chain of IgG1 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD137 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec4e polypeptide, wherein the molecule is an IgG type antibody, comprises a heavy chain of IgG1 isotype and a light chain of kappa isotype. In one embodiment, the bispecific antigen-binding molecule which is an anti-CD137 antibody comprising a Clec4e polypeptide, wherein the molecule is an IgG type antibody, comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec4e polypeptide, wherein the molecule is an IgG type antibody comprising a heavy chain of IgG2 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec4e polypeptide, wherein the molecule is an IgG type antibody comprising a heavy chain of IgG2 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD137 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec4e polypeptide, wherein the molecule is an IgG type antibody, comprises a heavy chain of IgG2 isotype and a light chain of kappa isotype. In one embodiment, the bispecific antigen-binding molecule which is an anti-CD137 antibody comprising a Clec4e polypeptide, wherein the molecule is an IgG type antibody, comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec4e polypeptide, wherein the molecule is an IgG type antibody comprising a heavy chain of IgG3 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec4e polypeptide, wherein the molecule is an IgG type antibody comprising a heavy chain of IgG3 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD137 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec4e polypeptide, wherein the molecule is an IgG type antibody, comprises a heavy chain of IgG3 isotype and a light chain of kappa isotype. In one embodiment, the bispecific antigen-binding molecule which is an anti-CD137 antibody comprising a Clec4e polypeptide, wherein the molecule is an IgG type antibody, comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific antigen-binding molecule which is an anti-CD137 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers. In embodiments, the bispecific antigen-binding molecule comprises two heavy chains and two lights chains. In one embodiment, provided is a bispecific antigen-binding molecule which is an anti-CD137 antibody comprising a Clec4e polypeptide, wherein the molecule is an IgG type antibody comprising a heavy chain of IgG4 isotype and a light chain of kappa isotype, or a pharmaceutical composition comprising the bispecific antigen-binding molecule and one or more pharmaceutically acceptable carriers. In one embodiment, provided is a bispecific antigen-binding molecule which is an anti-CD137 antibody comprising a Clec4e polypeptide, wherein the molecule is an IgG type antibody comprising a heavy chain of IgG4 isotype and a light chain of lambda isotype, or a pharmaceutical composition comprising the bispecific antigen-binding molecule and one or more pharmaceutically acceptable carriers. In embodiments, the pharmaceutical composition is in the form of a lyophilized formulation or aqueous solution. In certain further embodiments of such bispecific antigen-binding molecule, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antigen-binding molecules may have their first H-chain CH3 region and second H-chain CH3 region crosslinked by disulfide bonds. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific antigen-binding molecule which is an anti-CD137 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers for use in a method of treating or preventing a cancer disease, preferably wherein the cancer disease is a solid tumour. In embodiments, the bispecific antigen-binding molecule comprises two heavy chains and two lights chains. In one embodiment, the bispecific antigen-binding molecule which is an anti-CD137 antibody comprising a Clec4e polypeptide, wherein the molecule is an IgG type antibody, comprises a heavy chain of IgG4 isotype and a light chain of kappa isotype. In one embodiment, the bispecific antigen-binding molecule which is an anti-CD137 antibody comprising a Clec4e polypeptide, wherein the molecule is an IgG type antibody, comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD3 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD3 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Proliferating Cell Nuclear Antigen (PCNA) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD3 antibody comprises a heavy chain of IgG1 isotype and a light chain of kappa isotype. In one embodiment, the bispecific anti-Proliferating Cell Nuclear Antigen (PCNA) anti-CD3 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD3 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD3 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD3 antibody comprises a heavy chain of IgG2 isotype and a light chain of kappa isotype. In one embodiment, the bispecific anti-Proliferating Cell Nuclear Antigen (PCNA) anti-CD3 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD3 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD3 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD3 antibody comprises a heavy chain of IgG3 isotype and a light chain of kappa isotype. In one embodiment, the bispecific anti-Proliferating Cell Nuclear Antigen (PCNA) anti-CD3 antibody comprises a heavy chain of IgG3 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD3 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD3 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD3 antibody comprises a heavy chain of IgG4 isotype and a light chain of kappa isotype. In one embodiment, the bispecific anti-Proliferating Cell Nuclear Antigen (PCNA) anti-CD3 antibody comprises a heavy chain of IgG4 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD137 antibody comprising a heavy chain of IgG1 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD137 antibody comprising a heavy chain of IgG1 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiments, the present invention relates to a bispecific anti-Proliferating Cell Nuclear Antigen (PCNA) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD137 antibody comprises a heavy chain of IgG1 isotype and a light chain of kappa isotype. In one embodiment, the bispecific anti-Proliferating Cell Nuclear Antigen (PCNA) anti-CD137 antibody comprises a heavy chain of IgG1 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD137 antibody comprising a heavy chain of IgG2 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD137 antibody comprising a heavy chain of IgG2 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD137 antibody comprises a heavy chain of IgG2 isotype and a light chain of kappa isotype. In one embodiment, the bispecific anti-Proliferating Cell Nuclear Antigen (PCNA) anti-CD137 antibody comprises a heavy chain of IgG2 isotype and a light chain of lambda isotype. In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD137 antibody comprising a heavy chain of IgG3 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD137 antibody comprising a heavy chain of IgG3 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution.

In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD137 antibody comprises a heavy chain of IgG3 isotype and a light chain of kappa isotype. In one embodiment, the bispecific anti-Proliferating Cell Nuclear Antigen (PCNA) anti-CD137 antibody comprises a heavy chain of IgG3 isotype and a light chain of lambda isotype.

In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In one aspect, provided is a bispecific anti-Proliferating Cell Nuclear Antigen (PCNA) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers. In embodiments, the bispecific antibody comprises two heavy chains and two lights chains. In one embodiment, provided is a bispecific anti-Proliferating Cell Nuclear Antigen (PCNA) anti-CD137 antibody comprising a heavy chain of IgG4 isotype and a light chain of kappa isotype, or a pharmaceutical composition comprising the bispecific antibody and one or more pharmaceutically acceptable carriers. In one embodiment, provided is a bispecific anti-Proliferating Cell Nuclear Antigen (PCNA) anti-CD137 antibody comprising a heavy chain of IgG4 isotype and a light chain of lambda isotype, or a pharmaceutical composition comprising the bispecific antibody and one or more pharmaceutically acceptable carriers. In certain further embodiments of such bispecific antibody, it is possible that two Fab arms are linked with each other via one or more bonds, such as disulfide bonds. In embodiments, the amino acid residues from which the bonds between the antigen-binding domains originate are present within the variable regions or the CH1 regions. Exemplary embodiments are schematically shown in FIG. 3. In one preferred embodiment, the antibodies may have their first H-chain CH3 region and second H-chain CH3 region crosslinked by disulfide bonds. In embodiments, the pharmaceutical composition is in the form of a lyophilized formulation or aqueous solution. In embodiments, the pharmaceutical composition comprises one or more pharmaceutically acceptable carriers. In embodiments, the one or more pharmaceutically acceptable carriers is selected from buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), and interstitial drug dispersion agents such as soluble neutral-active hyaluronidase glycoproteins (sHASEGP), for example, human soluble PH-20 hyaluronidase glycoproteins, such as rHuPH20.

In embodiment, the present invention relates to a bispecific anti-Proliferating Cell Nuclear Antigen (PCNA) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers for use in a method of treating or preventing a cancer disease, preferably wherein the cancer disease is a solid tumour. In embodiments, the bispecific antibody comprises two heavy chains and two lights chains. In one embodiment, the bispecific anti-Proliferating Cell Nuclear Antigen (PCNA) anti-CD137 antibody comprises a heavy chain of IgG4 isotype and a light chain of kappa isotype. In one embodiment, the bispecific anti-Proliferating Cell Nuclear Antigen (PCNA) anti-CD137 antibody comprises a heavy chain of IgG4 isotype and a light chain of lambda isotype.

In embodiments, the cancer disease is selected from gastric cancer, head and neck cancer, esophageal cancer, colorectal cancer, lung cancer, mesothelioma, liver cancer, ovarian cancer, breast cancer, colon cancer, kidney cancer, skin cancer, muscle tumor, pancreatic cancer, prostate cancer, testicular cancer, uterine cancer, cholangiocarcinoma, Merkel cell carcinoma, bladder cancer, thyroid cancer, schwannoma, adrenal cancer, anal cancer, central nervous system tumor, neuroendocrine tissue tumor, penile cancer, pleural tumor, salivary gland tumor, vulvar cancer, thymoma, lymphoma, myeloid leukemia, pediatric cancer (Wilms tumor, neuroblastoma, sarcoma, hepatoblastoma, and germ cell tumor). In embodiments, the cancer disease is selected from gastric cancer, colorectal cancer, lung cancer, mesothelioma, liver cancer, breast cancer, skin cancer, lymphoma, and myeloid leukemia.

In embodiments of any of the further specific bispecific antibodies described above, the antigen-binding molecule is characterized in that the antigen-binding molecule is selected from the group consisting of:
A) an antibody capable of binding to F-actin, as well as to an antigen involved in a signal transmission in a cell;
B) an antibody capable of binding to F-actin, as well as to an immune cell receptor; or
C) an antibody capable of binding to PCNA, as well as to an antigen involved in a signal transmission in a cell;
D) an antibody capable of binding to PCNA, as well as to an immune cell receptor; or
E) an antibody capable of binding to SF3B3, as well as to an antigen involved in a signal transmission in a cell;
F) an antibody capable of binding to SF3B3, as well as to an immune cell receptor; or
G) an antibody capable of binding to phosphatidylserine, as well as to an antigen involved in a signal transmission in a cell;
H) an antibody capable of binding to phosphatidylserine, as well as to an immune cell receptor;
preferably wherein the antigen-binding molecule is selected from the group consisting of
   A-1) an antibody capable of binding to F-actin, as well as to CD3;
   A-2) an antibody capable of binding to F-actin, as well as to CD137;
   C-1) an antibody capable of binding to PCNA, as well as to CD3;
   C-2) an antibody capable of binding to PCNA, as well as to CD137;
   E-1) an antibody capable of binding to SF3B3, as well as to CD3;
   E-2) an antibody capable of binding to SF3B3, as well as to CD137;
   G-1) an antibody capable of binding to phosphatidylserine, as well as to CD3;
   and
   G-2) an antibody capable of binding to phosphatidylserine, as well as to CD137.

In embodiments of any of the further trispecific antibodies described above, the antigen-binding molecule is characterized in that the antigen-binding molecule is selected from the group consisting of:
A) an antibody capable of binding to F-actin, an antigen involved in a signal transmission in a cell and as well as to an immune receptor; or
B) an antibody capable of binding to PCNA, an antigen involved in a signal transmission in a cell and as well as to an immune receptor; or
C) an antibody capable of binding to SF3B3, an antigen involved in a signal transmission in a cell and as well as to an immune receptor; or
D) an antibody capable of binding to Phosphatidylserine (PS), an antigen involved in a signal transmission in a cell and as well as to an immune receptor;
preferably wherein the antigen-binding molecule is selected from the group consisting of
A) an antibody capable of binding to F-actin, as well as to CD3 and CD137;
B) an antibody capable of binding to PCNA, as well as to CD3 and CD137;
C) an antibody capable of binding to SF3B3, as well as to CD3 and CD137;
D) an antibody capable of binding to Phospatidylserine (PS), as well as to CD3 and CD137.

In further embodiments of any of the aspects herein, the antigen-binding molecule is characterized in that the molecule is selected from the group consisting of:
A1) an antibody comprising one or two first moieties capable of binding to SF3B3, especially wherein the first moietie(s) is/are Ig-type binding moieties, preferably attached to the Fc domain of the antibody, as well as two second moieties involved in a signal transmission in a cell, especially wherein the second moieties are Ig-type binding moieties;
B1) an antibody comprising one or two first moieties capable of binding to SF3B3, especially wherein the first moietie(s) is/are Ig-type binding moieties, preferably attached to the Fc domain of the antibody, as well as two second moieties which are an immune cell receptor antigen, especially wherein the second moieties are Ig-type binding moieties;
C1) an antibody comprising one or two first moieties capable of binding to SF3B3, especially wherein the first moietie(s) is/are Ig-type binding moieties, preferably attached to the Fc domain of the antibody, as well as one second moiety and one third moiety involved in a signal transmission in a cell or in separate cells, especially wherein the second and third moieties are Ig-type binding moieties;
D1) an antibody comprising one or two first moieties capable of binding to SF3B3, especially wherein the first moietie(s) is/are Ig-type binding moieties, preferably attached to the Fc domain of the antibody, as well as one second moiety and one third moiety which are immune cell receptor antigens, especially wherein the second and third moieties are Ig-type binding moieties;
E1) an antibody comprising one or two first moieties capable of binding to SF3B3, especially wherein the first moietie(s) is/are Ig-type binding moieties, preferably attached to the Fc domain of the antibody, as well as one second moiety is involved in a signal transmission in a cell, and one third moiety is an immune cell receptor antigen, especially wherein the second and third moieties are Ig-type binding moieties; or
A2) an antibody comprising one or two first moieties capable of binding to PCNA, especially wherein the first moietie(s) is/are Ig-type binding moieties, preferably attached to the Fc domain of the antibody, as well as two second moieties involved in a signal transmission in a cell, especially wherein the second moieties are Ig-type binding moieties;
B2) an antibody comprising one or two first moieties capable of binding to PCNA, especially wherein the first moietie(s) is/are Ig-type binding moieties, preferably attached to the Fc domain of the antibody, as well as two second moieties which are an immune cell receptor antigen, especially wherein the second moieties are Ig-type binding moieties;
C2) an antibody comprising one or two first moieties capable of binding to PCNA, especially wherein the first moietie(s) is/are Ig-type binding moieties, preferably attached to the Fc domain of the antibody, as well as one second moiety and one third moiety involved in a signal transmission in a cell or in separate cells, especially wherein the second and third moieties are Ig-type binding moieties;
D2) an antibody comprising one or two first moieties capable of binding to PCNA, especially wherein the first moietie(s) is/are Ig-type binding moieties, preferably attached to the Fc domain of the antibody, as well as one second moiety and one third moiety which are immune cell receptor antigens, especially wherein the second and third moieties are Ig-type binding moieties;
E2) an antibody comprising one or two first moieties capable of binding to PCNA, especially wherein the first moietie(s) is/are Ig-type binding moieties, preferably attached to the Fc domain of the antibody, as well as one second moiety is involved in a signal transmission in a cell, and one third moiety is an immune cell receptor antigen, especially wherein the second and third moieties are Ig-type binding moieties; or
A3) an antibody comprising one or two first moieties capable of binding to PS, especially wherein the first moietie(s) is/are Ig-type binding moieties, preferably attached to the Fc domain of the antibody, as well as two second moieties involved in a signal transmission in a cell, especially wherein the second moieties are Ig-type binding moieties;
B3) an antibody comprising one or two first moieties capable of binding to PS, especially wherein the first moietie(s) is/are Ig-type binding moieties, preferably attached to the Fc domain of the antibody, as well as two second moieties which are an immune cell receptor antigen, especially wherein the second moieties are Ig-type binding moieties;
C3) an antibody comprising one or two first moieties capable of binding to PS, especially wherein the first moietie(s) is/are Ig-type binding moieties, preferably attached to the Fc domain of the antibody, as well as one second moiety and one third moiety involved in a signal transmission in a cell or in separate cells, especially wherein the second and third moieties are Ig-type binding moieties;
D3) an antibody comprising one or two first moieties capable of binding to PS, especially wherein the first moietie(s) is/are Ig-type binding moieties, preferably attached to the Fc domain of the antibody, as well as one second moiety and one third moiety which are immune cell receptor antigens, especially wherein the second and third moieties are Ig-type binding moieties;
E3) an antibody comprising one or two first moieties capable of binding to PS, especially wherein the first moietie(s) is/are Ig-type binding moieties, preferably attached to the Fc domain of the antibody, as well as one second moiety is involved in a signal transmission in a cell, and one third moiety is an immune cell receptor antigen, especially wherein the second and third moieties are Ig-type binding moieties; or
A4) an antibody comprising one or two first moieties capable of binding to F-actin, especially wherein the first moietie(s) is/are Ig-type binding moieties, preferably attached to the Fc domain of the antibody, as well as two second moieties involved in a signal transmission in a cell, especially wherein the second moieties are Ig-type binding moieties;
B4) an antibody comprising one or two first moieties capable of binding to F-actin, especially wherein the first moietie(s) is/are Ig-type binding moieties, preferably attached to the Fc domain of the antibody, as well as two second moieties which are an immune cell receptor antigen, especially wherein the second moieties are Ig-type binding moieties;
C4) an antibody comprising one or two first moieties capable of binding to F-actin, especially wherein the first moietie(s) is/are Ig-type binding moieties, preferably attached to the Fc domain of the antibody, as well as one second moiety and one third moiety involved in a signal transmission in a cell or in separate cells, especially wherein the second and third moieties are Ig-type binding moieties;
D4) an antibody comprising one or two first moieties capable of binding to F-actin, especially wherein the first moietie(s) is/are Ig-type binding moieties, preferably attached to the Fc domain of the antibody, as well as one second moiety and one third moiety which are immune cell receptor antigens, especially wherein the second and third moieties are Ig-type binding moieties;
E4) an antibody comprising one or two first moieties capable of binding to F-actin, especially wherein the first moietie(s) is/are Ig-type binding moieties, preferably attached to the Fc domain of the antibody, as well as one second moiety is involved in a signal transmission in a cell, and one third moiety is an immune cell receptor antigen, especially wherein the second and third moieties are Ig-type binding moieties; or
a) an antibody comprising two first moieties capable of binding to F-actin, especially wherein the first moieties are binding polypeptides, preferably Clec9A polypeptides, preferably attached to the Fc domain of the antibody, as well as two second moieties involved in a signal transmission in a cell, especially wherein the second moieties are Ig-type binding moieties;
b) an antibody comprising two first moieties capable of binding to F-actin, especially wherein the first moieties are binding polypeptides, preferably Clec9A polypeptides, preferably attached to the Fc domain of the antibody, as well as two second moieties which are an immune cell receptor antigen, especially wherein the second moieties are Ig-type binding moieties;
c) an antibody comprising two first moieties capable of binding to F-actin, especially wherein the first moieties are Ig-type binding moieties, as well as two second moieties involved in a signal transmission in a cell, especially wherein the second moieties are Ig-type binding moieties;
d) an antibody comprising two first moieties capable of binding to F-actin, especially wherein the first moieties are Ig-type binding moieties, as well as two second moieties, which are an immune cell receptor antigen, especially wherein the second moieties are Ig-type binding moieties;
e) an antibody comprising two first moieties capable of binding to PCNA, especially wherein the first moieties are Ig-type binding moieties, as well as two second moieties involved in a signal transmission in a cell, especially wherein the second moieties are Ig-type binding moieties;
f) an antibody comprising two first moieties capable of binding to PCNA, especially wherein the first moieties are Ig-type binding moieties, as well as two second moieties, which are an immune cell receptor antigen, especially wherein the second moieties are Ig-type binding moieties;
g) an antibody comprising two first moieties capable of binding to SF3B3, especially wherein the first moieties are Ig-type binding moieties, as well as two second moieties involved in a signal transmission in a cell, especially wherein the second moieties are Ig-type binding moieties;
h) an antibody comprising two first moieties capable of binding to SF3B3, especially wherein the first moieties are Ig-type binding moieties, as well as two second moieties, which are an immune cell receptor antigen, especially wherein the second moieties are Ig-type binding moieties;
i) an antibody comprising two first moieties capable of binding to phosphatidylserine, especially wherein the first moieties are Ig-type binding moieties, as well as two second moieties involved in a signal transmission in a cell, especially wherein the second moieties are Ig-type binding moieties;
j) an antibody comprising two first moieties capable of binding to phosphatidylserine, especially wherein the first moieties are Ig-type binding moieties, as well as two second moieties, which are an immune cell receptor antigen, especially wherein the second moieties are Ig-type binding moieties;
k) an antibody comprising two first moieties capable of binding to SF3B3, especially wherein the first moieties are binding polypeptides, preferably Clec4e polypeptides, preferably attached to the Fc domain of the antibody, as well as two second moieties involved in a signal transmission in a cell, especially wherein the second moieties are Ig-type binding moieties;
l) an antibody comprising two first moieties capable of binding to SF3B3, especially wherein the first moieties are binding polypeptides, preferably Clec4e polypeptides, preferably attached to the Fc domain of the antibody, as well as two second moieties, which are an immune cell receptor antigen, especially wherein the second moieties are Ig-type binding moieties, preferably wherein the molecule is selected from the group consisting of
a1) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD3, especially wherein the second moieties are Ig-type binding moieties;
a2) an antibody comprising two first moieties capable of binding to F-actin, especially wherein the first moieties are binding polypeptides, preferably Clec9A polypeptides, preferably attached to the Fc domain of the antibody, as well as two second moieties capable of binding to CD137, especially wherein the second moieties are Ig-type binding moieties;
e1) an antibody comprising two first moieties capable of binding to PCNA, especially wherein the first moieties are Ig-type binding moieties, as well as two second moieties capable of binding to CD3, especially wherein the second moieties are Ig-type binding moieties;
e2) an antibody comprising two first moieties capable of binding to PCNA, especially wherein the first moieties are Ig-type binding moieties, as well as two second moieties capable of binding to CD137, especially wherein the second moieties are Ig-type binding moieties;
g1) an antibody comprising two first moieties capable of binding to SF3B3, especially wherein the first moieties are Ig-type binding moieties, as well as two second moieties capable of binding to CD3, especially wherein the second moieties are Ig-type binding moieties;
g2) an antibody comprising two first moieties capable of binding to SF3B3, especially wherein the first moieties are Ig-type binding moieties, as well as two second moieties capable of binding to CD137, especially wherein the second moieties are Ig-type binding moieties;
i1) an antibody comprising two first moieties capable of binding to phosphatidylserine, especially wherein the first moieties are Ig-type binding moieties, as well as two second moieties capable of binding to CD3, especially wherein the second moieties are Ig-type binding moieties;
i2) an antibody comprising two first moieties capable of binding to phosphatidylserine, specially wherein the first moieties are Ig-type binding moieties, as well as two second moieties capable of binding to CD137, especially wherein the second moieties are Ig-type binding moieties;
k1) an antibody comprising two first moieties capable of binding to SF3B3, especially wherein the first moieties are binding polypeptides, preferably Clec4e polypeptides, preferably attached to the Fc domain of the antibody, as well as two second moieties capable of binding to CD3, especially wherein the second moieties are Ig-type binding moieties;
k2) an antibody comprising two first moieties capable of binding to SF3B3, especially wherein the first moieties are binding polypeptides, preferably Clec4e polypeptides, preferably attached to the Fc domain of the antibody, as well as two second moieties capable of binding to CD137, especially wherein the second moieties are Ig-type binding moieties.

All documents cited herein are incorporated herein by reference.

The following are examples of methods and compositions of the present disclosure It is understood that various other embodiments may be practiced, given the general description provided above. The examples are provided to illustrate, but not to limit the present invention.

### EXAMPLES

### Example 1

### Expression and Purification of Recombinant Antibodies

Recombinant antibodies were expressed transiently using Expi293 cell line (Thermo Fisher, Carlsbad, CA, USA). Antibody purification was carried out using Protein G or A affinity chromatography and gel filtration. The combination of genes encoding heavy chains and light chains for each antibody used for co-transfection is summarized in Table 1 and 1-1. Each expression vector encoding antibody sequence is designed to be for mammalian expression system. Bispecific antibody preparation using Fab-arm exchange (FAE) was conducted according to a method described (Proc Natl Acad Sci USA. 2013 Mar. 26; 110 (13): 5145-5150). For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4:2411-2423).

TABLE 1

| Antibody name | VH | CH | VL | CL |
|---|---|---|---|---|
| IC17-mF18 | IC17HdK | mF18 | IC17L | mk1 |
| (anti-KLH) | (SEQ ID | (SEQ ID | (SEQ ID | (SEQ ID |
| | NO: 1) | NO: 3) | NO: 7) | NO: 8) |
| IC17HdK- | IC17HdK | mF18.G3S2.m | IC17L | mk1 |
| mF18-(G3S)2- | (SEQ ID | C9ECD57 | (SEQ ID | (SEQ ID |
| mC9CD57// | NO: 1) | (SEQ ID | NO: 7) | NO: 8) |
| IC17-mK1 | | NO: 4) | | |
| (anti-KLH- | | | | |
| Clec9A ECD) | | | | |
| IC17HdK- | IC17HdK | mF18.G3S2.m | IC17L | mk1 |
| mF18-(G3S)2- | (SEQ ID | C9.131 | (SEQ ID | (SEQ ID |
| mC9-131// | NO: 1) | (SEQ ID | NO: 7) | NO: 8) |
| IC17-mK1 | | NO: 5) | | |
| (anti-KLH- | | | | |
| Clec9A CTLD) | | | | |
| TR01H113- | TR01H113 | mF18 | TR01L0011 | mk1 |
| mF18/ | (SEQ ID | (SEQ ID | (SEQ ID | (SEQ ID |
| TR01L0011- | NO: 2) | NO: 3) | NO: 6) | NO: 8) |
| mk1 (anti-CD3) | | | | |
| TR01H113- | TR01H113 | mF18.G3S2.m | TR01L0011 | mk1 |
| mF18.G3S2.m | (SEQ ID | C9ECD57 | (SEQ ID | (SEQ ID |
| C9ECD57/ | NO: 2) | (SEQ ID | NO: 6) | NO: 8) |
| TR01L0011- | | NO: 4) | | |
| mk1 (anti-CD3- | | | | |
| Clec9A ECD) | | | | |
| TR01H113- | TR01H113 | mF18.G3S2.m | TR01L0011 | mk1 |
| mF18.G3S2.m | (SEQ ID | C9.131 | (SEQ ID | (SEQ ID |
| C9.131/ | NO: 2) | (SEQ ID | NO: 6) | NO: 8) |
| TR01L0011- | | NO: 5) | | |
| mk1 (anti-CD3- | | | | |
| Clec9A CTLD) | | | | |

TABLE 1-1

| Antibody | | | | | | | | |
|---|---|---|---|---|---|---|---|---|
| name | VHA | CHA | VLA | CLA | VHB | CHB | VLB | CLB |
| TX_3G4VH- | 3G4VH | mF18.Nhe1 | 3G4VL | mk1 | | | | |
| mF18.Nhe1/ | (SEQ ID | (SEQ ID | (SEQ | (SEQ | | | | |
| 3G4VL-mk1 | NO: 39) | NO: 48) | ID NO: | ID | | | | |
| (anti-PS) | | | 44) | NO: | | | | |
| | | | | 31) | | | | |
| TX_Mab1.5.1VH- | Mab1.5.1VH | mF18.Nhe1 | Mab1.5.1VL | mk1 | | | | |
| mF18.Nhe1/ | (SEQ ID | (SEQ ID | (SEQ ID | (SEQ | | | | |
| Mab1.5.1VL-mk1 | NO: 40) | NO: 48) | NO: 45) | ID | | | | |
| (anti-HSP90 | | | | NO: | | | | |
| (1.5.1)) | | | | 31) | | | | |
| TX_6H8VH- | 6H8VH | mF18.Nhe1 | 6H8VL | mk1 | | | | |
| mF18.Nhe1/ | (SEQ ID | (SEQ ID | (SEQ ID | (SEQ | | | | |
| 6H8VL-mk1 | NO: 41) | NO: 48) | NO: 46) | ID | | | | |
| (anti-HSP90 | | | | NO: | | | | |
| (6H8)) | | | | 31) | | | | |
| TX_3G4VH- | 3G4VH | mBS1001b | 3G4VL | mk1 | TR01H113 | mBS1001a | TR01L0011 | mk1 |
| mBS1001b/ | (SEQ ID | (SEQ ID | (SEQ ID | (SEQ | (SEQ ID | (SEQ ID | (SEQ ID | (SEQ |
| 3G4VL-mk1// | NO: 39) | NO: 49) | NO: 44) | ID | NO: 19) | NO: 50) | NO: 30) | ID |
| TR01H113- | | | | NO: | | | | NO: |
| mBS1001a/ | | | | 31) | | | | 31) |
| TR01L0011-mk1 | | | | | | | | |
| (anti-PS//CD3) | | | | | | | | |
| TX_Mab1.5.1VH- | Mab1.5.1VH | mBS1001b | Mab1.5.1VL | mk1 | TR01H113 | mBS1001a | TR01L0011 | mk1 |
| mBS1001b/ | (SEQ ID | (SEQ ID | (SEQ ID | (SEQ | (SEQ ID | (SEQ ID | (SEQ ID | (SEQ |
| Mab1.5.1VL- | NO: 40) | NO: 49) | NO: 45) | ID | NO: 19) | NO: 50) | NO: 30) | ID |
| mk1//TR01H113- | | | | NO: | | | | NO: |
| mBS1001a/ | | | | 31) | | | | 31) |
| TR01L0011-mk1 | | | | | | | | |
| (anti-HSP90 | | | | | | | | |
| (1.5.1)//CD3) | | | | | | | | |
| TX 6H8VH- | 6H8VH | mBS1001b | 6H8VL | mk1 | TR01H113 | mBS1001a | TR01L0011 | mk1 |
| mBS1001b/ | (SEQ ID | (SEQ ID | (SEQ ID | (SEQ | (SEQ ID | (SEQ ID | (SEQ ID | (SEQ |
| 6H8VL-mk1// | NO: 41) | NO: 49) | NO: 46) | ID | NO: 19) | NO: 50) | NO: 30) | ID |
| TR01H113- | | | | NO: | | | | NO: |
| mBS1001a/ | | | | 31) | | | | 31) |
| TR01L0011-mk1 | | | | | | | | |
| (anti-HSP90 | | | | | | | | |
| (6H8)//CD3) | | | | | | | | |
| TR01H113- | TR01H113 | mBS1001a | TR01L0011 | mk1 | IC17HdK | mBS1001b | IC17L | mk1 |
| mBS1001a/ | (SEQ ID | (SEQ ID | (SEQ ID | (SEQ | (SEQ ID | (SEQ ID | (SEQ ID | (SEQ |
| TR01L0011// | NO: 19) | NO: 50) | NO: 30) | ID | NO: 20) | NO: 49) | NO: 33) | ID |
| IC17HdK- | | | | NO: | | | | NO: |
| mBS1001b/ | | | | 31) | | | | 31) |
| IC17L | | | | | | | | |
| (anti-KLH// | | | | | | | | |
| CD3) | | | | | | | | |
| TX_ | MOR74801H. | SG181 | MOR74801L. | SK1 | | | | |
| MOR74801H. | GS (SEQ ID | (SEQ ID | GS2 (SEQ ID | (SEQ | | | | |
| GS-SG181/ | NO: 43) | NO: 51) | NO: 47) | ID | | | | |
| MOR74801L. | | | | NO: | | | | |
| GS2-SK1 (anti- | | | | 38) | | | | |
| CD137) | | | | | | | | |
| TX_ | MOR74801H. | SG181. | MOR74801L. | SK1 | | | | |
| MOR74801H.GS- | GS (SEQ ID | G3S2.mC9. | GS2 (SEQ ID | (SEQ | | | | |
| SG181.G3S2. | NO: 43) | 131 (SEQ | NO: 47) | ID | | | | |
| mC9.131/ | | ID NO: 52) | | NO: | | | | |
| MOR74801L. | | | | 38) | | | | |
| GS2-SK1 (anti- | | | | | | | | |
| CD137-Clec9A) | | | | | | | | |
| TX_ | MOR74801H. | SG181. | MOR74801L. | SK1 | 3G4VH | SG181.S3p | 3G4VL | SK1 |
| MOR74801H.GS- | GS (SEQ ID | S3n2 | GS2 (SEQ | (SEQ | (SEQ ID | (SEQ ID | (SEQ ID | (SEQ |
| SG181.S3n2/ | NO: 43) | (SEQ ID | ID NO: 47) | ID | NO: 39) | NO: 55 | NO: 44) | ID |
| MOR74801L. | | NO: 54) | | NO: | | | | NO: |
| GS2-SK1// | | | | 38) | | | | 38) |
| 3G4VH- | | | | | | | | |
| SG181.S3p/ | | | | | | | | |
| 3G4VL-SK1 | | | | | | | | |
| (anti-PS//CD137) | | | | | | | | |
| TX_ | MOR74801H. | SG181v11k | MOR74801L. | SK1 | IC17L | xSG181v11hG3 | IC17HdK | xSK1 |
| MOR74801H.GS- | GS (SEQ ID | (SEQ ID | GS2 (SEQ ID | (SEQ | (SEQ ID | (SEQ ID NO: | (SEQ ID | (SEQ |
| SG181v11k/ | NO: 43) | NO: 53) | NO: 47) | ID | NO: 33) | 56 | NO: 20) | ID |
| MOR74801L. | | | | NO: | | | | NO: |
| GS2-SK1//IC17L- | | | | 38) | | | | 57) |
| xSG181v11hG3/ | | | | | | | | |
| IC17HdK-xSK1 | | | | | | | | |
| (anti-KLH// | | | | | | | | |
| CD137) | | | | | | | | |

### Example 2

### Binding of Clec9A-Conjugated Antibody to Surface of Live and Dead Cells

Clec9A mediated binding of live and dead cells were examined by FACS analysis. Cell death of Ba/F3 cells was induced with 20 micro M mitomycin C (MMC) treatment for 24 h at 37° C., following which 2.5×10⁵ cells was used for staining of each sample. Briefly, cells were incubated with 1000x diluted zombie violet (ZV) dye (BioLegend, #423114) for 20 min at RT, washed and blocked with anti-mouse CD16/32 antibody (BioLegend #101320) at final concentration of 2 micro g/mL for 10 min on ice. Surface staining with AF488 labelled anti-keyhole limpet hemocyanin (KLH) antibodies (anti-KLH) unconjugated or conjugated with Clec9A ECD (SEQ ID NO: 9) or Clec-9A CTLD (SEQ ID NO: 10) was performed at final concentration of 10 micro g/mL for 30 min on ice. Annexin V (AnnV) staining was performed using a commercial kit following manufacturer's instructions (BioLegend #640930). Data was acquired on the BD LSRFortessa™ X-20 Flow Cytometer and analysed using Flowjo v10.4.2 software.

Binding of the Clec9A conjugated antibodies were observed mainly on ZV+AnnV+necrotic cells, while minimal to no binding was observed on ZV-AnnV+apoptotic cells (FIG. 4). ZV-AnnV-live cell population showed no binding. Together, the data suggest that necrotic cells or its component(s) specifically bind Clec9A and mediate clustering of its conjugated antibodies.

### Example 3

### CD3 Activation by Clec9A-Mediated Clustering

Cell death was induced in the mouse B cell line Ba/F3 by treatment with 5 micro M mitoxantrone (MTX) (Sigma, #M2305000) for 24 h, at 37° C., 5% CO₂ at a concentration of 1×10⁶ cells/mL. Untreated or MTX treated Ba/F3 cells were then harvested, washed in fresh culture media and co-cultured at ratio of 1 Ba/F3 cells to 5 Jurkat luciferase reporter T cells (Promega, #J1601). To assess the effect of Clec9A mediated T cell activation, the extracellular (ECD) domain of Clec9A (SEQ ID NO: 9) or C-type lectin domain (CTLD) of Clec9A (SEQ ID NO: 10) was conjugated to the C-terminus of the Fc region on anti-human CD3 antibody (anti-CD3). Clec9A conjugated or unconjugated control anti-CD3 antibodies were added to co-cultures of BaF3 and Jurkat T cell cultures for 24 h, and supernatant was assessed for luciferase activity using the Bio-Glo™ luciferase assay system (Promega, #G7941). Data and graphs were analysed using GraphPad Prism software v8.4.2. Fold induction was calculated against negative control of each condition.

In the presence of “live” Ba/F3 cells that were not treated with MTX, unconjugated anti-CD3 antibodies induced ~22 fold of relative luciferase signal at maximum, while only ~5-fold induction was observed with CD3-targeting antibodies conjugated to Clec9A (FIG. 5). However, in the presence of “dead” Ba/F3 cells treated with MTX, a maximum of ~54 fold and ~46 fold relative luciferase activity was induced by Clec9A-ECD and Clec9A-CTLD conjugated antibodies respectively. This is significantly more than the ~20 relative fold induction observed with unconjugated anti-CD3 antibodies. These data suggest the presence of dead cells mediate clustering of Clec9A conjugated anti-CD3 antibodies, resulting in immune T cell stimulation.

### Example 4

### Binding of Anti-Phosphatidylserine and Anti-HSP90 Antibodies to Surface of Live and Dead Cells

Phosphatidylserine (PS) and heat-shock protein 90 (HSP90) are sequestered in normal cells but exposed to the extracellular environment during cellular stress. To confirm this point, anti-PS antibody (U.S. Pat. No. 8,956,616B2) and anti-HSP90 antibody clones 1.5.1 (U.S. Pat. No. 9,068,987B2) and 6H8 (WO2011/129379) were used to detect respective targets on live and dead cells by flow cytometry. For detection of PS, Ba/F3 mouse pro-B cell line was first treated with 20 micro M mitomycin C (MMC) for 48 h to induce cell death. For detection of HSP90, FreeStyle™ 293-F (Thermofisher Scientific #R790-07) human embryonic kidney cell line was pre-treated with 100 micro M MMC for 72 h. In subsequent staining, cells were incubated with 1000x diluted zombie violet (ZV) dye (BioLegend, #423114) for 20 min at room temperature (RT), washed and blocked with anti-mouse CD16/32 antibody (BioLegend #101320) at final concentration of 2 micro g/mL or human FcR blocking reagent (#130-059-901) at 20× dilution for 10 min at RT. Surface staining with anti-PS (anti-PS (3G4)) or anti-HSP90 (anti-HSP90 (1.5.1) and anti-HSP90 (6H8))) was performed at a final antibody concentration of 10 micro g/mL for 30 min at RT. Data was acquired on the BD LSRFortessa™ X-20 Flow Cytometer and analysed using Flowjo v10.4.2 software.

Binding of anti-PS and anti-HSP90 antibodies were observed on ZV+gated dead cells that were treated with MMC, while no binding was detected on ZV-live cells (FIG. 6). Thus, surface expression of PS or HSP90 was specific only to dead cells.

### Example 5

### CD3 Activation by Phosphatidylserine (PS) and Heat Shock Protein (HSP)-Mediated Clustering

To investigate if dead cell specific surface expression of PS or HSP90 translates to dead cell specific immune cell activation, live or dead cells were co-cultured with CD3 expressing Jurkat reporter T cells (Promega, #J1600), in the presence of anti-PS//CD3 (anti-PS//CD3) or anti-HSP90//CD3 (anti-HSP90 (1.5.1)//CD3 or anti-HSP90 (6H8)//CD3) bispecific antibodies. For assessment of anti-PS//CD3 activity, Ba/F3 cells were pre-treated with 5 micro M mitoxantrone (MTX) (Sigma, #M2305000) for 48 h to induce cell death. Untreated or MTX treated Ba/F3 cells were then co-cultured with 5times the number of Jurkat reporter T cells. For assessment of anti-PS//HSP90 activity, untreated or MMC treated FreeStyle™ 293-F cells were co-cultured with an equal number of Jurkat reporter T cells. 24 h after antibody stimulation, luciferase activity was detected using the Bio-Glo™ luciferase assay system (Promega, #G7941) and GloMax (registered trademark) Explorer (#GM3500). Data and graphs were analysed using GraphPad Prism software v9.0.2. Fold induction was calculated relative to the no antibody treatment control of each co-culture condition.

In the presence of dead cells, anti-PS//CD3 antibody induced 16-fold and 70-fold luciferase signal at 5 and 25 nM antibody concentrations respectively (FIG. 7). This activity induction was significantly higher compared to co-cultures with live cells at the same antibody concentrations. Similarly, anti-HSP90//CD3 antibodies showed induction of luciferase activity in co-cultures with dead cells at 5 and 25 nM antibody concentration, but no relative fold induction when co-cultured with live cells (FIG. 7). This phenomenon was not dependent on antibody clonality, since both anti-HSP90 clones induced similar luciferase reporter activity only in the presence of dead cells. Together, the results are consistent with CD3 activation in a dead cell dependent manner, and suggests different dead cell associated molecules can be targeted for immune cell modulation.

### Example 6

### CD137 Activation by Clec9A-Mediated Clustering

To examine whether the concept of dead cell immune modulation can extend to effector targets other than CD3, the extracellular domain of Clec9A was conjugated to the C-terminus of Fc region on anti-human CD137 agonist antibody (CAS: 1417318-27-4) ((anti-CD137-Clec9A). Dead cell dependent activation of CD137 was assessed in live or MTX treated dead Ba/F3 cells co-cultured with CD137 Jurkat reporter T cells (Promega #J2332) at an effector to target ratio of 1:5. Cells were treated with Clec9A conjugated anti-CD137-Clec9A or control unconjugated anti-human CD137 antibody (anti-CD137) for 24 h before luciferase activity was detected and analysed.

In the presence of MTX treated dead Ba/F3 cells, induction of CD137 was observed in anti-CD137-Clec9A antibody treated conditions (FIG. 8). This induction was Clec9A dependent since unconjugated anti-CD137 antibody did not result in relative fold induction of luciferase activity. Notably, fold induction was also not observed in the presence of live Ba/F3 by anti-CD137-Clec9A antibody. These results suggest that conjugation of a dead cell binding moiety like Clec9A to CD137 agonist antibodies results in dead cell dependent immune activation, similar to observations with dead cell binding CD3 agonist antibodies. Hence, dead cell specificity may be conferred to distinct effector molecules through combining with dead cell binding moieties.

### Example 7

### CD137 Activation by Phosphatidylserine (PS)-Mediated Clustering

To investigate if dead cell specific surface expression of PS or HSP90 translates to dead cell specific immune cell activation via CD137, anti-PS//CD137 bispecific antibodies was assessed in a similar assay set up as described for anti-CD137-Clec9A. Anti-PS//CD137 induced enhanced reporter cell activity in the presence of dead cells when compared to co-cultures with live cells. In contrast, anti-KLH//CD137 antibodies, which do not bind PS, did not show any difference in reporter activity between live and dead cell co-cultures (FIG. 9). Together, the data shows that the concept of dead cell specific modulation can be applied to distinct effector targets and through the binding of different dead cell associated molecules.

### Example 8

### Clec9A-Fused Antibody Activates CD3 in a F-Actin Dependent Manner

In the present invention, dead cell dependent modulation by Clec9A conjugated antibodies was described. To confirm this phenomenon occurs through binding of filamentous actin (F-actin) on dead cells (Immunity. 2012 Apr. 20;36 (4): 646-57), activation of CD3 was examined in the presence or absence of F-actin. Briefly, recombinant F-actin (Cytoskeleton, #AKF99), reconstituted to 0.4 mg/mL, was coated onto wells of Nunc Maxisorp™ plates (Thermofisher Scientific #442404) at 4° C. overnight. Uncoated wells were filled with 50 micro L of general actin buffer (Cytoskeleton, #BSA01). The following day, wells were washed and blocked with Dulbecco's phosphate buffer saline (DPBS) supplemented with 5% bovine serum albumin (BSA, Sigma-Aldrich #A7888) for 1 h at room temperature (RT). After the blocking step, anti-CD3 antibodies, unconjugated (anti-CD3) or conjugated to Clec9A C-type lectin domain (anti-CD3-Clec9A) was titrated and added into F-actin coated or uncoated wells for 15 min at 37° C., before addition of 5 ×10⁴ Jurkat luciferase reporter T cells (Promega, #J1601) per well. Following incubation at 37° C., 5% CO₂ for 24 h, luciferase activity was assessed using the Bio-Glo™ luciferase assay system (Promega, #G7940) and GloMax (registered trademark) Explorer System (Promega #GM3500). Results were analyzed using GraphPad Prism software v9.0.2.

In the presence of F-actin, Clec9A conjugated CD3 antibodies induced significantly higher T cell reporter activation than in wells where F-actin was not coated (FIG. 10). This enhanced activation was dependent on Clec9A, since unconjugated CD3 antibodies do not show any difference in reporter activity between F-actin coated and uncoated wells. Therefore, it is conceivable that modulation of CD3 activity by anti-CD3-Clec9A antibody was dependent on the interaction between Clec9A and F-actin. This result confirms the mechanism of dead cell dependent activation by Clec9A conjugated antibodies.

### Example 9

### Expression and Purification of Recombinant Antibodies

Recombinant antibodies were expressed transiently using Expi293 cell line (Thermo Fisher, Carlsbad, CA, USA). Antibody purification was carried out using Protein G or A affinity chromatography and gel filtration. The combination of genes encoding heavy chains and light chains for each antibody used for co-transfection is summarized in Table 2. Each expression vector encoding antibody sequence is designed to be for mammalian expression system. Bispecific antibody preparation using Fab-arm exchange (FAE) was conducted according to a method described (Proc Natl Acad Sci USA. 2013 Mar. 26; 110 (13): 5145-5150). For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4:2411-2423).

TABLE 2

| Antibody | | | | | | | | |
|---|---|---|---|---|---|---|---|---|
| name | VHA | CHA | VLA | CLA | VHB | CHB | VLB | CLB |
| IC17HdK- | IC17HdK | SG181 | IC17L | SK1 | | | | |
| SG181/ | (SEQ ID | (SEQ | (SEQ | (SEQ | | | | |
| IC17L-SK1 | NO: 1) | ID NO: | ID NO: | ID NO: | | | | |
| (KLH- | | 51) | 7) | 38) | | | | |
| SG181) | | | | | | | | |
| IC17HdK- | IC17HdK | SG181. | IC17L | SK1 | TR01H113 | SG181. | TR01L0011 | SK1 |
| SG181. | (SEQ ID | S3n2 | (SEQ | (SEQ ID | (SEQ ID | S3p | (SEQ ID | (SEQ |
| S3n2/ | NO: 1) | (SEQ | ID NO: | NO: 38) | NO: 2) | (SEQ | NO: 6) | ID NO: |
| IC17L- | | ID NO: | 7) | | | ID NO: | | 38) |
| SK1// | | 54) | | | | 55) | | |
| TR01H113- | | | | | | | | |
| SG181.S3p/ | | | | | | | | |
| TR01L0011- | | | | | | | | |
| SK1 (anti- | | | | | | | | |
| KLH//CD3- | | | | | | | | |
| SG181) | | | | | | | | |
| JG6353VH.I- | JG6353VH. | SG181. | JG6353VL. | k0MTC | TR01H113 | SG181. | TR01L0011 | SK1 |
| SG181.S3n2/ | I (SEQ ID | S3n2 | L (SEQ ID | (SEQ ID | (SEQ ID | S3p | (SEQ ID | (SEQ |
| JG6353VL.L- | NO: 58) | (SEQ | NO: 59) | NO: 60) | NO: 2) | (SEQ | NO: 6) | ID NO: |
| k0MTC// | | ID NO: | | | | ID NO: | | 38) |
| TR01H113- | | 54) | | | | 55) | | |
| SG181.S3p/ | | | | | | | | |
| TR01L0011- | | | | | | | | |
| SK1 (anti- | | | | | | | | |
| SF3B3//CD3- | | | | | | | | |
| SG181) | | | | | | | | |
| PC10VH- | PC10VH | SG181. | PC10VL | SK1 | AN121HGB | SG181. | TR01L0011 | SK1 |
| SG181.S3n/ | (SEQ ID | S3n | (SEQ ID | (SEQ | (SEQ ID | S3p | (SEQ ID | (SEQ |
| PC10VL- | NO: 61) | (SEQ | NO: 65) | ID NO: | NO: 68) | (SEQ | NO: 6) | ID NO: |
| SK1// | | ID NO: | | 38) | | ID NO: | | 38) |
| AN121HGB- | | 64) | | | | 55) | | |
| SG181.S3p/ | | | | | | | | |
| TR01L0011- | | | | | | | | |
| SK1 (anti- | | | | | | | | |
| PCNA// | | | | | | | | |
| AN121- | | | | | | | | |
| SG181) | | | | | | | | |
| IC17HdK- | IC17HdK | SG181. | IC17L | SK1 | AN121HGB | SG181. | TR01L0011 | SK1 |
| SG181.S3n/ | (SEQ ID | S3n | (SEQ ID | (SEQ | (SEQ ID | S3p | (SEQ ID | (SEQ |
| IC17L-SK1// | NO: 1) | (SEQ | NO: 7) | ID NO: | NO: 68) | (SEQ | NO: 6) | ID NO: |
| AN121HGB- | | ID NO: | | 38) | | ID | | 38) |
| SG181.S3p/ | | 64) | | | | NO: | | |
| TR01L0011- | | | | | | 55) | | |
| SK1 (anti- | | | | | | | | |
| KLH// | | | | | | | | |
| AN121- | | | | | | | | |
| SG181) | | | | | | | | |
| 1N11VH- | IN11VH | SG181 | 1N11VL | SK1 | | | | |
| SG181/ | (SEQ ID | (SEQ | (SEQ ID | (SEQ | | | | |
| 1N11VL- | NO: | ID NO: | NO: 66) | ID NO: | | | | |
| SK1 | 62) | 51) | | 38) | | | | |
| (anti-PS | | | | | | | | |
| (1N11)- | | | | | | | | |
| SG181) | | | | | | | | |
| 8E5D12A9H- | 8E5D12A9H | SG181 | 8E5D12A9L | SK1 | | | | |
| SG181/ | (SEQ ID | (SEQ | (SEQ ID | (SEQ | | | | |
| 8E5D12A9L- | NO: 63) | ID NO: | NO: 67) | ID NO: | | | | |
| SK1 (anti- | | 51) | | 38) | | | | |
| actin | | | | | | | | |
| (8E5D12A9)- | | | | | | | | |
| SG181) | | | | | | | | |
| IN11VH- | 1N11VH | SG181. | 1N11VL | SK1 | AN121HGB | SG181. | TR01L0011 | SK1 |
| SG181.S3n/ | (SEQ ID | S3n | (SEQ ID | (SEQ | (SEQ ID | S3p | (SEQ ID | (SEQ |
| 1N11VL- | NO: 62) | (SEQ | NO: 66) | ID NO: | NO: 68) | (SEQ | NO: 6) | ID NO: |
| SK1// | | ID NO: | | 38) | | ID | | 38) |
| AN121HGB- | | 64) | | | | NO: | | |
| SG181.S3p/ | | | | | | 55 | | |
| TR01L0011- | | | | | | | | |
| SK1 (anti- | | | | | | | | |
| PS(1N11)// | | | | | | | | |
| AN121- | | | | | | | | |
| SG181) | | | | | | | | |
| MOR74801H. | MOR74801H. | SG181. | MOR74801L. | SK1 | 1N11VH | SG181. | 1N11VL | SK1 |
| GS-SG181. | GS (SEQ | S3n2 | GS2 (SEQ | (SEQ | (SEQ ID | S3p | (SEQ | (SEQ |
| S3n2/ | ID NO: 43) | (SEQ | ID NO: 47) | ID NO: | NO: 62) | (SEQ | ID NO: | ID NO: |
| MOR74801L. | | ID NO: | | 38) | | ID | 66) | 38) |
| GS2-SK1// | | 54) | | | | NO: | | |
| 1N11VH- | | | | | | 55 | | |
| SG181.S3p/ | | | | | | | | |
| 1N11VL- | | | | | | | | |
| SK1 (anti- | | | | | | | | |
| PS(1N11)// | | | | | | | | |
| CD137- | | | | | | | | |
| SG181) | | | | | | | | |
| MOR74801H. | MOR74801H. | SG181. | MOR74801L. | SK1 | IC17HdK | SG181. | IC17L | SK1 |
| GS-SG181. | GS (SEQ | S3n2 | GS2 (SEQ | (SEQ | (SEQ ID | S3p | (SEQ ID | (SEQ |
| S3n2/ | ID NO: 43) | (SEQ | ID NO: 47) | ID NO: | NO: 1) | (SEQ | NO: 7) | ID NO: |
| MOR74801L. | | ID NO: | | 38) | | ID | | 38) |
| GS2-SK1// | | 54) | | | | NO: | | |
| IC17HdK- | | | | | | 55) | | |
| SG181.S3p/ | | | | | | | | |
| IC17L-SK1 | | | | | | | | |
| (anti-KLH// | | | | | | | | |
| CD137- | | | | | | | | |
| SG181) | | | | | | | | |
| IC17HdK- | IC17HdK | SG181. | IC17L | SK1 | | | | |
| SG181. | (SEQ ID | G3S2. | (SEQ ID | (SEQ | | | | |
| G3S2.mC9. | NO: 1) | mC9. | NO: 7) | ID NO: | | | | |
| 131/IC17L- | | 131 | | 38) | | | | |
| SK1 (anti- | | (SEQ | | | | | | |
| KLH-SG181- | | ID NO: | | | | | | |
| Clec9A | | 52) | | | | | | |
| CTLD) | | | | | | | | |
| PC10VH- | PC10VH | SG181 | PC10VL | SK1 | | | | |
| SG181/ | (SEQ ID | (SEQ | (SEQ ID | (SEQ | | | | |
| PC10VL- | NO: 61) | ID NO: | NO: 65) | ID NO: | | | | |
| SK1 (anti- | | 51) | | 38) | | | | |
| PCNA- | | | | | | | | |
| SG181) | | | | | | | | |
| JG6353VH.I- | JG6353VH.I | SG181 | JG6353VL.L | k0MTC | | | | |
| SG181/ | (SEQ ID | (SEQ | (SEQ ID | (SEQ ID | | | | |
| JG6353VL.L- | NO: 58) | ID NO: | NO: 59) | NO: 60) | | | | |
| k0MTC | | 51) | | | | | | |
| (anti-SF3B3- | | | | | | | | |
| SG181) | | | | | | | | |

### Example 10

### Binding of Anti-SF3B3 and Anti-PCNA Antibodies to Surface of Live and Dead Cells

Splicing factor 3B subunit 3 (SF3B3), a component of the small nuclear ribonucleoprotein complex and proliferating cell nuclear antigen (PCNA), a DNA sliding clamp factor are primarily nuclear proteins in normal healthy cells. To determine if these nuclear localized proteins are localised at the surface of dead cells, SF3B3 antibody (Novus biologicals #NBP2-76869) or PCNA antibody (Thermofisher Scientific #13-3900) were used to detect the respective targets on live and dead cells by FACS analysis. For detection of binding to mouse dead cells, E.G7-OVA cell line (ATCC #CRL-2113) was first treated with 40 micromolar mitomycin C (MMC) for 48 h to induce cell death. For detection of binding to human dead cells, FreeStyle™ 293-F (Thermofisher Scientific #R790-07) human embryonic kidney cell line was pre-treated with MMC for 72 h. In subsequent staining steps, cells were incubated with 1000x diluted zombie violet (ZV) dye (BioLegend, #423114) for 20 min at room temperature (RT). Surface binding of primary antibodies was performed by incubation of cells with titrated SF3B3, PCNA or Keyhole limpet hemocyanin (KLH-SG181) isotype antibodies for 30 min at RT, followed by detection using Alexa Fluor (registered trademark)-488 conjugated secondary antibodies at a final concentration of 1.5 micro g/mL. Data was acquired on the BD LSRFortessa™ X-20 Flow Cytometer and analysed using Flowjo v10.4.2 software.

The results show that SF3B3 and PCNA were detected on the surface of ZV+gated dead cells, with increasing fluorescence signals at higher antibody concentrations (FIGS. 11A and 11B). No binding was detected when KLH isotype antibody was used. Moreover, these observations were reproduced on both mouse and human dead cells, suggesting the externalization of these antigens across different cell types and species. Importantly, surface SF3B3 and PCNA were not detected on ZV-live cells (FIGS. 11A and 11B). Therefore, surface localization of SF3B3 and PCNA was specific to dead cells and could suggest binding of these antigens for dead cell targeting.

### Example 11

### CD3 Activation by SF3B3-Mediated Clustering

To investigate if dead cell specific surface expression of SF3B3 translates to dead cell specific immune cell activation, live or dead cells were co-cultured with CD3 expressing Jurkat reporter T cells (Promega, #J1600), in the presence of anti-SF3B3//CD3 (anti-SF3B3//CD3-SG181) bispecific antibody. Ba/F3 cells were pre-treated with 5 micromolar mitoxantrone (MTX) (Sigma, #M2305000) for 48 h to induce cell death. Untreated live or MTX treated dead Ba/F3 cells were then co-cultured with 5 times the number of Jurkat reporter T cells. 24 h after antibody stimulation, luciferase activity was detected using the Bio-Glo™ luciferase assay system (Promega, #G7940) and GloMax™ Explorer (#GM3500). Data and graphs were analysed using GraphPad Prism software v9.0.2. Fold induction was calculated relative to the no antibody treatment control of each co-culture condition.

In dead cell co-cultures, anti-SF3B3//CD3 antibody induced 5.3-fold and 13.2-fold relative luciferase signal at 5 and 25 nanomolar antibody concentrations respectively (FIG. 12). This activity induction was significantly higher compared to co-cultures with live cells at the same antibody concentrations. Moreover, increased CD3 induction was observed compared to isotype control antibodies that do not bind SF3B3 (anti-KLH//CD3-SG181). Together, the results are consistent with significantly higher CD3 activation in the presence of dead cells.

### Example 12

### CD3 Activation by PCNA-Mediated Clustering

To investigate if dead cell specific surface expression of PCNA translates to dead cell specific immune cell activation, anti-PCNA//CD3 (anti-PCNA//AN121-SG181) bispecific antibody is used in a similar assay set up as described for CD3 activation by anti-SF3B3 antibody.

In dead cell co-cultures, anti-PCNA//CD3 antibody induced 2.9-fold and 6.9-fold relative luciferase signal at 5 and 25 nanomolar antibody concentrations respectively (FIG. 13). This activity induction was significantly higher compared to co-cultures with live cells at the same antibody concentrations. Moreover, increased CD3 induction was observed compared to isotype control antibodies that do not bind PCNA, anti-KLH//CD3 (anti-KLH//AN121-SG181). Together, the results are consistent with stronger CD3 activation in the presence of dead cells.

### Example 13

### CD137 Activation by SF3BS- and PCNA-Mediated Clustering

To investigate if dead cell specific surface expression of SF3B3 and PCNA translates to dead cell specific immune cell activation via CD137, anti-SF3B3//CD137 or anti-PCNA//CD137 bispecific antibodies are used in a similar assay set up as described for described for CD3 activation by anti-SF3B3 antibody.

### Example 14

### Binding of Anti-PS (1N11) and Anti-Actin Antibodies to Surface of Live and Dead Cells

In addition to the anti-PS antibody in Example 4, an additional clone, anti-PS (1N11)-SG181 (WO2018/187227) antibody was also evaluated. In parallel, an anti-actin (CN113845593) (anti-actin (8E5D12A9)-SG181) antibody was evaluated. Both antibodies were subjected to FACS analysis for detection of their respective antigens on live and dead cells. For detection of binding to mouse dead cells, E.G7-OVA cell line (ATCC #CRL-2113) was first treated with 40 micro M mitomycin C (MMC) for 48 h to induce cell death. For detection of binding to human dead cells, FreeStyle™ 293-F (Thermofisher Scientific #R790-07) human embryonic kidney cell line was pre-treated with MMC for 72 h. In subsequent staining steps, cells were incubated with 1000x diluted zombie violet (ZV) dye (BioLegend, #423114) for 20 min at room temperature (RT). Surface binding of primary antibodies was performed by incubation of cells with titrated anti-PS (1N11) or anti-actin antibodies for 30 min at RT, followed by detection using Alexa Fluor (registered trademark)-488 conjugated secondary antibodies at a final concentration of 1.5 micro g/mL. Data was acquired on the BD LSRFortessa™ X-20 Flow Cytometer and analysed using Flowjo v10.4.2 software. Data and graphs were plotted using GraphPad Prism software v9.0.2.

The results show that anti-PS (1N11) and anti-actin (8E5D12A9) antibodies bound to the surface of ZV+gated dead cells while binding signals were significantly lower on ZV-live cells (FIGS. 14A and 14B). These observations were reproduced on both mouse and human cells.

### Example 15

### CD3 and CD137 Activation by PS (1N11)-Mediated Clustering

To investigate if anti-PS (1N11) mediated binding could also induce dead cell specific immune cell activation, anti-PS (1N11)/CD3 (anti-PS (1N11)//AN121-SG181) bispecific antibody was used in similar assay setup as described for CD3 activation by anti-SF3B3 antibody.

In dead cell co-cultures, anti-PS (1N11)//CD3 antibody induced 149.4-fold and 158.6-fold relative luciferase signal at 5 and 25 nanomolar antibody concentrations respectively (FIG. 15A). This activity induction was significantly higher compared to co-cultures with live cells at the same antibody concentrations. Moreover, increased CD3 induction was observed compared to isotype control antibodies that do not bind PS, anti-KLH//CD3 (anti-KLH//AN121-SG181).

To examine whether anti-PS (1N11) mediated binding could also induce dead cell dependent CD137 activation, anti-PS (1N11)//CD137 (anti-PS (1N11)//CD137-SG181) bispecific antibody activity was assessed. Live or mitoxantrone (MTX) (Sigma, #M2305000) treated dead Ba/F3 cells was co-cultured with CD137 Jurkat reporter T cells (Promega #J2332) at an effector to target ratio of 1:5 and treated with anti-PS (1N11)//CD137 or control antibody (anti-KLH//CD137-SG181) for 24 h before luciferase activity was detected and analysed using the Bio-Glo™ luciferase assay system (Promega, #G7940) and GloMax™ Explorer (#GM3500). Data and graphs were analysed using GraphPad Prism software v9.0.2. Fold induction was calculated relative to the no antibody treatment control of each co-culture condition.

Anti-PS (1N11)//CD137 antibody induced enhanced reporter cell activity in the presence of dead cells when compared to co-cultures with live cells. In dead cell co-cultures, anti-PS (1N11)//CD137 antibody induced 6-fold and 18.4-fold relative luciferase signal at 5 and 25 nanomolar antibody concentrations respectively (FIG. 15B). In contrast, anti-KLH//CD137 antibody, which do not bind PS, did not show any difference in reporter activity between live and dead cell co-cultures nor induce much reporter signal. Together, the results suggest anti-PS (1N11) mediated binding can induce dead cell specific immune cell activation.

### Example 16

### In Vivo Distribution of Dead Cell Binding Molecules in Muscle Injury Model

Dying or dead cells are often abundant at damaged tissues and disease sites. To determine if dead cell binding molecules could specifically accumulate at these sites in vivo, cardiotoxin (CTX) muscle injury model was used. 0.01 mg cardiotoxin (Latoxan, #11061-96-4) in 150 micro L PBS was injected into the right gastrocnemius muscle of 6 to 7-week-old male C57BL/6NTac mice while an equal volume of PBS was injected into the contralateral leg. 1 day after CTX injection, anti-KLH-SG181-Clec9A CTLD, anti-PS (1N11)-SG181, anti-PCNA-SG181, anti-SF3B3-SG181 or control KLH-SG181 antibodies were injected at a dose of 10 nmol/kg of mice by the intravenous (I.V.) route. 1 day following antibody administration, injured muscles were harvested and fixed for at least 24 h in 10% neutral buffered formalin (Sigma #HT501128). Immunohistochemistry was performed to detect accumulation of dead cell targeting molecules. Specifically, formalin fixed paraffin embedded tissues were cut onto slides and stained using the EnVision FLEX, High pH kit (Agilent #K8000). Antigen retrieval was performed using working concentration of En Vision FLEX Target Retrieval Solution, High pH at 97° C. for 20 min. For antibody staining, anti-human IgG antibody (Abcam #ab109489) was used at a dilution of 1:200, followed by secondary antibody staining with anti-rabbit IgG (Abcam #97051) at a dilution of 1:1000. Slides were scanned at 20× objective magnification using ScanScope AT Turbo (Leica Microsystems K.K.) and analysed using Aperio ImageScope software v12.4.6.5003 (Leica Biosystems).

The results show that all dead cell targeting molecules tested accumulated at inflamed and damaged areas of the muscle (FIG. 16). The accumulation was specific to damaged sites since non-damaged areas of muscle showed significantly weaker staining intensity. In addition, negative control anti-KLH antibody showed much weaker staining compared to dead cell targeting molecules.

Altogether, the data provided evidence of distribution of dead cell targeting molecules to specific sites experiencing cell death or tissue damage. The present invention provides a molecule that binds to a component or a portion thereof of a cell that is exposed to an extracellular environment such as a cell death or cell injury, and demonstrated their potential to act specifically in a tissue where a cell death is observed, or a disease tissue where cell damage is observed and the like.

### INDUSTRIAL APPLICABILITY

The present invention provides a molecule which binds to a component or a portion thereof of a cell which is exposed to a extracellular environment due to a cell death (e.g. a filament or a histone which forms a cytoskeleton or a nuclear skeleton), which is observed specifically in an affected tissue (e.g., TME), and concurrently binds to a target molecule (e.g., a membrane protein) on or in a cell (e.g., an immune cell, an affected cell such as a tumor cell, an autoreactive cell, and a virus-infected cell), which acts specifically in a tissue where a cell death is observed such as affected tissue, abnormal tissue or the like.

1. An antigen-binding molecule which comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
a) Splicing Factor 3B Subunit 3 (SF3B3); and
b) Proliferating Cell Nuclear Antigen (PCNA).

2. The antigen-binding molecule of claim 1, wherein the antigen-binding molecule comprises at least one third moiety that binds to a third antigen; wherein (i) said third antigen is different from the first and second antigen, or wherein (ii) said third antigen is different from the first but same as the second antigen; preferably wherein said third antigen is a membrane protein of an immune cell, or a tumour cell.

3. The antigen-binding molecule of claim 1 or 2, wherein said first antigen is exposed to an extracellular environment due to cell damage, particularly due to cell death.

4. The antigen-binding molecule of any one of claims 1 to 3, wherein the first moiety is selected from the group consisting of:
A) an Ig type binding moiety; or
B) a binding polypeptide, particularly wherein the said binding polypeptide is a non-Ig-type binding moiety.

5. The antigen-binding molecule of claim 4, wherein
A) in case that the first antigen is Splicing Factor 3B Subunit 3 (SF3B3), the first moiety is an Ig type binding moiety, or
B) in case that the first antigen is Splicing Factor 3B Subunit 3 (SF3B3), the first moiety is a Clec4e polypeptide, or
C) in case that the first antigen is Proliferating Cell Nuclear Antigen (PCNA), the first moiety is an Ig type binding moiety.

6. The antigen-binding molecule of any one of claims 1 to 5, wherein the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and/or the third antigen is a membrane protein involved in a signal transmission in a cell, preferably a signal transmission receptor.

7. The antigen-binding molecule of any one of claims 1 to 6, wherein the second and/or the third antigen is a membrane protein of a tumour cell.

8. The antigen-binding molecule of any one of claims 1 to 7, wherein the second and/or the third moiety is an Ig type binding moiety.

9. The antigen-binding molecule of any one of claims 1 to 8, wherein the antigen-binding molecule is an antibody, in particular an IgG type antibody.

10. The antigen-binding molecule of claim 9, wherein the antibody is capable of binding to a membrane protein of an immune cell or a tumour cell and further comprises, preferably linked to the Fc region, at least one binding polypeptide, particularly wherein the said binding polypeptide is selected from a Clec9A polypeptide and a Clec4e polypeptide;
especially wherein the antibody is selected from the group consisting of
an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

11. The antigen-binding molecule of any one of claims 1 to 8, wherein the antigen-binding molecule is an antibody, in particular an IgG type antibody, wherein said first moiety is linked to the Fc region, and is an Ig type binding moiety,
particularly wherein the antibody is selected from the group consisting of:
(A) an anti-CD3 antibody;
(B) an anti-CD137 antibody; and
(C) a bispecific anti-CD3 anti-CD137 antibody.

12. The antigen-binding molecule of claim 9, wherein the antibody is a bispecific antibody,
particularly wherein the antigen binding molecule is a bispecific antibody directed against a membrane protein of an immune cell, or a tumour cell and against a first antigen selected from the group consisting of: a) Splicing Factor 3B Subunit 3 (SF3B3);
and b) Proliferating Cell Nuclear Antigen (PCNA),
especially wherein the antibody is selected from the group consisting of
a bispecific anti-SF3B3 anti-CD3 antibody,
a bispecific anti-SF3B3 anti-CD137 antibody,
a bispecific anti-PCNA anti-CD3 antibody, and
a bispecific anti-PCNA anti-CD137 antibody.

13. The antigen-binding molecule of claim 9, wherein the antibody is a trispecific antibody,
particularly wherein the antigen binding molecule is a trispecific antibody directed
i) against a membrane protein of an immune cell, and against a membrane protein of a tumour cell; and against a first antigen selected from the group consisting of: a) Splicing Factor 3B Subunit 3 (SF3B3); and b) Proliferating Cell Nuclear Antigen (PCNA), or
ii) against a first membrane protein of an immune cell, and a second membrane protein of an immune cell different from the first membrane protein; and against a first antigen selected from the group consisting of: a) Splicing Factor 3B Subunit 3 (SF3B3); and b) Proliferating Cell Nuclear Antigen (PCNA), or
iii) against a first membrane protein of a tumour cell, and a second membrane protein of a tumour cell different from the first membrane protein; and against a first antigen selected from the group consisting of: a) Splicing Factor 3B Subunit 3 (SF3B3); and b) Proliferating Cell Nuclear Antigen (PCNA), especially wherein the antibody is selected from the group consisting of a trispecific anti-SF3B3 anti-CD3 anti-CD137 antibody, and a trispecific anti-PCNA anti-CD3 anti-CD137 antibody.

14. A pharmaceutical composition comprising an antigen-binding molecule of any one of claims 1 to 13.

15. The antigen-binding molecule of any one of claims 1 to 13 or the pharmaceutical composition according to claim 14 for use in medicine.

16. The antigen-binding molecule of any one of claims 1 to 13 or the pharmaceutical composition of claim 14 for use in a method of treating or preventing a medical condition, preferably wherein the medical condition is selected from the group consisting of a cancer and an immune, preferably an autoimmune, disease.

17. A method of screening an antigen-binding molecule, which comprises the steps of:
(a) selecting one or more nucleic acid(s) encoding at least one first moiety wherein the first moiety binds to a first antigen, and wherein the first antigen is a damage-associated molecular pattern (DAMP), wherein the first antigen is selected from the group consisting of: a) Splicing Factor 3B Subunit 3 (SF3B3); and b) Proliferating Cell Nuclear Antigen (PCNA);
(b) selecting one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) optionally providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c), thereby obtaining the antigen-binding molecule.

18. A method of producing an antigen-binding molecule, which comprises the steps of:
(a) providing one or more nucleic acid(s) encoding at least one first moiety wherein the first moiety binds to a first antigen, and wherein the first antigen is a damage-associated molecular pattern (DAMP), wherein the first antigen is selected from the group consisting of: a) Splicing Factor 3B Subunit 3 (SF3B3); and b) Proliferating Cell Nuclear Antigen (PCNA);
(b) providing one or more nucleic acid(s) encoding at least one second moiety wherein the second moiety binds to a second antigen and wherein the second antigen is different from the first antigen, preferably wherein said second antigen is a membrane protein of an immune cell, or a tumour cell
(b1) optionally providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and optionally the at least one third moiety provided in (b1) is/are linked; and
(d) producing the antigen-binding molecule using the one or more nucleic acid(s) prepared in (c) thereby obtaining the antigen-binding molecule.

19. A method of conferring a signal transmission or blockage in a cell comprising contacting an antigen-binding molecule with a cell, or comprising administering to a subject an antigen-binding molecule, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
a) Splicing Factor 3B Subunit 3 (SF3B3); and
b) Proliferating Cell Nuclear Antigen (PCNA).

20. A method of activating or preventing an immune response comprising contacting an antigen-binding molecule with a cell, or comprising administering to a subject an antigen-binding molecule, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the first antigen is selected from the group consisting of:
a) Splicing Factor 3B Subunit 3 (SF3B3); and
b) Proliferating Cell Nuclear Antigen (PCNA).

21. A bispecific anti-Phosphatidylserine (PS) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

22. The bispecific antibody or pharmaceutical composition of claim 20 for use in a method of treating or preventing a cancer disease, preferably wherein the cancer disease is a solid tumour.

23. A bispecific anti-Phosphatidylserine (PS) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

24. The bispecific antibody or pharmaceutical composition of claim 23 for use in a method of treating or preventing a cancer disease, preferably wherein the cancer disease is a solid tumour.

25. A bispecific anti-Phosphatidylserine (PS) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

26. The bispecific antibody or pharmaceutical composition of claim 25 for use in a method of treating or preventing a cancer disease, preferably wherein the cancer disease is a solid tumour.

27. A bispecific anti-Phosphatidylserine (PS) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

28. The bispecific antibody or pharmaceutical composition of claim 27 for use in a method of treating or preventing a cancer disease, preferably wherein the cancer disease is a solid tumour.

29. A bispecific anti-Phosphatidylserine (PS) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

30. The bispecific antibody or pharmaceutical composition of claim 29 for use in a method of treating or preventing a cancer disease, preferably wherein the cancer disease is a solid tumour.

31. A bispecific anti-Phosphatidylserine (PS) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

32. The bispecific antibody or pharmaceutical composition of claim 31 for use in a method of treating or preventing a cancer disease, preferably wherein the cancer disease is a solid tumour.

33. A bispecific anti-Phosphatidylserine (PS) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

34. The bispecific antibody or pharmaceutical composition of claim 33 for use in a method of treating or preventing a cancer disease, preferably wherein the cancer disease is a solid tumour.

35. A bispecific anti-Phosphatidylserine (PS) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

36. The bispecific antibody or pharmaceutical composition of claim 35 for use in a method of treating or preventing a cancer disease, preferably wherein the cancer disease is a solid tumour.

37. A bispecific anti-F-Actin anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

38. The bispecific antibody or pharmaceutical composition of claim 37 for use in a method of treating or preventing a cancer disease, preferably wherein the cancer disease is a solid tumour.

39. A bispecific anti-F-Actin anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

40. The bispecific antibody or pharmaceutical composition of claim 39 for use in a method of treating or preventing a cancer disease, preferably wherein the cancer disease is a solid tumour.

41. A bispecific anti-F-Actin anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

42. The bispecific antibody or pharmaceutical composition of claim 41 for use in a method of treating or preventing a cancer disease, preferably wherein the cancer disease is a solid tumour.

43. A bispecific anti-F-Actin anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

44. The bispecific antibody or pharmaceutical composition of claim 43 for use in a method of treating or preventing a cancer disease, preferably wherein the cancer disease is a solid tumour.

45. A bispecific anti-F-Actin anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

46. The bispecific antibody or pharmaceutical composition of claim 45 for use in a method of treating or preventing a cancer disease, preferably wherein the cancer disease is a solid tumour.

47. A bispecific anti-F-Actin anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

48. The bispecific antibody or pharmaceutical composition of claim 47 for use in a method of treating or preventing a cancer disease, preferably wherein the cancer disease is a solid tumour.

49. A bispecific anti-F-Actin anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

50. The bispecific antibody or pharmaceutical composition of claim 49 for use in a method of treating or preventing a cancer disease, preferably wherein the cancer disease is a solid tumour.

51. A bispecific anti-F-Actin anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

52. The bispecific antibody or pharmaceutical composition of claim 51 for use in a method of treating or preventing a cancer disease, preferably wherein the cancer disease is a solid tumour.

53. A bispecific antigen-binding molecule which is an anti-CD3 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

54. The bispecific molecule or pharmaceutical composition of claim 53 for use in a method of treating or preventing a cancer disease, preferably wherein the cancer disease is a solid tumour.

55. A bispecific antigen-binding molecule which is an anti-CD3 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

56. The bispecific molecule or pharmaceutical composition of claim 55 for use in a method of treating or preventing a cancer disease, preferably wherein the cancer disease is a solid tumour.

57. A bispecific antigen-binding molecule which is an anti-CD3 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

58. The bispecific molecule or pharmaceutical composition of claim 57 for use in a method of treating or preventing a cancer disease, preferably wherein the cancer disease is a solid tumour.

59. A bispecific antigen-binding molecule which is an anti-CD3 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

60. The bispecific molecule or pharmaceutical composition of claim 59 for use in a method of treating or preventing a cancer disease, preferably wherein the cancer disease is a solid tumour.

61. A bispecific antigen-binding molecule which is an anti-CD137 antibody comprising a Clec9A polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

62. The bispecific molecule or pharmaceutical composition of claim 61 for use in a method of treating or preventing a cancer disease, preferably wherein the cancer disease is a solid tumour.

63. A bispecific antigen-binding molecule which is an anti-CD137 antibody comprising a Clec9A polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

64. The bispecific molecule or pharmaceutical composition of claim 63 for use in a method of treating or preventing a cancer disease, preferably wherein the cancer disease is a solid tumour.

65. A bispecific antigen-binding molecule which is an anti-CD137 antibody comprising a Clec9A polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

66. The bispecific molecule or pharmaceutical composition of claim 65 for use in a method of treating or preventing a cancer disease, preferably wherein the cancer disease is a solid tumour.

67. A bispecific antigen-binding molecule which is an anti-CD137 antibody comprising a Clec9A polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

68. The bispecific molecule or pharmaceutical composition of claim 67 for use in a method of treating or preventing a cancer disease, preferably wherein the cancer disease is a solid tumour.

69. A bispecific anti-Splicing Factor 3B Subunit 3 (SF3B3) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

70. The bispecific antibody or pharmaceutical composition of claim 69 for use in a method of treating or preventing a cancer disease, preferably wherein the cancer disease is a solid tumour.

71. A bispecific anti-Splicing Factor 3B Subunit 3 (SF3B3) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

72. The bispecific antibody or pharmaceutical composition of claim 71 for use in a method of treating or preventing a cancer disease, preferably wherein the cancer disease is a solid tumour.

73. A bispecific anti-Splicing Factor 3B Subunit 3 (SF3B3) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

74. The bispecific antibody or pharmaceutical composition of claim 73 for use in a method of treating or preventing a cancer disease, preferably wherein the cancer disease is a solid tumour.

75. A bispecific anti-Splicing Factor 3B Subunit 3 (SF3B3) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

76. The bispecific antibody or pharmaceutical composition of claim 75 for use in a method of treating or preventing a cancer disease, preferably wherein the cancer disease is a solid tumour.

77. A bispecific anti-Splicing Factor 3B Subunit 3 (SF3B3) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

78. The bispecific antibody or pharmaceutical composition of claim 77 for use in a method of treating or preventing a cancer disease, preferably wherein the cancer disease is a solid tumour.

79. A bispecific anti-Splicing Factor 3B Subunit 3 (SF3B3) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

80. The bispecific antibody or pharmaceutical composition of claim 79 for use in a method of treating or preventing a cancer disease, preferably wherein the cancer disease is a solid tumour.

81. A bispecific anti-Splicing Factor 3B Subunit 3 (SF3B3) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

82. The bispecific antibody or pharmaceutical composition of claim 81 for use in a method of treating or preventing a cancer disease, preferably wherein the cancer disease is a solid tumour.

83. A bispecific anti-Splicing Factor 3B Subunit 3 (SF3B3) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

84. The bispecific antibody or pharmaceutical composition of claim 83 for use in a method of treating or preventing a cancer disease, preferably wherein the cancer disease is a solid tumour.

85. A bispecific antigen-binding molecule which is an anti-CD3 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

86. The bispecific molecule or pharmaceutical composition of claim 85 for use in a method of treating or preventing a cancer disease, preferably wherein the cancer disease is a solid tumour.

87. A bispecific antigen-binding molecule which is an anti-CD3 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

88. The bispecific molecule or pharmaceutical composition of claim 87 for use in a method of treating or preventing a cancer disease, preferably wherein the cancer disease is a solid tumour.

89. A bispecific antigen-binding molecule which is an anti-CD3 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

90. The bispecific molecule or pharmaceutical composition of claim89 for use in a method of treating or preventing a cancer disease, preferably wherein the cancer disease is a solid tumour.

91. A bispecific antigen-binding molecule which is an anti-CD3 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

92. The bispecific molecule or pharmaceutical composition of claim 91 for use in a method of treating or preventing a cancer disease, preferably wherein the cancer disease is a solid tumour.

93. A bispecific antigen-binding molecule which is an anti-CD137 antibody comprising a Clec4e polypeptide, wherein the molecule is an IgG type antibody comprising a heavy chain of IgG1 isotype and a light chain of kappa isotype or of lambda isotype, or a pharmaceutical composition comprising the bispecific antigen-binding molecule and one or more pharmaceutically acceptable carriers.

94. The bispecific molecule or pharmaceutical composition of claim 93 for use in a method of treating or preventing a cancer disease, preferably wherein the cancer disease is a solid tumour.

95. A bispecific antigen-binding molecule which is an anti-CD137 antibody comprising a Clec4e polypeptide, wherein the molecule is an IgG type antibody comprising a heavy chain of IgG2 isotype and a light chain of kappa isotype or of lambda isotype, or a pharmaceutical composition comprising the bispecific antigen-binding molecule and one or more pharmaceutically acceptable carriers.

96. The bispecific molecule or pharmaceutical composition of claim 95 for use in a method of treating or preventing a cancer disease, preferably wherein the cancer disease is a solid tumour.

97. A bispecific antigen-binding molecule which is an anti-CD137 antibody comprising a Clec4e polypeptide, wherein the molecule is an IgG type antibody comprising a heavy chain of IgG3 isotype and a light chain of kappa isotype or of lambda isotype, or a pharmaceutical composition comprising the bispecific antigen-binding molecule and one or more pharmaceutically acceptable carriers.

98. The bispecific molecule or pharmaceutical composition of claim 97 for use in a method of treating or preventing a cancer disease, preferably wherein the cancer disease is a solid tumour.

99. A bispecific antigen-binding molecule which is an anti-CD137 antibody comprising a Clec4e polypeptide, wherein the molecule is an IgG type antibody comprising a heavy chain of IgG4 isotype and a light chain of kappa isotype or of lambda isotype, or a pharmaceutical composition comprising the bispecific antigen-binding molecule and one or more pharmaceutically acceptable carriers.

100. The bispecific molecule or pharmaceutical composition of claim 99 for use in a method of treating or preventing a cancer disease, preferably wherein the cancer disease is a solid tumour.

101. A bispecific anti-Proliferating Cell Nuclear Antigen (PCNA) anti-CD3 antibody comprising a heavy chain of IgG1 isotype and a light chain of kappa isotype or of lambda isotype, or a pharmaceutical composition comprising the bispecific antibody and one or more pharmaceutically acceptable carriers.

102. The bispecific antibody or pharmaceutical composition of claim 101 for use in a method of treating or preventing a cancer disease, preferably wherein the cancer disease is a solid tumour.

103. A bispecific anti-Proliferating Cell Nuclear Antigen (PCNA) anti-CD3 antibody comprising a heavy chain of IgG2 isotype and a light chain of kappa isotype or of lambda isotype, or a pharmaceutical composition comprising the bispecific antibody and one or more pharmaceutically acceptable carriers.

104. The bispecific antibody or pharmaceutical composition of claim 103 for use in a method of treating or preventing a cancer disease, preferably wherein the cancer disease is a solid tumour.

105. A bispecific anti-Proliferating Cell Nuclear Antigen (PCNA) anti-CD3 antibody comprising a heavy chain of IgG3 isotype and a light chain of kappa isotype or of lambda isotype, or a pharmaceutical composition comprising the bispecific antibody and one or more pharmaceutically acceptable carriers.

106. The bispecific antibody or pharmaceutical composition of claim 105 for use in a method of treating or preventing a cancer disease, preferably wherein the cancer disease is a solid tumour.

107. A bispecific anti-Proliferating Cell Nuclear Antigen (PCNA) anti-CD3 antibody comprising a heavy chain of IgG4 isotype and a light chain of kappa isotype or of lambda isotype, or a pharmaceutical composition comprising the bispecific antibody and one or more pharmaceutically acceptable carriers.

108. The bispecific antibody or pharmaceutical composition of claim 107 for use in a method of treating or preventing a cancer disease, preferably wherein the cancer disease is a solid tumour.

109. A bispecific anti-Proliferating Cell Nuclear Antigen (PCNA) anti-CD137 antibody comprising a heavy chain of IgG1 isotype and a light chain of kappa isotype or of lambda isotype, or a pharmaceutical composition comprising the bispecific antibody and one or more pharmaceutically acceptable carriers.

110. The bispecific antibody or pharmaceutical composition of claim 109 for use in a method of treating or preventing a cancer disease, preferably wherein the cancer disease is a solid tumour.

111. A bispecific anti-Proliferating Cell Nuclear Antigen (PCNA) anti-CD137 antibody comprising a heavy chain of IgG2 isotype and a light chain of kappa isotype or of lambda isotype, or a pharmaceutical composition comprising the bispecific antibody and one or more pharmaceutically acceptable carriers.

112. The bispecific antibody or pharmaceutical composition of claim 111 for use in a method of treating or preventing a cancer disease, preferably wherein the cancer disease is a solid tumour.

113. A bispecific anti-Proliferating Cell Nuclear Antigen (PCNA) anti-CD137 antibody comprising a heavy chain of IgG3 isotype and a light chain of kappa isotype or of lambda isotype, or a pharmaceutical composition comprising the bispecific antibody and one or more pharmaceutically acceptable carriers.

114. The bispecific antibody or pharmaceutical composition of claim 113 for use in a method of treating or preventing a cancer disease, preferably wherein the cancer disease is a solid tumour.

115. A bispecific anti-Proliferating Cell Nuclear Antigen (PCNA) anti-CD137 antibody comprising a heavy chain of IgG4 isotype and a light chain of kappa isotype or of lambda isotype, or a pharmaceutical composition comprising the bispecific antibody and one or more pharmaceutically acceptable carriers.

116. The bispecific antibody or pharmaceutical composition of claim 115 for use in a method of treating or preventing a cancer disease, preferably wherein the cancer disease is a solid tumour.

117. An antigen-binding molecule which comprises:
(1) at least one first moiety that binds to a first antigen, and
(2) at least one second moiety that binds to a second antigen;
wherein said first antigen is a damage-associated molecular pattern (DAMP), and wherein said second antigen is different from the first antigen,
preferably wherein said second antigen is a membrane protein of an immune cell, or a tumour cell, and
wherein the molecule comprises at least one third moiety that binds to a third antigen;
wherein said third antigen is different from the first antigen; preferably wherein said third antigen is a membrane protein of an immune cell, or a tumour cell, and
wherein the first antigen is selected from the group consisting of:
a) F-actin;
b) Phosphatidylserine (PS);
c) Splicing Factor 3B Subunit 3 (SF3B3); and
d) Proliferating Cell Nuclear Antigen (PCNA).

118. The antigen-binding molecule of claim 117, wherein (i) said third antigen is different from the first and second antigen, or wherein (ii) said third antigen is different from the first but same as the second antigen.

119. The antigen-binding molecule of any one of claims 117 to 118, wherein the first moiety is selected from the group consisting of:
A) an Ig type binding moiety; or
B) a binding polypeptide, particularly wherein the said binding polypeptide is a non-Ig-type binding moiety.

120. The antigen-binding molecule of claim 119, wherein
A) in case that the first antigen is Splicing Factor 3B Subunit 3 (SF3B3), the first moiety is an Ig type binding moiety, or
B) in case that the first antigen is Splicing Factor 3B Subunit 3 (SF3B3), the first moiety is a Clec4e polypeptide, or
C) in case that the first antigen is Proliferating Cell Nuclear Antigen (PCNA), the first moiety is an Ig type binding moiety, or
D) in case that the first antigen is F-actin, the first moiety is an Ig type binding moiety, or
E) in case that the first antigen is F-actin, the first moiety is a Clec9A polypeptide, or
F) in case that the first antigen is PS, the first moiety is an Ig type binding moiety.

121. The antigen-binding molecule of any one of claims 117 to 120, wherein the second and/or the third antigen is a membrane protein of an immune cell, in particular a membrane protein in a cell membrane of a T cell selected from the group consisting of T cell receptor, CD3, CD137, CD40, CTLA4, a costimulatory molecule or a coinhibitory molecule;
especially wherein the second and/or the third antigen is a membrane protein involved in a signal transmission in a cell, preferably a signal transmission receptor.

122. The antigen-binding molecule of any one of claims 117 to 121, wherein the second and/or the third antigen is a membrane protein of a tumour cell.

123. The antigen-binding molecule of any one of claims 117 to 122, wherein the second and/or third moiety is an Ig type binding moiety.

124. The antigen-binding molecule of any one of claims 117 to 123, wherein the antigen-binding molecule is an antibody, in particular an IgG type antibody.

125. The antigen-binding molecule of claim 124, wherein the antibody is capable of binding to a membrane protein of an immune cell or a tumour cell and further comprises, preferably linked to the Fc region, at least one binding polypeptide, particularly wherein the said binding polypeptide is selected from a Clec9A polypeptide and a Clec4e polypeptide;
especially wherein the antibody is selected from the group consisting of an anti-CD3 antibody comprising a Clec9A polypeptide,
an anti-CD137 antibody comprising a Clec9A polypeptide,
an anti-CD3 antibody comprising a Clec4e polypeptide, and
an anti-CD137 antibody comprising a Clec4e polypeptide.

126. The antigen-binding molecule of claim 125, wherein the antibody is a trispecific antibody,
particularly wherein the antigen binding molecule is a trispecific antibody directed
i) against a membrane protein of an immune cell, and against a membrane protein of a tumour cell; and
against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
ii) against a first membrane protein of an immune cell, and a second membrane protein of an immune cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA), or
iii) against a first membrane protein of a tumour cell, and a second membrane protein of a tumour cell different from the first membrane protein; and against a first antigen selected from the group consisting of: a) F-actin; b) Phosphatidylserine (PS); c) Splicing Factor 3B Subunit 3 (SF3B3); and d) Proliferating Cell Nuclear Antigen (PCNA),
especially wherein the antibody is selected from the group consisting of
a trispecific anti-SF3B3 anti-CD3 anti-CD137 antibody,
a trispecific anti-PCNA anti-CD3 anti-CD137 antibody,
a trispecific anti-PS anti-CD3 anti-CD137 antibody
a trispecific anti-F-Actin anti-CD3 anti-CD137 antibody
a bispecific anti-CD3 anti-CD137 antibody comprising a Clec9A polypeptide, and a bispecific anti-CD3 anti-CD137 antibody comprising a Clec4e polypeptide.

127. A method of screening an antigen-binding molecule, which comprises the steps of:
(a) selecting one or more nucleic acid(s) encoding at least one first moiety wherein the first moiety binds to a first antigen, and wherein the first antigen is a damage-associated molecular pattern (DAMP), wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) selecting one or more nucleic acid(s) encoding at least one second moiety wherein the second moiety binds to a second antigen, and wherein the second antigen is different from the first antigen, preferably wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule wherein the at least one first moiety provided in (a) and the at least one second moiety provided in (b) and the at least one third moiety provided in (b1) is/are linked; and
(d) producing the antigen-binding molecule using the one or more nucleic acid(s) prepared in (c), thereby obtaining the antigen-binding molecule.

128. A method of producing of an antigen-binding molecule, which comprises the steps of:
(a) providing one or more nucleic acid(s) encoding at least one first moiety wherein the first moiety binds to a first antigen, and wherein the first antigen is a damage-associated molecular pattern (DAMP); wherein the first antigen is selected from the group consisting of: a) F-actin; b) Phosphatidylserine (PS); c) Splicing Factor 3B Subunit 3 (SF3B3); and d) Proliferating Cell Nuclear Antigen (PCNA);
(b) providing one or more nucleic acid(s) encoding at least one second moiety wherein the second moiety binds to a second antigen, and wherein the second antigen is different from the first antigen; preferably wherein said second antigen is a membrane protein of an immune cell, or a tumour cell;
(b1) providing one or more nucleic acid(s) encoding at least one third moiety wherein the third moiety binds to a third antigen, wherein the third antigen is different from the first antigen, preferably wherein said third antigen is a membrane protein of an immune cell, or a tumour cell;
(c) obtaining one or more nucleic acid(s) encoding an antigen-binding molecule the at least one first moiety provided in (a) and the at least one second moiety provided in (b) and the at least one third moiety provided in (b1) is/are linked; and
(d) producing the antigen-binding molecule using the one or more nucleic acid(s) prepared in (c) thereby obtaining the antigen-binding molecule.

129. A method of conferring a signal transmission or blockage in a cell comprising contacting an antigen-binding molecule with a cell, or comprising administering to a subject an antigen-binding molecule, wherein the antigen-binding molecule comprises:
(1) at least one first moiety that binds to a first antigen, and
(2) at least one second moiety that binds to a second antigen;
wherein said first antigen is a damage-associated molecular pattern (DAMP), and wherein said second antigen is different from the first antigen,
preferably wherein said second antigen is a membrane protein of an immune cell, or a tumour cell, and
wherein the molecule comprises at least one third moiety that binds to a third antigen;
wherein said third antigen is different from the first antigen; preferably wherein said third antigen is a membrane protein of an immune cell, or a tumour cell, and
wherein the first antigen is selected from the group consisting of:
a) F-actin;
b) Phosphatidylserine (PS);
c) Splicing Factor 3B Subunit 3 (SF3B3); and
d) Proliferating Cell Nuclear Antigen (PCNA).

130. A method of activating or preventing an immune response comprising contacting an antigen-binding molecule with a cell, or comprising administering to a subject an antigen-binding molecule, wherein the antigen binding molecule comprises:
(1) at least one first moiety that binds to a first antigen, and
(2) at least one second moiety that binds to a second antigen;
wherein said first antigen is a damage-associated molecular pattern (DAMP), and
wherein said second antigen is different from the first antigen,
preferably wherein said second antigen is a membrane protein of an immune cell, or a tumour cell, and
wherein the molecule comprises at least one third moiety that binds to a third antigen;
wherein said third antigen is different from the first antigen; preferably wherein said third antigen is a membrane protein of an immune cell, or a tumour cell, and
wherein the first antigen is selected from the group consisting of:
a) F-actin;
b) Phosphatidylserine (PS);
c) Splicing Factor 3B Subunit 3 (SF3B3); and
d) Proliferating Cell Nuclear Antigen (PCNA).


SEQUENCE LISTING (68 sequences)

SEQ ID NO: 1 - protein, 116 aa, synthetic construct
  REGION 1..116: note = An artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 2 - protein, 122 aa, synthetic construct
  REGION 1..122: note = An artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 3 - protein, 324 aa, synthetic construct
  REGION 1..324: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - protein, 540 aa, synthetic construct
  REGION 1..540: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGSGGGSKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 5 - protein, 466 aa, synthetic construct
  REGION 1..466: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGSGGGSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 6 - protein, 112 aa, synthetic construct
  REGION 1..112: note = An artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 7 - protein, 107 aa, synthetic construct
  REGION 1..107: note = An artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 8 - protein, 107 aa, synthetic construct
  REGION 1..107: note = An artificially synthesized sequence
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 208 aa, synthetic construct
  REGION 1..208: note = An artificially synthesized sequence
KFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 10 - protein, 134 aa, synthetic construct
  REGION 1..134: note = An artificially synthesized sequence
GSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 11 - protein, 4 aa, synthetic construct
  REGION 1..4: note = An artificially synthesized sequence
GGGS

SEQ ID NO: 12 - protein, 4 aa, synthetic construct
  REGION 1..4: note = An artificially synthesized sequence
SGGG

SEQ ID NO: 13 - protein, 5 aa, synthetic construct
  REGION 1..5: note = An artificially synthesized sequence
GGGGS

SEQ ID NO: 14 - protein, 5 aa, synthetic construct
  REGION 1..5: note = An artificially synthesized sequence
SGGGG

SEQ ID NO: 15 - protein, 6 aa, synthetic construct
  REGION 1..6: note = An artificially synthesized sequence
GGGGGS

SEQ ID NO: 16 - protein, 6 aa, synthetic construct
  REGION 1..6: note = An artificially synthesized sequence
SGGGGG

SEQ ID NO: 17 - protein, 7 aa, synthetic construct
  REGION 1..7: note = An artificially synthesized sequence
GGGGGGS

SEQ ID NO: 18 - protein, 7 aa, synthetic construct
  REGION 1..7: note = An artificially synthesized sequence
SGGGGGG

SEQ ID NO: 19 - protein, 122 aa, synthetic construct; identical to SEQ ID NO: 2
  REGION 1..122: note = An artificially synthesized sequence

SEQ ID NO: 20 - protein, 116 aa, synthetic construct; identical to SEQ ID NO: 1
  REGION 1..116: note = An artificially synthesized sequence

SEQ ID NO: 21 - protein, 324 aa, synthetic construct; identical to SEQ ID NO: 3
  REGION 1..324: note = An artificially synthesized sequence

SEQ ID NO: 22 - protein, 553 aa, synthetic construct
  REGION 1..553: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCAITDFFPEDITVEWQWNGQPAENYKNTQPIMDTDGSYFVVSKLNVQKSNWEAGNTFTCSVLHEGLHNHHTEKSLSHSPGKGGGSGGGSKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCIGGGGSDYKDDDDK

SEQ ID NO: 23 - protein, 479 aa, synthetic construct
  REGION 1..479: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCAITDFFPEDITVEWQWNGQPAENYKNTQPIMDTDGSYFVVSKLNVQKSNWEAGNTFTCSVLHEGLHNHHTEKSLSHSPGKGGGSGGGSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCIGGGGSDYKDDDDK

SEQ ID NO: 24 - protein, 540 aa, synthetic construct; identical to SEQ ID NO: 4
  REGION 1..540: note = An artificially synthesized sequence

SEQ ID NO: 25 - protein, 466 aa, synthetic construct; identical to SEQ ID NO: 5
  REGION 1..466: note = An artificially synthesized sequence

SEQ ID NO: 26 - protein, 553 aa, synthetic construct
  REGION 1..553: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLWCMITDFFPEDITVEWQWNGQPAENYKNTQPIMDTDGSYFVYSKLNVQKSNWEAGNTFTCSVLHEGLHNHHTEKSLSHSPGKGGGSGGGSKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCIGGGGSHHHHHHHH

SEQ ID NO: 27 - protein, 208 aa, synthetic construct; identical to SEQ ID NO: 9
  REGION 1..208: note = An artificially synthesized sequence

SEQ ID NO: 28 - protein, 134 aa, synthetic construct; identical to SEQ ID NO: 10
  REGION 1..134: note = An artificially synthesized sequence

SEQ ID NO: 29 - protein, 479 aa, synthetic construct
  REGION 1..479: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLWCMITDFFPEDITVEWQWNGQPAENYKNTQPIMDTDGSYFVYSKLNVQKSNWEAGNTFTCSVLHEGLHNHHTEKSLSHSPGKGGGSGGGSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCIGGGGSHHHHHHHH

SEQ ID NO: 30 - protein, 112 aa, synthetic construct; identical to SEQ ID NO: 6
  REGION 1..112: note = An artificially synthesized sequence

SEQ ID NO: 31 - protein, 107 aa, synthetic construct; identical to SEQ ID NO: 8
  REGION 1..107: note = An artificially synthesized sequence

SEQ ID NO: 32 - protein, 336 aa, synthetic construct
  REGION 1..336: note = An artificially synthesized sequence
ASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSPDYKDDDDK

SEQ ID NO: 33 - protein, 107 aa, synthetic construct; identical to SEQ ID NO: 7
  REGION 1..107: note = An artificially synthesized sequence

SEQ ID NO: 34 - protein, 107 aa, synthetic construct; identical to SEQ ID NO: 7
  REGION 1..107: note = An artificially synthesized sequence

SEQ ID NO: 35 - protein, 107 aa, synthetic construct [sequence omitted]

SEQ ID NO: 36 - protein, 116 aa, synthetic construct; identical to SEQ ID NO: 1
  REGION 1..116: note = An artificially synthesized sequence

SEQ ID NO: 37 - protein, 336 aa, synthetic construct
  REGION 1..336: note = An artificially synthesized sequence
ASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSPHHHHHHHH

SEQ ID NO: 38 - protein, 107 aa, synthetic construct [sequence omitted]

SEQ ID NO: 39 - protein, 120 aa, synthetic construct
  REGION 1..120: note = An artificially synthesized sequence
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSS

SEQ ID NO: 40 - protein, 117 aa, synthetic construct
  REGION 1..117: note = An artificially synthesized sequence
QVQLKESGAELVKPGASVKLSCTASGFNIKDYYMHWVKQRTEQGLEWIGRIDPEDGETKYAPKFQGKATITADTSSNTAYLQLSSLTSEDTAVYYCARSYDYAMDYWGQGTTVTVST

SEQ ID NO: 41 - protein, 117 aa, synthetic construct
  REGION 1..117: note = An artificially synthesized sequence
QVQLQQSGPELVKPGASVRISCKASGYTFTSYYIHWVKQRPGQGLEWIGWIYPGNVNTKYNEKFKGKATLTADKSSSTAYMQLSSLTSEDSAVYFCARYGNYPFAYWGQGTLVTVSA

SEQ ID NO: 42 - protein, 120 aa, synthetic construct; identical to SEQ ID NO: 39
  REGION 1..120: note = An artificially synthesized sequence

SEQ ID NO: 43 - protein, 116 aa, synthetic construct
  REGION 1..116: note = An artificially synthesized sequence
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 44 - protein, 107 aa, synthetic construct
  REGION 1..107: note = An artificially synthesized sequence
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELK

SEQ ID NO: 45 - protein, 106 aa, synthetic construct
  REGION 1..106: note = An artificially synthesized sequence
QIVLTQSPALMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 46 - protein, 112 aa, synthetic construct
  REGION 1..112: note = An artificially synthesized sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPTFGGGTKLEIK

SEQ ID NO: 47 - protein, 108 aa, synthetic construct
  REGION 1..108: note = An artificially synthesized sequence
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 48 - protein, 324 aa, synthetic construct; identical to SEQ ID NO: 3
  REGION 1..324: note = An artificially synthesized sequence

SEQ ID NO: 49 - protein, 324 aa, synthetic construct
  REGION 1..324: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMRTDGSYFVYSKLNVQKSNWEAGNTFTCSVLHEGLHNHHTEKSLSHSPGK

SEQ ID NO: 50 - protein, 324 aa, synthetic construct
  REGION 1..324: note = An artificially synthesized sequence
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVKEVSKVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSELNVQKSNWEAGNTFTCSVLHEGLHNHHTEKSLSHSPGK

SEQ ID NO: 51 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 470 aa, synthetic construct
  REGION 1..470: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 53 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQEGNVFSCSVMHEALHNHYTQESLSLSP

SEQ ID NO: 55 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 56 - protein, 330 aa, synthetic construct
  REGION 1..330: note = An artificially synthesized sequence
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSP

SEQ ID NO: 57 - protein, 107 aa, synthetic construct
  REGION 1..107: note = An artificially synthesized sequence
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 112 aa, synthetic construct
QSVEESGGRLVTPGTPLTLTCTVSGFSLSNYPMTWVRQAPGKGLEWIGIIDARGIIGYASWAKGRFTISKTSTTVDLKMTRLTTEDTATYFCARDTSTGNIWGPGTLVTVSL

SEQ ID NO: 59 - protein, 111 aa, synthetic construct
ADVVMTQTPASVEAAVGATVTIKCQASHSIRTYLSWYQQKPGQPPKLLIYSGSYVPSGVPSRFKGSGSGTEFTLTISDLECADAATYYCQQDATSLDVYNTFGGGTEVVVK

SEQ ID NO: 60 - protein, 107 aa, synthetic construct
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 61 - protein, 122 aa, synthetic construct
  REGION 1..122: note = An artificially synthesized sequence
QSTLKESGPGILQPSQTLSLTCSFSGFSLSTSGMGVGWIRQPSGKGLEWLAHIWWDDDKRYNPALKSRLTISKDTSSHQVFLKIASVDTADTATYYCARMAIKRDYYAIDYWGQGTSVTVSS

SEQ ID NO: 62 - protein, 119 aa, synthetic construct
  REGION 1..119: note = An artificially synthesized sequence
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARSEWSLAFDIWGQGTMVTVSS

SEQ ID NO: 63 - protein, 118 aa, synthetic construct
  REGION 1..118: note = An artificially synthesized sequence
EVQLQQSGPELVKPGASVKMSCKASGYTFTDFYMKWVKQSHGKSLEWIGDINPNKGGTTYNLKFKGKATLTVDKSSSTAYMQLNSLTSEDSAVYYCARDDYDAWLAYWGQGTLVTVSA

SEQ ID NO: 64 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 65 - protein, 107 aa, synthetic construct
  REGION 1..107: note = An artificially synthesized sequence
DIQMTQSPASQSASLGESVTITCLASQTIGTWLQWYQQKPGKSPQLLIYAATSLADGVPSRFSGSGSGTKFSFKISSLQAEDFVSYYCQQLYSTPYTFGGGTKLEIK

SEQ ID NO: 66 - protein, 110 aa, synthetic construct
  REGION 1..110: note = An artificially synthesized sequence
QPVLTQPPSASGTPGQRVTIPCSGSSSNIGGNDVYWYQQVPGMAPKLLIYRNHQRPSGVPDRFSGSKSGTSASLAISGLRSEDEADYYCAAWDDSLGGVVFGGGTKVTVL

SEQ ID NO: 67 - protein, 112 aa, synthetic construct
  REGION 1..112: note = An artificially synthesized sequence
DIVMTQAAPSVPVTPGESVSISCRSSKSLLHKNGNTYLFWFLQRPGQSPQLLIYRVSNLASGVPDRFSGSGSGTAFTLRISRVEAEDVGIYYCMQHLEFPLTFGAGTKLELK

SEQ ID NO: 68 - protein, 128 aa, synthetic construct
  REGION 1..128: note = An artificially synthesized sequence
QVQLVESGGGLVQPGRSLRLSCAASGFTFSNAWMHWVRQAPGKGLEWVAQIKDKANGYNAYYAESVKGRFTISRDDSKNSIYLQMNSLKTEDTAVYYCRYVHYTTYAGFSYKFGVDAWGQGTTVTVSS